(19) United States
(12) Patent Application Publication
(10) Pub. No.: US 2026/0199521 A1
(43) Pub. Date: Jul. 16, 2026

(54) GENE EDITING FOR INTERVERTEBRAL, INTRA- AND PERIDISCAL THERAPY AND ASSOCIATED SPINAL DISORDERS

(71) Applicant: ORTHOBIO THERAPEUTICS, INC., Vail, CO (US)

(72) Inventors: Peter J. MILLETT, Edwards, CO (US); Matthew J. ALLEN, Swaffham Bulbeck, Cambs (GB); George GENTSCH, Cambridge (GB)

(21) Appl. No.: 18/855,980

(22) PCT Filed: Apr. 12, 2023
(86) PCT No.: PCT/US2023/065684

§ 371 Date: Oct. 10, 2024

(51) Int. Cl. A61K 48/00; A61K 38/46; C12N 9/22; C12N 15/11; C12N 15/86

(52) U.S. Cl. CPC A61K 48/005; A61K 38/465; C12N 9/226; C12N 15/11; C12N 15/86; C12N 2310/20; C12N 2750/14143

(57) ABSTRACT

The present disclosure provides compositions and methods for treating and preventing localized nociception, inflammation, or morphological changes associated with joint disease or illness, back or spine conditions or disorders, and musculoskeletal diseases or dysfunction.

### CROSS REFERENCE TO RELATED APPLICATIONS

This application claims priority to U.S. Provisional Patent Application Nos. 63/362,858, filed Apr. 12, 2022, 63/334,476, filed Apr. 25, 2022, 63/342,471, filed May 16, 2022, and 63/495,461, filed Apr. 11, 2023, the contents of which are hereby incorporated by reference herein, in their entireties, for all purposes.

### BACKGROUND OF THE DISCLOSURE

Spinal conditions or disorders, including low back pain, or neck pain associated with intervertebral disc degeneration (IDD) (also known as degenerative disc disease (DDD)), disc herniation, spinal stenosis, spondylosis, spondylolisthesis, infection (discospondylitis), and neuropathies such as discogenic pain, radiculopathy, sciatica, or post-herpetic neuralgia.

Low back pain (LBP) is a major cause of morbidity and disability worldwide for which few long-term options for amelioration currently exist. Andersson, G. B. (1999). Lancet 354, 581-585. Low back pain (LBP) is the single leading cause of disability worldwide having a global lifetime prevalence of 38.9%. In a recent survey of 126 countries, LBP was identified as the leading cause of worldwide productivity loss and of years lived with disability. Wu, A. et al. (2020). Annals of Translational Medicine 8, 299. Presently available treatments include surgical or less invasive options that often fail to offer long-term palliation. Ju, D. G., et al. (2022). Global Spine Journal 12(5), 756-764. All vertebrate species are affected by back or spine conditions or disorders, including working animals, domestic pets, and their owners. All suffer from the associated discomfort, pain, and disability, depending on the degree of disease progression.

Spinal conditions or disorders, such as low back pain, are complex diseases characterized by a multitude of inputs contributing to a progressive course of disability. Among these contributors are genetic predispositions, lifestyle factors, alterations in mechanical loading, structural/morphological irregularities (e.g., disc herniations or calcifications), inflammation, and changes in the localized cellular environment (e.g., alterations in cellular phenotype, viability, vascularization and/or innervation). Peng, B. G. (2013). World Journal of Orthopedics 4(2), 42-52. Each contributing factor is driven by differential expression of various gene products, including at least pro-inflammatory cytokines, growth factors, pain signaling molecules and other effector biomolecules. New methods and compositions to treat this disease are acutely needed.

### BRIEF SUMMARY OF THE DISCLOSURE

Provided herein are compositions and methods for treating or preventing localized nociception, inflammation, or morphological changes associated with spinal conditions or disorders, are disclosed herein. Additionally, methods for gene-editing cells, including, but not limited to the cells constituting the nucleus pulposus, annulus fibrosus and disc-associated nociceptors, and uses of gene-edited cells to ameliorate symptoms of diseases, such as discogenic disorders, are disclosed herein.

### BRIEF DESCRIPTION OF THE DRAWINGS

The presently disclosed embodiments will be further explained with reference to the attached drawings. The drawings shown are not necessarily to scale, with emphasis instead generally being placed upon illustrating the principles of the presently disclosed embodiments.

FIG. 1 illustrates SEQ ID NOs: 1-48, the crRNA sequences generated by the bioinformatic methods herein described that target human ADAM17 to modify and/or ablate expression of its encoded products. Additional information includes the chromosomal genomic coordinates (assembly hg38) of the edit site and a score, summarizing several predicted performance metrics.

FIG. 2 illustrates SEQ ID NOs: 49-96, the crRNA sequences generated by the bioinformatic methods herein described that target human ADAMTS1 to modify and/or ablate expression of its encoded products. Additional information includes the chromosomal genomic coordinates (assembly hg38) of the edit site and a score, summarizing several predicted performance metrics.

FIG. 3 illustrates SEQ ID NOs: 97-144, the crRNA sequences generated by the bioinformatic methods herein described that target human ADAMTS5 to modify and/or ablate expression of its encoded products. Additional information includes the chromosomal genomic coordinates (assembly hg38) of the edit site and a score, summarizing several predicted performance metrics.

FIG. 4 illustrates SEQ ID NOs: 145-192, the crRNA sequences generated by the bioinformatic methods herein described that target human ADM to modify and/or ablate expression of its encoded products. Additional information includes the chromosomal genomic coordinates (assembly hg38) of the edit site and a score, summarizing several predicted performance metrics.

FIG. 5 illustrates SEQ ID NOs: 193-240, the crRNA sequences generated by the bioinformatic methods herein described that target human ATP1A1 to modify and/or ablate expression of its encoded products. Additional information includes the chromosomal genomic coordinates (assembly hg38) of the edit site and a score, summarizing several predicted performance metrics.

FIG. 6 illustrates SEQ ID NOs: 241-281, the crRNA sequences generated by the bioinformatic methods herein described that target human BDNF to modify and/or ablate expression of its encoded products. Additional information includes the chromosomal genomic coordinates (assembly hg38) of the edit site and a score, summarizing several predicted performance metrics.

FIG. 7 illustrates SEQ ID NOs: 282-301, the crRNA sequences generated by the bioinformatic methods herein described that target human CALCA to modify and/or ablate expression of its encoded products. Additional information includes the chromosomal genomic coordinates (assembly hg38) of the edit site and a score, summarizing several predicted performance metrics.

FIG. 8 illustrates SEQ ID NOs: 302-318, the crRNA sequences generated by the bioinformatic methods herein described that target human CALCB to modify and/or ablate expression of its encoded products. Additional information includes the chromosomal genomic coordinates (assembly hg38) of the edit site and a score, summarizing several predicted performance metrics.

FIG. 9 illustrates SEQ ID NOs: 319-340, the crRNA sequences generated by the bioinformatic methods herein described that target human CALCRL to modify and/or ablate expression of its encoded products. Additional information includes the chromosomal genomic coordinates (assembly hg38) of the edit site and a score, summarizing several predicted performance metrics.

FIG. 10 illustrates SEQ ID NOs: 341-357, the crRNA sequences generated by the bioinformatic methods herein described that target human CCL2 to modify and/or ablate expression of its encoded products. Additional information includes the chromosomal genomic coordinates (assembly hg38) of the edit site and a score, summarizing several predicted performance metrics.

FIG. 11 illustrates SEQ ID NOs: 358-374, the crRNA sequences generated by the bioinformatic methods herein described that target human CCL3 to modify and/or ablate expression of its encoded products. Additional information includes the chromosomal genomic coordinates (assembly hg38) of the edit site and a score, summarizing several predicted performance metrics.

FIG. 12 illustrates SEQ ID NOs: 375-391, the crRNA sequences generated by the bioinformatic methods herein described that target human CCL5 to modify and/or ablate expression of its encoded products. Additional information includes the chromosomal genomic coordinates (assembly hg38) of the edit site and a score, summarizing several predicted performance metrics.

FIG. 13 illustrates SEQ ID NOs: 392-408, the crRNA sequences generated by the bioinformatic methods herein described that target human CCL7 to modify and/or ablate expression of its encoded products. Additional information includes the chromosomal genomic coordinates (assembly hg38) of the edit site and a score, summarizing several predicted performance metrics.

FIG. 14 illustrates SEQ ID NOs: 409-425, the crRNA sequences generated by the bioinformatic methods herein described that target human CCL20 to modify and/or ablate expression of its encoded products. Additional information includes the chromosomal genomic coordinates (assembly hg38) of the edit site and a score, summarizing several predicted performance metrics.

FIG. 15 illustrates SEQ ID NOs: 426-473, the crRNA sequences generated by the bioinformatic methods herein described that target human CCN2 to modify and/or ablate expression of its encoded products. Additional information includes the chromosomal genomic coordinates (assembly hg38) of the edit site and a score, summarizing several predicted performance metrics.

FIG. 16 illustrates SEQ ID NOs: 474-517, the crRNA sequences generated by the bioinformatic methods herein described that target human CCR7 to modify and/or ablate expression of its encoded products. Additional information includes the chromosomal genomic coordinates (assembly hg38) of the edit site and a score, summarizing several predicted performance metrics.

FIG. 17 illustrates SEQ ID NOs: 518-534, the crRNA sequences generated by the bioinformatic methods herein described that target human CRCP to modify and/or ablate expression of its encoded products. Additional information includes the chromosomal genomic coordinates (assembly hg38) of the edit site and a score, summarizing several predicted performance metrics.

FIG. 18 illustrates SEQ ID NOs: 535-551, the crRNA sequences generated by the bioinformatic methods herein described that target human CXCL1 to modify and/or ablate expression of its encoded products. Additional information includes the chromosomal genomic coordinates (assembly hg38) of the edit site and a score, summarizing several predicted performance metrics.

FIG. 19 illustrates SEQ ID NOs: 552-568, the crRNA sequences generated by the bioinformatic methods herein described that target human CXCL2 to modify and/or ablate expression of its encoded products. Additional information includes the chromosomal genomic coordinates (assembly hg38) of the edit site and a score, summarizing several predicted performance metrics.

FIG. 20 illustrates SEQ ID NOs: 569-585, the crRNA sequences generated by the bioinformatic methods herein described that target human CXCL3 to modify and/or ablate expression of its encoded products. Additional information includes the chromosomal genomic coordinates (assembly hg38) of the edit site and a score, summarizing several predicted performance metrics.

FIG. 21 illustrates SEQ ID NOs: 586-602, the crRNA sequences generated by the bioinformatic methods herein described that target human CXCL5 to modify and/or ablate expression of its encoded products. Additional information includes the chromosomal genomic coordinates (assembly hg38) of the edit site and a score, summarizing several predicted performance metrics.

FIG. 22 illustrates SEQ ID NOs: 603-619, the crRNA sequences generated by the bioinformatic methods herein described that target human CXCL6 to modify and/or ablate expression of its encoded products. Additional information includes the chromosomal genomic coordinates (assembly hg38) of the edit site and a score, summarizing several predicted performance metrics.

FIG. 23 illustrates SEQ ID NOs: 620-636, the crRNA sequences generated by the bioinformatic methods herein described that target human CXCL8 to modify and/or ablate expression of its encoded products. Additional information includes the chromosomal genomic coordinates (assembly hg38) of the edit site and a score, summarizing several predicted performance metrics.

FIG. 24 illustrates SEQ ID NOs: 637-655, the crRNA sequences generated by the bioinformatic methods herein described that target human CXCR1 to modify and/or ablate expression of its encoded products. Additional information includes the chromosomal genomic coordinates (assembly hg38) of the edit site and a score, summarizing several predicted performance metrics.

FIG. 25 illustrates SEQ ID NOs: 656-672, the crRNA sequences generated by the bioinformatic methods herein described that target human CXCR2 to modify and/or ablate expression of its encoded products. Additional information includes the chromosomal genomic coordinates (assembly hg38) of the edit site and a score, summarizing several predicted performance metrics.

FIG. 26 illustrates SEQ ID NOs: 673-720, the crRNA sequences generated by the bioinformatic methods herein described that target human FGF2 to modify and/or ablate expression of its encoded products. Additional information includes the chromosomal genomic coordinates (assembly hg38) of the edit site and a score, summarizing several predicted performance metrics.

FIG. 27 illustrates SEQ ID NOs: 721-768, the crRNA sequences generated by the bioinformatic methods herein described that target human FGFR1 to modify and/or ablate expression of its encoded products. Additional information includes the chromosomal genomic coordinates (assembly hg38) of the edit site and a score, summarizing several predicted performance metrics.

FIG. 28 illustrates SEQ ID NOs: 769-786, the crRNA sequences generated by the bioinformatic methods herein described that target human IL1A to modify and/or ablate expression of its encoded products. Additional information includes the chromosomal genomic coordinates (assembly hg38) of the edit site and a score, summarizing several predicted performance metrics.

FIG. 29 illustrates SEQ ID NOs: 787-805, the crRNA sequences generated by the bioinformatic methods herein described that target human IL1B to modify and/or ablate expression of its encoded products. Additional information includes the chromosomal genomic coordinates (assembly hg38) of the edit site and a score, summarizing several predicted performance metrics.

FIG. 30 illustrates SEQ ID NOs: 806-839, the crRNA sequences generated by the bioinformatic methods herein described that target human IL1R1 to modify and/or ablate expression of its encoded products. Additional information includes the chromosomal genomic coordinates (assembly hg38) of the edit site and a score, summarizing several predicted performance metrics.

FIG. 31 illustrates SEQ ID NOs: 840-887, the crRNA sequences generated by the bioinformatic methods herein described that target human IL1RAP to modify and/or ablate expression of its encoded products. Additional information includes the chromosomal genomic coordinates (assembly hg38) of the edit site and a score, summarizing several predicted performance metrics.

FIG. 32 illustrates SEQ ID NOs: 888-911, the crRNA sequences generated by the bioinformatic methods herein described that target human IL4 to modify and/or ablate expression of its encoded products. Additional information includes the chromosomal genomic coordinates (assembly hg38) of the edit site and a score, summarizing several predicted performance metrics.

FIG. 33 illustrates SEQ ID NOs: 912-928, the crRNA sequences generated by the bioinformatic methods herein described that target human IL6 to modify and/or ablate expression of its encoded products. Additional information includes the chromosomal genomic coordinates (assembly hg38) of the edit site and a score, summarizing several predicted performance metrics.

FIG. 34 illustrates SEQ ID NOs: 929-963, the crRNA sequences generated by the bioinformatic methods herein described that target human IL6R to modify and/or ablate expression of its encoded products. Additional information includes the chromosomal genomic coordinates (assembly hg38) of the edit site and a score, summarizing several predicted performance metrics.

FIG. 35 illustrates SEQ ID NOs: 964-990, the crRNA sequences generated by the bioinformatic methods herein described that target human IL6ST to modify and/or ablate expression of its encoded products. Additional information includes the chromosomal genomic coordinates (assembly hg38) of the edit site and a score, summarizing several predicted performance metrics.

FIG. 36 illustrates SEQ ID NOs: 991-1007, the crRNA sequences generated by the bioinformatic methods herein described that target human IL10 to modify and/or ablate expression of its encoded products. Additional information includes the chromosomal genomic coordinates (assembly hg38) of the edit site and a score, summarizing several predicted performance metrics.

FIG. 37 illustrates SEQ ID NOs: 1008-1055, the crRNA sequences generated by the bioinformatic methods herein described that target human IL10RA to modify and/or ablate expression of its encoded products. Additional information includes the chromosomal genomic coordinates (assembly hg38) of the edit site and a score, summarizing several predicted performance metrics.

FIG. 38 illustrates SEQ ID NOs: 1056-1082, the crRNA sequences generated by the bioinformatic methods herein described that target human IL10RB to modify and/or ablate expression of its encoded products. Additional information includes the chromosomal genomic coordinates (assembly hg38) of the edit site and a score, summarizing several predicted performance metrics.

FIG. 39 illustrates SEQ ID NOs: 1083-1104, the crRNA sequences generated by the bioinformatic methods herein described that target human IL13 to modify and/or ablate expression of its encoded products. Additional information includes the chromosomal genomic coordinates (assembly hg38) of the edit site and a score, summarizing several predicted performance metrics.

FIG. 40 illustrates SEQ ID NOs: 1105-1130, the crRNA sequences generated by the bioinformatic methods herein described that target human IL13RA1 to modify and/or ablate expression of its encoded products. Additional information includes the chromosomal genomic coordinates (assembly hg38) of the edit site and a score, summarizing several predicted performance metrics.

FIG. 41 illustrates SEQ ID NOs: 1131-1147, the crRNA sequences generated by the bioinformatic methods herein described that target human IL13RA2 to modify and/or ablate expression of its encoded products. Additional information includes the chromosomal genomic coordinates (assembly hg38) of the edit site and a score, summarizing several predicted performance metrics.

FIG. 42 illustrates SEQ ID NOs: 1148-1173, the crRNA sequences generated by the bioinformatic methods herein described that target human IL17A to modify and/or ablate expression of its encoded products. Additional information includes the chromosomal genomic coordinates (assembly hg38) of the edit site and a score, summarizing several predicted performance metrics.

FIG. 43 illustrates SEQ ID NOs: 1174-1221, the crRNA sequences generated by the bioinformatic methods herein described that target human IL17RA to modify and/or ablate expression of its encoded products. Additional information includes the chromosomal genomic coordinates (assembly hg38) of the edit site and a score, summarizing several predicted performance metrics.

FIG. 44 illustrates SEQ ID NOs: 1222-1238, the crRNA sequences generated by the bioinformatic methods herein described that target human IL18 to modify and/or ablate expression of its encoded products. Additional information includes the chromosomal genomic coordinates (assembly hg38) of the edit site and a score, summarizing several predicted performance metrics.

FIG. 45 illustrates SEQ ID NOs: 1239-1262, the crRNA sequences generated by the bioinformatic methods herein described that target human IL18R1 to modify and/or ablate expression of its encoded products. Additional information includes the chromosomal genomic coordinates (assembly hg38) of the edit site and a score, summarizing several predicted performance metrics.

FIG. 46 illustrates SEQ ID NOs: 1263-1310, the crRNA sequences generated by the bioinformatic methods herein described that target human IL18RAP to modify and/or ablate expression of its encoded products. Additional information includes the chromosomal genomic coordinates (assembly hg38) of the edit site and a score, summarizing several predicted performance metrics.

FIG. 47 illustrates SEQ ID NOs: 1311-1343, the crRNA sequences generated by the bioinformatic methods herein described that target human MMP1 to modify and/or ablate expression of its encoded products. Additional information includes the chromosomal genomic coordinates (assembly hg38) of the edit site and a score, summarizing several predicted performance metrics.

FIG. 48 illustrates SEQ ID NOs: 1344-1391, the crRNA sequences generated by the bioinformatic methods herein described that target human MMP2 to modify and/or ablate expression of its encoded products. Additional information includes the chromosomal genomic coordinates (assembly hg38) of the edit site and a score, summarizing several predicted performance metrics.

FIG. 49 illustrates SEQ ID NOs: 1392-1417, the crRNA sequences generated by the bioinformatic methods herein described that target human MMP3 to modify and/or ablate expression of its encoded products. Additional information includes the chromosomal genomic coordinates (assembly hg38) of the edit site and a score, summarizing several predicted performance metrics.

FIG. 50 illustrates SEQ ID NOs: 1418-1436, the crRNA sequences generated by the bioinformatic methods herein described that target human MMP7 to modify and/or ablate expression of its encoded products. Additional information includes the chromosomal genomic coordinates (assembly hg38) of the edit site and a score, summarizing several predicted performance metrics.

FIG. 51 illustrates SEQ ID NOs: 1437-1474, the crRNA sequences generated by the bioinformatic methods herein described that target human MMP8 to modify and/or ablate expression of its encoded products. Additional information includes the chromosomal genomic coordinates (assembly hg38) of the edit site and a score, summarizing several predicted performance metrics.

FIG. 52 illustrates SEQ ID NOs: 1475-1497, the crRNA sequences generated by the bioinformatic methods herein described that target human MMP10 to modify and/or ablate expression of its encoded products. Additional information includes the chromosomal genomic coordinates (assembly hg38) of the edit site and a score, summarizing several predicted performance metrics.

FIG. 53 illustrates SEQ ID NOs: 1498-1541, the crRNA sequences generated by the bioinformatic methods herein described that target human MMP12 to modify and/or ablate expression of its encoded products. Additional information includes the chromosomal genomic coordinates (assembly hg38) of the edit site and a score, summarizing several predicted performance metrics.

FIG. 54 illustrates SEQ ID NOs: 1542-1568, the crRNA sequences generated by the bioinformatic methods herein described that target human MMP13 to modify and/or ablate expression of its encoded products. Additional information includes the chromosomal genomic coordinates (assembly hg38) of the edit site and a score, summarizing several predicted performance metrics.

FIG. 55 illustrates SEQ ID NOs: 1569-1585, the crRNA sequences generated by the bioinformatic methods herein described that target human MRGPRX2 to modify and/or ablate expression of its encoded products. Additional information includes the chromosomal genomic coordinates (assembly hg38) of the edit site and a score, summarizing several predicted performance metrics.

FIG. 56 illustrates SEQ ID NOs: 1586-1628, the crRNA sequences generated by the bioinformatic methods herein described that target human NGF to modify and/or ablate expression of its encoded products. Additional information includes the chromosomal genomic coordinates (assembly hg38) of the edit site and a score, summarizing several predicted performance metrics.

FIG. 57 illustrates SEQ ID NOs: 1629-1676, the crRNA sequences generated by the bioinformatic methods herein described that target human NGFR to modify and/or ablate expression of its encoded products. Additional information includes the chromosomal genomic coordinates (assembly hg38) of the edit site and a score, summarizing several predicted performance metrics.

FIG. 58 illustrates SEQ ID NOs: 1677-1724, the crRNA sequences generated by the bioinformatic methods herein described that target human NTF3 to modify and/or ablate expression of its encoded products. Additional information includes the chromosomal genomic coordinates (assembly hg38) of the edit site and a score, summarizing several predicted performance metrics.

FIG. 59 illustrates SEQ ID NOs: 1725-1746, the crRNA sequences generated by the bioinformatic methods herein described that target human NTF4 to modify and/or ablate expression of its encoded products. Additional information includes the chromosomal genomic coordinates (assembly hg38) of the edit site and a score, summarizing several predicted performance metrics.

FIG. 60 illustrates SEQ ID NOs: 1747-1794, the crRNA sequences generated by the bioinformatic methods herein described that target human NTRK1 to modify and/or ablate expression of its encoded products. Additional information includes the chromosomal genomic coordinates (assembly hg38) of the edit site and a score, summarizing several predicted performance metrics.

FIG. 61 illustrates SEQ ID NOs: 1795-1842, the crRNA sequences generated by the bioinformatic methods herein described that target human NTRK2 to modify and/or ablate expression of its encoded products. Additional information includes the chromosomal genomic coordinates (assembly hg38) of the edit site and a score, summarizing several predicted performance metrics.

FIG. 62 illustrates SEQ ID NOs: 1843-1859, the crRNA sequences generated by the bioinformatic methods herein described that target human RAMP1 to modify and/or ablate expression of its encoded products. Additional information includes the chromosomal genomic coordinates (assembly hg38) of the edit site and a score, summarizing several predicted performance metrics.

FIG. 63 illustrates SEQ ID NOs: 1860-1907, the crRNA sequences generated by the bioinformatic methods herein described that target human SCN1A to modify and/or ablate expression of its encoded products. Additional information includes the chromosomal genomic coordinates (assembly hg38) of the edit site and a score, summarizing several predicted performance metrics.

FIG. 64 illustrates SEQ ID NOs: 1908-1955, the crRNA sequences generated by the bioinformatic methods herein described that target human SCN2A to modify and/or ablate expression of its encoded products. Additional information includes the chromosomal genomic coordinates (assembly hg38) of the edit site and a score, summarizing several predicted performance metrics.

FIG. 65 illustrates SEQ ID NOs: 1956-2003, the crRNA sequences generated by the bioinformatic methods herein described that target human SCN3A to modify and/or ablate expression of its encoded products. Additional information includes the chromosomal genomic coordinates (assembly hg38) of the edit site and a score, summarizing several predicted performance metrics.

FIG. 66 illustrates SEQ ID NOs: 2004-2051, the crRNA sequences generated by the bioinformatic methods herein described that target human SCN4A to modify and/or ablate expression of its encoded products. Additional information includes the chromosomal genomic coordinates (assembly hg38) of the edit site and a score, summarizing several predicted performance metrics.

FIG. 67 illustrates SEQ ID NOs: 2052-2099, the crRNA sequences generated by the bioinformatic methods herein described that target human SCN5A to modify and/or ablate expression of its encoded products. Additional information includes the chromosomal genomic coordinates (assembly hg38) of the edit site and a score, summarizing several predicted performance metrics.

FIG. 68 illustrates SEQ ID NOs: 2100-2147, the crRNA sequences generated by the bioinformatic methods herein described that target human SCN8A to modify and/or ablate expression of its encoded products. Additional information includes the chromosomal genomic coordinates (assembly hg38) of the edit site and a score, summarizing several predicted performance metrics.

FIG. 69 illustrates SEQ ID NOs: 2148-2195, the crRNA sequences generated by the bioinformatic methods herein described that target human SCN9A to modify and/or ablate expression of its encoded products. Additional information includes the chromosomal genomic coordinates (assembly hg38) of the edit site and a score, summarizing several predicted performance metrics.

FIG. 70 illustrates SEQ ID NOs: 2196-2243, the crRNA sequences generated by the bioinformatic methods herein described that target human SCN10A to modify and/or ablate expression of its encoded products. Additional information includes the chromosomal genomic coordinates (assembly hg38) of the edit site and a score, summarizing several predicted performance metrics.

FIG. 71 illustrates SEQ ID NOs: 2244-2291, the crRNA sequences generated by the bioinformatic methods herein described that target human SCN11A to modify and/or ablate expression of its encoded products. Additional information includes the chromosomal genomic coordinates (assembly hg38) of the edit site and a score, summarizing several predicted performance metrics.

FIG. 72 illustrates SEQ ID NOs: 2292-2308, the crRNA sequences generated by the bioinformatic methods herein described that target human TACT to modify and/or ablate expression of its encoded products. Additional information includes the chromosomal genomic coordinates (assembly hg38) of the edit site and a score, summarizing several predicted performance metrics.

FIG. 73 illustrates SEQ ID NOs: 2309-2325, the crRNA sequences generated by the bioinformatic methods herein described that target human TAC3 to modify and/or ablate expression of its encoded products. Additional information includes the chromosomal genomic coordinates (assembly hg38) of the edit site and a score, summarizing several predicted performance metrics.

FIG. 74 illustrates SEQ ID NOs: 2326-2373, the crRNA sequences generated by the bioinformatic methods herein described that target human TACR1 to modify and/or ablate expression of its encoded products. Additional information includes the chromosomal genomic coordinates (assembly hg38) of the edit site and a score, summarizing several predicted performance metrics.

FIG. 75 illustrates SEQ ID NOs: 2374-2421, the crRNA sequences generated by the bioinformatic methods herein described that target human TACR2 to modify and/or ablate expression of its encoded products. Additional information includes the chromosomal genomic coordinates (assembly hg38) of the edit site and a score, summarizing several predicted performance metrics.

FIG. 76 illustrates SEQ ID NOs: 2422-2469, the crRNA sequences generated by the bioinformatic methods herein described that target human TACR3 to modify and/or ablate expression of its encoded products. Additional information includes the chromosomal genomic coordinates (assembly hg38) of the edit site and a score, summarizing several predicted performance metrics.

FIG. 77 illustrates SEQ ID NOs: 2470-2509, the crRNA sequences generated by the bioinformatic methods herein described that target human TIMP1 to modify and/or ablate expression of its encoded products. Additional information includes the chromosomal genomic coordinates (assembly hg38) of the edit site and a score, summarizing several predicted performance metrics.

FIG. 78 illustrates SEQ ID NOs: 2510-2557, the crRNA sequences generated by the bioinformatic methods herein described that target human TIMP3 to modify and/or ablate expression of its encoded products. Additional information includes the chromosomal genomic coordinates (assembly hg38) of the edit site and a score, summarizing several predicted performance metrics.

FIG. 79 illustrates SEQ ID NOs: 2558-2574, the crRNA sequences generated by the bioinformatic methods herein described that target human TNF to modify and/or ablate expression of its encoded products. Additional information includes the chromosomal genomic coordinates (assembly hg38) of the edit site and a score, summarizing several predicted performance metrics.

FIG. 80 illustrates SEQ ID NOs: 2575-2622, the crRNA sequences generated by the bioinformatic methods herein described that target human TNFRSF1A to modify and/or ablate expression of its encoded products. Additional information includes the chromosomal genomic coordinates (assembly hg38) of the edit site and a score, summarizing several predicted performance metrics.

FIG. 81 illustrates SEQ ID NOs: 2623-2670, the crRNA sequences generated by the bioinformatic methods herein described that target human TNFRSF1B to modify and/or ablate expression of its encoded products. Additional information includes the chromosomal genomic coordinates (assembly hg38) of the edit site and a score, summarizing several predicted performance metrics.

FIG. 82 illustrates SEQ ID NOs: 2671-2718, the crRNA sequences generated by the bioinformatic methods herein described that target human YAP1 to modify and/or ablate expression of its encoded products. Additional information includes the chromosomal genomic coordinates (assembly hg38) of the edit site and a score, summarizing several predicted performance metrics.

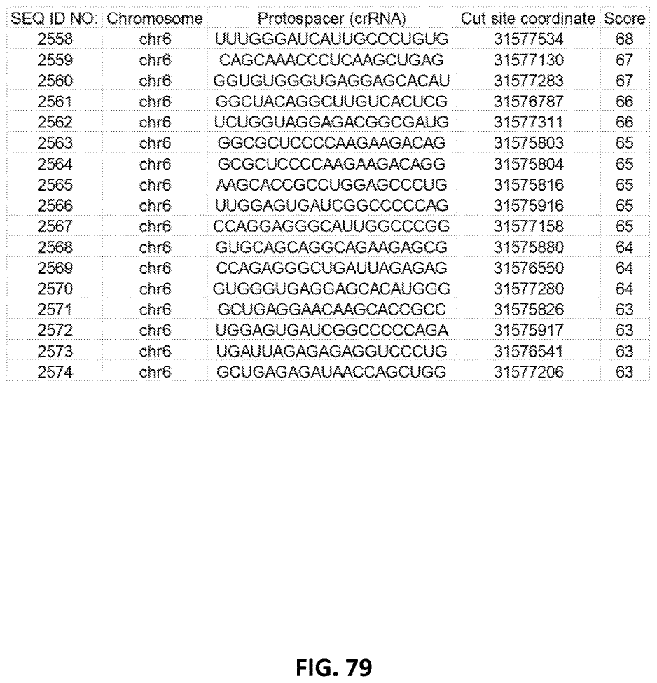

FIGS. 79A, 79B, 79C, 79D, 79E, 79F, 79G, and 79H collectively illustrate SEQ ID NOs: 3186-3349, (A-D) the crRNA sequences generated by the bioinformatic methods herein described that target human IL1RAP to generate a genetic knockout, a soluble decoy receptor, a membrane-bound decoy receptor or other form and (E-H) additional information regarding the chromosome 3 genomic coordinates (assembly hg38) of the bound DNA, DNA strand targeted, exon targeted, and several predicted performance metrics.

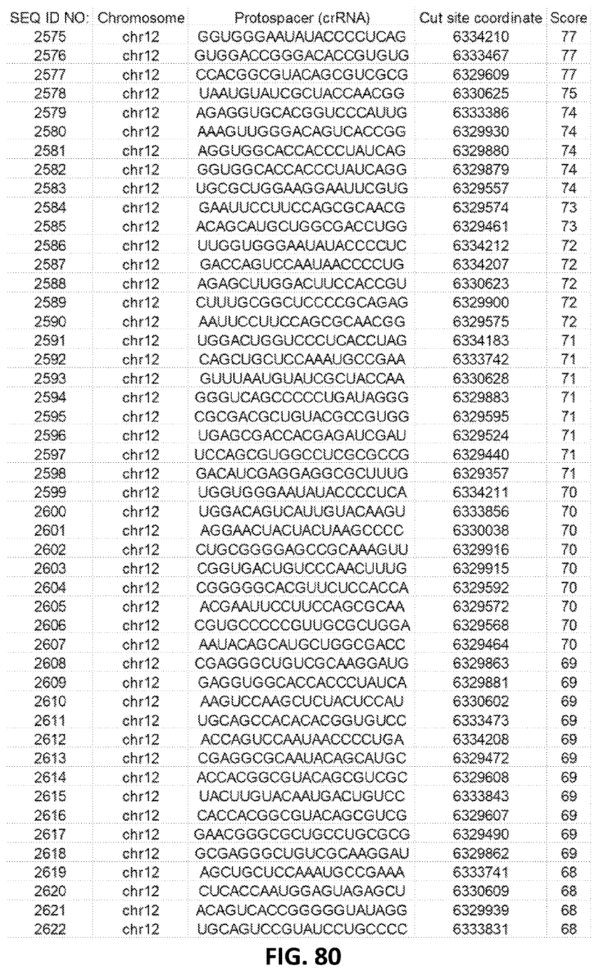

FIGS. 80A, 80B, 80C, 80D, 80E, and 80F collectively illustrate SEQ ID NOs: 3350-3485 (A-C) the crRNA sequences generated by the bioinformatic methods herein described that target canine IL1RAP to generate a genetic knockout, a soluble decoy receptor, a membrane-bound decoy receptor, or other form and (D-F) additional information includes the chromosome 34 genomic coordinates (assembly canFam3) of the bound DNA, the DNA strand targeted, the exon targeted, and several predicted performance metrics.

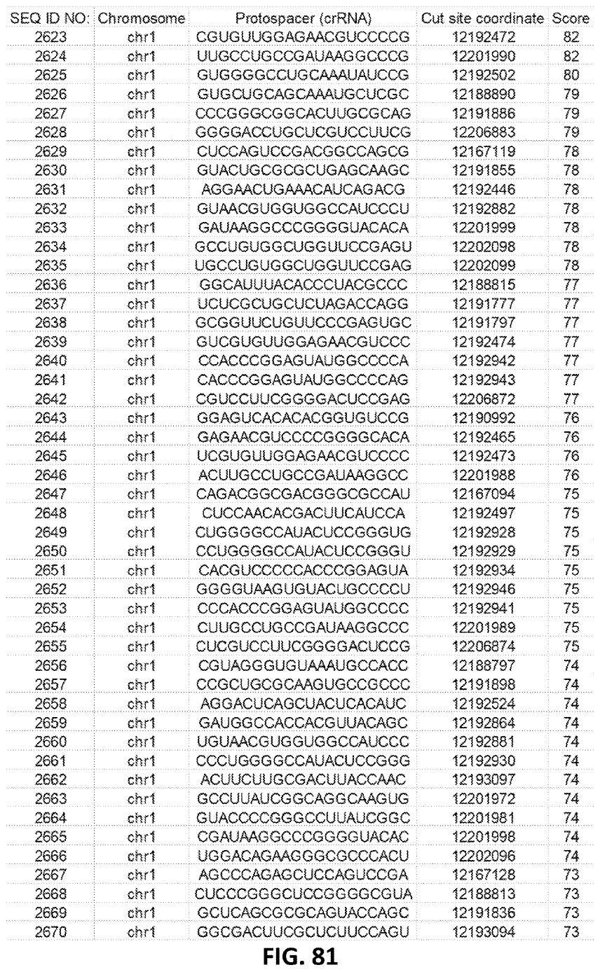

FIGS. 81A, 81B, and 81C collectively illustrate SEQ ID NOs: 3485-3561, the crRNA sequences generated by the bioinformatic methods herein described that target equine IL1RAP to generate (A) a genetic knockout, (B) a soluble decoy receptor, or (C) a membrane-bound decoy receptor. Additional information includes the chromosome 19 genomic coordinates (assembly equCab3) of the bound DNA, the DNA strand targeted, the exon targeted, and several predicted performance metrics.

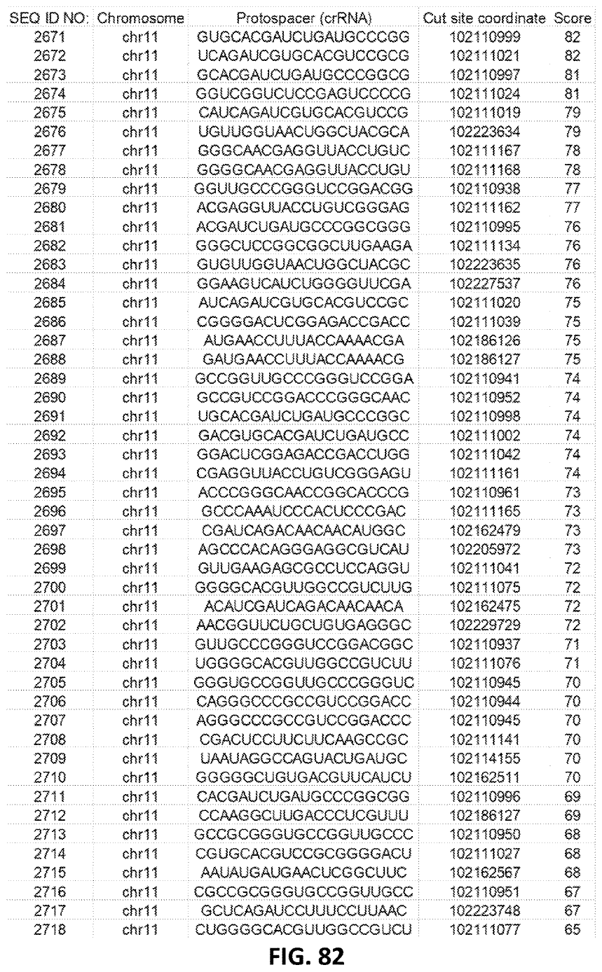

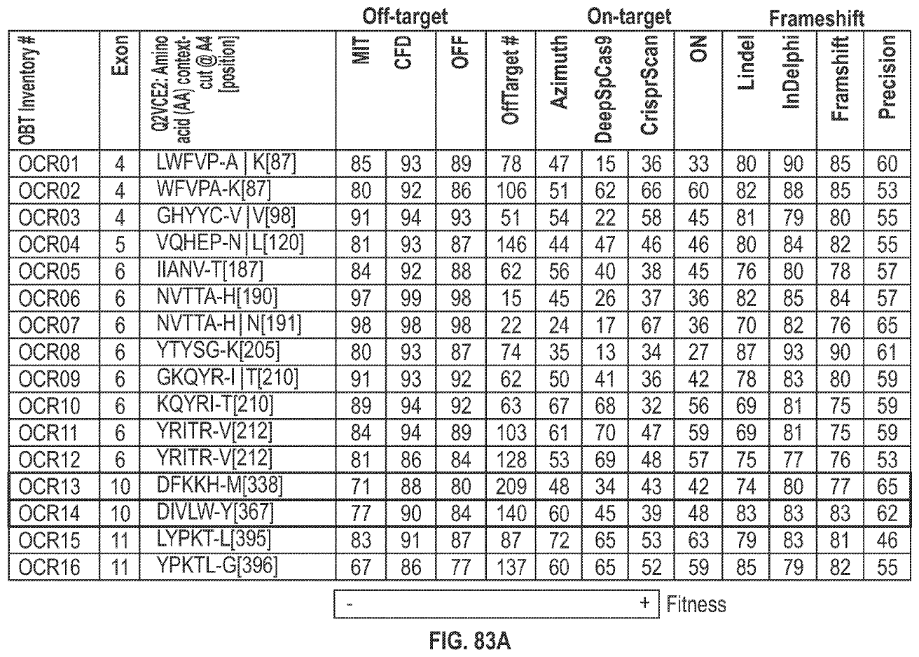

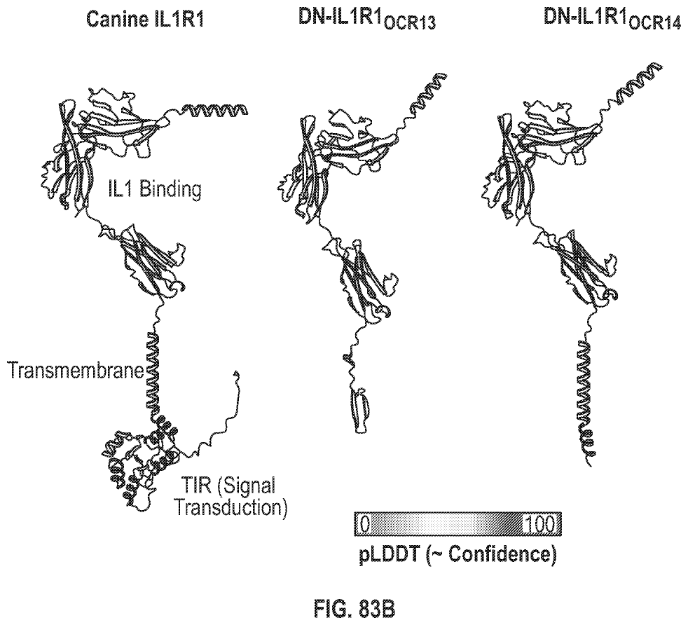

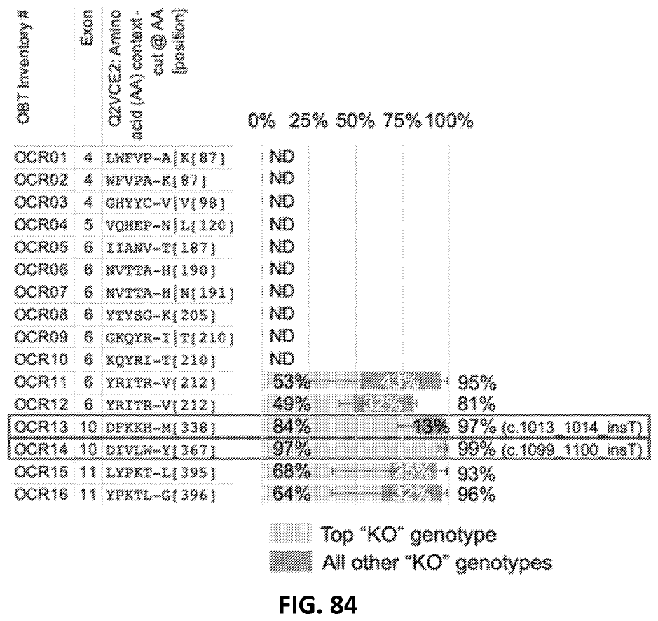

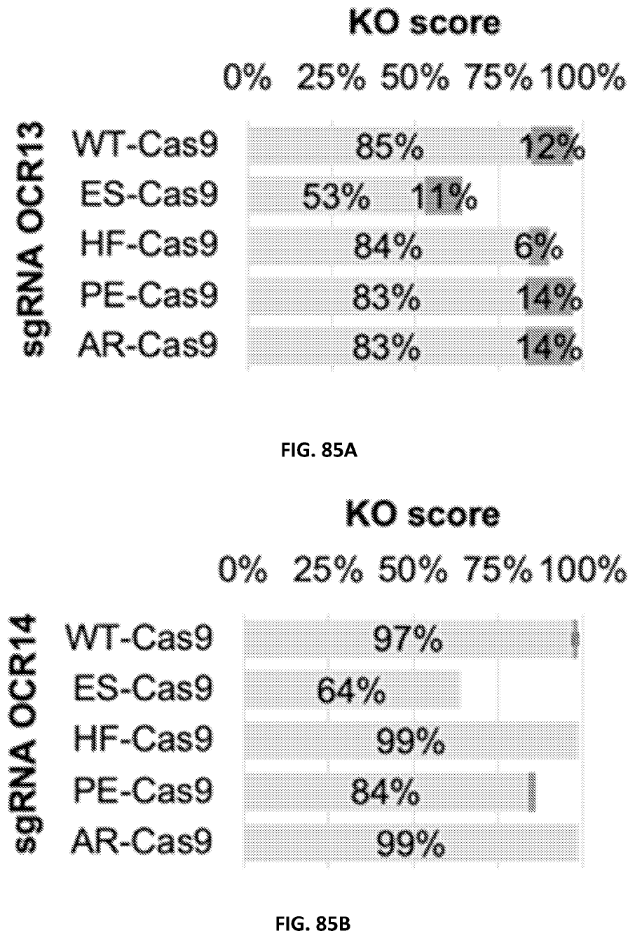

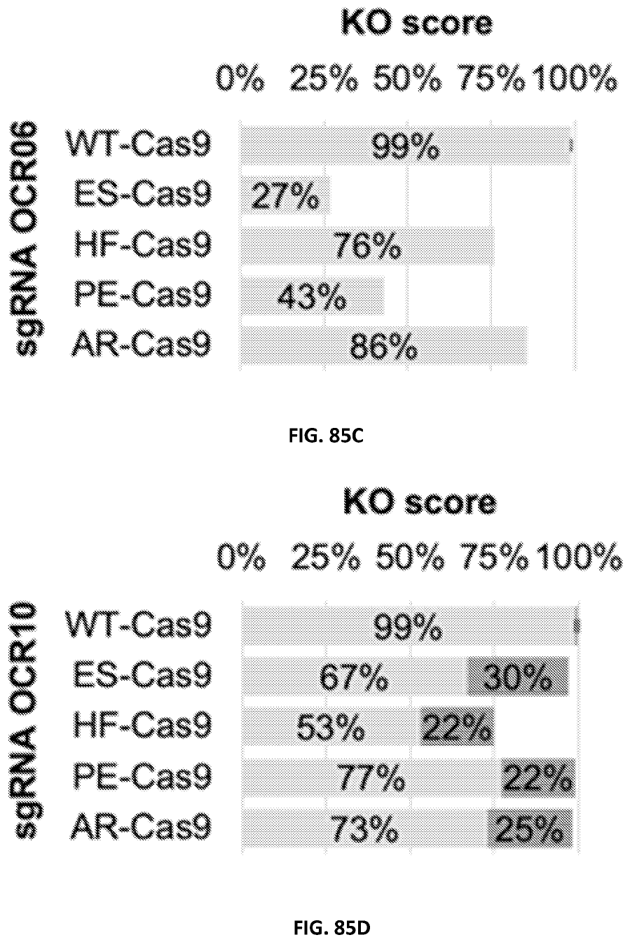

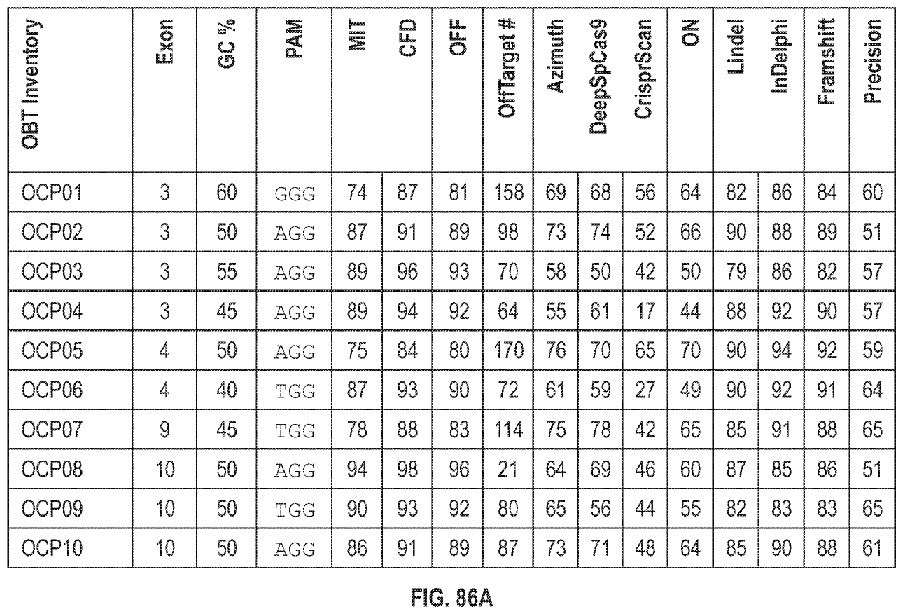

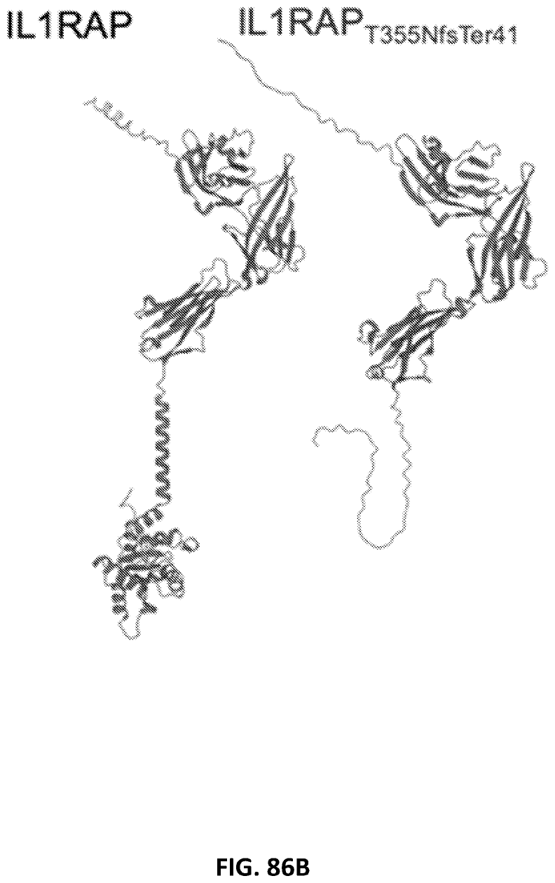

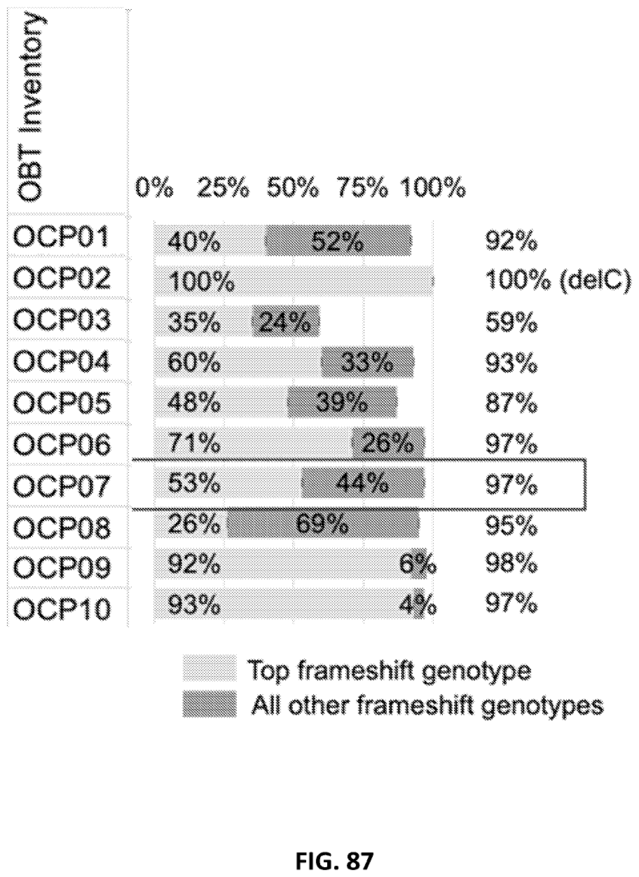

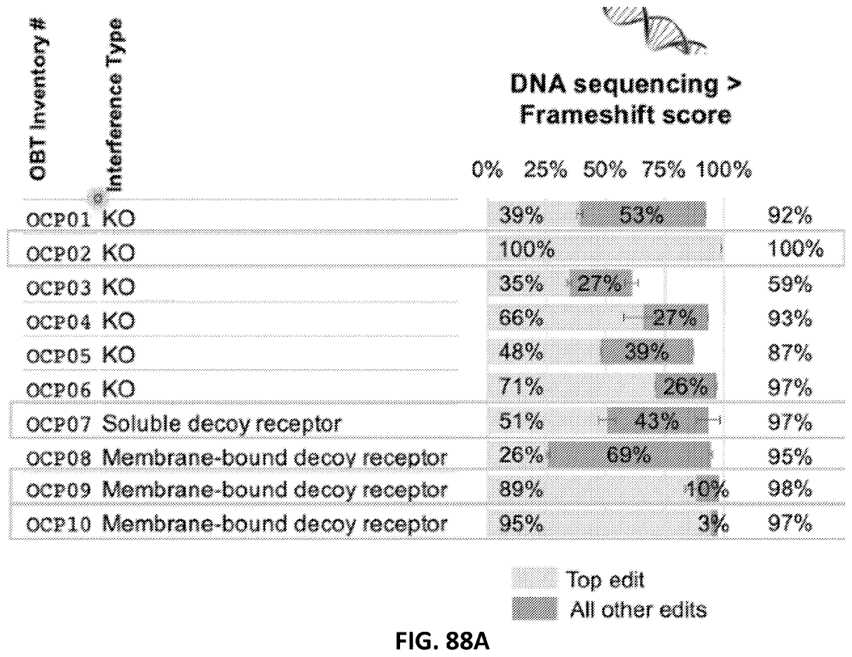

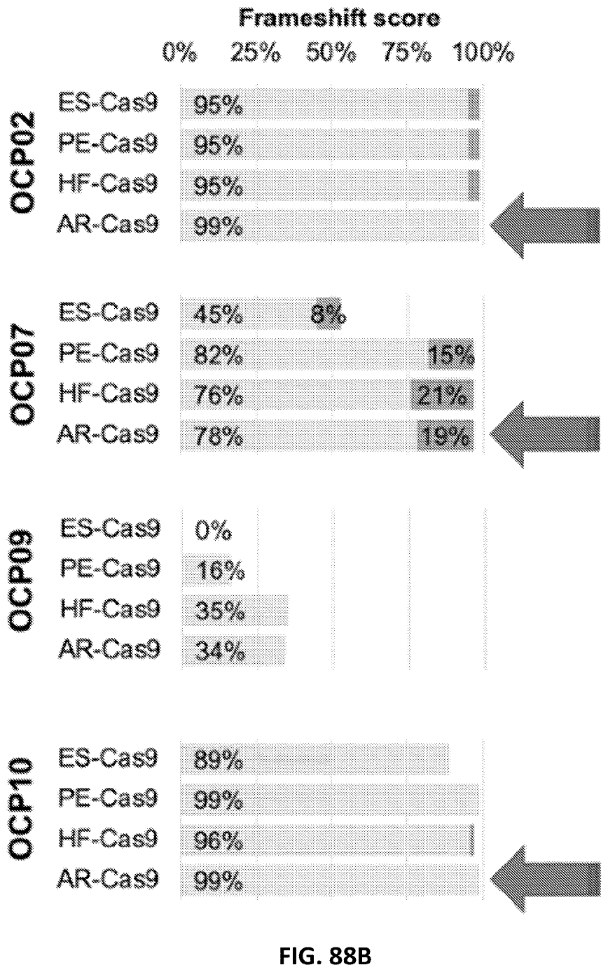

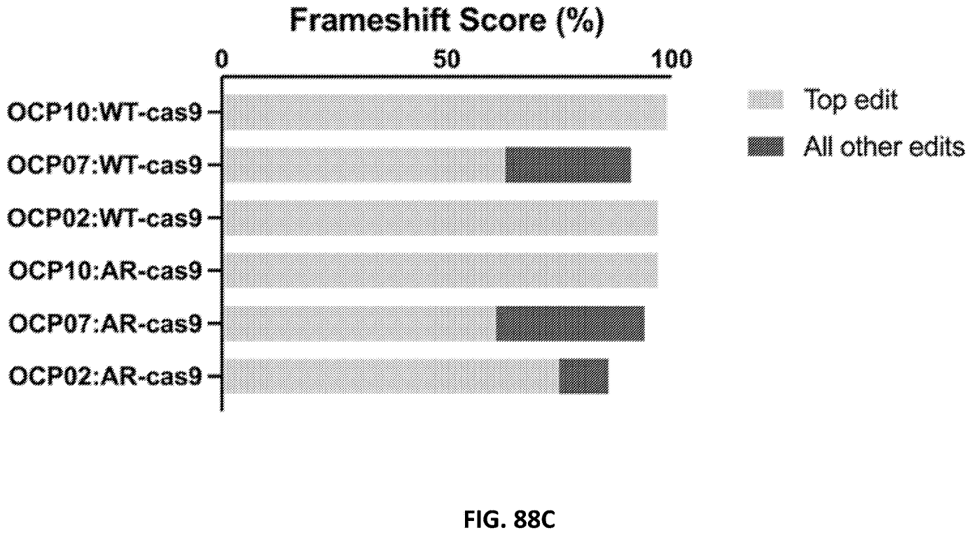

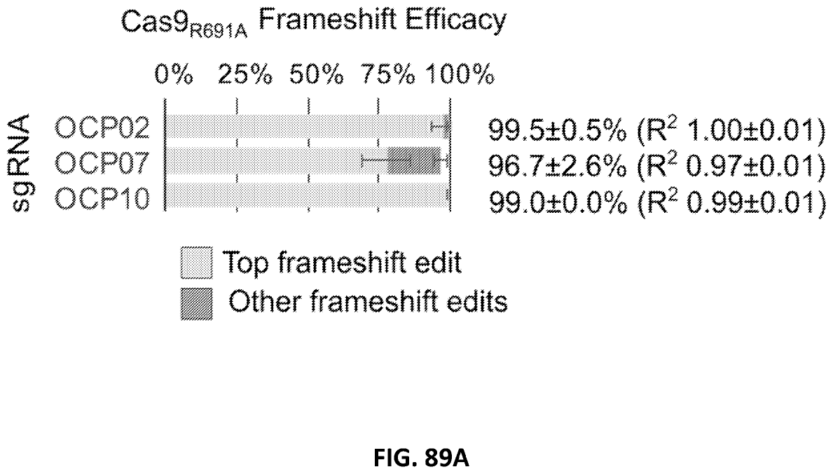

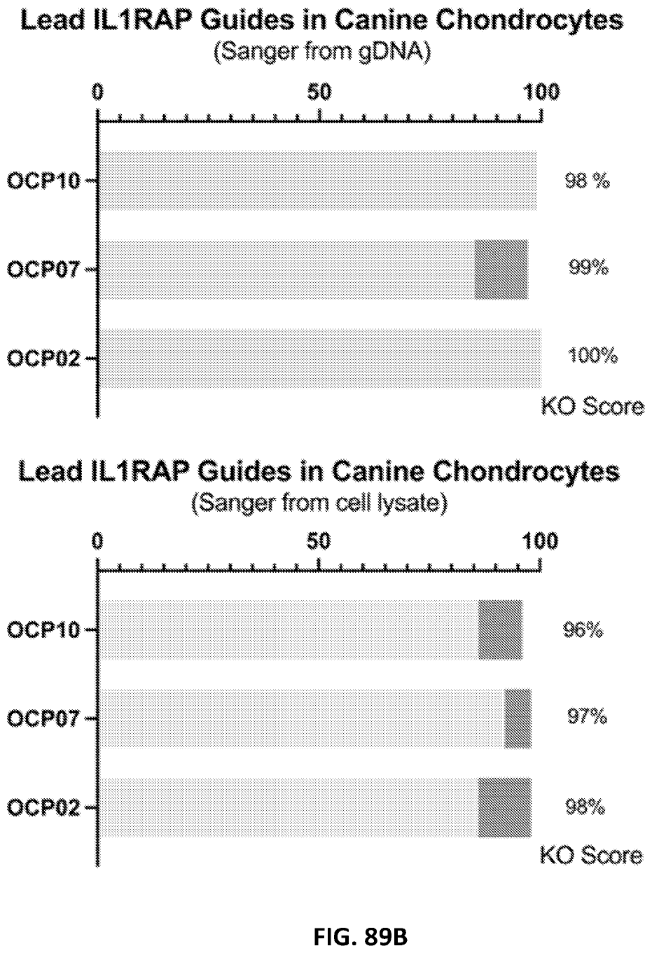

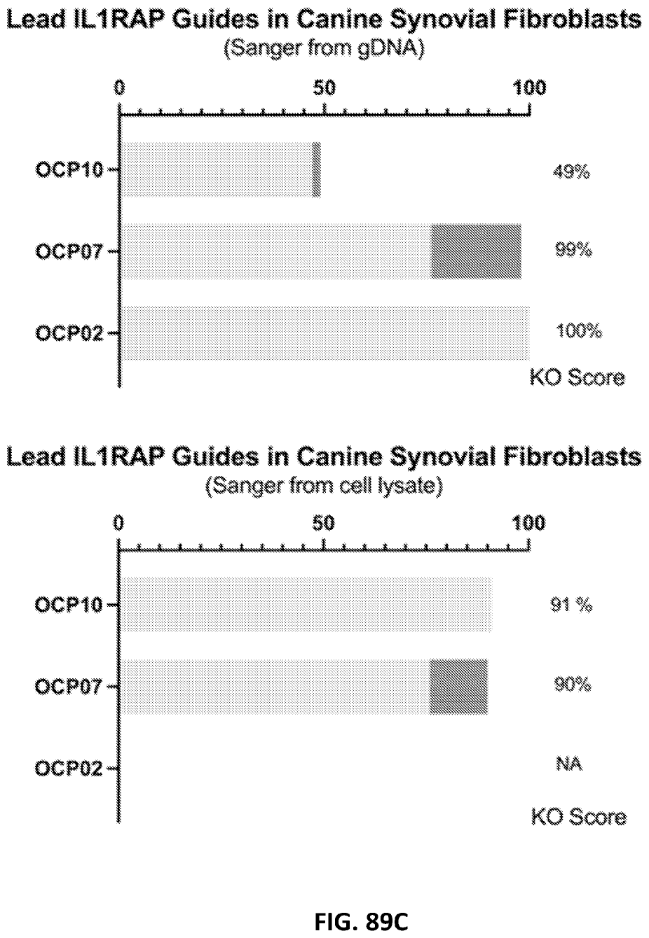

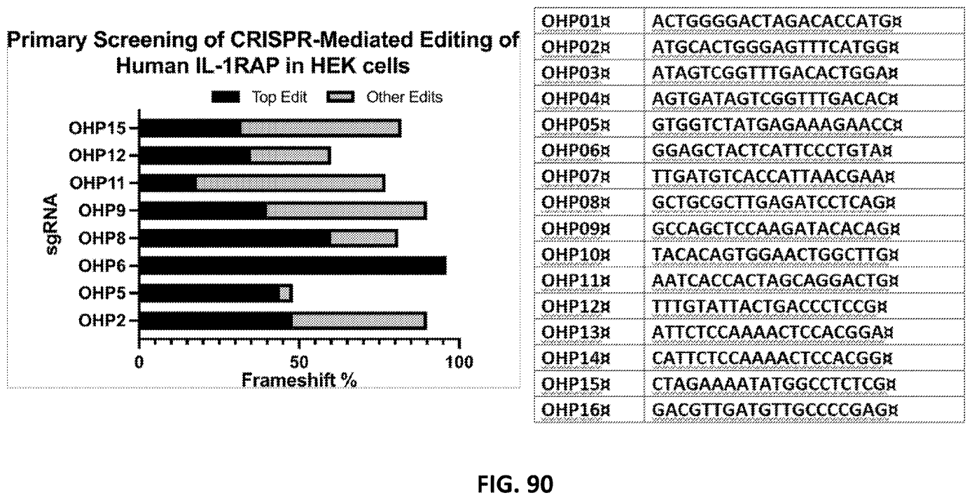

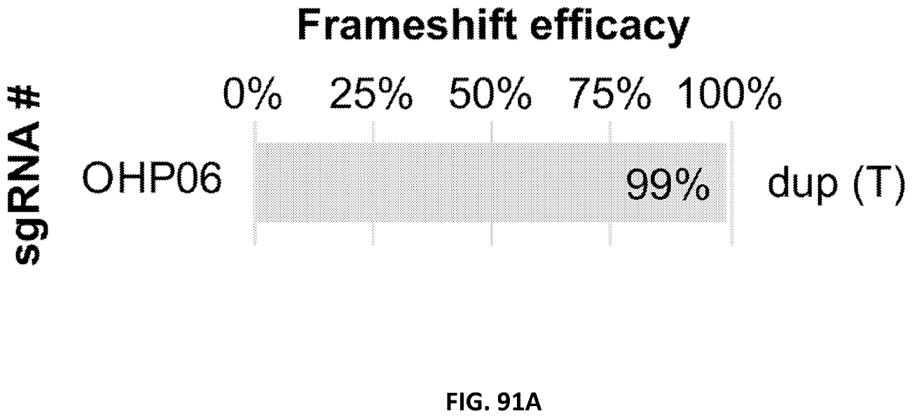

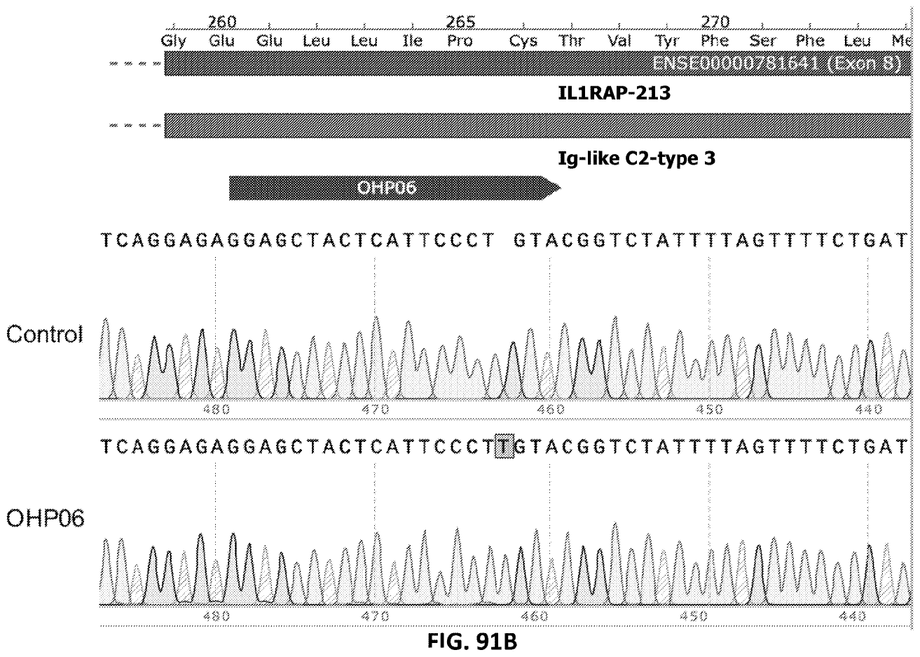

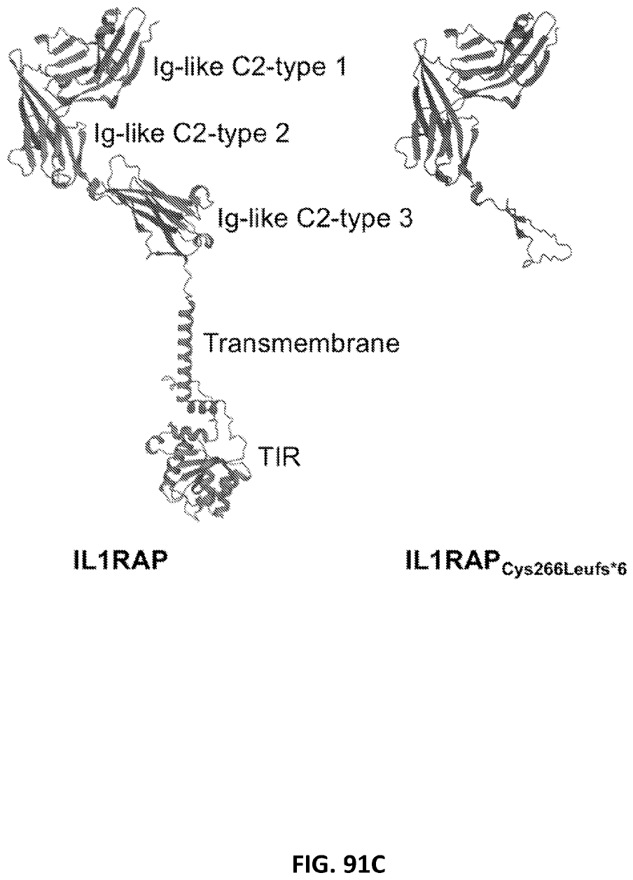

FIGS. 82A, 82B, and 82C collectively illustrate SEQ ID NOs: 3562-3636, the crRNA sequences generated by the bioinformatic methods herein described that target feline IL1RAP to generate (A) a genetic knockout, (B) a soluble decoy receptor, or (C) a membrane-bound decoy receptor. Additional information includes where the chromosome C2 genomic coordinates (assembly felCat9) bound, DNA strand targeted, exon targeted, and several predicted performance metrics.

FIGS. 84A, 84B, 84C, 84D, 84E, and 84F collectively illustrate SEQ ID NOs: 3637-3780 (A-D) the crRNA sequences generated by the bioinformatic methods herein described that target human IL1R1 to generate a genetic knockout, a soluble decoy receptor, a membrane-bound decoy receptor, or other form and (E-H) additional information regarding the chromosome 2 genomic coordinates (assembly hg38) of the bound DNA, DNA strand targeted, exon targeted, and several predicted performance metrics.

FIGS. 85A, 85B, 85C, 85D, 85E, 85F, 85G, and 85H collectively illustrate SEQ ID NOs: 3781-3931 (A-D) the crRNA sequences generated by the bioinformatic methods herein described that target canine IL1R1 to generate a genetic knockout, a soluble decoy receptor, or a membrane-bound decoy receptor, or other form and (E-H) additional information includes the chromosome 10 genomic coordinates (assembly canFam3) of the bound DNA, the DNA strand targeted, the exon targeted, and several predicted performance metrics.

FIGS. 86A, 86B, and 86C collectively illustrate SEQ ID NOs: 3932-4005, the crRNA sequences generated by the bioinformatic methods herein described that target equine IL1R1 to generate (A) a genetic knockout, (B) a soluble decoy receptor, or (C) a membrane-bound decoy receptor. Additional information includes the chromosome 15 genomic coordinates (assembly equCab3) of the bound DNA, the DNA strand targeted, the exon targeted, and several predicted performance metrics.

FIGS. 87A, 87B, and 87C collectively illustrate SEQ ID NOs: 4006-4076, the crRNA sequences generated by the bioinformatic methods herein described that target feline IL1R1 to generate (A) a genetic knockout, (B) a soluble decoy receptor, or (C) a membrane-bound decoy receptor. Additional information includes the chromosome A3 genomic coordinates (assembly felCat9) of the bound DNA, the DNA strand targeted, the exon targeted, and several predicted performance metrics.

FIG. 88 illustrates SEQ ID NOs: XXXX-XXXX, the crRNA sequences generated by the bioinformatic methods herein described that target the human IL1RA gene to modify and/or ablate expression of its encoded products. Additional information includes the chromosomal genomic coordinates (assembly hg38) of the edit site and a score, summarizing several predicted performance metrics.

FIG. 89 illustrates SEQ ID NOs: XXXX-XXXX, the crRNA sequences generated by the bioinformatic methods herein described that target the human IL1RB gene to modify and/or ablate expression of its encoded products. Additional information includes the chromosomal genomic coordinates (assembly hg38) of the edit site and a score, summarizing several predicted performance metrics.

FIGS. 90A and 90B illustrate the design of exemplary sgRNAs that target canine IL1R1, including (A) a summary of select sgRNAs based on off-target risks, on-target efficacy, and frameshift likelihood and (B) AlphaFold2 models of wild-type and decoy IL1R1 receptors, as predicted to be generated by OCR13 and OCR14.

FIG. 91 illustrates the in vitro performance of the tested sgRNA candidates that target canine IL1RAP, as deduced from Sanger traces. ND, not determined.

FIGS. 92A, 92B, 92C, and 92D illustrate the effect of various SpCas9 variants on the in-vitro editing performance of select candidate sgRNAs that target canine IL1R1.

FIGS. 93A and 93B illustrate the design of exemplary sgRNAs that target canine IL1RAP, including (A) a summary of select sgRNAs based on off-target risks, on-target efficiency and frameshift likelihood and (B) AlphaFold2-predicted models of the 3D structure of normal and OCP07-edited IL1RAP.

FIG. 94 illustrates the in vitro performance of the tested sgRNA candidates that target canine IL1RAP, as deduced from Sanger traces.

FIGS. 95A, 95B, 95C illustrate (A) select sgRNAs targeting IL1RAP for testing and their editing efficacy with wildtype SpCas9 (WT-Cas9), (B) the effect of the indicated SpCas9 variants on editing efficacy in canine monocytes and (C) a comparison of editing efficacy between AR-Cas9 and WT-Cas9 in canine synovial fibroblasts.

FIGS. 96A, 96B, and 96C illustrate the editing efficacy of the indicated IL1RAP-directed sgRNAs in (A) canine monocytes, (B) canine chondrocytes and (C) canine synovial fibroblasts.

FIG. 97 illustrates results of a primary screen of hIL1RAP-targeted sgRNAs in HEK cells. Synthetic sgRNA candidates were paired with wild-type SpCas9 protein and electroporated into HEK cells. After two days, cells were genotyped by inferring CRISPR edits from Sanger sequencing traces (ICE v3). Top and other ICE-predicted (R²>0.90) edits contributing to translational frameshifts are summarised here. Due to its high editing efficacy and precision (i.e. only one edit was detectable) sgRNA OHP06 was selected first for an on-target activity study in primary nucleus pulposus cells.

FIGS. 98A, 98B and 98C collectively illustrate the CRISPR editing of the human IL1RAP gene in nucleus pulposus cells of the intervertebral disc. (A) Synthetic single guide RNA (sgRNA) OHP06 was paired with wild-type SpCas9 protein, electroporated into nucleus pulposus cells, which were genotyped after two to three days in culture. The CRISPR-mediated frameshift in the coding sequence of the human IL1RAP gene was inferred from Sanger sequencing traces using ICE software (v1.2 and v3). The average frameshift efficacy was 94% (R^{2′}0.90) with most detected edits (93%) being one single T duplication (dup). Error bars show the standard deviation from the mean. (B) Panel shows the position of sgRNA OHP06 in exon 8 of the human IL1RAP gene encoding Ig-like C2-type 3 and aligned Sanger traces from control and CRISPR-edited nucleus pulposus cells. The duplicated T is framed. (C) AlphaFold2-PTM (with template from pdb70 database and otherwise default settings) was used to predict the 3D structures of wild-type and CRISPR-edited IL1RAP. The T duplication caused a frameshift (fs) at amino acid position 266 converting Cysteine (Cys) into Leucine (Leu) and prematurely terminating translation at codon position 6 as counted from the first changed amino acid to the premature stop codon (Cys266Leufs*6). The mutant IL1RAP lacks the last of three Ig-like C2-type domains as well as the transmembrane and TIR domain.

### DETAILED DESCRIPTION OF THE DISCLOSURE

### I. Introduction

Provided herein are compositions and methods for silencing the the translation of one or more proteins in an animal in need thereof to treat a disease, illness or condition associated with localized pain (i.e., nociception). In some embodiments, this pain is localized to the back and/or spine. In some embodiments, the pain arises from a discogenic disorder (e.g., IDD).

In some embodiments, pain is ameliorated by silencing of a nociception signaling protein (or its cognate receptor) via CRISPR editing of the gene encoding the protein (or receptor). In some embodiments, the CRISPR editing results in ablation of atransmembrane domain of a pain receptor (i.e., generation of a soluble decoy receptor). In some embodiments, the CRISPR editing results in ablation of the cytoplasmic domain of a pain receptor (i.e., generation of a membrane-bound decoy receptor). In particular embodiments, compositions and methods are provided to gene-edit (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain.

### II. Definitions

Provided herein are compositions and methods for silencing the translation of one or more proteins in an animal in need thereof to treat a disease, illness or condition associated with localized pain (e.g., nociception). In some embodiments, this pain is localized to the back and/or spine. In some embodiments, the pain arises from a discogenic disorder (e.g., DDD).

In some embodiments, pain is ameliorated by silencing of a nociception signaling protein (or its cognate receptor) via CRISPR editing of the gene encoding the protein (or receptor). In some embodiments, the CRISPR editing results in ablation of a transmembrane domain of a pain receptor (i.e., generation of a soluble decoy receptor). In some embodiments, the CRISPR editing results in ablation of the cytoplasmic domain of a pain receptor (i.e., generation of a membrane-bound decoy receptor). In particular embodiments, compositions and methods are provided to gene-edit (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain.

### Definitions

Unless defined otherwise, all technical and scientific terms used herein have the same meaning as is commonly understood by one of skill in the art to which this disclosure belongs. All patents and publications referred to herein are incorporated by reference in their entireties.

The term “FGF2 gene” refers to a mammalian gene encoding a Fibroblast growth factor 2 polypeptide. Non-limiting examples of FGF2 genes include: NCBI Gene ID: 2247 [human], NCBI Gene ID: 403857 [canine], NCBI Gene ID: 100033955 [equine], NCBI Gene ID: 100135772 [feline], as well as synonymous and non-synonymous sequence variants thereof. Non-limiting examples of gene products encoded by an FGF2 gene include: UniProt: P09038; NP_001348594.1 [human], XP_038421156.1 [canine], NP_001182150.1 [equine], XP_044911834.1 [feline]), as well as sequence variants, isoforms encoded by alternative splicing, and various glycoforms thereof. Canonically, the proteins encoded by the genes listed above act as ligands for the FGF receptors FGFR1, FGFR2, FGFR3 and FGFR4 in addition to strongly binding heparin and integrins. Additionally, FGF2 signaling is thought to impact localized nociception via at least its pro-angiogenic activity and has been implicated in pain perception related to at least IVD degeneration and at joint lesions. In some instances, and merely for the sake of disambiguation, a prefix is added when referring to the protein or gene of a particular species (with h, c, e, and f referring to the human, canine, equine, and feline forms, respectively).

The term “FGFR1 gene” refers to a mammalian gene encoding a Fibroblast Growth Factor Receptor 1 polypeptide. Non-limiting examples of FGFR1 genes include: NCBI Gene ID: 2260 [human], NCBI Gene ID: 100856477 [canine], NCBI Gene ID: 100057614 [equine], NCBI Gene ID: 101086055 [feline] as well as synonymous and non-synonymous sequence variants thereof. Non-limiting examples of gene products encoded by an FGFR1 gene include: UniProt: P11362; NP_001167534.1 [human], XP_038545782.1 [canine], XP_023486323.1 [equine], XP_011279822.1 [feline], as well as sequence variants, isoforms encoded by alternative splicing, and various glycoforms thereof. Canonically, the proteins encoded by the genes listed above are tyrosine-protein kinases that act as cell-surface receptor for fibroblast growth factors. In that role, they play an essential role in the regulation of embryonic development, cell proliferation, differentiation and migration. In some instances, and merely for the sake of disambiguation, a prefix is added when referring to the protein or gene of a particular species (with h, c, e, and f referring to the human, canine, equine, and feline forms, respectively).

The term “CCN2 gene” refers to a mammalian gene encoding a Cellular Communication Network Factor 2 polypeptide (also known as Connective Tissue Growth Factor, CTGF). Non-limiting examples of CCN2 genes include: NCBI Gene ID: 1490 [human], NCBI Gene ID: 476202 [canine], NCBI Gene ID: 100073098 [equine], NCBI Gene ID: 101094598 [feline], as well as synonymous and non-synonymous sequence variants thereof. Non-limiting examples of gene products encoded by a CCN2 gene include: UniProt: P29279; NP_001892.2 [human], XP_038321343.1 [canine], XP_023506869.1 [equine], XP_023110145.1 [feline], as well as sequence variants, isoforms encoded by alternative splicing, and various glycoforms thereof. Canonically, the proteins encoded by the genes listed above are mitogens secreted by vascular endothelial cells and are related to chondrocyte proliferation and differentiation, cell adhesion in many cell types. In some instances, and merely for the sake of disambiguation, a prefix is added when referring to the protein or gene of a particular species (with h, c, e, and f referring to the human, canine, equine, and feline forms, respectively).

The term “ADAMTS5 gene” refers to a mammalian gene encoding an ADAM Metallopeptidase with Thrombospondin Type 1 Motif 5 polypeptide. Non-limiting examples of ADAMTS5 genes include: NCBI Gene ID: 11096 [human], NCBI Gene ID: 487713 [canine], NCBI Gene ID: 100066005 [equine], NCBI Gene ID: 101085063 [feline], as well as synonymous and non-synonymous sequence variants thereof. Non-limiting examples of gene products encoded by an ADAMTS5 gene include: UniProt: Q9UNA0; NP_008969.2 [human], XP_038299214.1 [canine], XP_023485737.1 [equine], XP_023094603.1 [feline], as well as sequence variants, isoforms encoded by alternative splicing, and various glycoforms thereof. Canonically, members of the family share several distinct protein modules, including a propeptide region, a metalloproteinase domain, a disintegrin-like domain, and a thrombospondin type 1 (TS) motif with individual members of the family differing in the number of C-terminal TS motifs. ADAMTS5 has two unique C-terminal domains. Once proteolytically processed to generate the mature enzyme, ADAMTS5 functions as an aggrecanase that cleaves aggrecan, a major proteoglycan of cartilage, and may mediate cartilage destruction in osteoarthritis. In some instances, and merely for the sake of disambiguation, a prefix is added when referring to the protein or gene of a particular species (with h, c, e, and f, referring to the human, canine, equine, and feline forms, respectively).

The term “ADAMTS1 gene” refer to a mammalian gene encoding an ADAM Metallopeptidase with Thrombospondin Type 1 Motif 1 polypeptide. Non-limiting examples of ADAMTS1 genes include: NCBI Gene ID: 9510 [human], NCBI Gene ID: 100686153 [canine], NCBI Gene ID: 791251 [equine], NCBI Gene ID: 101085309 [feline], as well as synonymous and non-synonymous sequence variants thereof. Non-limiting examples of gene products encoded by an ADAMTS1 gene include: UniProt: Q9UHI8; NP_008919.3 [human], XP_038374156.1 [canine], XP_023485736.1 [equine], XP_019695041.3 [feline], as well as sequence variants, isoforms encoded by alternative splicing, and various glycoforms thereof. Canonically, members of the family share several distinct protein modules, including a propeptide region, a metalloproteinase domain, a disintegrin-like domain, and a thrombospondin type 1 (TS) motif with individual members of the family differing in the number of C-terminal TS motifs. ADAMTS1 contains two disintegrin loops and three C-terminal TS motifs. The protein has anti-angiogenic activity and functions as an aggrecanase that cleaves aggrecan, a major proteoglycan of cartilage, and may be involved in its turnover and has been associated with various inflammatory processes. In some instances, and merely for the sake of disambiguation, a prefix is added when referring to the protein or gene of a particular species (with h, c, e, and f, referring to the human, canine, equine, and feline forms, respectively).

The term “MMP1 gene” refers to a mammalian gene encoding a Matrix Metalloproteinase 1 polypeptide. Non-limiting examples of MMP1 genes include: NCBI Gene ID: 4312 [human], NCBI Gene ID: 489428 [canine], NCBI Gene ID: 100033896 [equine], NCBI Gene ID: 101084217 [feline], as well as synonymous and non-synonymous sequence variants thereof. Non-limiting examples of gene products encoded by an MMP1 gene include: UniProt: P03956; NP_001139410.1 [human], XP_038521018.1 [canine], NP_001075316.1 [equine], XP_003992365.2 [feline], as well as sequence variants, isoforms encoded by alternative splicing, and various glycoforms thereof. Canonically, MMP1 is proteolytically processed from a preproprotein to generate the mature protease. This secreted protease breaks down the interstitial collagens, including types I, II, and III. In some instances, and merely for the sake of disambiguation, a prefix is added when referring to the protein or gene of a particular species (with h, c, e, and f, referring to the human, canine, equine, and feline forms, respectively).

The term “MMP2 gene” refers to a mammalian gene encoding a Matrix Metalloproteinase 2 polypeptide. Non-limiting examples of MMP2 genes include: NCBI Gene ID: 4313 [human], NCBI Gene ID: 403733 [canine], NCBI Gene ID: 100033948 [equine], NCBI Gene ID: 101098838 [feline], as well as synonymous and non-synonymous sequence variants thereof. Non-limiting examples of gene products encoded by an MMP2 gene include: UniProt: P08253; NP_001121363.1 [human], XP_038515255.1 [canine], XP_023492775.1 [equine], XP_003998091.2 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP2 is a gelatinase A, type IV collagenase, that contains three fibronectin type II repeats in its catalytic site that allow binding of denatured type IV and V collagen and elastin. In some instances, and merely for the sake of disambiguation, a prefix is added when referring to the protein or gene of a particular species (with h, c, e, and f referring to the human, canine, equine, and feline forms, respectively).

The term “MMP3 gene” refers to a mammalian gene encoding a Matrix Metalloproteinase 3 polypeptide. Non-limiting examples of MMP3 genes include: NCBI Gene ID: 4314 [human], NCBI Gene ID: 403733 [canine], NCBI Gene ID: 100034195 [equine], NCBI Gene ID: 493666 [feline], as well as synonymous and non-synonymous sequence variants thereof. Non-limiting examples of gene products encoded by an MMP3 gene include: UniProt: P08254; NP_002413.1 [human], NP_001002967.1 [canine], NP_001075964.1 [equine], XP_003992356.2 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP3 is an enzyme that degrades fibronectin, laminin, collagens III, IV, IX, and X, and cartilage proteoglycans and is thought to be involved in wound repair, progression of atherosclerosis, and tumor initiation. In some instances, and merely for the sake of disambiguation, a prefix is added when referring to the protein or gene of a particular species (with h, c, e, and f referring to the human, canine, equine, and feline forms, respectively).

The term “MMP7 gene” refers to a mammalian gene encoding a Matrix Metalloproteinase 7 polypeptide. Non-limiting examples of MMP7 genes include: NCBI Gene ID: 4316 [human], NCBI Gene ID: 489432 [canine], NCBI Gene ID: 100068985 [equine], NCBI Gene ID: 727698 [feline], as well as synonymous and non-synonymous sequence variants thereof. Non-limiting examples of gene products encoded by an MMP7 gene include: UniProt: P09237; NP_002414.1 [human], NP_001229655.1 [canine], XP_001498859.1 [equine], XP_003992352.1 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP7 is proteolytically processed to generate the mature protease, which breaks down proteoglycans, fibronectin, elastin and casein in addition to activating procollegnase. In some instances, and merely for the sake of disambiguation, a prefix is added when referring to the protein or gene of a particular species (with h, c, e, and f referring to the human, canine, equine, and feline forms, respectively).

The term “MMP8 gene” refers to a mammalian gene encoding a Matrix Metalloproteinase 8 polypeptide. Non-limiting examples of MMP8 genes include: NCBI Gene ID: 4317 [human], NCBI Gene ID: 489429 [canine], NCBI Gene ID: 100069005 [equine], NCBI Gene ID: 101080995 [feline], as well as synonymous and non-synonymous sequence variants thereof. Non-limiting examples of gene products encoded by an MMP8 gene include: UniProt: P22894; NP_001291370.1 [human], XP_038521019.1 [canine], XP_005611595.1 [equine], XP_003992354.3 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP8 is an enzyme that degrades interstitial collagens. In some instances, and merely for the sake of disambiguation, a prefix is added when referring to the protein or gene of a particular species (with h, c, e, and f referring to the human, canine, equine, and feline forms, respectively).

The term “MMP10 gene” refers to a mammalian gene encoding a Matrix Metalloproteinase 10 polypeptide. Non-limiting examples of MMP10 genes include: NCBI Gene ID: 4319 [human], NCBI Gene ID: 100146442 [equine], NCBI Gene ID: 101081247 [feline], as well as synonymous and non-synonymous sequence variants thereof. Non-limiting examples of gene products encoded by an MMP10 gene include: UniProt: P09238; NP_002416.1 [human], XP_005614947.1 [equine], XP_003992355.2 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP10 is an enzyme that degrades fibronectin, and type I, III, IV, and V gelatins. In some instances, and merely for the sake of disambiguation, a prefix is added when referring to the protein or gene of a particular species (with h, c, e, and f referring to the human, canine, equine, and feline forms, respectively).

The term “MMP12 gene” refers to a mammalian gene encoding a Matrix Metalloproteinase 12 polypeptide. Non-limiting examples of MMP12 genes include: NCBI Gene ID: 4321 [human], NCBI Gene ID: 611789 [canine], NCBI Gene ID: 100069047 [equine], NCBI Gene ID: 101084472 [feline], as well as synonymous and non-synonymous sequence variants thereof. Non-limiting examples of gene products encoded by an MMP12 gene include: UniProt: P39900; NP_002417.2 [human], NP_001274067.1 [canine], XP_001498924.2 [equine], XP_003992366.2 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP12 is an enzyme with significant elastolytic activity and may be involved in tissue injury and remodeling. In some instances, and merely for the sake of disambiguation, a prefix is added when referring to the protein or gene of a particular species (with h, c, e, and f, referring to the human, canine, equine, and feline forms, respectively).

The term “MMP13 gene” refers to a mammalian gene encoding a Matrix Metalloproteinase 13 polypeptide. Non-limiting examples of MMP13 genes include: NCBI Gene ID: 4322 [human], NCBI Gene ID: 403763 [canine], NCBI Gene ID: 100009711 [equine], NCBI Gene ID: 493679 [feline], as well as synonymous and non-synonymous sequence variants thereof. Non-limiting examples of gene products encoded by an MMP13 gene include: UniProt: P45452; NP_002418.1 [human], XP_038521017.1 [canine], NP_001075273.1 [equine], XP_023094811.2 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP13 is an enzyme that degrades various types of collagen and has been implicated in wound healing, tissue remodeling, cartilage degradation, bone development, bone mineralization and ossification. In some instances, and merely for the sake of disambiguation, a prefix is added when referring to the protein or gene of a particular species (with h, c, e, and f, referring to the human, canine, equine, and feline forms, respectively).

The term “TIMP1 gene” refers to a mammalian gene encoding a TIMP Metallopeptidase Inhibitor 1 polypeptide. Non-limiting examples of TIMP1 genes include: NCBI Gene ID: 7076 [human], NCBI Gene ID: 403816 [canine], NCBI Gene ID: 100034220 [equine], NCBI Gene ID: 101095886 [feline], as well as synonymous and non-synonymous sequence variants thereof. Non-limiting examples of gene products encoded by a TIMP1 gene include: UniProt: P01033; NP_003245.1 [human], NP_001003182.1 [canine], XP_023488949.1 [equine], XP_023105059.1 [feline], as well as sequence variants, isoforms encoded by alternative splicing, and various glycoforms thereof. Canonically, the protein encoded by this gene functions by forming one to one complexes with target metalloproteinases, such as collagenases, irreversibly inactivating through binding to their catalytic zinc cofactor. TIMP1 acts on MMP1, MMP2, MMP3, MMP7, MMP8, MMP9, MMP10, MMP11, MMP12, MMP13 and MMP16, but not on MMP14 and has been shown to act as a growth factor regulating cell differentiation, migration and cell death. In some instances, and merely for the sake of disambiguation, a prefix is added when referring to the protein or gene of a particular species (with h, c, e, and f referring to the human, canine, equine, and feline forms, respectively).

The term “TIMP3 gene” refers to a mammalian gene encoding a TIMP Metallopeptidase Inhibitor 3 polypeptide. Non-limiting examples of TIMP3 genes include: NCBI Gene ID: 7078 [human], NCBI Gene ID: 481289 [canine], NCBI Gene ID: 100033947 [equine], NCBI Gene ID: 101091215 [feline], as well as synonymous and non-synonymous sequence variants thereof. Non-limiting examples of gene products encoded by a TIMP3 gene include: UniProt: P35625; NP_000353.1 [human], NP_001271368.1 [canine], NP_001075339.1 [equine], XP_003989265.1 [feline], as well as sequence variants, isoforms encoded by alternative splicing, and various glycoforms thereof. Canonically, the protein encoded by this gene complexes with metalloproteinases (such as collagenases) to irreversibly inactivate them by binding to their catalytic zinc cofactor. TIMP3 is known to act on MMP1, MMP2, MMP3, MMP7, MMP9, MMP13, MMP14 and MMP15. In some instances, and merely for the sake of disambiguation, a prefix is added when referring to the protein or gene of a particular species (with h, c, e, and f referring to the human, canine, equine, and feline forms, respectively).

The term “CXCL1 gene” refers to a mammalian gene encoding a C—X—C Motif Chemokine Ligand 1 polypeptide. Non-limiting examples of CXCL1 genes include: NCBI Gene ID: 2919 [human], NCBI Gene ID: 100034121 [equine], NCBI Gene ID: 102901432 [feline], as well as synonymous and non-synonymous sequence variants thereof. Non-limiting examples of gene products encoded by a CXCL1 gene include: UniProt: P09341; NP_001502.1 [human], NP_001296409.1 [equine], XP_023108817.2 [feline], as well as sequence variants, isoforms encoded by alternative splicing, and various glycoforms thereof. Canonically, the protein encoded by this gene has chemotactic activity for neutrophils and may play a role inflammation. In some instances, and merely for the sake of disambiguation, a prefix is added when referring to the protein or gene of a particular species (with h, c, e, and f referring to the human, canine, equine, and feline forms, respectively).

The term “CXCL2 gene” refers to a mammalian gene encoding a C—X—C Motif Chemokine Ligand 2 polypeptide. Non-limiting examples of CXCL2 genes include: NCBI Gene ID: 2920 [human], NCBI Gene ID: 100233237 [equine], as well as synonymous and non-synonymous sequence variants thereof. Non-limiting examples of gene products encoded by a CXCL2 gene include: UniProt: P19875, Q9UPB8; NP_002080.1 [human], NP_001137427.1 [equine], as well as sequence variants, isoforms encoded by alternative splicing, and various glycoforms thereof. Canonically, the protein encoded by this gene has antimicrobial function via its regulation of inflammatory and immunoregulatory processes. CXCL2 is expressed at the site of inflammation and has been shown to suppress proliferation of hematopoietic progenitor cells. In some instances, and merely for the sake of disambiguation, a prefix is added when referring to the protein or gene of a particular species (with h, c, e, and f referring to the human, canine, equine, and feline forms, respectively).

The term “CXCL3 gene” refers to a mammalian gene encoding a C—X—C Motif Chemokine Ligand 3 polypeptide. Non-limiting examples of CXCL3 genes include: NCBI Gene ID: 2921 [human] NCBI Gene ID: 100056258 [equine], as well as synonymous and non-synonymous sequence variants thereof. Non-limiting examples of gene products encoded by a CXCL3 gene include: UniProt: P19876, Q4W5H9; NP_002081.2 [human], NP_001137265.1 [equine], as well as sequence variants, isoforms encoded by alternative splicing, and various glycoforms thereof. Canonically, the protein encoded by this gene is a secreted growth factor that signals through the G-protein coupled receptor, CXCR2 and plays a role in inflammation and as a chemoattractant for neutrophils. In some instances, and merely for the sake of disambiguation, a prefix is added when referring to the protein or gene of a particular species (with h, c, e, and f referring to the human, canine, equine, and feline forms, respectively).

The term “CXCL5 gene” refers to a mammalian gene encoding a C—X—C Motif Chemokine Ligand 5 polypeptide. Non-limiting examples of CXCL5 genes include: NCBI Gene ID: 6374 [human], as well as synonymous and non-synonymous sequence variants thereof. Non-limiting examples of gene products encoded by a CXCL5 gene include: UniProt: P19876, Q4W5H9; NP_002081.2 [human], UniProt: P97885 [rat], UniProt: P50228 [mouse] as well as sequence variants, isoforms encoded by alternative splicing, and various glycoforms thereof. Canonically, the protein encoded by this gene is thought to interact with the G-protein coupled receptor, CXCR2 to promote angiogenesis, remodel connective tissues and recruit neutrophils. In some instances, and merely for the sake of disambiguation, a prefix is added when referring to the protein or gene of a particular species (with h, c, e, and f referring to the human, canine, equine, and feline forms, respectively).

The term “CXCL6 gene” refers to a mammalian gene encoding a C—X—C Motif Chemokine Ligand 6 polypeptide. Non-limiting examples of CXCL6 genes include: NCBI Gene ID: 6372 [human], NCBI Gene ID: 106557449 [canine], NCBI Gene ID: 100033988 [equine], NCBI Gene ID: 101094593 [feline], as well as synonymous and non-synonymous sequence variants thereof. Non-limiting examples of gene products encoded by a CXCL6 gene include: UniProt: P80162; NP_002984.1 [human], XP_038541813.1 [canine], NP_001075355.2 [equine], XP_003985379.3 [feline] as well as sequence variants, isoforms encoded by alternative splicing, and various glycoforms thereof. Canonically, the protein encoded by this gene is a chemotactic factor for neutrophils and exhibits antibacterial activity. In some instances, and merely for the sake of disambiguation, a prefix is added when referring to the protein or gene of a particular species (with h, c, e, and f, referring to the human, canine, equine, and feline forms, respectively).

The term “CXCL8 gene” refers to a mammalian gene encoding a C—X—C Motif Chemokine Ligand 8 polypeptide. Non-limiting examples of CXCL8 genes include: NCBI Gene ID: 3576 [human], NCBI Gene ID: 403850 [canine], NCBI Gene ID: 100037400 [equine], NCBI Gene ID: 493836 [feline], as well as synonymous and non-synonymous sequence variants thereof. Non-limiting examples of gene products encoded by a CXCL8 gene include: UniProt: P10145; NP_000575.1 [human], NP_001003200.1 [canine], NP_001077420.2 [equine], NP_001009281.1 [feline], as well as sequence variants, isoforms encoded by alternative splicing, and various glycoforms thereof. Canonically, the protein encoded by this gene is secreted by mononuclear macrophages, neutrophils, eosinophils, T lymphocytes, epithelial cells, and fibroblasts and functions as a chemotactic factor that guides neutrophils to the site of infection. CXCL8 also participates with other cytokines in the proinflammatory signaling cascade. In some instances, and merely for the sake of disambiguation, a prefix is added when referring to the protein or gene of a particular species (with h, c, e, and f, referring to the human, canine, equine, and feline forms, respectively).

The term “CCL2 gene” refers to a mammalian gene encoding a C—C Motif Chemokine Ligand 2″ polypeptide. Non-limiting examples of CCL2 genes include: NCBI Gene ID: 6347 [human], NCBI Gene ID: 403981 [canine], NCBI Gene ID: 100034136 [equine], NCBI Gene ID: 100127112 [feline], as well as synonymous and non-synonymous sequence variants thereof. Non-limiting examples of gene products encoded by a CCL2 gene include: UniProt: P13500; NP_002973.1 [human], NP_001003297.1 [canine], NP_001075400.1 [equine], XP_003996605.1 [feline], as well as sequence variants, isoforms encoded by alternative splicing, and various glycoforms thereof. Canonically, the protein encoded by this gene acts as a ligand for CCR2, which induces chemotactic activity for monocytes and basophils (but not neutrophils or eosinophils). In some instances, and merely for the sake of disambiguation, a prefix is added when referring to the protein or gene of a particular species (with h, c, e, and f, referring to the human, canine, equine, and feline forms, respectively).

The term “CCL3 gene” refers to a mammalian gene encoding a C—C Motif Chemokine Ligand 3 polypeptide. Non-limiting examples of CCL3 genes include: NCBI Gene ID: 6348 [human], NCBI Gene ID: 448787 [canine], NCBI Gene ID: 100057909 [equine], NCBI Gene ID: 100302540 [feline], as well as synonymous and non-synonymous sequence variants thereof. Non-limiting examples of gene products encoded by a CCL3 gene include: UniProt: P10147; NP_002974.1 [human], NP_001005251.2 [canine], NP_001108413.1 [equine], NP_001157129.1 [feline], as well as sequence variants, isoforms encoded by alternative splicing, and various glycoforms thereof. Canonically, the protein encoded by this gene plays a role in inflammatory responses through binding to the receptors CCR1, CCR4 and CCR5. In some instances, and merely for the sake of disambiguation, a prefix is added when referring to the protein or gene of a particular species (with h, c, e, and f referring to the human, canine, equine, and feline forms, respectively).

The term “CCL5 gene” refers to a mammalian gene encoding a C—C Motif Chemokine Ligand 5 polypeptide. Non-limiting examples of CCL5 genes include: NCBI Gene ID: 6352 [human], NCBI Gene ID: 403522 [canine], NCBI Gene ID: 100033925 [equine], NCBI Gene ID: 493689 [feline], as well as synonymous and non-synonymous sequence variants thereof. Non-limiting examples of gene products encoded by a CCL5 gene include: UniProt: P13501; NP_001265665.1 [human], NP_001003010.1 [canine], NP_001075332.1 [equine], NP_001009827.1 [feline]) as well as sequence variants, isoforms encoded by alternative splicing, and various glycoforms thereof. Canonically, the protein encoded by this gene functions as a chemoattractant for blood monocytes, memory T helper cells and eosinophils, induces the release of histamine from basophils, and activates eosinophils. In some instances, and merely for the sake of disambiguation, a prefix is added when referring to the protein or gene of a particular species (with h, c, e, and f, referring to the human, canine, equine, and feline forms, respectively).

The term “CCL7 gene” refers to a mammalian gene encoding a C—C Motif Chemokine Ligand 7 polypeptide. Non-limiting examples of CCL7 genes include: NCBI Gene ID: 6354 [human], NCBI Gene ID: 491148 [canine], NCBI Gene ID: 100071714 [equine], NCBI Gene ID: 101096931 [feline], as well as synonymous and non-synonymous sequence variants thereof. Non-limiting examples of gene products encoded by a CCL7 gene include: UniProt: P80098; NP_006264.2 [human], NP_001010960.1 [canine], XP_005597638.1 [equine], XP_044900774.1 [feline], as well as sequence variants, isoforms encoded by alternative splicing, and various glycoforms thereof. Canonically, the protein encoded by this gene is a secreted chemokine which attracts macrophages during inflammation and metastasis and is an in vivo substrate of MMP2. In some instances, and merely for the sake of disambiguation, a prefix is added when referring to the protein or gene of a particular species (with h, c, e, and f, referring to the human, canine, equine, and feline forms, respectively).

The term “CCL20 gene” refers to a mammalian gene encoding a C—C Motif Chemokine Ligand 20 polypeptide. Non-limiting examples of CCL20 genes include: NCBI Gene ID: 6364 [human], NCBI Gene ID: 448790 [canine], NCBI Gene ID: 100629808 [equine], NCBI Gene ID: 101089032 [feline], as well as synonymous and non-synonymous sequence variants thereof. Non-limiting examples of gene products encoded by a CCL20 gene include: UniProt: P78556; NP_001123518.1 [human], NP_001005254.1 [canine], XP_003365179.2 [equine], XP_003991274.2 [feline], as well as sequence variants, isoforms encoded by alternative splicing, and various glycoforms thereof. Canonically, the protein encoded by this gene is involved in inflammatory processes and displays chemotactic activity for lymphocytes. In some instances, and merely for the sake of disambiguation, a prefix is added when referring to the protein or gene of a particular species (with h, c, e, and f, referring to the human, canine, equine, and feline forms, respectively).

The term “CXCR1 gene” refers to a mammalian gene encoding a C—X—C Motif Chemokine Receptor 1 polypeptide. Non-limiting examples of CXCR1 genes include: NCBI Gene ID: 3577 [human], NCBI Gene ID: 478906 [canine], NCBI Gene ID: 100058291 [equine], NCBI Gene ID: 101085650 [feline], as well as synonymous and non-synonymous sequence variants thereof. Non-limiting examples of gene products encoded by a CXCR1 gene include: UniProt: P25024; NP_000625.1 [human], XP_038303849.1 [canine], XP_001491062.1 [equine], XP_011283865.1 [feline], as well as sequence variants, isoforms encoded by alternative splicing, and various glycoforms thereof. Canonically, the protein encoded by this gene is a receptor for IL8 and transduces signaling to mediate neutrophil migration to sites of inflammation, among other activities. In some instances, and merely for the sake of disambiguation, a prefix is added when referring to the protein or gene of a particular species (with h, c, e, and f, referring to the human, canine, equine, and feline forms, respectively).

The term “CXCR2 gene” refers to a mammalian gene encoding a C—X—C Motif Chemokine Receptor 2 polypeptide. Non-limiting examples of CXCR2 genes include: NCBI Gene ID: 3579 [human], NCBI Gene ID: 478905 [canine], NCBI Gene ID: 100055552 [equine], NCBI Gene ID: 101085396 [feline], as well as synonymous and non-synonymous sequence variants thereof. Non-limiting examples of gene products encoded by a CXCR2 gene include: e.g., UniProt: P25025; NP_001161770.1 [human], NP_001003151.2 [canine], XP_005610662.1 [equine], XP_044890398.1 [feline], as well as sequence variants, isoforms encoded by alternative splicing, and various glycoforms thereof. Canonically, the protein encoded by this gene is a receptor for IL8 and transduces signaling to mediate neutrophil migration to sites of inflammation, among other activities. In some instances, and merely for the sake of disambiguation, a prefix is added when referring to the protein or gene of a particular species (with h, c, e, and f, referring to the human, canine, equine, and feline forms, respectively).

The term “CCR7 gene” refers to a mammalian gene encoding a C—C Motif Chemokine Receptor 7 polypeptide. Non-limiting examples of CCR7 genes include: NCBI Gene ID: 1236 [human], NCBI Gene ID: 491011 [canine], NCBI Gene ID: 100067673 [equine], NCBI Gene ID: 101084327 [feline], as well as synonymous and non-synonymous sequence variants thereof. Non-limiting examples of gene products encoded by a CCR7 gene include: UniProt: P32248; NP_001288643.1 [human], XP_038403305.1 [canine], XP_001500231.1 [equine], XP_003996882.1 [feline], as well as sequence variants, isoforms encoded by alternative splicing, and various glycoforms thereof. Canonically, the protein encoded by this gene controls the migration of memory T cells to inflamed tissues, as well as stimulate dendritic cell maturation. Signals mediated by this receptor may also function in chronic inflammation pathogenesis. In some instances, and merely for the sake of disambiguation, a prefix is added when referring to the protein or gene of a particular species (with h, c, e, and f, referring to the human, canine, equine, and feline forms, respectively).

The term “ADAM17 gene” refers to a mammalian gene encoding an ADAM Metallopeptidase Domain 17 polypeptide. Non-limiting examples of ADAM17 genes include: NCBI Gene ID: 6868 [human], NCBI Gene ID: 475662 [canine], NCBI Gene ID: 100072496 [equine], NCBI Gene ID: 101089004 [feline], as well as synonymous and non-synonymous sequence variants thereof. Non-limiting examples of gene products encoded by a ADAM17 gene include: UniProt: P78536; NP_001369706.1 [human], NP 001273795.1 [canine], NP_001295481.1 [equine], XP_003984558.1 [feline], as well as sequence variants, isoforms encoded by alternative splicing, and various glycoforms thereof. Canonically, the protein encoded by these genes is proteolytically processed to generate a mature protease, which functions by shedding the ectodomain of tumor necrosis factor-alpha, thereby releasing soluble tumor necrosis factor-alpha from its membrane-bound precursor. In some instances, and merely for the sake of disambiguation, a prefix is added when referring to the protein or gene of a particular species (with h, c, e, and f referring to the human, canine, equine, and feline forms, respectively).

The term “TNF gene” refers to a mammalian gene encoding a Tumor Necrosis Factor polypeptide. Non-limiting examples of TNF genes include: NCBI Gene ID: 7124 [human], NCBI Gene ID: 403922 [canine], NCBI Gene ID: 100033834 [equine], NCBI Gene ID: 493755 [feline], as well as synonymous and non-synonymous sequence variants thereof. Non-limiting examples of gene products encoded by a TNF gene include: UniProt: P01375; NP_000585.2 [human], NP_001003244.4 [canine], NP_001075288.2 [equine], NP_001009835.1 [feline], as well as sequence variants, isoforms encoded by alternative splicing, and various glycoforms thereof. Canonically, the protein encoded by these genes is a multifunctional proinflammatory cytokine that is mainly secreted by macrophages and can bind (and therefore function through) its receptors TNFRSF1A and TNFRSF1B. In some instances, and merely for the sake of disambiguation, a prefix is added when referring to the protein or gene of a particular species (with h, c, e, and f referring to the human, canine, equine, and feline forms, respectively).

The term “TNFRSF1A gene” refers to a mammalian gene encoding a Tumor Necrosis Factor Receptor 1 polypeptide. Non-limiting examples of TNFRSF1A genes include: NCBI Gene ID: 7132 [human], NCBI Gene ID: 403634 [canine], NCBI Gene ID: 100059548 [equine], NCBI Gene ID: 493957 [feline], as well as synonymous and non-synonymous sequence variants thereof. Non-limiting examples of gene products encoded by a TNFRSF1A gene include: UniProt: P19438; NP_001056.1 [human], XP_038295153.1 [canine], XP_023498787.1 [equine], NP_001009361.1 [feline], as well as sequence variants, isoforms encoded by alternative splicing, and various glycoforms thereof. Canonically, the proteins encoded by the genes listed above are transmembrane receptor proteins capable of binding Tumor Necrosis Factor Alpha (TNFA) or lymphotoxin alpha (LTA), its principal ligand. Upon binding to TNFA, the receptor trimerizes and is activated, transmitting intracellular signaling cascades with role in various processes, including apoptosis and inflammation. See generally, Ward-Kavanagh, L. K., et al. (2016). Immunity, 44(5), 1005-1019. In some instances, and merely for the sake of disambiguation, a prefix is added when referring to the protein or gene of a particular species (with h, c, e, and f referring to the human, canine, equine, and feline forms, respectively).

The term “TNFRSF1B gene” refers to a mammalian gene encoding a Tumor Necrosis Factor Receptor 2 polypeptide. Non-limiting examples of TNFRSF1B genes include: NCBI Gene ID: 7133 [human], NCBI Gene ID: 487437 [canine], NCBI Gene ID: 100055840 [equine], NCBI Gene ID: 101080392 [feline], as well as synonymous and non-synonymous sequence variants thereof. Non-limiting examples of gene products encoded by a TNFRSF1B gene include: UniProt: P20333; XP_011540362.1 [human], XP_038387905.1 [canine], XP_023491528.1 [equine], XP_023113905.2 [feline], as well as sequence variants, isoforms encoded by alternative splicing, and various glycoforms thereof. Canonically, the proteins encoded by the genes listed above are transmembrane receptor proteins capable of binding TNFA or LTA and are implicated in pro-survival pathways through downstream activation of NFkB pathway. See generally, Ward-Kavanagh, L. K., et al. (2016). Immunity. 44(5), 1005-1019. In some instances, and merely for the sake of disambiguation, a prefix is added when referring to the protein or gene of a particular species (with h, c, e, and f, referring to the human, canine, equine, and feline forms, respectively).

The term “IL4 gene” refers to a mammalian gene encoding an Interleukin 4 polypeptide. Non-limiting examples of IL4 genes include: NCBI Gene ID: 3565 [human], NCBI Gene ID: 403785 [canine], NCBI Gene ID: 100034225 [equine], NCBI Gene ID: 751514 [feline], as well as synonymous and non-synonymous sequence variants thereof. Non-limiting examples of gene products encoded by a IL4 gene include: UniProt: P05112; NP_000580.1 [human], NP 001003159.1 [canine], NP_001075988.1 [equine], NP_001036804.1 [feline], as well as sequence variants, isoforms encoded by alternative splicing, and various glycoforms thereof. Canonically, the protein encoded by these genes is a pleiotropic cytokine produced by activated T cells and is considered an important cytokine for tissue repair, counterbalancing the effects of proinflammatory type 1 cytokines, though it also promotes allergic airway inflammation and mediates acute inflammation, among other activities. In some instances, and merely for the sake of disambiguation, a prefix is added when referring to the protein or gene of a particular species (with h, c, e, and f, referring to the human, canine, equine, and feline forms, respectively).

The term “IL4R gene” refers to a mammalian gene encoding an Interleukin 4 Receptor polypeptide. Non-limiting examples of IL4R genes include: NCBI Gene ID: 3566 [human], NCBI Gene ID: 489957 [canine], NCBI Gene ID: 791252 [equine], NCBI Gene ID: 101096277 [feline], as well as synonymous and non-synonymous sequence variants thereof. Non-limiting examples of gene products encoded by a IL4R gene include: UniProt: P24394; NP_000409.1 [human], NP_001003159.1 [canine], XP_005598791.2 [equine], XP_023102076.2 [feline], as well as sequence variants, isoforms encoded by alternative splicing, and various glycoforms thereof. Canonically, the protein encoded by these genes is a type I transmembrane protein that can bind interleukin 4 and interleukin 13 to regulate IgE production and promote differentiation of Th2 cells, among other activities. In some instances, and merely for the sake of disambiguation, a prefix is added when referring to the protein or gene of a particular species (with h, c, e, and f referring to the human, canine, equine, and feline forms, respectively).

The term “IL6 gene” refers to a mammalian gene encoding an Interleukin 6 polypeptide. Non-limiting examples of IL6 genes include: NCBI Gene ID: 3569 [human], NCBI Gene ID: 403985 [canine], NCBI Gene ID: 100034196 [equine], NCBI Gene ID: 493687 [feline], as well as synonymous and non-synonymous sequence variants thereof. Non-limiting examples of gene products encoded by a IL6 gene include: UniProt: P05231; NP_000591.1 [human], NP_001003301.1 [canine], NP_001075965.2 [equine], NP_001009211.2 [feline], as well as sequence variants, isoforms encoded by alternative splicing, and various glycoforms thereof. Canonically, the protein encoded by these genes is a cytokine that functions in inflammation and the maturation of B cells that is primarily produced at sites of acute and chronic inflammation, where it is secreted into the serum and induces a transcriptional inflammatory response through interleukin 6 receptor. In some instances, and merely for the sake of disambiguation, a prefix is added when referring to the protein or gene of a particular species (with h, c, e, and f referring to the human, canine, equine, and feline forms, respectively).

The term “IL6R gene” refers to a mammalian gene encoding an Interleukin-6 Receptor polypeptide. Non-limiting examples of IL6R genes include: NCBI Gene ID: 3560 [human], NCBI Gene ID: 612271 [canine], NCBI Gene ID: 102148787 [equine], NCBI Gene ID: 101085689 [feline], as well as synonymous and non-synonymous sequence variants thereof. Non-limiting examples of gene products encoded by a IL6R gene include: UniProt: P08887; CAA41231.1 [human], XP_038527979.1 [canine], XP_023496854.1 [equine], XP_023103841.2 [feline], as well as sequence variants, isoforms encoded by alternative splicing, and various glycoforms thereof. Canonically, the proteins encoded by the genes listed above are transmembrane proteins capable of binding to interleukin-6, its native ligand. This binding event triggers intracellular signaling events that result in pro-inflammatory responses. See generally, Wolf, J., et al. (2014). Cytokine, 70(1), 11-20. In some instances, and merely for the sake of disambiguation, a prefix is added when referring to the protein or gene of a particular species (with h, c, e, and f referring to the human, canine, equine, and feline forms, respectively).

The term “IL6ST gene” refers to a mammalian gene encoding an Interleukin-6 Receptor polypeptide. Non-limiting examples of IL6ST genes include: NCBI Gene ID: 3572 [human], NCBI Gene ID: 403545 [canine], NCBI Gene ID: 100051700 [equine], NCBI Gene ID: 101089832 [feline], as well as synonymous and non-synonymous sequence variants thereof. Non-limiting examples of gene products encoded by a IL6ST gene include: UniProt: P40189 [human], A0A8I3QPC9 [canine], F7CFP8 [equine], M3WE28 [feline], as well as sequence variants, isoforms encoded by alternative splicing, and various glycoforms thereof. Canonically, the proteins encoded by the genes listed above are transmembrane proteins capable of binding to the interleukin-6 receptor (IL6R) when the latter is bound by its canonical interleukin-6 ligand (IL6). This binding event triggers intracellular signaling events that result in pro-inflammatory responses. See generally, Wolf, J., et al. (2014). Cytokine 70(1), 11-20. In some instances, and merely for the sake of disambiguation, a prefix is added when referring to the protein or gene of a particular species (with h, c, e, and f referring to the human, canine, equine, and feline forms, respectively).

The term “IL10 gene” refers to a mammalian gene encoding an Interleukin 10 polypeptide. Non-limiting examples of IL10 genes include: NCBI Gene ID: 3586 [human], NCBI Gene ID: 403628 [canine], NCBI Gene ID: 100034187 [equine], NCBI Gene ID: 493683 [feline], as well as synonymous and non-synonymous sequence variants thereof. Non-limiting examples of gene products encoded by a IL10 gene include: UniProt: P22301; NP_000563.1 [human], NP_001003077.1 [canine], NP_001075959.1 [equine], NP_001009209.1 [feline], as well as sequence variants, isoforms encoded by alternative splicing, and various glycoforms thereof. Canonically, the protein encoded by these genes is a pleiotropic cytokine that regulates inflammation and acts on many immune cell types through binding to its heterodimeric receptor composed of IL10RA and IL10RB, thereby activating downstream signaling cascades, such as the JAK-STAT pathway. In some instances, and merely for the sake of disambiguation, a prefix is added when referring to the protein or gene of a particular species (with h, c, e, and f referring to the human, canine, equine, and feline forms, respectively).

The term “IL10RA gene” refers to a mammalian gene encoding a Interleukin 10 Receptor Alpha polypeptide. Non-limiting examples of IL10RA genes include: NCBI Gene ID: 3587 [human], NCBI Gene ID: 610823 [canine], NCBI Gene ID: 100071172 [equine], NCBI Gene ID: 101087601 [feline], as well as synonymous and non-synonymous sequence variants thereof. Non-limiting examples of gene products encoded by a IL10RA gene include: UniProt: Q13651; NP_001549.2 [human], XP_038520677.1 [canine], XP_014596783.1 [equine], XP_003992449.1 [feline], as well as sequence variants, isoforms encoded by alternative splicing, and various glycoforms thereof. Canonically, the protein encoded by these genes is, upon forming a heterodimer with IL10RB, a regulator of pro-inflammatory signaling through the binding of its ligand IL-10. In some instances, and merely for the sake of disambiguation, a prefix is added when referring to the protein or gene of a particular species (with h, c, e, and f, referring to the human, canine, equine, and feline forms, respectively).

The term “IL10RB gene” refers to a mammalian gene encoding an Interleukin 10 Receptor Beta polypeptide. Non-limiting examples of IL10RB genes include: NCBI Gene ID: 3588 [human], NCBI Gene ID: 478404 [canine], NCBI Gene ID: 100052549 [equine], NCBI Gene ID: 101090038 [feline], as well as synonymous and non-synonymous sequence variants thereof. Non-limiting examples of gene products encoded by a IL10RB gene include: UniProt: Q08334; NP_000619.3 [human], XP_038299308.1 [canine], XP_023485821.1 [equine], XP_003991512.2 [feline], as well as sequence variants, isoforms encoded by alternative splicing, and various glycoforms thereof. Canonically, the protein encoded by these genes is, upon forming a heterodimer with IL10RA, a regulator of pro-inflammatory signaling through the binding of its ligand IL-10. In some instances, and merely for the sake of disambiguation, a prefix is added when referring to the protein or gene of a particular species (with h, c, e, and f, referring to the human, canine, equine, and feline forms, respectively).

The term “IL13 gene” refers to a mammalian gene encoding an Interleukin 13 polypeptide. Non-limiting examples of IL13 genes include: NCBI Gene ID: 3596 [human], NCBI Gene ID: 442990 [canine], NCBI Gene ID: 100034113 [equine], NCBI Gene ID: 101084678 [feline], as well as synonymous and non-synonymous sequence variants thereof. Non-limiting examples of gene products encoded by a IL13 gene include: UniProt: P35225; NP_001341920.1 [human], NP 001003384.1 [canine], NP_001137263.1 [equine], NP_001009209.1 [feline], as well as sequence variants, isoforms encoded by alternative splicing, and various glycoforms thereof. Canonically, the protein encoded by these genes regulates of the production of pro-inflammatory cytokines and chemokines. In some instances, and merely for the sake of disambiguation, a prefix is added when referring to the protein or gene of a particular species (with h, c, e, and f referring to the human, canine, equine, and feline forms, respectively).

The term “IL13RA1 gene” refers to a mammalian gene encoding an Interleukin 13 Receptor Alpha 1 polypeptide. Non-limiting examples of IL13RA1 genes include: NCBI Gene ID: 3597 [human], NCBI Gene ID: 403623 [canine], NCBI Gene ID: 100055312 [equine], NCBI Gene ID: 101091351 [feline], as well as synonymous and non-synonymous sequence variants thereof. Non-limiting examples of gene products encoded by a IL13RA1 gene include: UniProt: P78552; NP_001551.1 [human], XP_038306633.1 [canine], XP_023490026.1 [equine], XP_023104651.1 [feline], as well as sequence variants, isoforms encoded by alternative splicing, and various glycoforms thereof. Canonically, the protein encoded by these genes is a low affinity binding partner of IL13 and comprises a functional receptor once associated with of IL13RA2. Once bound to IL13, the receptor complex stimulates the production of pro-inflammatory cytokines and chemokines. In some instances, and merely for the sake of disambiguation, a prefix is added when referring to the protein or gene of a particular species (with h, c, e, and f referring to the human, canine, equine, and feline forms, respectively).

The term “IL13RA2 gene” refers to a mammalian gene encoding an Interleukin 13 Receptor Alpha 2 polypeptide. Non-limiting examples of IL13RA2 genes include: NCBI Gene ID: 3598 [human], NCBI Gene ID: 403622 [canine], NCBI Gene ID: 100057673 [equine], NCBI Gene ID: 101100114 [feline], as well as synonymous and non-synonymous sequence variants thereof. Non-limiting examples of gene products encoded by a IL13RA2 gene include: UniProt: Q14627; NP_000631.1 [human], NP_001003075.1 [canine], XP_023489189.1 [equine], XP_044906881.1 [feline], as well as sequence variants, isoforms encoded by alternative splicing, and various glycoforms thereof. Canonically, the protein encoded by these genes is a high affinity binding partner of IL13 but lacks a cytoplasmic domain. Along with IL13RA1, it forms a functional receptor that stimulates the production of pro-inflammatory cytokines and chemokines. In some instances, and merely for the sake of disambiguation, a prefix is added when referring to the protein or gene of a particular species (with h, c, e, and f referring to the human, canine, equine, and feline forms, respectively).

The term “IL17A gene” refers to a mammalian gene encoding an Interleukin 17A polypeptide. Non-limiting examples of IL17A genes include: NCBI Gene ID: 3605 [human], NCBI Gene ID: 481837 [canine], NCBI Gene ID: 100034142 [equine], NCBI Gene ID: 101095339 [feline], as well as synonymous and non-synonymous sequence variants thereof. Non-limiting examples of gene products encoded by a IL17A gene include: UniProt: Q16552; NP_002181.1 [human], NP_001159350.1 [canine], NP_001137264.1 [equine], XP_006931878.1 [feline], as well as sequence variants, isoforms encoded by alternative splicing, and various glycoforms thereof. Canonically, the protein encoded by these genes is an inflammatory cytokine that activates the NF kappa B signaling pathway through interactions with its heterodimeric receptor complex of IL17RA and IL17RC, thereby activating transcription of various chemokines, cytokines and other factors. In some instances, and merely for the sake of disambiguation, a prefix is added when referring to the protein or gene of a particular species (with h, c, e, and f referring to the human, canine, equine, and feline forms, respectively).

The term “IL17RA gene” refers to a mammalian gene encoding an Interleukin 17 Receptor A polypeptide. Non-limiting examples of IL17RA genes include: NCBI Gene ID: 23765 [human], NCBI Gene ID: 486759 [canine], NCBI Gene ID: 100055511 [equine], NCBI Gene ID: 101095588 [feline], as well as synonymous and non-synonymous sequence variants thereof. Non-limiting examples of gene products encoded by a IL17RA gene include: UniProt: Q96F46; NP_001276834.1 [human], XP_038295433.1 [canine], XP_005610881.1 [equine], XP_023112364.2 [feline], as well as sequence variants, isoforms encoded by alternative splicing, and various glycoforms thereof. Canonically, the protein encoded by these genes is a transmembrane protein that binds to IL17A with low affinity as part of a multimeric receptor complex. With its ligand, IL17RA is implicated in many inflammatory conditions. In some instances, and merely for the sake of disambiguation, a prefix is added when referring to the protein or gene of a particular species (with h, c, e, and f referring to the human, canine, equine, and feline forms, respectively).

The term “IL18 gene” refers to a mammalian gene encoding an Interleukin 18 polypeptide. Non-limiting examples of IL18 genes include: NCBI Gene ID: 3606 [human], NCBI Gene ID: 403796 [canine], NCBI Gene ID: 100034216 [equine], NCBI Gene ID: 493688 [feline], as well as synonymous and non-synonymous sequence variants thereof. Non-limiting examples of gene products encoded by a IL18 gene include: UniProt: Q14116; NP_001230140.1 [human], XP_038520002.1 [canine], XP_005611483.1 [equine], NP_001009213.2 [feline], as well as sequence variants, isoforms encoded by alternative splicing, and various glycoforms thereof. Canonically, the protein encoded by these genes is a pro-inflammatory cytokine that regulates inflammatory signaling through the NF kappa B pathway when engaged with its receptor and co-receptor, IL18R1 and IL18RAP. In some instances, and merely for the sake of disambiguation, a prefix is added when referring to the protein or gene of a particular species (with h, c, e, and f referring to the human, canine, equine, and feline forms, respectively).

The term “IL18R1 gene” refers to a mammalian gene encoding an Interleukin 18 Receptor 1 polypeptide. Non-limiting examples of IL18R1 genes include: NCBI Gene ID: 8809 [human], NCBI Gene ID: 611438 [canine], NCBI Gene ID: 100058269 [equine], NCBI Gene ID: 493938 [feline], as well as synonymous and non-synonymous sequence variants thereof. Non-limiting examples of gene products encoded by a IL18R1 gene include: UniProt: Q13478; NP_001269328.1 [human], XP_038536128.1 [canine], XP_023474273.1 [equine], NP_001009863.2 [feline], as well as sequence variants, isoforms encoded by alternative splicing, and various glycoforms thereof. Canonically, the protein encoded by these genes is an essential component for transducing IL18-mediated pro-inflammatory signaling. In some instances, and merely for the sake of disambiguation, a prefix is added when referring to the protein or gene of a particular species (with h, c, e, and f, referring to the human, canine, equine, and feline forms, respectively).

The term “IL18RAP gene” refers to a mammalian gene encoding an Interleukin 18 Receptor Accessory Protein polypeptide. Non-limiting examples of IL18RAP genes include: NCBI Gene ID: 8807 [human], NCBI Gene ID: 481327 [canine], NCBI Gene ID: 100050212 [equine], NCBI Gene ID: 101084868 [feline], as well as synonymous and non-synonymous sequence variants thereof. Non-limiting examples of gene products encoded by a IL18RAP gene include: UniProt: Q53TU5; NP_001380415.1 [human], XP_038536125.1 [canine], XP_014586460.1 [equine], XP_019682529.2 [feline], as well as sequence variants, isoforms encoded by alternative splicing, and various glycoforms thereof. Canonically, the protein encoded by these genes is an accessory protein that enhances the signal transduction of IL18-mediated pro-inflammatory signaling. In some instances, and merely for the sake of disambiguation, a prefix is added when referring to the protein or gene of a particular species (with h, c, e, and f, referring to the human, canine, equine, and feline forms, respectively).

The term “NGF gene” refers to a mammalian gene encoding a Nerve Growth Factor polypeptide. Non-limiting examples of NGF genes include: NCBI Gene ID: 4803 [human], NCBI Gene ID: 403402 [canine], NCBI Gene ID: 100065669 [equine], NCBI Gene ID: 100144611 [feline], as well as synonymous and non-synonymous sequence variants thereof. Non-limiting examples of gene products encoded by a NGF gene include: UniProt: P01138; NP_002497.2 [human], XP_038546347.1 [canine], XP_001496237.2 [equine], XP_044889256.1 [feline], as well as sequence variants, isoforms encoded by alternative splicing, and various glycoforms thereof. Canonically, this secreted protein forms a functional homodimer that is incorporated into a larger complex and has nerve growth stimulating activity. The complex is also involved in the regulation of growth and the differentiation of sympathetic and certain sensory neurons. In some instances, and merely for the sake of disambiguation, a prefix is added when referring to the protein or gene of a particular species (with h, c, e, and f, referring to the human, canine, equine, and feline forms, respectively).

The term “NGFR gene” refers to a mammalian gene encoding a Nerve Growth Factor Recepto polypeptide. Non-limiting examples of NGFR genes include: NCBI Gene ID: 4804 [human], NCBI Gene ID: 491071 [canine], NCBI Gene ID: 100069694 [equine], NCBI Gene ID: 101101519 [feline], as well as synonymous and non-synonymous sequence variants thereof. Non-limiting examples of gene products encoded by a NGFR gene include: UniProt: P08138; NP_002498.1 [human], XP_038531049.1 [canine], XP_023508464.1 [equine], XP_023099534.1 [feline], as well as sequence variants, isoforms encoded by alternative splicing, and various glycoforms thereof. Canonically, the protein encoded by these genes contains four 40-amino acid repeats within its extracellular domain with 6 cysteine residues at conserved positions followed by a serine/threonine-rich region. This cysteine-rich region contains the nerve growth factor binding domain and allows for signal transduction once bound. In some instances, and merely for the sake of disambiguation, a prefix is added when referring to the protein or gene of a particular species (with h, c, e, and f, referring to the human, canine, equine, and feline forms, respectively).

The term “NTF3 gene” refers to a mammalian gene encoding a Neurotrophin-3 polypeptide. Non-limiting examples of NTF3 genes include: NCBI Gene ID: 4908 [human], NCBI Gene ID: 493963 [canine], NCBI Gene ID: 100051839 [equine], NCBI Gene ID: 486731 [feline], as well as synonymous and non-synonymous sequence variants thereof. Non-limiting examples of gene products encoded by a NTF3 gene include: UniProt: P20783; NP_001096124.1 [human], XP_038293846.1 [canine], XP_023498780.1 [equine], NP_001009367.1 [feline], as well as sequence variants, isoforms encoded by alternative splicing, and various glycoforms thereof. Canonically, the protein encoded by these genes controls survival and differentiation of neurons. In some instances, and merely for the sake of disambiguation, a prefix is added when referring to the protein or gene of a particular species (with h, c, e, and f, referring to the human, canine, equine, and feline forms, respectively).

The term “NTF4 gene” refers to a mammalian gene encoding a Neurotrophin-4 polypeptide. Non-limiting examples of NTF4 genes include: NCBI Gene ID: 4909 [human], NCBI Gene ID: 611987 [canine], NCBI Gene ID: 100054859 [equine], NCBI Gene ID: 101100428 [feline], as well as synonymous and non-synonymous sequence variants thereof. Non-limiting examples of gene products encoded by a NTF4 gene include: UniProt: P34130; NP_001382418.1 [human], NP 001177358.2 [canine], XP_023505846.1 [equine], XP_023101354.2 [feline], as well as sequence variants, isoforms encoded by alternative splicing, and various glycoforms thereof. Canonically, the protein encoded by these genes is proteolytically processed to a mature form, which can promote survival of neurons through binding of its cognate receptor. Dysregulation of this protein is observed in various neurological disorders. In some instances, and merely for the sake of disambiguation, a prefix is added when referring to the protein or gene of a particular species (with h, c, e, and f, referring to the human, canine, equine, and feline forms, respectively).

The term “NTRK1 gene” refers to a mammalian gene encoding a Neurotrophic Receptor Tyrosine Kinase 1 polypeptide. Non-limiting examples of NTRK1 genes include: NCBI Gene ID: 4914 [human], NCBI Gene ID: 490404 [canine], NCBI Gene ID: 100064594 [equine], NCBI Gene ID: 101081603 [feline], as well as synonymous and non-synonymous sequence variants thereof. Non-limiting examples of gene products encoded by a NTRK11 gene include: UniProt: P04629; NP_001007793.1 [human], XP_038527745.1 [canine], XP_023496742.1 [equine], XP_023103311.1 [feline], as well as sequence variants, isoforms encoded by alternative splicing, and various glycoforms thereof. Canonically, the protein encoded by these genes is a membrane-bound receptor that binds neutrophin and signals through the MAPK pathway to regulate cell differentiation, among other functions. In some instances, and merely for the sake of disambiguation, a prefix is added when referring to the protein or gene of a particular species (with h, c, e, and f, referring to the human, canine, equine, and feline forms, respectively).

The term “NTRK2 gene” refers to a mammalian gene encoding a Neurotrophic Receptor Tyrosine Kinase 2 polypeptide. Non-limiting examples of NTRK2 genes include: NCBI Gene ID: 4915 [human], NCBI Gene ID: 484147 [canine], NCBI Gene ID: 100061700 [equine], NCBI Gene ID: 101101347 [feline], as well as synonymous and non-synonymous sequence variants thereof. Non-limiting examples of gene products encoded by a NTRK2 gene include: UniProt: Q16620; NP_001007098.1 [human], XP_038510982.1 [canine], XP_023482906.1 [equine], XP_023097987.1 [feline], as well as sequence variants, isoforms encoded by alternative splicing, and various glycoforms thereof. Canonically, the protein encoded by these genes is a membrane-bound receptor that binds neutrophin and signals through the MAPK pathway to regulate cell differentiation, among other functions. In some instances, and merely for the sake of disambiguation, a prefix is added when referring to the protein or gene of a particular species (with h, c, e, and f referring to the human, canine, equine, and feline forms, respectively).

The term “BDNF gene” refers to a mammalian gene encoding a Brain-Derived Neurotrophic Factor polypeptide. Non-limiting examples of BDNF genes include: NCBI Gene ID: 627 [human], NCBI Gene ID: 403461 [canine], NCBI Gene ID: 100009689 [equine], NCBI Gene ID: 493690 [feline], as well as synonymous and non-synonymous sequence variants thereof. Non-limiting examples of gene products encoded by a BDNF gene include: UniProt: P23560; NP_001137277.1 [human], NP_001002975.1 [canine], NP_001075256.1 [equine], NP_001009828.1 [feline], as well as sequence variants, isoforms encoded by alternative splicing, and various glycoforms thereof. Canonically, the protein encoded by these genes is proteolytically processed to a mature form, which can promote survival of neurons through binding of its cognate receptor. Dysregulation of this protein is observed in various neurological disorders. In some instances, and merely for the sake of disambiguation, a prefix is added when referring to the protein or gene of a particular species (with h, c, e, and f, referring to the human, canine, equine, and feline forms, respectively).

The term “SCN1A gene” refers to a mammalian gene encoding a Sodium Voltage-Gated Channel Alpha 1 polypeptide. Non-limiting examples of SCN1A genes include: NCBI Gene ID: 6323 [human], NCBI Gene ID: 478775 [canine], NCBI Gene ID: 100052059 [equine], NCBI Gene ID: 101081823 [feline], as well as synonymous and non-synonymous sequence variants thereof. Non-limiting examples of gene products encoded by a SCN1A gene include: UniProt: P35498; NP_001159435.1 [human], XP_038302870.1 [canine], XP_023478839.1 [equine], XP_019693764.1 [feline], as well as sequence variants, isoforms encoded by alternative splicing, and various glycoforms thereof. Canonically, the protein encoded by these genes mediates the voltage-dependent sodium ion permeability of excitable membranes and is involved in sensory perception of mechanical pain (i.e., activation in somatosensory neurons has been shown to induce pain without neurogenic inflammation). In some instances, and merely for the sake of disambiguation, a prefix is added when referring to the protein or gene of a particular species (with h, c, e, and f, referring to the human, canine, equine, and feline forms, respectively).

The term “SCN2A gene” refers to a mammalian gene encoding a Sodium Voltage-Gated Channel Alpha 2 polypeptide. Non-limiting examples of SCN2A genes include: NCBI Gene ID: 6326 [human], NCBI Gene ID: 478773 [canine], NCBI Gene ID: 100051816 [equine], NCBI Gene ID: 101080472 [feline], as well as synonymous and non-synonymous sequence variants thereof. Non-limiting examples of gene products encoded by a SCN2A gene include: UniProt: Q99250; NP_001035232.1 [human], XP_038302857.1 [canine], XP_023478830.1 [equine], XP_023115179.1 [feline], as well as sequence variants, isoforms encoded by alternative splicing, and various glycoforms thereof. Canonically, the protein encoded by these genes mediates the voltage-dependent sodium ion permeability of excitable membranes. In some instances, and merely for the sake of disambiguation, a prefix is added when referring to the protein or gene of a particular species (with h, c, e, and f referring to the human, canine, equine, and feline forms, respectively).

The term “SCN3A gene” refers to a mammalian gene encoding a Sodium Voltage-Gated Channel Alpha 3 polypeptide. Non-limiting examples of SCN3A genes include: NCBI Gene ID: 6328 [human], NCBI Gene ID: 478772 [canine], NCBI Gene ID: 100061941 [equine], NCBI Gene ID: 101082587 [feline], as well as synonymous and non-synonymous sequence variants thereof. Non-limiting examples of gene products encoded by a SCN3A gene include: UniProt: Q9NY46; NP_001075145.1 [human], XP_038302852.1 [canine], XP_023478823.1 [equine], XP_019693750.1 [feline], as well as sequence variants, isoforms encoded by alternative splicing, and various glycoforms thereof. Canonically, the protein encoded by these genes is a subunit of voltage-gated sodium channels and is responsible for propagation of action potentials in neurons and muscle tissue. In some instances, and merely for the sake of disambiguation, a prefix is added when referring to the protein or gene of a particular species (with h, c, e, and f, referring to the human, canine, equine, and feline forms, respectively).

The term “SCN4A gene” refers to a mammalian gene encoding a Sodium Voltage-Gated Channel Alpha 4 polypeptide. Non-limiting examples of SCN4A genes include: NCBI Gene ID: 6328 [human], NCBI Gene ID: 119873250 [canine], NCBI Gene ID: 100049793 [equine], NCBI Gene ID: 101098669 [feline], as well as synonymous and non-synonymous sequence variants thereof. Non-limiting examples of gene products encoded by a SCN4A gene include: UniProt: Q9NY46; NP_001075145.1 [human], XP_038531923.1 [canine], NP_001075230.2 [equine], XP_006940553.1 [feline], as well as sequence variants, isoforms encoded by alternative splicing, and various glycoforms thereof. Canonically, the protein encoded by these genes is a subunit of voltage-gated sodium channels and is responsible for propagation of action potentials in neurons and muscle tissue. In some instances, and merely for the sake of disambiguation, a prefix is added when referring to the protein or gene of a particular species (with h, c, e, and f, referring to the human, canine, equine, and feline forms, respectively).

The term “SCN5A gene” refers to a mammalian gene encoding a Sodium Voltage-Gated Channel Alpha 5 polypeptide. Non-limiting examples of SCN5A genes include: NCBI Gene ID: 6331 [human], NCBI Gene ID: 403497 [canine], NCBI Gene ID: 100034027 [equine], NCBI Gene ID: 101100994 [feline], as well as synonymous and non-synonymous sequence variants thereof. Non-limiting examples of gene products encoded by a SCN5A gene include: UniProt: Q14524; NP_000326.2 [human], NP_001002994.1 [canine], NP_001157367.1 [equine], XP_044893792.1 [feline], as well as sequence variants, isoforms encoded by alternative splicing, and various glycoforms thereof. Canonically, the protein encoded by these genes is a subunit of voltage-gated sodium channels and is found primarily in cardiac muscle and is responsible for the initial upstroke of the action potential in an electrocardiogram. In some instances, and merely for the sake of disambiguation, a prefix is added when referring to the protein or gene of a particular species (with h, c, e, and f, referring to the human, canine, equine, and feline forms, respectively).

The term “SCN8A gene” refers to a mammalian gene encoding a Sodium Voltage-Gated Channel Alpha 8 polypeptide. Non-limiting examples of SCN8A genes include: NCBI Gene ID: 6335 [human], NCBI Gene ID: 477604 [canine], NCBI Gene ID: 100052777 [equine], NCBI Gene ID: 101096578 [feline], as well as synonymous and non-synonymous sequence variants thereof. Non-limiting examples of gene products encoded by a SCN8A gene include: UniProt: Q9UQD0; NP_001171455.1 [human], XP_038294063.1 [canine], XP_023499351.1 [equine], XP_023112849.1 [feline], as well as sequence variants, isoforms encoded by alternative splicing, and various glycoforms thereof. Canonically, the protein encoded by these genes is the ion pore subunit of the voltage-gated sodium channel and is essential for rapid membrane depolarization during neuronal action potentials. In some instances, and merely for the sake of disambiguation, a prefix is added when referring to the protein or gene of a particular species (with h, c, e, and f, referring to the human, canine, equine, and feline forms, respectively).

The term “SCN9A gene” refers to a mammalian gene encoding a Sodium Voltage-Gated Channel Alpha 9 polypeptide. Non-limiting examples of SCN9A genes include: NCBI Gene ID: 6335 [human], NCBI Gene ID: 100855710 [canine], NCBI Gene ID: 100052120 [equine], NCBI Gene ID: 101082841 [feline], as well as synonymous and non-synonymous sequence variants thereof. Non-limiting examples of gene products encoded by a SCN9A gene include: UniProt: Q15858; NP_001352465.1 [human], XP_038302872.1 [canine], XP_023478844.1 [equine], XP_044889827.1 [feline], as well as sequence variants, isoforms encoded by alternative splicing, and various glycoforms thereof. Canonically, the protein encoded by these genes is a voltage-dependent sodium ion channel that has been associated with various pain disorders, especially in the development of inflammatory pain. In some instances, and merely for the sake of disambiguation, a prefix is added when referring to the protein or gene of a particular species (with h, c, e, and f, referring to the human, canine, equine, and feline forms, respectively).

The term “SCN10A gene” refers to a mammalian gene encoding a Sodium Voltage-Gated Channel Alpha 10 polypeptide. Non-limiting examples of SCN10A genes include: NCBI Gene ID: 6336 [human], NCBI Gene ID: 477026 [canine], NCBI Gene ID: 100055493 [equine], NCBI Gene ID: 101085569 [feline], as well as synonymous and non-synonymous sequence variants thereof. Non-limiting examples of gene products encoded by a SCN10A gene include: UniProt: Q9Y5Y9; NP_001280235.2 [human], NP_001003203.1 [canine], XP_014587037.1 [equine], XP_044893784.1 [feline], as well as sequence variants, isoforms encoded by alternative splicing, and various glycoforms thereof. Canonically, the protein encoded by these genes is a membrane-spanning subunit of voltage-dependent sodium channels that may be involved in the onset of pain associated with neuropathies. In some instances, and merely for the sake of disambiguation, a prefix is added when referring to the protein or gene of a particular species (with h, c, e, and f, referring to the human, canine, equine, and feline forms, respectively).

The term “SCN11A gene” refers to a mammalian gene encoding a Sodium Voltage-Gated Channel Alpha 11 polypeptide. Non-limiting examples of SCN11A genes include: NCBI Gene ID: 11280 [human], NCBI Gene ID: 485593 [canine], NCBI Gene ID: 100068480 [equine], NCBI Gene ID: 101085312 [feline], as well as synonymous and non-synonymous sequence variants thereof. Non-limiting examples of gene products encoded by a SCN11A gene include: UniProt: Q9UI33; NP_001336182.1 [human], XP038426400.1 [canine], XP_001916634.3 [equine], XP_044893782.1 [feline], as well as sequence variants, isoforms encoded by alternative splicing, and various glycoforms thereof. Canonically, the protein encoded by these genes is a membrane-spanning subunit of voltage-dependent sodium channels and is highly expressed in nociceptive neurons of dorsal root ganglia and trigeminal ganglia. Mutations in the SCN11A gene have been associated with various pain disorders. In some instances, and merely for the sake of disambiguation, a prefix is added when referring to the protein or gene of a particular species (with h, c, e, and f, referring to the human, canine, equine, and feline forms, respectively).

The term “TAC1 gene” refers to a mammalian gene encoding a Tachykinin Precursor 1 polypeptide. Non-limiting examples of TAC1 genes include: NCBI Gene ID: 6863 [human], NCBI Gene ID: 475239 [canine], NCBI Gene ID: 100052324 [equine], NCBI Gene ID: 101095481 [feline], as well as synonymous and non-synonymous sequence variants thereof. Non-limiting examples of gene products encoded by a TAC1 gene include: UniProt: P20366; NP_003173.1 [human], XP_038541905.1 [canine], XP_014594521.1 [equine], XP_003982840.1 [feline], as well as sequence variants, isoforms encoded by alternative splicing, and various glycoforms thereof. Canonically, the protein encoded by these genes is a precursor for four products of the tachykinin peptide hormone family-substance P, neurokinin A, neuropeptide K and neuropeptide gamma. These hormones are thought to function as neurotransmitters that interact with nerve receptors and smooth muscle cells. In some instances, and merely for the sake of disambiguation, a prefix is added when referring to the protein or gene of a particular species (with h, c, e, and f, referring to the human, canine, equine, and feline forms, respectively).

The term “TAC3 gene” refers to a mammalian gene encoding a Tachykinin Precursor 3 polypeptide. Non-limiting examples of TAC3 genes include: NCBI Gene ID: 6866 [human], NCBI Gene ID: 607315 [canine], NCBI Gene ID: 100052722 [equine], NCBI Gene ID: 101089368 [feline], as well as synonymous and non-synonymous sequence variants thereof. Non-limiting examples of gene products encoded by a TAC3 gene include: UniProt: Q9UHF0; NP_001171525.1 [human], UniProt: A0A8I3N7Z8; NP_001362511.2 canine], XP_023499603.1 [equine], XP_019690663.1 [feline], as well as sequence variants, isoforms encoded by alternative splicing, and various glycoforms thereof. Canonically, the protein encoded by these genes is proteolytically processed to generate a mature peptide, which is primarily expressed in the central and peripheral nervous systems and functions as a neurotransmitter. This peptide is the ligand for the neurokinin-3 receptor. These hormones are thought to function as neurotransmitters that interact with nerve receptors and smooth muscle cells. In some instances, and merely for the sake of disambiguation, a prefix is added when referring to the protein or gene of a particular species (with h, c, e, and f referring to the human, canine, equine, and feline forms, respectively).

The term “TACR1 gene” refers to a mammalian gene encoding a Tachykinin Receptor 1 polypeptide. Non-limiting examples of TACR1 genes include: NCBI Gene ID: 6869 [human], NCBI Gene ID: 403815 [canine], NCBI Gene ID: 100053491 [equine], NCBI Gene ID: 101090094 [feline], as well as synonymous and non-synonymous sequence variants thereof. Non-limiting examples of gene products encoded by a TACR1 gene include: UniProt: P25103; NP_001049.1 [human], NP_001012637.1 canine], XP_001499730.1 [equine], XP_003984209.1 [feline], as well as sequence variants, isoforms encoded by alternative splicing, and various glycoforms thereof. Canonically, the protein encoded by these genes is the receptor for the tachykinin substance P, also referred to as neurokinin 1. TACR1 activates a phosphatidylinositol-calcium second messenger system and can also bind substance K and neuromedin-K with less affinity. In some instances, and merely for the sake of disambiguation, a prefix is added when referring to the protein or gene of a particular species (with h, c, e, and f referring to the human, canine, equine, and feline forms, respectively).

The term “TACR2 gene” refers to a mammalian gene encoding a Tachykinin Receptor 2 polypeptide. Non-limiting examples of TACR2 genes include: NCBI Gene ID: 6865 [human], NCBI Gene ID: 489020 [canine], NCBI Gene ID: 100034168 [equine], NCBI Gene ID: 101094541 [feline]], as well as synonymous and non-synonymous sequence variants thereof. Non-limiting examples of gene products encoded by a TACR2 gene include: UniProt: P21452; NP_001048.2 [human], NP 001012635.1 [canine], XP_001502752.2 [equine], XP_044896003.1 [feline], as well as sequence variants, isoforms encoded by alternative splicing, and various glycoforms thereof. Canonically, the protein encoded by these genes is the receptor for the tachykinin substance K, also referred to as neurokinin A. TACR2 activates a phosphatidylinositol-calcium second messenger system and can also bind neuromedin-K and substance P with less affinity. In some instances, and merely for the sake of disambiguation, a prefix is added when referring to the protein or gene of a particular species (with h, c, e, and f referring to the human, canine, equine, and feline forms, respectively).

The term “TACR3 gene” refers to a mammalian gene encoding a Tachykinin Receptor 3 polypeptide. Non-limiting examples of TACR3 genes include: NCBI Gene ID: 6870 [human], NCBI Gene ID: 403814 [canine], NCBI Gene ID: 100073088 [equine], NCBI Gene ID: 101093603 [feline], as well as synonymous and non-synonymous sequence variants thereof. Non-limiting examples of gene products encoded by a TACR3 gene include: UniProt: P29371; NP_001050.1 [human], NP_001091010.1 [canine], XP_023492571.1 [equine], XP_003985169.3 [feline], as well as sequence variants, isoforms encoded by alternative splicing, and various glycoforms thereof. Canonically, the protein encoded by these genes is the receptor for the tachykinin neurokinin 3, also referred to as neurokinin B or neuromedin-K. TACR3 activates a phosphatidylinositol-calcium second messenger system and can also bind substance K and substance P with less affinity. In some instances, and merely for the sake of disambiguation, a prefix is added when referring to the protein or gene of a particular species (with h, c, e, and f referring to the human, canine, equine, and feline forms, respectively).

The term “MRGPRX2 gene” refers to a mammalian gene encoding a MAS related GPR family member X2 polypeptide. Non-limiting examples of MRGPRX2 genes include: NCBI Gene ID: 117194 [human], NCBI Gene ID: 485410 [canine], NCBI Gene ID: 100071950 [equine], NCBI Gene ID: 101097092 [feline]) or an encoded gene product (e.g., UniProt: Q96LB1; NP_001290544.1 [human], XP_038285538.1 [canine], XP_023501936.1 [equine], XP_003993155.1 [feline], as well as sequence variants, isoforms encoded by alternative splicing, and various glycoforms thereof. Canonically, the protein encoded by these genes enables G protein-coupled receptor activity and neuropeptide binding activity and is involved in mast cell degranulation and positive regulation of cytokinesis. In some instances, and merely for the sake of disambiguation, a prefix is added when referring to the protein or gene of a particular species (with h, c, e, and f referring to the human, canine, equine, and feline forms, respectively).

The term “ATP1A1 gene” refers to a mammalian gene encoding a ATPase Na+/K+ transporting subunit alpha 1 polypeptide. Non-limiting examples of ATP1A1 genes include: NCBI Gene ID: 476 [human], NCBI Gene ID: 403992 [canine], NCBI Gene ID: 100034139 [equine], NCBI Gene ID: 101083695 [feline], as well as synonymous and non-synonymous sequence variants thereof. Non-limiting examples of gene products encoded by a ATP1A1 gene include: UniProt: P05023; NP_000692.2 [human], NP_001376153.1 [canine], NP_001108004.2 [equine], XP_011283388.1 [feline], as well as sequence variants, isoforms encoded by alternative splicing, and various glycoforms thereof. Canonically, the protein encoded by these genes is an integral membrane protein subunit of the complex responsible for establishing and maintaining the electrochemical gradients of Na and K ions across a plasma membrane, which is essential for osmoregulation and electrical excitability of nerve and muscle. In some instances, and merely for the sake of disambiguation, a prefix is added when referring to the protein or gene of a particular species (with h, c, e, and f referring to the human, canine, equine, and feline forms, respectively).

The term “CALCA gene” refers to a mammalian gene encoding a Calcitonin Related Polypeptide Alpha polypeptide. Non-limiting examples of CALCA genes include: NCBI Gene ID: 796 [human], NCBI Gene ID: 403946 [canine], NCBI Gene ID: 100033906 [equine], NCBI Gene ID: 101095582 [feline], as well as synonymous and non-synonymous sequence variants thereof. Non-limiting examples of gene products encoded by a CALCA gene include: UniProt: P01258; NP_001029124.1 [human], NP 001300719.1 [canine], NP_001075323.1 [equine], XP_019667660.1 [feline], as well as sequence variants, isoforms encoded by alternative splicing, and various glycoforms thereof. Canonically, this gene encodes multiple gene products, such as calcitonin, calcitonin gene-related peptide and katacalcin, through tissue-specific alternative RNA splicing of the gene transcripts and cleavage of inactive precursor proteins. The proteins are involved in calcium regulation, regulate phosphorus metabolism, and function as a vasodilator, among other functions. In some instances, and merely for the sake of disambiguation, a prefix is added when referring to the protein or gene of a particular species (with h, c, e, and f referring to the human, canine, equine, and feline forms, respectively).

The term “CALCB gene” refers to a mammalian gene encoding a Calcitonin Related Polypeptide Beta polypeptide. Non-limiting examples of CALCB genes include: NCBI Gene ID: 797 [human], NCBI Gene ID: 403415 [canine], NCBI Gene ID: 100034126 [equine], NCBI Gene ID: 101094539 [feline], as well as synonymous and non-synonymous sequence variants thereof. Non-limiting examples of gene products encoded by a CALCB gene include: UniProt: P10092; NP_000719.1 [human], NP_001002948.1 [canine], NP_001075397.1 [equine], XP_044894937.1 [feline], as well as sequence variants, isoforms encoded by alternative splicing, and various glycoforms thereof. Canonically, the protein encoded by these genes acts as a vasodilator and a neurotransmitter, among other functions. In some instances, and merely for the sake of disambiguation, a prefix is added when referring to the protein or gene of a particular species (with h, c, e, and f referring to the human, canine, equine, and feline forms, respectively).

The term “CALCRL gene” refers to a mammalian gene encoding a Calcitonin Receptor Like Receptor polypeptide. Non-limiting examples of CALCRL genes include: NCBI Gene ID: 10203 [human], NCBI Gene ID: 488438 [canine], NCBI Gene ID: 100054281 [equine], NCBI Gene ID: 101086333 [feline], as well as synonymous and non-synonymous sequence variants thereof. Non-limiting examples of gene products encoded by a CALCRL gene include: UniProt: Q16602; NP_001258680.1 [human], XP_038303202.1 [canine], XP_023477941.1 [equine]. XP_011283721.1 [feline], as well as sequence variants, isoforms encoded by alternative splicing, and various glycoforms thereof. Canonically, the protein encoded by these genes comprises the receptor for CGRP (with RAMP1) and receptor for ADM (with RAMP2/3) and activates adenylyl cyclase. In some instances, and merely for the sake of disambiguation, a prefix is added when referring to the protein or gene of a particular species (with h, c, e, and f, referring to the human, canine, equine, and feline forms, respectively).

The term “RAMP1 gene” refers to a mammalian gene encoding a Receptor Activity Modifying Protein 1 polypeptide. Non-limiting examples of RAMP1 genes include: NCBI Gene ID: 10267 [human], NCBI Gene ID: 607163 [canine], NCBI Gene ID: 100066550 [equine], NCBI Gene ID: 101092133 [feline], as well as synonymous and non-synonymous sequence variants thereof. Non-limiting examples of gene products encoded by a RAMP1 gene include: UniProt: 060894; NP_005846.1 [human], XP_038291846.1 [canine], XP_023498460.1 [equine], XP_044890618.1 [feline], as well as sequence variants, isoforms encoded by alternative splicing, and various glycoforms thereof. Canonically, the protein encoded by these genes is required to transport calcitonin-receptor-like receptor (CRLR) to the plasma membrane and, with CRLR, functions as a CGRP receptor. In some instances, and merely for the sake of disambiguation, a prefix is added when referring to the protein or gene of a particular species (with h, c, e, and f, referring to the human, canine, equine, and feline forms, respectively).

The term “ADM gene” refers to a mammalian gene encoding an Adrenomedullin polypeptide. Non-limiting examples of ADM genes include: NCBI Gene ID: 133 [human], NCBI Gene ID: 403817 [canine], NCBI Gene ID: 100033857 [equine], NCBI Gene ID: 101087095 [feline], as well as synonymous and non-synonymous sequence variants thereof. Non-limiting examples of gene products encoded by a ADM gene include: UniProt: P35318; NP_001115.1 [human], NP_001003183.1 [canine], NP_001157351.1 [equine], XP_044894880.1 [feline], as well as sequence variants, isoforms encoded by alternative splicing, and various glycoforms thereof. Canonically, the protein encoded by this gene is a 52 aa peptide with several functions, including vasodilation, regulation of hormone secretion, promotion of angiogenesis. In some instances, and merely for the sake of disambiguation, a prefix is added when referring to the protein or gene of a particular species (with h, c, e, and f, referring to the human, canine, equine, and feline forms, respectively).

The term “CRCP gene” refers to a mammalian gene encoding a CGRP Receptor Component polypeptide. Non-limiting examples of CRCP genes include: NCBI Gene ID: 27297 [human], NCBI Gene ID: 479705 [canine], NCBI Gene ID: 100061681 [equine], NCBI Gene ID: 101084503 [feline], as well as synonymous and non-synonymous sequence variants thereof. Non-limiting examples of gene products encoded by a CRCP gene include: UniProt: 075575; NP_001035737.1 [human], XP_038523718.1 [canine], XP_001493592.3 [equine], XP_044903465.1 [feline], as well as sequence variants, isoforms encoded by alternative splicing, and various glycoforms thereof. Canonically, the protein encoded by this gene is an accessory protein for the CGRP receptor that modulates CGRP responsiveness in a variety of tissues. In some instances, and merely for the sake of disambiguation, a prefix is added when referring to the protein or gene of a particular species (with h, c, e, and f, referring to the human, canine, equine, and feline forms, respectively).

The term “YAP1 gene” refers to a mammalian gene encoding a Yes1-Associated Protein polypeptide. Non-limiting examples of YAP1 genes include: NCBI Gene ID: 10413 [human], NCBI Gene ID: 479465 [canine], NCBI Gene ID: 100068834 [equine], NCBI Gene ID: 101101408 [feline], as well as synonymous and non-synonymous sequence variants thereof. Non-limiting examples of gene products encoded by a YAP1 gene include: UniProt: P46937; NP_001123617.1 [human], XP_038521022.1 [canine], XP_023500466.1 [equine], XP_044894121.1 [feline], as well as sequence variants, isoforms encoded by alternative splicing, and various glycoforms thereof. Canonically, the protein encoded by this gene is involved in development, growth, repair and homeostasis. In some instances, and merely for the sake of disambiguation, a prefix is added when referring to the protein or gene of a particular species (with h, c, e, and f, referring to the human, canine, equine, and feline forms, respectively).

The term “IL1RAP gene” refers to a mammalian gene encoding an Interleukin 1 Receptor Accessory Protein polypeptide. Non-limiting examples of ILRAP1 genes include: NCBI Gene ID: 3556 [human], NCBI Gene ID: 488126 [canine], NCBI Gene ID: 100068726 [equine], NCBI Gene ID: 101094125 [felinE], as well as synonymous and non-synonymous sequence variants thereof. Non-limiting examples of gene products encoded by a IL1RAP gene include: UniProt: Q9NPH3; NP_002173.1 [human], XP_038318680.1 [canine], XP_001498597.2 [equine], XP_044893081.1 [feline], as well as sequence variants, isoforms encoded by alternative splicing, and various glycoforms thereof. Canonically, the proteins encoded by the genes listed above are capable of associating with IL1R1 bound to IL1 to form the high affinity interleukin-1 receptor complex that mediates interleukin-1-dependent activation of NF-kappa-B and other signaling pathways through the recruitment of adapter molecules such as TOLLIP, MYD88, and IRAK1 or IRAK2 via TIR-TIR interactions with the cytoplasmic domains of receptor/coreceptor subunits. In some instances, and merely for the sake of disambiguation, a prefix is added when referring to the protein or gene of a particular species (with h, c, e, and f, referring to the human, canine, equine, and feline forms, respectively).

The term “IL1R1 gene” refers to a mammalian gene encoding an Interleukin I receptor type 1 polypeptide. Non-limiting examples of IL1R1 genes include: NCBI Gene ID: 3554 [human], NCBI Gene ID: 481328 [canine], NCBI Gene ID: 100009699 [equine], NCBI Gene ID: 101080705 [feline], as well as synonymous and non-synonymous sequence variants thereof. Non-limiting examples of gene products encoded by a IL1R1 gene include: UniProt: P14778; NP_001307909.1 [human], XP_038536135.1 [canine], NP_001075263.2 [equine], XP_023107327.2 [feline], as well as sequence variants, isoforms encoded by alternative splicing, and various glycoforms thereof. Canonically, the proteins encoded by the genes listed above are capable of binding all forms of the pro-inflammatory cytokine interleukin 1 (IL1 or IL1) to mediate interleukin-1-dependent activation of NF-kappa-B, MAPK and other signaling pathways. This intracellular signaling involves the recruitment of adapter molecules such as TOLLIP, MYD88, and IRAK1 or IRAK2 via TIR-TIR interactions with the cytoplasmic domains of receptor/coreceptor subunits. IL1R1 can also bind the Interleukin 1 receptor antagonist (IL1Ra or IL1Ra or IL1RN), which prevents association with IL1RAP to form a signaling-competent complex. In some instances, and merely for the sake of disambiguation, a prefix is added when referring to the protein or gene of a particular species (with h, c, e, and f, referring to the human, canine, equine, and feline forms, respectively).

The term “IL1A gene” refers to a mammalian gene encoding a Interleukin 1 Alpha polypeptide. Non-limiting examples of IL1A genes include: NCBI Gene ID: 3552 [human], NCBI Gene ID: 403782 [canine], NCBI Gene ID: 100064969 [equine], NCBI Gene ID: 493944 [feline], as well as synonymous and non-synonymous sequence variants thereof. Non-limiting examples of gene products encoded by a IL1A gene include: UniProt: P01583; NP_000566.3 [human], NP_001003157.2 [canine], NP_001075969.2 [equine], NP_001009351.1 [feline], as well as sequence variants, isoforms encoded by alternative splicing, and various glycoforms thereof. Canonically, the proteins encoded by the genes listed above are pro-inflammatory cytokines that signal through interaction with IL1R1 and IL1RAP to activate various pathways, including MAPK, JNK and NF-kappa B. In some instances, and merely for the sake of disambiguation, a prefix is added when referring to the protein or gene of a particular species (with h, c, e, and f referring to the human, canine, equine, and feline forms, respectively).

The term “IL1B gene” refers to a mammalian gene encoding an Interleukin 1 Beta polypeptide. Non-limiting examples of IL1B genes include: NCBI Gene ID: 3553 [human], NCBI Gene ID: 403974 [canine], NCBI Gene ID: 100034237 [equine], NCBI Gene ID: 768274 [feline], as well as synonymous and non-synonymous sequence variants thereof. Non-limiting examples of gene products encoded by an IL1B gene include: UniProt: P01584; NP_000567.1 [human], NP_001033060.1 [canine], NP_001075995.1 [equine], NP_001070882.1 [feline], as well as sequence variants, isoforms encoded by alternative splicing, and various glycoforms thereof. Canonically, the protein encoded by the genes listed above is a major mediator of the inflammatory response and pyrogen that signals through interaction with IL1R1 and IL1RAP. In the central nervous system (CNS) IL1B has been shown to contribute to inflammatory pain hypersensitivity, among other pathologies. In some instances, and merely for the sake of disambiguation, a prefix is added when referring to the protein or gene of a particular species (with h, c, e, and f referring to the human, canine, equine, and feline forms, respectively).

The term “treatment” refers to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. For example, a composition, method, or system of the present disclosure may be administered as a prophylactic treatment to a subject that has a predisposition for a given condition (e.g., arthritis). “Treatment”, as used herein, covers any treatment of a disease in a mammal, particularly in a human, canine, feline, or equine, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms.

“Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine. It is understood that compositions and methods of the present disclosure are applicable to treat all mammals, including, but not limited to human, canine, feline, equine, and bovine subjects.

The term “therapeutically effective” refers to the amount of a composition or combination of compositions as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular composition(s) chosen, the dosing regimen to be followed, whether the composition is administered in combination with other compositions or compounds, timing of administration, the tissue to which it is administered, and the physical delivery system in which the composition is carried.

A “spinal condition or disorder” includes, but is not limited to, low back pain, neck pain, discogenic disorders, adolescent idiopathic scoliosis, adult degenerative scoliosis, cervical degenerative disc disease, cervical disc herniation, cervical myelopathy, cervical stenosis, compression fractures, degenerative spondylolisthesis, isthmic spondylolisthesis, low back sprains and strains, lumbar degenerative disc disease, lumbar disc hemiation, lumbar stenosis, neck sprain (whiplash) and strain, neck strain, osteoporosis, and whiplash. Generally, such disorders or conditions contribute to or cause localized nociception, inflammation, or morphological changes (e.g., fibrosis, degeneration, osteolysis, osteogenesis) at the cervical, thoracic, lumbar or sacral spine, or surrounding tissues.

“Low back pain” is defined as measurable or discernible pain or discomfort (either chronic or sporadic) in a given subject, encompassing at least the lumbar-spinal region of a mammal. The pain may present as being localized to the lower back (e.g., muscle ache) or as shooting, burning, stinging, and/or radiating sensations throughout the subject's back and/or extremities. The pain may be idiopathic or may be associated with one or more (diagnosed or undiagnosed) underlying conditions including, but not limited to degenerative disc disease, chronic inflammation, arthritis, osteoporosis, trauma (e.g., post-surgical), infection (e.g., discospondylitis), neuropathies, musculo-skeletal abnormalities (e.g., slipped discs or spinal stenosis or spondylolisthesis), herniated nucleus pulposus (HNP), annular ligament tears, facet joint arthritis, radicular nerve compression, and/or other degenerative disorders.

“Neck pain” is defined as measurable or discernable pain or discomfort associated with the cervical spine or adjacent ligaments, muscles, and/or tendons. The pain may manifest as localized pain in the neck or shooting, stinging, burning, and/or radiating sensations throughout the back or extremities, including, but not limited to, the subject's head, shoulders, arms, legs, and/or back. Neck pain may be idiopathic or associated with one or more (diagnosed or undiagnosed) underlying conditions, including, but not limited to, degenerative disc disease, rheumatoid arthritis, osteoporosis, fibromyalgia, chronic inflammation, infection (e.g., discospondylitis), herniated disc, spondylosis, spinal stenosis, cervical compressive myelopathy, whiplash, and/or other disorders.

The term “polynucleotide,” “nucleotide,” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, rRNA, tRNA, lncRNA, RNA antagomirs, and inhibitory DNA or RNA (RNAi, e.g., small or short hairpin (sh)RNA, microRNA (miRNA), aptamers, small or short interfering (si)RNA, trans-splicing RNA, or antisense RNA). Polynucleotides also include non-coding RNA, which include for example, but are not limited to, RNAi, miRNAs, lncRNAs, RNA antagomirs, aptamers, and any other non-coding RNAs known to those of skill in the art. Polynucleotides include naturally occurring, synthetic, and intentionally altered or modified polynucleotides as well as analogues and derivatives. The term “polynucleotide” also refers to a polymeric form of nucleotides of any length, including deoxyribonucleotides or ribonucleotides, or analogs thereof, and is synonymous with nucleic acid sequence. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment as described herein encompassing a polynucleotide encompasses both the double-stranded form and each of two complementary single-stranded forms known or predicted to make up the double-stranded form. Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

The term “gene” or “nucleotide sequence encoding a polypeptide” refers to the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons). For example, a gene includes a polynucleotide containing at least one open reading frame capable of encoding a particular protein or polypeptide after being transcribed and translated.

The term “extracellular domain” and “ectodomain” may be used interchangeably and, when referring to transmembrane cellular receptors, is defined as the portion of the protein that is exposed to the extracellular environment and is able to engage with and/or bind a ligand.

The term “cytoplasmic domain” and “intracellular domain” may be used interchangeably and, when referring to transmembrane receptors, define the portion of the protein that is exposed to the cytoplasm. In many instances, these portions of the proteins comprise signaling domains to recruit and associate with various intracellular factors. Following engagement with a ligand via the extracellular domain, the interaction effects changes that may result in new association, dissociation or recruitment of various cytoplasmic factors that aid in transducing a signal.

The term “transmembrane domain,” which may be abbreviated as “TM,” as it refers to transmembrane receptors, is defined as the portion of the protein is embedded within the plasma membrane (i.e., not exposed to either the extracellular environment or the cytosol). Transmembrane domains are generally of a more hydrophobic character than either the extracellular or cytoplasmic portions and often adopt higher order helical structures. Though its primary role is an anchor, ligand-induced conformational changes to particular receptors have been shown to impact the transmembrane domain such that it is integral to the subsequent intracellular signaling.

The term “receptor” refers to a protein capable of binding another cognate protein (i.e., its ligand) with high affinity. This receptor-ligand interaction may be 1:1, or result in multimerization, wherein numerous proteins aggregate to bind one or more ligands. Receptors are generally present at the cell surface, such that they may most efficiently encounter a ligand and initiate intracellular signaling.

The term “intracellular signaling” refers to cellular changes that result due to events occurring at the cell surface. Typically, a soluble ligand binds its receptor at the cell surface, which can induce changes in the receptor, such that associated intracellular factors are also affected. These factors may then impact others within the cell, and this cascade continues until, in many cases, a particular factor is able to alter gene expression in the nucleus in response to the stimulus at the surface.

The term “RNA-guided nuclease” refers to an enzyme capable of breaking the backbone of, for example, a DNA molecule. The activity of RNA-guided nucleases is directed by a nucleic acid molecule (i.e., guide RNA). Once properly oriented to form a functional ribonucleoprotein complex, the enzyme locates a specific position within a target nucleic acid (e.g., a gene or locus) via sequence complementarity with a portion of the guide RNA. Non-exhaustive examples of RNA-guided nucleases include Cas9, Cas12 and Cas12a (previously known as Cpf1).

The term “Cas9” refers to an RNA-guided, double-stranded DNA-binding nuclease protein or nickase protein, or a variant thereof and may be used to refer to either naturally-occurring or recombinant Cas9 nucleases variants (e.g., ES-Cas9, HF-Cas9, PE-Cas9, and AR-Cas9). The wildtype Cas9 nuclease has two functional domains, e.g., RuvC and HNH, that simultaneously cut both strands of double stranded DNA, resulting in a double-strand break. Cas9 enzymes described herein may comprise a HNH or HNH-like nuclease domain and/or a RuvC or RuvC-like nuclease domain without impacts on the ability to induce double-strand breaks in genomic DNA (e.g., at a target locus) when both functional domains are active. The Cas9 enzyme may comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species.

As used herein, “PAM” refers to a Protospacer Adjacent Motif and is necessary for an RNA-guided nuclease to bind a target nucleic acid. In many instances, the PAM directly abuts the complementary sequence in the target. Naturally-occurring Cas9, for example, molecules recognize specific PAM sequences (see, e.g., Table 1). In some embodiments, a Cas9 molecule has the same PAM specificities as a naturally occurring Cas9 molecule. In other embodiments, a Cas9 molecule has a PAM specificity not associated with a naturally occurring Cas9 molecule. In other embodiments, a Cas9 molecule's PAM specificity is not associated with the naturally occurring Cas9 molecule to which it has the closest sequence homology. For example, a naturally occurring Cas9 molecule can be altered such that the PAM sequence recognition is altered to decrease off target sites, improve specificity, or eliminate a PAM recognition requirement. In an embodiment, a Cas9 molecule may be altered (e.g., to lengthen a PAM recognition sequence, improve Cas9 specificity to high level of identity, to decrease off target sites, and/or increase specificity). In an embodiment, the length of the PAM recognition sequence is at least 4, 5, 6, 7, 8, 9, 10 or 15 amino acids in length. In some embodiments, a Cas9 molecule may be altered to ablate PAM recognition.

The term “guide RNA,” “gRNA” or “sgRNA” may be used interchangeably and refer to an RNA molecule, preferably a synthetic RNA molecule, composed of a targeting (crRNA) sequence and scaffold. These molecules, once loaded onto a functional RNA-guided nuclease can direct sequence-specific cleavage of a target nucleic acid.

An sgRNA can be administered or formulated, e.g., as a synthetic RNA, or as a nucleic acid comprising a sequence encoding the gRNA, which is then expressed in the target cells. As would be evident to one of ordinary skill in the art, various tools may be used in the design and/or optimization of an sgRNA in order to, for example, increase specificity and/or precision of genomic editing at a particular site.

In general, candidate sgRNAs may be designed and identified by first locating suitable PAMs within a genomic sequence. Then additional calculations may be utilized to predict on-target and off-target efficiencies. Available tools to aid in the initial design and modeling of candidate sgRNAs include, without limitation, CRISPRseek, CRISPRverse, CRISPR Design Tool, Cas-OFFinder, E-CRISP, ChopChop, CasOT, CRISPR direct, CRISPOR, BREAKING-CAS, CrispRGold, and CCTop. See, e.g., Safari, F. et al. (2017). Current Pharmaceutical Biotechnology 18(13), 1038-54 and Hoberecht, L. et al. (2022). Nature Communications 13, 6568, which are incorporated by reference herein in its entirety for all purposes. Such tools are also described, for example, in PCT Publication No. WO2014093701A1 and Liu, G. et al. (2020). Computational and Structural Biotechnology Journal 18, 35-44, each of which is incorporated by reference herein in its entirety for all purposes. Candidate sgRNAs may be further assessed by experimental screening or other methodologies.

The term “CRISPR RNA” or “crRNA” refer to the portion of an sgRNA molecule with complementarity to the target nucleic acid.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The term “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

The term “pharmaceutically acceptable excipient” is intended to include vehicles and carriers capable of being co-administered with a compound to facilitate the performance of its intended function. The use of such media for pharmaceutically active substances is well known in the art. Examples of such vehicles and carriers include solutions, solvents, dispersion media, delay agents, emulsions and the like. Any other conventional carrier suitable for use with the multi-binding compounds also falls within the scope of the present disclosure.

As used herein, the term “a”, “an”, or “the” generally is construed to cover both the singular and the plural forms.

The term “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by The term “about” or “approximately” depends on the particular system under study, and can be readily appreciated by one of ordinary skill in the art. Moreover, as used herein, The term “about” and “approximately” mean that compositions, amounts, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate,” whether or not expressly stated to be such. It is noted that embodiments of very different sizes, shapes and dimensions may employ the described arrangements.

The term “substantially” as used herein can refer to a majority of, or mostly, as in at least about 50%, 60%, 70%, 80%, 90%, 95%, 96%, 97%, 98%, 99%, 99.5%, 99.9%, 99.99% or at least about 99.999% or more.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed methods and compositions. All compositions, methods, and kits described herein that embody the present disclosure can, in alternate embodiments, be more specifically defined by any of the transitional terms “comprising,” “consisting essentially of,” and “consisting of.”

### III. Methods

### A. CRISPR

Unless otherwise defined, all technical and scientific terms used herein have the same meaning as commonly understood by one of ordinary skill in the art to which this disclosure belongs. Although methods and materials similar or equivalent to those described herein may be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

In one aspect, the present disclosure encompasses compositions relating to clustered regularly interspaced short palindromic repeats (CRISPR) and CRISPR-associated RNA-guided nucleases and associated methods, components, and compositions (hereafter, CRISPR/Cas systems). Such systems minimally require at least one isolated or non-naturally-occurring RNA-guided nuclease (e.g., a Cas9 protein) and at least one isolated or non-naturally-occurring guide RNA (e.g., an sgRNA) to effectuate augmentation of a nucleic acid sequence (e.g., genomic DNA).

In some embodiments, a CRISPR/Cas system effectuates the alteration of a targeted gene or locus in a eukaryotic cell by effecting an alteration of the sequence at a target position, e.g., by creating an insertion or deletion (collectively, an indel) or a nucleotide substitution resulting in a truncation, nonsense mutation, missense mutation, or other type of loss-of-function of an encoded product of, for example, a gene for (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v). In some embodiments, a CRISPR/Cas system of the present disclosure provides for the alteration of a gene and/or encoded product of a gene, such that the altered product has a resultant loss-of-function and becomes a dominant negative or decoy (e.g., a transmembrane receptor incapable of initiating intracellular signaling or a soluble receptor).

In one aspect, CRISPR/Cas systems effectuate changes to the sequence of a nucleic acid through nuclease activity. For example, in the case of genomic DNA, the RNA-guided-nuclease locates a target position within a targeted gene or locus by sequence complementarity with the target genomic sequence (e.g., CRISPR RNA (crRNA) or a complementary component of a synthetic single guide RNA (sgRNA)) and cleaves the genomic DNA upon recognition of a particular, nuclease-specific motif called the protospacer adjacent motif (PAM). See generally, Collias, D., & Beisel, C. L. (2021). Nature Communications, 12(1), 1-12.

Nuclease activity induces a double-strand break (DSB) in the case of genomic DNA. Endogenous cellular mechanisms of DSB repair, namely non-homologous end joining (NHEJ), microhomology-mediated end joining (MMEJ), and homologous recombination, result in erroneous repair at a given target position with some calculable frequency as a result of interference from said components of the CRISPR/Cas system, thereby introducing substitutions or indels into the genomic DNA. See generally Scully, R., et al. (2019). Nature Reviews Molecular Cell Biology, 20(11), 698-714. At some frequency, these indels and/or substitutions may result in frameshifts, nonsense mutations (i.e., early stop codons) or truncations that impact the availability of gene products, such as mRNA and/or protein. In certain embodiments, the CRISPR/Cas system may induce a homology-directed repair (HDR) mechanism leading to insertions of non-random sequences at a target position through the use of templates (e.g., an HDR template) provided to the cell as part of the system along with the nuclease and gRNA. See Bloh, K., & Rivera-Torres, N. (2021). International Journal of Molecular Sciences, 22(8), 3834.

In general, the minimum requirements of the CRISPR/Cas system will be dependent upon the nuclease (e.g., Cas protein) provided therewith. To this extent, these nucleases have been functionally divided into Types I, III, and V, which all fall into Class 1 and Types II, IV, and VI that are grouped into Class 2.

### Class 1 CRISPR Cas Systems:

The exact components, compositions, and methods for effectuating a change in a targeted nucleic acid sequence using a Class 1 CRISPR/Cas system will vary, but should minimally include: a nuclease (selected from at least Types I, and III), at least one guide RNA selected from 1) sgRNA or 2) a combination of crRNA and tracrRNA. These CRISPR/Cas systems have been categorized together as Class 1 CRISPR/Cas systems due to their similarities in requirements and mode of action within a eukaryotic cell. To this end, compositions, components, and methods among Class 1 constituents may be considered functionally interchangeable, and the following details, provided merely for exemplary purposes, do not represent an exhaustive list of class members:

Cas3 (see Table 1) is the prototypical Type I DNA nuclease that functions as the effector protein as part of a larger complex (the Cascade complex comprising Cse1, Cse2), that is capable of genome editing. See generally He, L., et al. (2020). Genes, 11(2), 208. Unlike other CRISPR/Cas systems, Type I systems localize to the DNA target without the Cas3 nuclease via the Cascade complex, which then recruits Cas3 to cleave DNA upon binding and locating the 3′ PAM. The Cascade complex is also responsible for processing crRNAs such that they can be used to guide it to the target position. Because of this functionality, Cascade has the ability to process multiple arrayed crRNAs from a single molecule. See. Luo, M. (2015). Nucleic Acids Research, 43(1), 674-681. As such, Type I system may be used to edit multiple targeted genes or loci from a single molecule.

Because the natural Cas3 substrate is ssDNA, its function in genomic editing is thought to be as a nickase; however, when targeted in tandem, the resulting edit is a result of blunt end cuts to opposing strands to approximate a blunt-cutting endonuclease, such as Cas9. See Pickar-Oliver, A., & Gersbach, C. A. (2019). Nature Reviews Molecular Cell Biology, 20(8), 490-507.

Like Type I nucleases, the Type III system relies upon a complex of proteins to effect nucleic acid cleavage. Particularly, Cas10 possesses the nuclease activity to cleave ssDNA in prokaryotes. See Tamulaitis, G. Trends in Microbiology, 25(1), 49-61 (2017). Interestingly, this CRISPR/Cas system, native to archaea, exhibits dual specificity and targets both ssDNA and ssRNA. Aside from this change, the system functions much like Type I in that the crRNA targets an effector complex (similar to Cascade) in a sequence-dependent manner. Similarly, the effector complex processes crRNAs prior to association. The dual nature of this nuclease makes its applications to genomic editing potentially more powerful, as both genomic DNA and, in some cases, mRNAs with the same sequence may be targeted to silence particular targeted genes.

### Class 2 CRISPR Cas Systems:

The exact components, compositions, and methods for effectuating a change in a targeted nucleic acid sequence using a Class 2 CRISPR/Cas system will vary but should minimally include: a nuclease (selected from at least Types II, and V), at least one guide RNA selected from 1) sgRNA or 2) a combination of crRNA and tracrRNA. These CRISPR/Cas systems have been categorized together as Class 2 CRISPR/Cas systems due to their similarities in requirements and mode of action within a eukaryotic cell. To this end, compositions, components, and methods among Class 2 constituents may be considered functionally interchangeable, and the following details, provided merely for exemplary purposes, do not represent an exhaustive list of class members:

Type II nucleases are the best-characterized CRISPR/Cas systems, particularly the canonical genomic editing nuclease Cas9 (see Table 1). Multiple Cas9 proteins, derived from various bacterial species, have been isolated. The primary distinction between these nucleases is the PAM, a required recognition site within the targeted dsDNA. After association with a gRNA molecule, the crRNA (or targeting domain of a sgRNA) orients the nuclease at the proper position, but the protein's recognition of the PAM is what induces a cleavage event near that site, resulting in a blunt DSB.

In addition to the naturally-derived Cas9 proteins, several engineered variants have similarly been reported. These range from Cas9 with enhanced specific (i.e., less off-target activity), such as espCas9. Others have been catalytically modified via point mutations in the RuvC (e.g., D10A) and HNH (e.g., H840A) domains such that they induce only single-strand breaks (i.e., Cas9 nickases). See Frock, R. et al. (2015). Nature Biotechnology, 33(2), 179-186. These have also been shown to be less error-prone in editing. Such mitigation of off-target effects becomes paramount when selecting for a desired insertion (i.e., a knock in mutation, in which a desired nucleotide sequence is introduced into a target nucleic acid molecule) rather than a deletion. Indeed, less off-target effects may aid in the preferred DNA repair mechanism (HDR, in most instances for knock in mutations). See generally Naeem, M., et al. (2020). Cells, 9(7), 1608.

Additional exemplary further engineered variants of canonical Cas proteins (e.g., mutants, chimeras, and include the following (each of which are hereby incorporated by reference in their entireties for all purposes): WO2015035162A2, WO2019126716A1, WO2019126774A1, WO2014093694A1, WO2014150624A1, US20190225955A1, U.S. Pat. No. 11,427,818, U.S. patent Ser. No. 11/242,542, U.S. patent Ser. No. 11/098,297, U.S. patent Ser. No. 10/876,100, U.S. patent Ser. No. 10/767,193, U.S. patent Ser. No. 10/494,621, and U.S. patent Ser. No. 10/100,291.

For the avoidance of doubt, SpCas9 collectively refers to any one of the group consisting of espCas9 (also referred to herein as ES-Cas9 or esCas9), HF-Cas9, PE-Cas9, ARCas9 (also referred to as AR-Cas9), SpCas9-D1135E, SpCas9-HF1, HypaCas9, HiFiCas9, xCas9-3.6, xCas9-3.7, Sniper-Cas9, evoCas9, SpartaCas, LZ3Cas9, miCas9, and SuperFi-Cas9. Additional examples of Cas9 variants disclosed in the following are hereby incorporated by reference in their entireties for all purposes: Huang, X., et al. (2022). Cells, 11(14), 2186.

Like the canonical Cas9 systems, Type V nucleases only require a synthetic sgRNA with a targeting domain complementary to a genomic sequence to carry out genomic editing. These nucleases contain a RuvC domain but lack the HNH domain of Type II nucleases. Further, Cas12, for example, leaves a staggered cut in the dsDNA substrate distal to the PAM, as compared to Cas9's blunt cut next to the PAM. Both Cas12a, also known as Cpf1, and Cas12b, also known as C2c1 (see Table 1), act as part of larger complex of two gRNA-associated nucleases that acts on dsDNA as a quaternary structure, nicking each strand simultaneously. See Zetsche, B. et al. (2015). Cell, 163(3):759-771; see also Liu, L. et al. (2017). Molecular Cell, 65(2):310-322. Additionally, Cas12b (C2c1) is a highly accurate nuclease with little tolerance for mismatches. See Yang, H. et al. (2016). Cell, 167(7):1814-1828.e12.

TABLE 1
Exemplary list of Cas nucleases and their requirements

| | | Type of end | |
|---|---|---|---|
| | | generated | Spacer |
| Nuclease | PAM | (nucleic acid | length |
| (Species) | (5′→3′) | target) | (nt) |
| Cas9 (S. pyogenes) | NGG | Blunt (dsDNA) | 20 |
| Cas9 (S. aureus) | NNGRRT | Blunt (dsDNA) | 20 |
| Cas9 (C. jejuni) | NNNNRYAC | Blunt (dsDNA) | 22 |
| Cas9 (S. thermophilus) | NNAGAAW | Blunt (dsDNA) | 20 |
| Cas9 (N. meningitidis) | NNNNGATT | Blunt (dsDNA) | 24 |
| Cas9 (F. novicida) | NGG | Blunt (dsDNA) | 21 |
| Cas12a | TTTV | 5′ staggered | 23-25 |
| (L. bacterium) | | (dsDNA/ssDNA) | |
| Cas12a | TTTN | 5′ staggered | 24 |
| (Acidaminococcus | | (dsDNA/ssDNA) | |
| sp.) | | | |
| Cas3 (E. coli) | CTT/CCT/ | None/blunt | 32 |
| | CAT/CTC | (ssDNA) | |

See generally Wang, J., Zhang, C., & Feng, B. (2020). Journal of Cellular and Molecular Medicine, 24(6), 3256-3270, where N=any nucleotide; R=any purine (A or G); Y=any pyrimidine (C or T); W=A or T; V=A, C or G.

In one aspect, the CRISPR/Cas system of the present disclosure comprises at least one RNA-guided nuclease (e.g. a Cas protein) derived from one or more of the following selected bacterial genera: Corynebacterium, Sutterella, Legionella, Treponema, Filifactor, Eubacterium, Streptococcus, Lactobacillus, Mycoplasma, Bacteroides, Flavobacterium, Spirochaeta, Azospirillum, Gluconacetobacter, Neisseria, Roseburia, Parvibaculum, Nitratifractor, Campylobacter, Pseudomonas, Streptomyces, Staphylococcus, Francisella, Acidaminococcus, Lachnospiraceae, Leptotrichia, and Prevotella. In some embodiments, the Cas protein is derived from Deltaproteobacteria or Planctomycetes bacterial species.

Some aspects of the present disclosure provide strategies, methods, compositions, and treatment modalities for altering a targeted sequence within a gene locus (e.g., altering the sequence of wild type and/or of a mutant sequence within a cell or within a mammal) by insertion or deletion of one or more nucleotides mediated by an RNA-guided nuclease and one or more guide RNAs (gRNAs), resulting in loss of function of the targeted gene product. In some embodiments, the loss of function results in “knocking out” the gene of interest (i.e., generation of a “knock out”) by ablating gene expression. In some embodiments, the loss function results in a non-functional gene product (i.e., a gene product without all functionality of the wildtype gene product). In some embodiments, the loss of function results in expression of gene product with different characteristics (e.g., different binding affinity or different cellular localization).

In certain embodiments, the targeted gene is selected from (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v). In some embodiments, any region of the targeted gene (e.g., a promoter region, a 5′ untranslated region, a 3′ untranslated region, an exon, an intron, or an exon/intron border) is targeted by an RNA-guided nuclease to alter the gene. In some embodiments, a non-coding region of the targeted gene (e.g., an enhancer region, a promoter region, an intron, 5′ UTR, 3′ UTR, polyadenylation signal) is targeted to alter the gene.

### CRISPR Guide RNAs:

In one aspect, the CRISPR/Cas system of the present disclosure further provides a gRNA molecule (e.g., an isolated or non-naturally occurring RNA molecule) that interacts with the RNA-guided nuclease. In certain embodiments, the gRNA is an sgRNA comprising a crRNA sequence (also commonly referred to as a spacer sequence) comprising a nucleotide sequence which is complementary to a sequence in a target nucleic acid. In some embodiments, the sgRNA further comprises an RNA scaffolding portion (tracrRNA) that interacts with the RNA-guided nuclease, such that the crRNA is positioned to scan a target nucleic acid for complementarity. In some embodiments, the system is further, optionally, comprised of an oligonucleotide—an HDR template with homology to either side of the target position. See Bloh, K., & Rivera-Torres, N. (2021). International Journal of Molecular Sciences, 22(8):3834.

In an embodiment, the RNA-guided nuclease and sgRNA are configured to orient an associated nuclease such that a cleavage event, (e.g., a double strand break or a single strand break) occurs sufficiently close to a complementary sequence in the targeted nucleic acid, thereby facilitating an alteration in the nucleic acid sequence. In some embodiments, the crRNA is 20 nucleotides in length. In some embodiments, the crRNA is 14, 15, 16, 17, 18, 19, 20, 21, 22, 23, 24, 25, 26, 27, 28, 29, or 30 nucleotides in length.

In some embodiments, the crRNA orients the RNA-guided nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides away from the complementary sequence in the targeted nucleic acid. The double- or single-strand break may be positioned upstream or downstream of the complementary sequence in the targeted nucleic acid. In some embodiments, the cleavage event occurs within a targeted gene. In some embodiments, the cleavage event occurs upstream of a targeted gene.

In certain embodiments, a second gRNA molecule, comprising a second crRNA orients a second RNA-guided nuclease, such that a cleavage event occurs sufficiently close to a complementary sequence in the targeted nucleic acid, thereby facilitating an alteration in the nucleic acid sequence. In some embodiments, the first gRNA and the second gRNA promote a cleavage event within a single targeted gene. In some embodiments, the first gRNA and the second gRNA promote a cleavage event within different targeted genes. In some embodiments, the second crRNA is 20 nucleotides in length. In some embodiments, the second crRNA is 14, 15, 16, 17, 18, 19, 20, 21, 22, 23, 24, 25, 26, 27, 28, 29, or 30 nucleotides in length.

In some embodiments, the second crRNA orients the RNA-guided nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides away from the complementary sequence in the targeted nucleic acid. The double- or single-strand break may be positioned upstream or downstream of the complementary sequence in the targeted nucleic acid. In some embodiments, the cleavage event occurs within a targeted gene. In some embodiments, the cleavage event occurs upstream of a targeted gene.

In some embodiments, the targeting domains of the first gRNA and the second gRNA are configured such that a cleavage event is positioned, independently for each of the gRNA molecules, within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of the others cleavage event. In some embodiments, the first gRNA and the second gRNA molecules alter the targeted nucleic acid sequences simultaneously. In some embodiments, the first gRNA and the second gRNA molecules alter the targeted nucleic acid sequences sequentially.

In some embodiments, a single-strand break is accompanied by a second single-strand break, positioned by the crRNA of a first gRNA and a second gRNA, respectively. For example, the crRNA may orient the associated RNA-guided nucleases such that a cleavage event, (e.g., the two single-strand breaks), are positioned within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of one another. In some embodiments, a first crRNA and a second crRNA are configured to orient associated RNA-guided nucleases such that, for example, two single-strand breaks occurs at the same position, or within 1, 2, 3, 4, 5, 10, 15, 20, 25, 30, 35, 40, 45, 50 nucleotides of one another, on opposing strands of genomic DNA, thereby essentially approximating a double strand break.

In some embodiments a nucleic acid encodes a second sgRNA molecule. In some embodiments, a nucleic acid encodes a third sgRNA molecule. In some embodiments, a nucleic acid encodes a fourth sgRNA molecule.

In certain embodiments, a nucleic acid may comprise (a) a sequence encoding a first sgRNA, comprising a crRNA that is complementary with a sequence in a targeted gene, (b) a sequence encoding a second sgRNA, comprising a crRNA that is complementary with a sequence in a second targeted gene, and (c) a sequence encoding an RNA-guided nuclease (e.g., Cas9). Optionally, (d) and (e) are sequences encoding a third sgRNA and a fourth sgRNA, respectively. In some embodiments, the second targeted gene is the same as the first targeted gene. In other embodiments, the second targeted gene is different from the first targeted gene. In some embodiments, (a), (b), and (c) are encoded within the same nucleic acid molecule (e.g., the same vector). In some embodiments, (a) and (b) are encoded within the same nucleic acid molecule. In some embodiments, (a), (b) and (d) are encoded within the same nucleic acid molecule. In some embodiments, (a), (b) and (e) are encoded within the same nucleic acid molecule. In some embodiments, (a), (b), (d) and (e) are encoded within the same nucleic acid molecule. In some embodiments, (a), (b), and (c) are encoded within separate nucleic acid molecules. When more than two sgRNAs are used, any combination of (a), (b), (c), (d) and (e) may be encoded within a single or separate nucleic acid molecules.

In one aspect, the nucleic acid molecules (i.e., those encoding (a), (b), (c), (d) or (e)) are delivered to a target cell (i.e., any combination of the encoded RNA-guided nuclease of (c) and at least one encoded gRNA molecule of (a), (b), (d), or (e) contact a target cell). In some embodiments, said nucleic acid molecules are delivered to a target cell in vivo. In other embodiments, said nucleic acid molecules are delivered to a target cell ex vivo. In some embodiments, said nucleic acid molecules are delivered to a target cell in vitro. In certain embodiments, said nucleic acid molecules are delivered to a target cell as DNA. In other embodiments, said nucleic acid molecules are delivered to a target cell as RNA (e.g., mRNA). In some embodiments, the products of said nucleic acid molecules are delivered as an assembled ribonucleoprotein (RNP).

In some embodiments, contacting a target cell comprises delivering said RNA-guided nuclease of (c), as a protein with at least one said nucleic acid molecules selected from (a), (b), (d), and (e). In some embodiments, contacting a target cell comprises delivering said encoded RNA-guided nuclease of (c), as DNA with at least one said nucleic acid molecules selected from (a), (b), (d), and (e). In some embodiments, contacting a target cell comprises delivering said encoded RNA-guided nuclease of (c), as mRNA with at least one said nucleic acid molecules selected from (a), (b), (d), and (e).

In certain embodiments, CRISPR components are delivered to a target cell via nanoparticles. Exemplary nanoparticles that may be used with all CRISPR/Cas systems disclosed herein include, at least, lipid nanoparticles or liposomes, hydrogel nanoparticles, metalorganic nanoparticles, gold nanoparticles, magnetic nanoparticles and virus-like particles. See generally Xu, C. F. et al. (2021). Advanced Drug Delivery Reviews, 168:3-29.

### B. TALEN

In one aspect, the present disclosure contemplates use of methods, components, and compositions relating to Transcription Activator-Like Effector Nucleases (TALENs) to effectuate augmentation of a ‘nucleic acid sequence (e.g., a targeted gene.

TALE stands for “Transcription Activator-Like Effector” proteins, which include TALENs (“Transcription Activator-Like Effector Nucleases”). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Custom-designed TALE arrays are also commercially available through Cellectis Bioresearch (Paris, France), Transposagen Biopharmaceuticals (Lexington, KY, USA), and Life Technologies (Grand Island, NY, USA). TALE and TALEN methods suitable for use in the present disclosure are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein. Non-limiting examples of genes that may be silenced or inhibited by permanently gene-editing via a TALE method include (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v). In an aspect, the disclosure provides compositions for up-regulation of protein receptors (including wildtype or genetically edited), including those that bind to anti-inflammatory cytokines via a TALE method.

Examples of systems, methods, and compositions for altering the expression of a target gene sequence by a TALE method, and which may be used in accordance with embodiments of the present disclosure, are described in U.S. Pat. No. 8,586,526, which is incorporated by reference herein.

### C. Zinc-Finger Nucleases (ZFN)

In one aspect, the present disclosure contemplates use of methods, components, and compositions relating to zinc-finger nucleases (ZFNs) to effectuate augmentation of a ‘nucleic acid sequence (e.g., a targeted gene).

An individual zinc finger contains approximately 30 amino acids in a conserved ββα configuration. Several amino acids on the surface of the α-helix typically contact 3 bp in the major groove of DNA, with varying levels of selectivity. Zinc fingers have two protein domains. The first domain is the DNA binding domain, which includes eukaryotic transcription factors and contain the zinc finger. The second domain is the nuclease domain, which includes the FokI restriction enzyme and is responsible for the catalytic cleavage of DNA.

The DNA-binding domains of individual ZFNs typically contain between three and six individual zinc finger repeats and can each recognize between 9 and 18 base pairs. If the zinc finger domains are specific for their intended target site then even a pair of 3-finger ZFNs that recognize a total of 18 base pairs can, in theory, target a single locus in a mammalian genome. One method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Alternatively, selection-based approaches, such as oligomerized pool engineering (OPEN) can be used to select for new zinc-finger arrays from randomized libraries that take into consideration context-dependent interactions between neighboring fingers. Engineered zinc fingers are available commercially; Sangamo Biosciences (Richmond, CA, USA) has developed a propriety platform (CompoZr@) for zinc-finger construction in partnership with Sigma-Aldrich (St. Louis, MO, USA).

Non-limiting examples of genes that may be silenced or inhibited by permanently gene-editing via a zinc finger method include (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v). Non-limiting examples of genes that may be augmented such that their resultant products function as decoys or dominant negatives by permanently gene-editing via a zinc finger method include. In an aspect, the disclosure provides compositions for up-regulation of protein receptors (including wildtype or genetically edited), including those that bind to anti-inflammatory cytokines via a zinc finger method.

Examples of systems, methods, and compositions for altering the expression of a target gene sequence by a zinc finger method, which may be used in accordance with embodiments of the present disclosure, are described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, which are incorporated by reference herein.

Other examples of systems, methods, and compositions for altering the expression of a target gene sequence by a zinc finger method, which may be used in accordance with embodiments of the present disclosure, are described in Beane, et al., Mol. Therapy, 2015, 23 1380-1390, the disclosure of which is incorporated by reference herein.

### IV. Spinal Conditions or Disorders

### A. Introduction

Spinal conditions or disorders, including low back pain, cervical pain, sacral pain, thoracic pain. And pain or inflammation associated with discogenic disorders e.g., degenerative disc disease (DDD), are a major cause of morbidity and disability worldwide for which few long-term options for amelioration currently exist. Andersson G B. Epidemiological features of chronic low-back pain. Lancet. 1999; 354:581-585. Presently available treatments include surgical or less invasive options that often fail to offer long-term palliation. Ju, et al. Global Spine Journal (2020): 2192568220963058. All vertebrate species are affected by spinal conditions or disorders, including working animals, domestic pets, and their owners. All suffer from the associated discomfort, pain, and disability, depending on the degree of disease progression.

Spinal conditions or disorders, such as low back pain, are complex diseases characterized by a multitude of inputs contributing to a progressive course of disability. Among these contributors are morphological irregularities (e.g., disc disruptions), inflammation, changes in the localized cellular environment (e.g., vascularization and/or innervation) and degenerative changes. Peng, Bao-Gan. World Journal of Orthopedics 4.2 (2013): 42. Each contributing factor is driven by differential expression of various gene products, including at least pro-inflammatory cytokines, growth factors, pain signaling molecules, and other effector biomolecules. There is a pressing need for new methods and compositions to treat this spectrum of disease and its associated disability.

The present disclosure provides compositions and methods for spinal conditions or disorders. In some embodiments, said conditions or disorders are treated by editing a gene for any one of (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain.

In some embodiments, the condition or disorder is low back pain. In some embodiments, the condition or disorder is neck pain. In some embodiments, such conditions or disorders include disorders of the intervertebral discs (IVDs). In some embodiments, the condition or disorder is DDD. In some embodiments, the condition or disorder is a tear in the annulus fibrosis (annular ligament tear). In some embodiments, the condition or disorder is a herniation of the nucleous pulposus (HNP) or herniated disc. In some embodiments, the condition or disorder is arthritis of the facet joints of the spine. In some embodiments, the condition or disorder is spondylosis. In some embodiments, the condition or disorder is painful scoliosis. In some embodiments, the condition or disorder is spinal stenosis.

Among the advantages of the present disclosure over treatments currently available for mammals afflicted with spinal conditions or disorders include the period of relief from symptoms. Upon local administration to the spine, a disc (e.g., vertebral disc), or an intradiscal space (e.g., intradiscal injection) and subsequent genetic editing of a cell (e.g., a chondrocyte, a tenocyte, an osteocyte, a monocyte, a macrophage or the cells of the nucleus pulposus or annulus fibrosus), pro-inflammatory signaling is silenced through the targeted gene for the life of that cell. By contrast, biologic treatments require periodic dosing, which may magnify the impact of any side effects, which can be severe. Among various genetic approaches, the present disclosure is also superior due to the ability to target either a particular ligand or receptor depending on whether the issue is more systemic (i.e., throughout the back or spine, wherein targeting a circulating ligand may be advantageous) or localized (wherein targeting, for instance, a proinflammatory receptor may calm nociception). In either instance, the formulations of locally administered compositions disclosed herein are preferred over widespread (i.e., affecting multiple organ systems or intentionally spreading via blood circulation) ablation of gene expression altogether.

### B. Low Back Pain

In one aspect, the present disclosure encompasses treatments for low back pain. In some embodiments, the low back pain treatment comprises a therapeutically effective amount of a Clustered Regularly Interspaced Short Palindromic Repeats (CRISPR) gene-editing system, the system comprising an RNA-guided nuclease or a nucleic acid encoding an RNA-guided nuclease and at least one guide RNA or a nucleic acid encoding at least one guide RNA targeting a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, TL4, TL6, IL6ST, IL10, TL13, IL17A, TL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain in a mammal, e.g., a human, dog, horse, or cat.

### C. Neck Pain

In one aspect, the present disclosure encompasses treatments for neck pain. In some embodiments, the neck pain treatment comprises a therapeutically effective amount of a Clustered Regularly Interspaced Short Palindromic Repeats (CRISPR) gene-editing system, the system comprising an RNA-guided nuclease or a nucleic acid encoding an RNA-guided nuclease and at least one guide RNA or a nucleic acid encoding at least one guide RNA targeting a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain in a mammal, e.g., a human, dog, horse, or cat.

### D. DDD

In one aspect, the present disclosure encompasses treatments for degenerative disc disease (DDD). In some embodiments, the DDD treatment comprises a therapeutically effective amount of a Clustered Regularly Interspaced Short Palindromic Repeats (CRISPR) gene-editing system, the system comprising an RNA-guided nuclease or a nucleic acid encoding an RNA-guided nuclease and at least one guide RNA or a nucleic acid encoding at least one guide RNA targeting a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain in a mammal, e.g., a human, dog, horse, or cat.

### E. Annular Ligament Tear

In one aspect, the present disclosure encompasses treatments for a tear in the annulus fibrosis (i.e., an annular ligament tear). In some embodiments, the annular ligament tear treatment comprises a therapeutically effective amount of a Clustered Regularly Interspaced Short Palindromic Repeats (CRISPR) gene-editing system, the system comprising an RNA-guided nuclease or a nucleic acid encoding an RNA-guided nuclease and at least one guide RNA or a nucleic acid encoding at least one guide RNA targeting a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain in a mammal, e.g., a human, dog, horse, or cat.

### F. HNP Herniation (Herniated Disc)

In one aspect, the present disclosure encompasses treatments for hemiation of the nucleous pulposus (HNP) or herniated disc. In some embodiments, the HNP herniation/herniated disc treatment comprises a therapeutically effective amount of a Clustered Regularly Interspaced Short Palindromic Repeats (CRISPR) gene-editing system, the system comprising an RNA-guided nuclease or a nucleic acid encoding an RNA-guided nuclease and at least one guide RNA or a nucleic acid encoding at least one guide RNA targeting a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain in a mammal, e.g., a human, dog, horse, or cat.

### G. Facet Joint Arthritis

In one aspect, the present disclosure encompasses treatments for arthritis of the facet joints of the spine. In some embodiments, the facet joint arthritis treatment comprises a therapeutically effective amount of a Clustered Regularly Interspaced Short Palindromic Repeats (CRISPR) gene-editing system, the system comprising an RNA-guided nuclease or a nucleic acid encoding an RNA-guided nuclease and at least one guide RNA or a nucleic acid encoding at least one guide RNA targeting a gene for: (i) one or more growth factors or growth factor receptors (e.g., FGF2, CCN2, NGF, NTF3, NTF4, BDNF, FGFR1, NGFR, NTRK11, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain in a mammal, e.g., a human, dog, horse, or cat.

### H. IVD Disease

In one aspect, the present disclosure encompasses treatments for diseases of the intervertebral disc. In some embodiments, the IVD disease treatment comprises a a therapeutically effective amount of a Clustered Regularly Interspaced Short Palindromic Repeats (CRISPR) gene-editing system, the system comprising an RNA-guided nuclease or a nucleic acid encoding an RNA-guided nuclease and at least one guide RNA or a nucleic acid encoding at least one guide RNA targeting a gene for: (i) one or more growth factors or growth factor receptors (e.g., FGF2, CCN2, NGF, NTF3, NTF4, BDNF, FGFR1, NGFR, NTRK11, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain in a mammal, e.g., a human, dog, horse, or cat.

### L. Spondylosis

In one aspect, the present disclosure encompasses treatments for spondylosis. In some embodiments, the spondylosis treatment comprises a therapeutically effective amount of a Clustered Regularly Interspaced Short Palindromic Repeats (CRISPR) gene-editing system, the system comprising an RNA-guided nuclease or a nucleic acid encoding an RNA-guided nuclease and at least one guide RNA or a nucleic acid encoding at least one guide RNA targeting a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5. CXCL6, CXCL8, CCL2, CCL3. CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain in a mammal, e.g., a human, dog, horse, or cat.

### J. Painful Scoliosis

In one aspect, the present disclosure encompasses treatments for painful scoliosis. In some embodiments, the painful scoliosis treatment comprises a therapeutically effective amount of a Clustered Regularly Interspaced Short Palindromic Repeats (CRISPR) gene-editing system, the system comprising an RNA-guided nuclease or a nucleic acid encoding an RNA-guided nuclease and at least one guide RNA or a nucleic acid encoding at least one guide RNA targeting a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain in a mammal, e.g., a human, dog, horse, or cat.

### K. Spinal Stenosis

In one aspect, the present disclosure encompasses treatments for spinal stenosis. In some embodiments, the spinal stenosis treatment comprises a therapeutically effective amount of a Clustered Regularly Interspaced Short Palindromic Repeats (CRISPR) gene-editing system, the system comprising an RNA-guided nuclease or a nucleic acid encoding an RNA-guided nuclease and at least one guide RNA or a nucleic acid encoding at least one guide RNA targeting a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain in a mammal, e.g., a human, dog, horse, or cat.

### V. Delivery

### A. Viral Vectors

In one aspect, the present disclosure encompasses methods of delivery of a CRISPR gene-editing system targeting a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) using one or more recombinant viral particle.

In some embodiments, the one of more viral vectors comprise a recombinant virus selected from a retrovirus, an adenovirus, an adeno-associated virus, a lentivirus, and a herpes simplex virus-1. In some embodiments, the one of more viral vectors comprise a recombinant adeno-associated virus (AAV). In some embodiments, the recombinant AAV is of serotype 5 (AAV5). In some embodiments, the recombinant AAV is of serotype 6 (AAV6). In some embodiments, the one or more viral vectors comprise: a first viral vector comprising a first nucleic acid, in the one or more nucleic acids, encoding the Cas protein; and a second viral vector comprising a second nucleic acid, in the one or more nucleic acids, encoding the at least one guide RNA. In some embodiments, the one or more viral vectors comprise a viral vector comprising a single nucleic acid, wherein the single nucleic acid encodes the Cas9 protein and the at least one guide RNA.

### 1. Adeno-Associated Virus (AAV)

A viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. For a review see Muzyczka et al., Curr. Topics in Micro. and Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). The identification of Staphylococcus aureus (SaCas9) and other smaller Cas9 enzymes that can be packaged into adeno-associated viral (AAV) vectors that are highly stable and effective in vivo, easily produced, approved by FDA, and tested in multiple clinical trials, paves new avenues for therapeutic gene editing.

According to particular embodiments, a CRISPR gene-editing system targeting a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) comprise a recombinant AAV vector. In some embodiments, the CRISPR gene-editing system is encoded by a nucleic acid, wherein the nucleic acid is a recombinant AAV genome. In some embodiments, the AAV vector is selected from an AAV1 vector, an AAV2 vector, an AAV3 vector, an AAV4 vector, an AAV5 vector, an AAV6 vector, an AAV7 vector, an AAV8 vector, an AAV9 vector, and an AAV10 vector.

In some aspects, the AAV vector comprises a serotype selected from the group consisting of AAV1, AAV1(Y705+731F+T492V), AAV2(Y444+500+730F+T491V), AAV3(Y705+731F), AAV4, AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), AAV-ShH10, and AAV-DJ/8. In some aspects, the AAV vector comprises a serotype selected from the group consisting of: AAV1, AAV5, AAV6, AAV6 (Y705F/Y731F/T492V), AAV8, AAV9, and AAV9 (Y731F).

In one aspect, use of the CRISPR gene-editing system further comprising one or more AAV vectors is therapeutic. In some embodiments, use of the system treats one or more spinal conditions or disorders. In some embodiments, the condition or disorder is low back pain. In some embodiments, the condition or disorder is neck pain. In some embodiments, such conditions or disorders include disorders of the intervertebral discs (IVDs). In some embodiments, the condition or disorder is DDD. In some embodiments, the condition or disorder is a tear in the annulus fibrosis (annular ligament tear). In some embodiments, the condition or disorder is a herniation of the nucleous pulposus (HNP) or herniated disc. In some embodiments, the condition or disorder is arthritis of the facet joints of the spine. In some embodiments, the condition or disorder is spondylosis. In some embodiments, the condition or disorder is painful scoliosis. In some embodiments, the condition or disorder is spinal stenosis.

### 2. Lentivirus

In some aspects, the viral vector is a lentivirus. In an aspect, the lentivirus is selected from the group consisting of: human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein.

In one aspect, use of the CRISPR gene-editing system further comprising one or more lentiviral vectors is therapeutic. In some embodiments, use of the system treats one or more spinal conditions or disorders. In some embodiments, the condition or disorder is low back pain. In some embodiments, the condition or disorder is neck pain. In some embodiments, such conditions or disorders include disorders of the intervertebral discs (IVDs). In some embodiments, the condition or disorder is DDD. In some embodiments, the condition or disorder is a tear in the annulus fibrosis (annular ligament tear). In some embodiments, the condition or disorder is a herniation of the nucleous pulposus (HNP) or herniated disc. In some embodiments, the condition or disorder is arthritis of the facet joints of the spine. In some embodiments, the condition or disorder is spondylosis. In some embodiments, the condition or disorder is painful scoliosis. In some embodiments, the condition or disorder is spinal stenosis.

### B. Lipid Nanoparticles (LNP)

In some embodiments, a CRISPR gene-editing system is delivered by a nanoparticle. Without wishing to be bound by any particular theory, in certain embodiments, nucleic acids, when present in the nanoparticle, are resistant in aqueous solution to degradation with a nuclease. In other embodiments, proteins are protected from protease degradation. In some embodiments, proteins and nucleic acids encapsulated by nanoparticles are capable of penetrating the cellular plasma membrane.

Lipid nanoparticles comprising nucleic acids and their method of preparation is disclosed in at least WO2017/019935, WO2017/049074, WO2017/201346, WO2017/218704, WO2018/006052, WO2018/013525, WO2018/089540, WO2018/119115, WO2018/126084, WO2018/157009, WO2018/170336, WO2018/222890, WO2019/046809, WO2019/089828, WO2020/061284, WO2020/061317, WO2020/081938, WO2020/097511, WO2020/097520, WO2020/097540, WO2020/097548, WO2020/214946, WO2020/219941, WO2020/232276, WO2020/227615, WO2020/061295, WO2021/007278, WO2021/016430, WO2021/021988, EP Patent No. EP 2 972 360, US20200155691, US20200237671, U.S. Pat. Nos. 8,058,069, 8,492,359, 8,822,668, 9,364,435, 9,404,127, 9,504,651, 9,593,077, 9,738,593, 9,868,691, 9,868,692, 9,950,068, 10,138,213, 10,166,298, 10,221,127, 10,238,754, 10,266,485, 10,383,952, 10,730,924, 10,766,852, 11,079,379, 11,141,378 and 11,246,933, which are incorporated herein by reference in their entirety for all purposes.

### Lipid Nanoparticle Compositions

In some embodiments, the largest dimension of a nanoparticle composition is 1 micrometer or shorter (e.g., 1 micrometer, 900 nm, 800 nm, 700 nm, 600 nm, 500 nm, 400 nm, 300 nm, 200 nm, 175 nm, 150 nm, 125 nm, 100 nm, 75 nm, 50 nm, or shorter), e.g., when measured by dynamic light scattering (DLS), transmission electron microscopy, scanning electron microscopy, or another method. Nanoparticle compositions include, for example, lipid nanoparticles (LNPs), liposomes, lipid vesicles, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers may be functionalized and/or crosslinked to one another. Lipid bilayers may include one or more ligands, proteins, or channels. In various embodiments, lipid nanoparticles described herein have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic.

In certain embodiments, the lipid nanoparticles described herein comprise one or more components, including a lipid component, and (optionally) a structural component. The lipid component comprises lipids selected from ionizable and/or cationic lipids (i.e., lipids that may have a positive or partial positive charge at physiological pH), neutral lipids (e.g., phospholipids, or sphingolipids), and polymer-conjugated lipids (e.g., PEGylated lipids). In some embodiments, the lipid component comprises a single ionizable lipid. In other embodiments, the lipid component comprises 2, 3, 4, 5, 6, 7, 8, 9, 10, 15, or 20 ionizable lipids. In some embodiments, the lipid component comprises a single neutral lipid. In other embodiments, the lipid component comprises 2, 3, 4, 5, 6, 7, 8, 9, 10, 15, or 20 neutral lipids. In some embodiments, the lipid com-ponent comprises a single polymer-conjugated lipid. In other embodiments, the lipid component comprises 2, 3, 4, 5, 6, 7, 8, 9, 10, 15, or 20 polymer-conjugated lipids. In some embodiments, the structural component comprises a single structural lipid. In other embodiments, the structural component comprises 2, 3, 4, 5, 6, 7, 8, 9, 10, 15, or 20 structural lipids. In some embodiments, the lipid component comprises at least one cationic lipid, at least one neutral lipid, and at least one polymer-conjugated lipid. The present disclosure contemplates that the lipid component may comprise any combination of the foregoing constituents.

### Ionizable/Cationic Lipids

In some embodiments, the lipid component comprises an ionizable lipid. In some embodiments, the ionizable lipid is anionic. In other embodiments, the ionizable lipid is a cationic lipid. In some embodiments, the lipid component comprises cationic lipids including, but not limited to, a cationic lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, SM-102, LP01 and mixtures thereof.

Non-exhaustive and non-limiting examples of cationic lipids include:

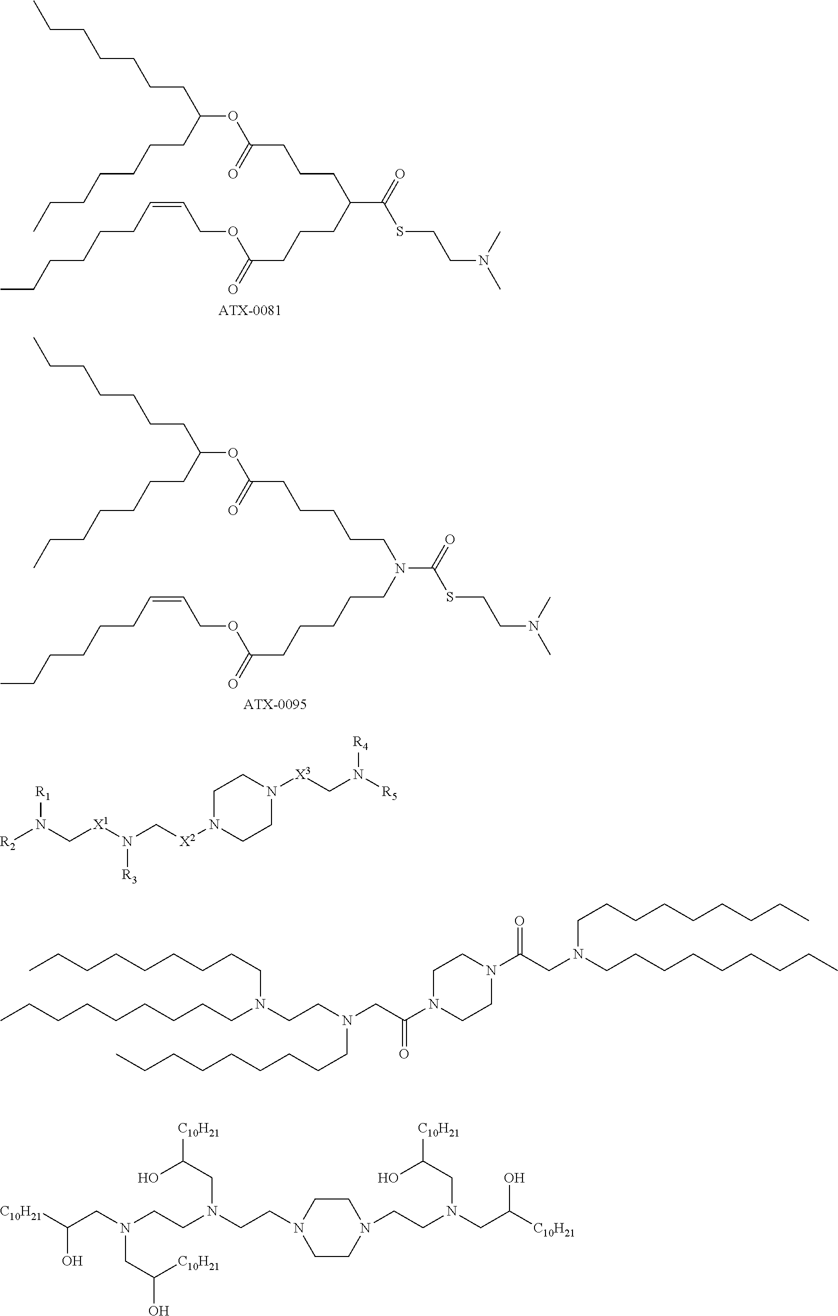

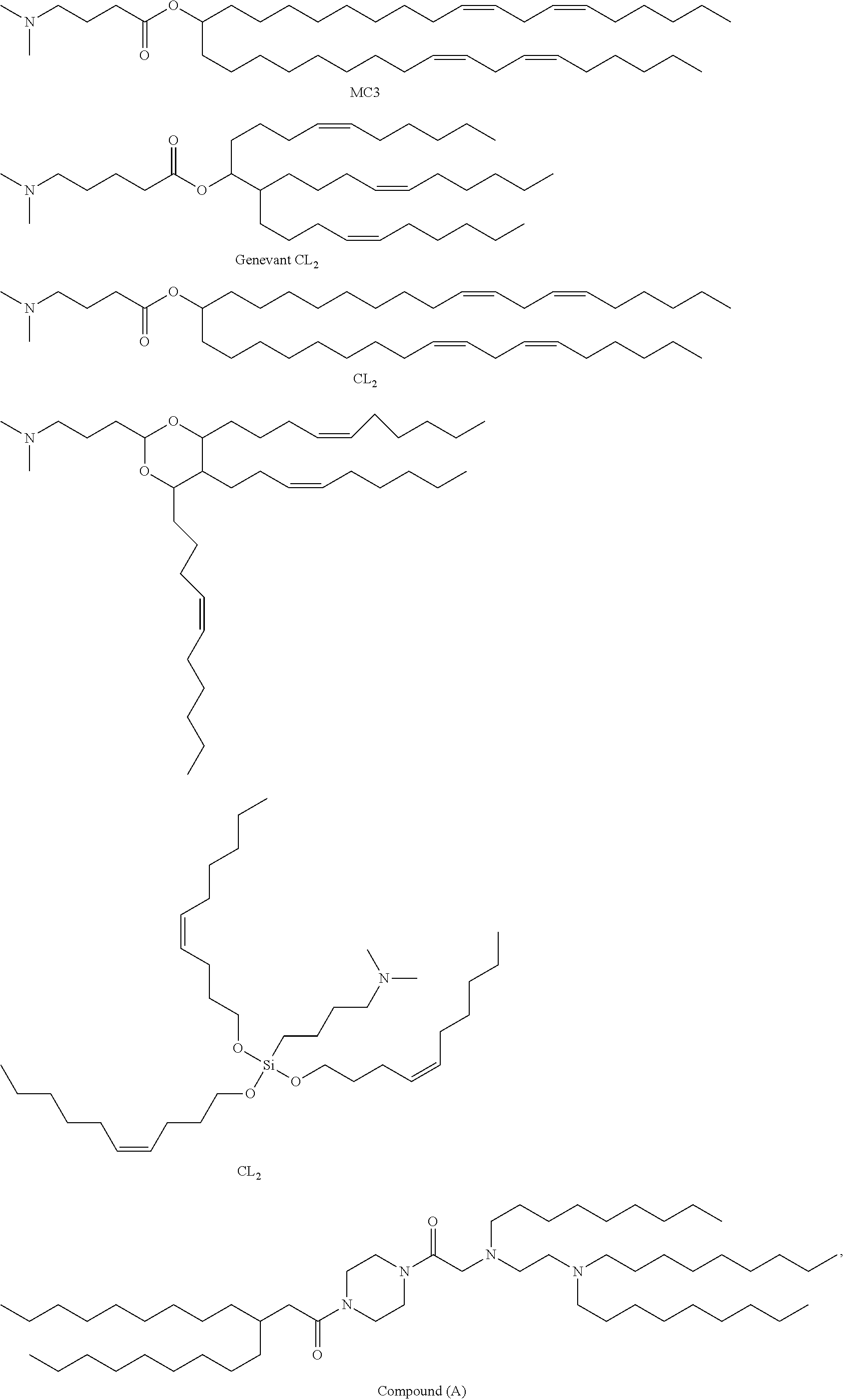

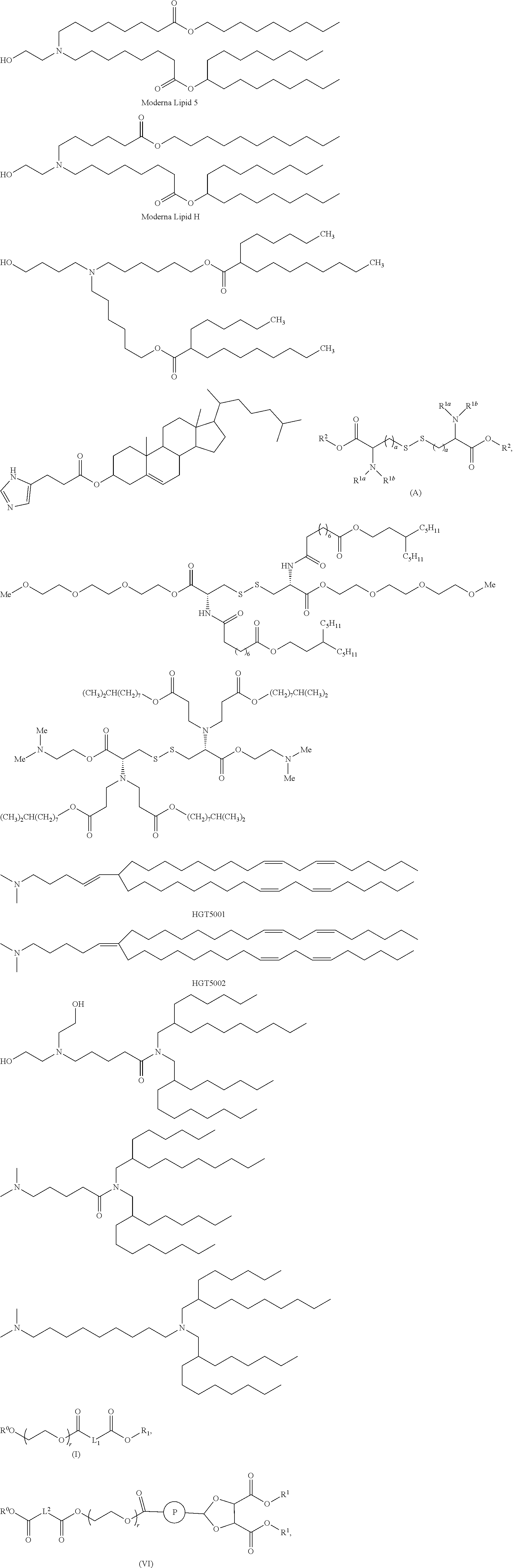

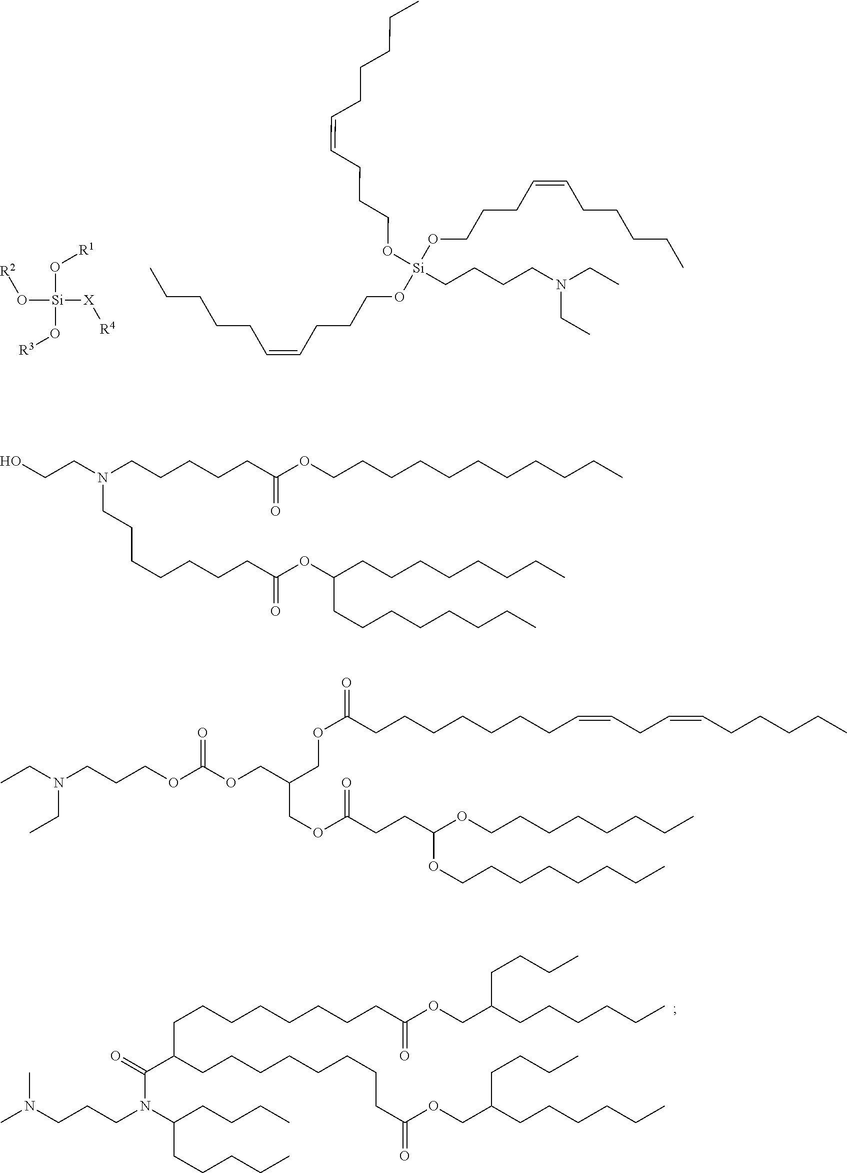

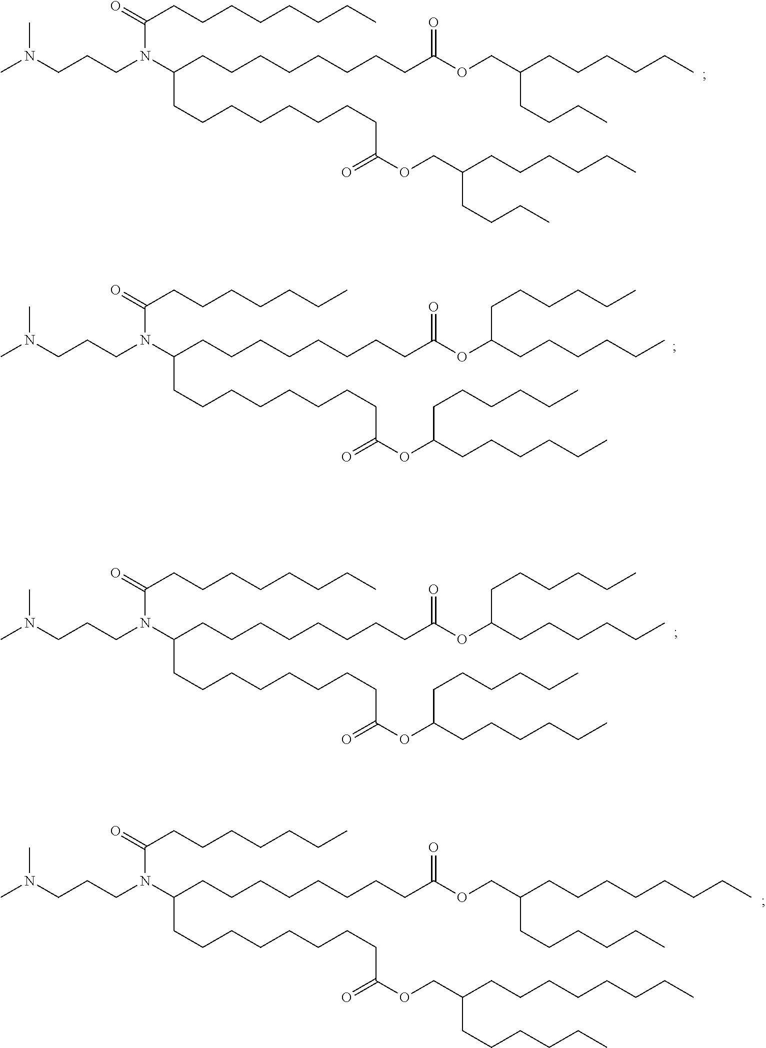

### Neutral Lipids/Phospholipids

In some embodiments, the lipid component further comprises neutral lipids including, but not limited to, a phospholipid selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and mixtures thereof.

### Polymer-Conjugated Lipids

In some embodiments, the lipid component further comprises polymer-conjugated lipids, including, but not limited to, a PEGylated lipid selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG₂₀₀₀-c-DMG, PEG-DLPE, PEG-DMPE, PEG-DPPC, PEG-DMA or a PEG-DSPE lipid.

Non-exhaustive and non-limiting examples of PEG lipids include:

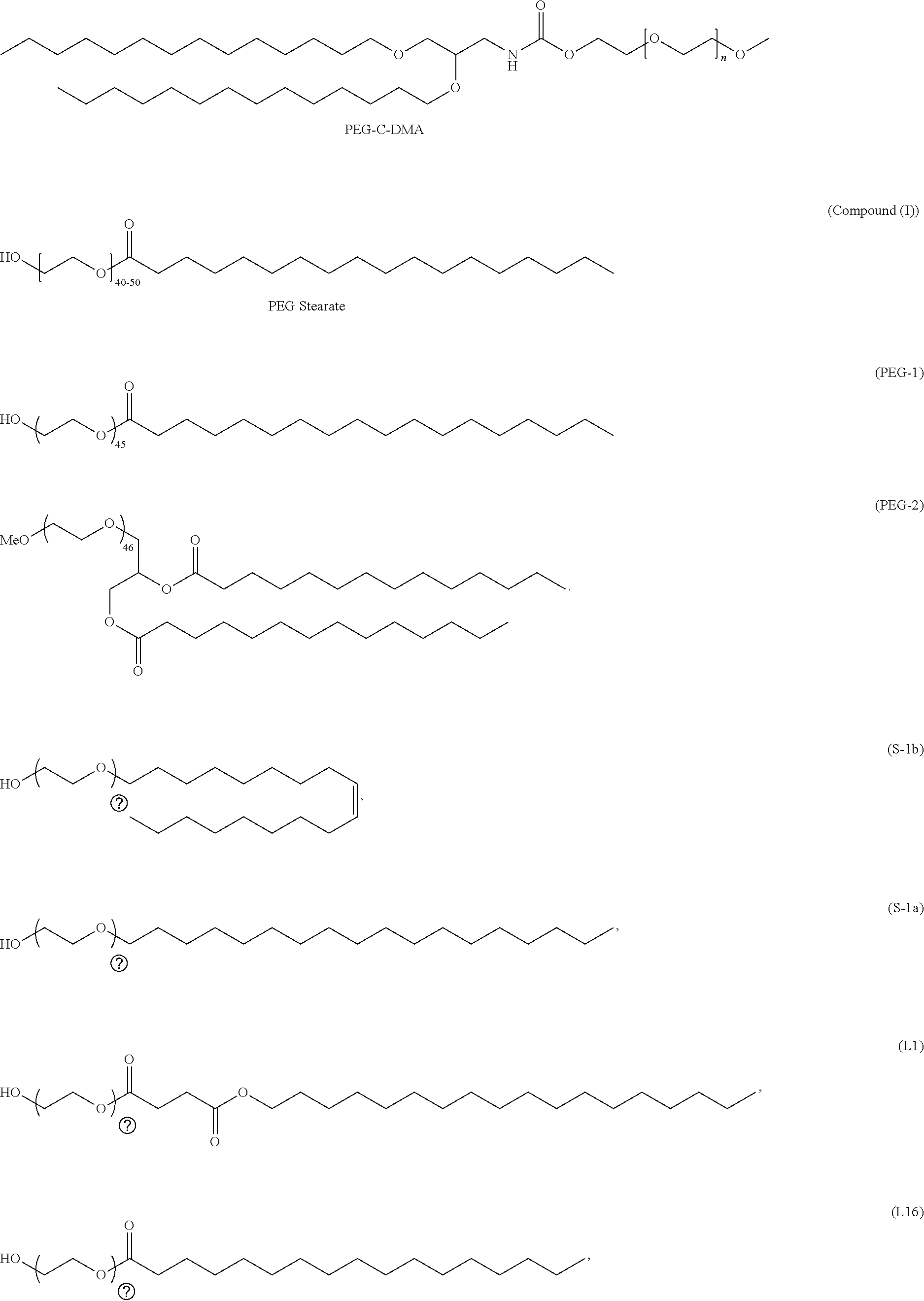

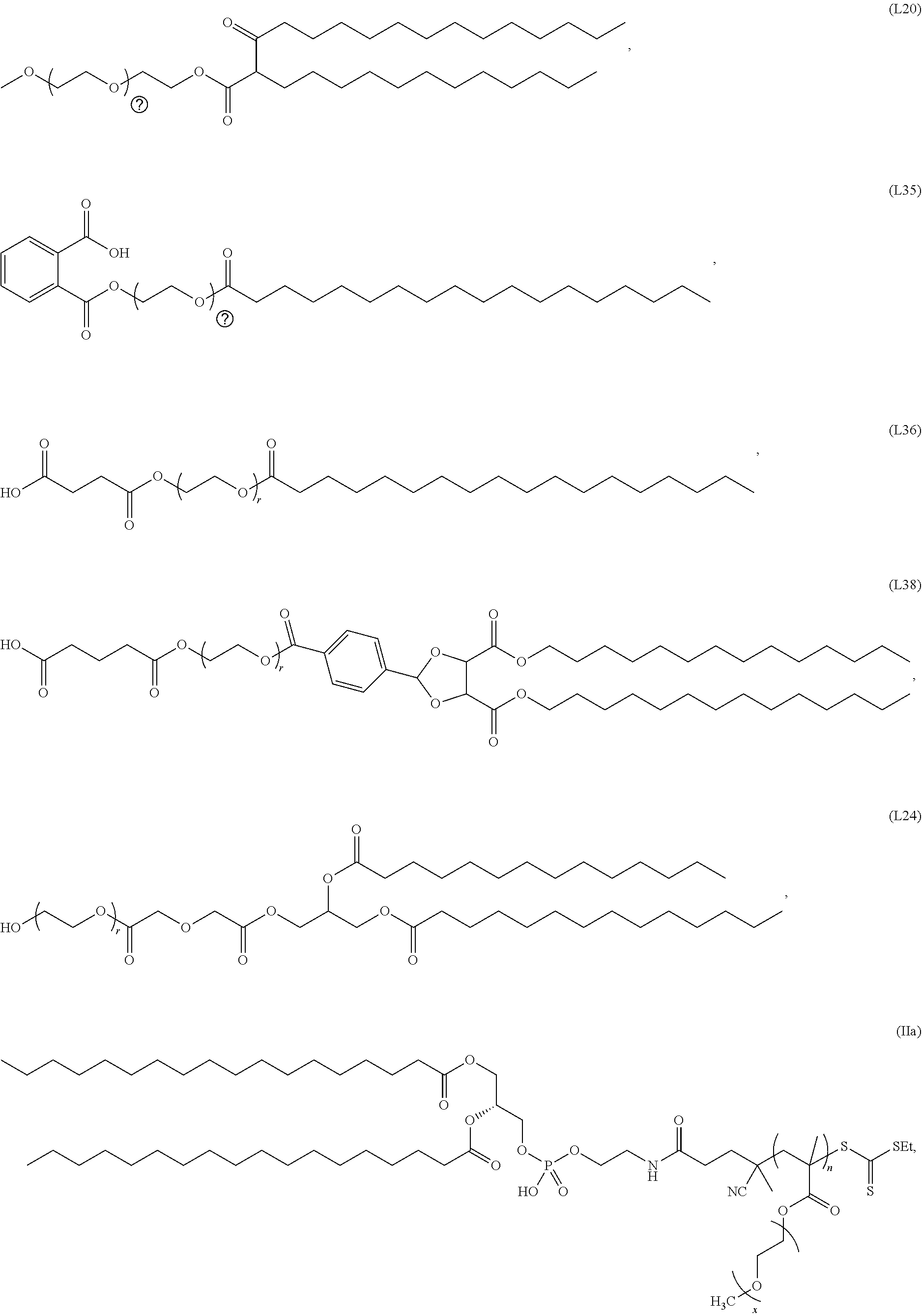

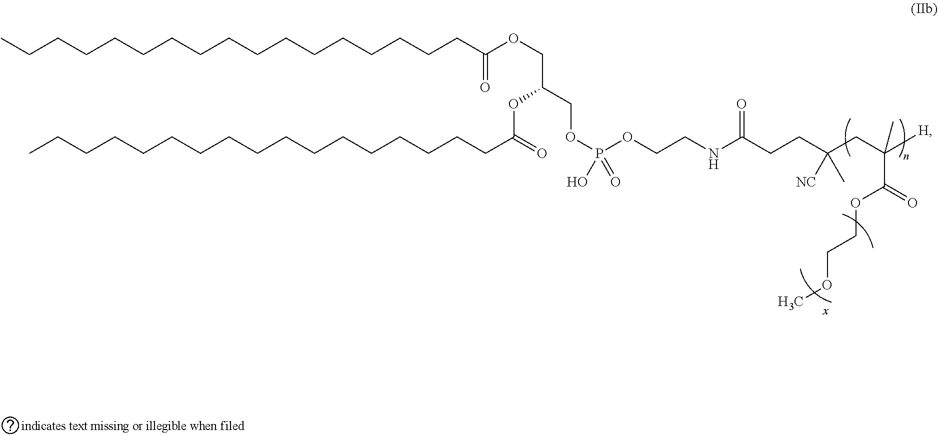

### Structural Lipids/Sterols

In some embodiments, the LNP further comprises a structural component. See generally Patel, S., et al. (2020). Nature Communications, 11(1), 1-13. In some embodiments, the structural component comprises a sterol including, but not limited to, a sterol selected from the group consisting of cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9, 11-dehydroergosterol, tomatidine, tomatine, α-tocopherol, and mixtures thereof. In other embodiments, the structural lipid includes cholesterol and a corticosteroid (e.g., prednisolone, dexamethasone, prednisone, and hydrocortisone), or a combination thereof.

Non-exhaustive and non-limiting examples of structural lipids include: lipids comprise about 1, 2, 3, 4, 5, 6, 7, 8, 9, 10, 11, 12, 13, 14, 15, 16, 17, 18, 19, 20, 25, or 30 mol % of the lipid component.

In some embodiments, the polymer-conjugated lipids comprise between about 0 and about 15 mol % of the lipid component. In other embodiments, the polymer-conjugated lipids comprise between about 0.5 and about 10 mol % of the lipid component. In various embodiments, the polymer-conjugated lipids comprise about 0.5, 1, 1.5, 2, 2.5, 3, 3.5, 4, 4.5, 5, 5.5, 6, 6.5, 7, 7.5, 8, 8.5 9, 9.5, 10, or 15 mol % of the lipid component.

In some embodiments, the structural component comprises about 17.5 mol % to about 50 mol % of the lipid component. In other embodiments, the structural component comprises about 30 to about 40 mol % of the lipid component. In various embodiments, the structural component comprises about 17.5, 20, 22.5, 25, 27.5, 30, 31, 32, 33, 34, 35, 36, 37, 38, 39, 40, 41, 42, 43, 44, 45, 46, 47, 48, 49, or 50 mol % of the lipid component.

The structural component may alternatively be expressed as a ratio relative to the lipid component. In some embodiments, the structural component is in a ratio of about 1:1 with the lipid component (sterol:lipids). In other embodiments, the structural component is in a ratio of about 1:5 with the lipid component (sterol:lipids). In various embodiments, the structural component is in a ratio of about 1:1, 1:2, 1:3, 1:4,1:5, 1:6, 1:7, 1:8, 1:9, 1:10, 1:15, 1:20, or 1:25 with the lipid component (sterol:lipids).

Nanoparticle compositions may be designed for one or more specific applications or targets. For example, a nanoparticle composition may be designed to deliver a therapeutic and/or prophylactic such as an RNA to a particular cell, tissue, organ, or system or group thereof in a mammal's body. Physiochemical properties of nanoparticle compositions may be altered in order to increase selectivity for particular bodily targets. For instance, particle sizes may be adjusted based on the fenestration sizes of different organs. The therapeutic and/or prophylactic included in a nanoparticle composition may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a nanoparticle composition may include an mRNA encoding a polypeptide of interest capable of being translated within a cell to produce the polypeptide of interest. Such a composition may be designed to be specifically delivered to a particular organ. In some embodiments, a composition may be de-signed to be specifically delivered to a mammalian joint.

The amount of a therapeutic and/or prophylactic in a nanoparticle composition may depend on the size, composition, desired target and/or application, or other properties of the nanoparticle composition as well as on the properties of the therapeutic and/or prophylactic. For example, the amount of an RNA useful in a nanoparticle composition may depend on the size, sequence, and other characteristics of the RNA. The relative amounts of a therapeutic and/or prophylactic and other elements (e.g., lipids) in a nanoparticle composition may also vary. In some embodiments, the wt/wt ratio of the lipid component to a therapeutic and/or prophylactic in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a nanoparticle composition may, for example, be measured using absorption spectroscopy (e.g., ultraviolet-visible spectroscopy).

In some embodiments, the therapeutic and/or prophylactic comprises a nucleic acid component. In some embodiments, the nucleic acid component comprises RNA including, but not limited to, RNA selected from the group consisting of messenger RNA (mRNA), CRISPR RNA (crRNA), tracrRNA, single-guide RNA (sgRNA), short interfering RNA (siRNA), antisense oligonucleotides (ASO), and mixtures thereof. In other embodiments, the nucleic acid component comprises DNA including, but not limited to, DNA selected from the group consisting of linear DNA, plasmid DNA, antisense oligonucleotide, and mixtures thereof.

In some embodiments, a nanoparticle composition includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

In some embodiments, the nucleic acid component is comprised of a modified nucleic acid. For example, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

In certain embodiments, the present disclosure comprises methods for treating back or spine conditions or disorders. In other embodiments, the present disclosure comprises methods for treating discogenic disorders. In some embodiments, the present disclosure comprises methods for treating localized nociception, inflammation, or morphological changes associated with back or spine conditions or disorders in a subject in need thereof, the method comprising administering a therapeutically effective amount of a CRISPR-Cas composition encapsulated within or associated with a lipid nanoparticle (LNP), wherein the composition comprises one or more non-naturally occurring polynucleotides encoding a Cas9 protein and at least one sgRNA. In some embodiments, LNPs are administered intradiscally. In other embodiments, LNPs are administered epidurally. In some embodiments, LNPs are administered peridiscally. In some embodiments, LNPs are administered perivertebrally.

### Physical Properties

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, such as particle size, polydispersity index, and zeta potential.

The mean size of a nanoparticle composition may be between 10 nm and 1 micrometer, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition may be from about 70 nm to about 100 nm. In a particular embodiment, the mean size may be about 80 nm. In other embodiments, the mean size may be about 100 nm.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic and/or prophylactic in a solution containing the nanoparticle composition before and after breaking up the nanoparticle composition with one or more organic solvents or detergents. Fluorescence may be used to measure the amount of free therapeutic and/or prophylactic (e.g., RNA) in a solution. For the nanoparticle compositions described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A nanoparticle composition may optionally comprise one or more coatings. For example, a nanoparticle composition may be formulated in a capsule, film, or tablet having a coating. A capsule, film, or tablet including a composition described herein may have any useful size, tensile strength, hardness, or density.

Unless otherwise defined, all technical and scientific terms used herein have the same meaning as commonly understood by one of ordinary skill in the art to which this disclosure belongs. Although methods and materials similar or equivalent to those described herein may be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

In some embodiments, the CRISPR gene-editing system comprises one or more RNA-containing compositions. In some embodiments, the CRISPR gene-editing system further comprises one or more nanoparticles. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA). In some embodiments, said one or more nanoparticles are lipid nanoparticles (LNP).

In some embodiments, the CRISPR gene-editing system comprises one or more LNPs collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more LNPs comprises a first plurality of LNP encapsulating the RNA-guided nuclease or a nucleic acid encoding an RNA-guided nuclease and a second plurality of LNP encapsulating the at least one guide RNA or a nucleic acid encoding at least one guide RNA.

In some embodiments, the one or more LNP comprises a component selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)--octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and a mixture thereof.

In some embodiments, the one or more LNP comprises a component selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and a mixture thereof.

In some embodiments, the one or more LNP comprises a component selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA, a PEG-DSPE lipid, and a mixture thereof.

In some embodiments, the one or more LNP comprises a component selected from the group consisting of a cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9, 11-dehydroergosterol, tomatidine, tomatine, α-tocopherol, dexamethasone and a mixture thereof.

In some embodiments, use of the CRISPR gene-editing system further comprising one or more LNPs to target a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) is therapeutic.

In one aspect, use of the CRISPR gene-editing system further comprising one or more LNPs is therapeutic. In some embodiments, use of the system treats one or more spinal conditions or disorders. In some embodiments, the condition or disorder is low back pain. In some embodiments, the condition or disorder is neck pain. In some embodiments, such conditions or disorders include disorders of the intervertebral discs (IVDs). In some embodiments, the condition or disorder is DDD. In some embodiments, the condition or disorder is a tear in the annulus fibrosis (annular ligament tear). In some embodiments, the condition or disorder is a hemiation of the nucleous pulposus (HNP) or herniated disc. In some embodiments, the condition or disorder is arthritis of the facet joints of the spine. In some embodiments, the condition or disorder is spondylosis. In some embodiments, the condition or disorder is painful scoliosis. In some embodiments, the condition or disorder is spinal stenosis.

### C. Virus-Like Particles

In one aspect, the present disclosure encompasses means for delivering a CRISPR gene-editing system to a mammalian cell via a virus-like particle (VLP). In some embodiments, a CRISPR gene-editing system is delivered by a VLP. Without wishing to be bound by any particular theory, in certain embodiments, nucleic acids, when present in the particle, are resistant in aqueous solution to degradation with a nuclease. In other embodiments, proteins are protected from protease degradation while present in the particle. In some embodiments, proteins and nucleic acids encapsulated by VLPs are capable of penetrating the cellular plasma membrane.

In some embodiments, the CRISPR gene-editing system comprises one or more RNA-containing compositions. In some embodiments, the CRISPR gene-editing system further comprises one or more VLPs. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA).

In some embodiments, the CRISPR gene-editing system comprises one or more virus-like particles collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more virus-like particles comprises a first plurality of virus-like particles encapsulating the RNA-guided nuclease or a nucleic acid encoding an RNA-guided nuclease and a second plurality of virus-like particles encapsulating the at least one guide RNA or a nucleic acid encoding at least one guide RNA.

In some embodiments, use of the CRISPR gene-editing system further comprising one or more LNPs to target a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) is therapeutic.

In one aspect, use of the CRISPR gene-editing system further comprising one or more VLPs is therapeutic. In some embodiments, use of the system treats one or more spinal conditions or disorders. In some embodiments, the condition or disorder is low back pain. In some embodiments, the condition or disorder is neck pain. In some embodiments, such conditions or disorders include disorders of the intervertebral discs (IVDs). In some embodiments, the condition or disorder is DDD. In some embodiments, the condition or disorder is a tear in the annulus fibrosis (annular ligament tear). In some embodiments, the condition or disorder is a hemiation of the nucleous pulposus (HNP) or herniated disc. In some embodiments, the condition or disorder is arthritis of the facet joints of the spine. In some embodiments, the condition or disorder is spondylosis. In some embodiments, the condition or disorder is painful scoliosis. In some embodiments, the condition or disorder is spinal stenosis.

### D. Miscellaneous Modes of Delivery

### 1. Liposomes

In some embodiments, a CRISPR gene-editing system targeting a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) is entrapped in liposomes bearing positive charges on their surface (e.g., lipofectins), which can be tagged with antibodies against cell surface antigens of the target cells. These delivery vehicles can also be used to deliver Cas9 protein/gRNA complexes.

In some embodiments, the CRISPR gene-editing system comprises one or more RNA-containing compositions. In some embodiments, the CRISPR gene-editing system further comprises one or more liposomes. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA).

In some embodiments, wherein the composition comprises one or more liposomes collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more liposomes comprises a first plurality of liposomes encapsulating the RNA-guided nuclease or a nucleic acid encoding an RNA-guided nuclease and a second plurality of liposomes encapsulating the at least one guide RNA or a nucleic acid encoding at least one guide RNA.

In one aspect, use of the CRISPR gene-editing system further comprising one or more liposomes is therapeutic. In some embodiments, use of the system treats one or more spinal conditions or disorders. In some embodiments, the condition or disorder is low back pain. In some embodiments, the condition or disorder is neck pain. In some embodiments, such conditions or disorders include disorders of the intervertebral discs (IVDs). In some embodiments, the condition or disorder is DDD. In some embodiments, the condition or disorder is a tear in the annulus fibrosis (annular ligament tear). In some embodiments, the condition or disorder is a hemiation of the nucleous pulposus (HNP) or herniated disc. In some embodiments, the condition or disorder is arthritis of the facet joints of the spine. In some embodiments, the condition or disorder is spondylosis. In some embodiments, the condition or disorder is painful scoliosis. In some embodiments, the condition or disorder is spinal stenosis.

### 2. Lipid Nanocrystals (LNC)

In one aspect, the present disclosure encompasses means for delivering a CRISPR gene-editing system to a mammalian cell via a lipid nanocrystal (LNC). In some embodiments, a CRISPR gene-editing system is delivered by a LNC. Without wishing to be bound by any particular theory, in certain embodiments, nucleic acids, when present in the nanocrystal, are resistant in aqueous solution to degradation with a nuclease. In other embodiments, proteins are protected from protease degradation while present in the nanocrystal. In some embodiments, proteins and nucleic acids encapsulated by nanocrystal are capable of penetrating the cellular plasma membrane.

In some embodiments, the CRISPR gene-editing system comprises one or more RNA-containing compositions. In some embodiments, the CRISPR gene-editing system further comprises one or more nanocrystals. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA). In some embodiments, said one or more nanocrystals are lipid nanocrystals (LNC).

In some embodiments, the CRISPR gene-editing system comprises one or more LNCs collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more LNCs comprises a first plurality of LNC encapsulating the RNA-guided nuclease or a nucleic acid encoding an RNA-guided nuclease and a second plurality of LNC encapsulating the at least one guide RNA or a nucleic acid encoding at least one guide RNA.

In some embodiments, use of the CRISPR gene-editing system further comprising one or more LNCs to target a gene for: (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) is therapeutic.

In one aspect, use of the CRISPR gene-editing system further comprising one or more LNCs is therapeutic. In some embodiments, use of the system treats one or more spinal conditions or disorders. In some embodiments, the condition or disorder is low back pain. In some embodiments, the condition or disorder is neck pain. In some embodiments, such conditions or disorders include disorders of the intervertebral discs (IVDs). In some embodiments, the condition or disorder is DDD. In some embodiments, the condition or disorder is a tear in the annulus fibrosis (annular ligament tear). In some embodiments, the condition or disorder is a herniation of the nucleous pulposus (HNP) or herniated disc. In some embodiments, the condition or disorder is arthritis of the facet joints of the spine. In some embodiments, the condition or disorder is spondylosis. In some embodiments, the condition or disorder is painful scoliosis. In some embodiments, the condition or disorder is spinal stenosis.

### VI. Pharmaceutical Compositions

In one aspect, the present disclosure encompasses pharmaceutical compositions comprising a CRISPR gene-editing system for treatment of a mammal in need thereof. In some embodiments, the CRISPR gene-editing system targets a gene selected from (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v). In some embodiments, the mammal is selected from a human, a dog, a horse, and a cat.

### A. FGF2

In one aspect, methods and pharmaceutical compositions are provided for treating a back or spine condition or disorder by administering a therapeutically effective amount of a CRISPR pharmaceutical composition that targets an FGF2 gene to a subject in need thereof. In some embodiments, the CRISPR pharmaceutical composition comprises an RNA-guided nuclease and an sgRNA targeting the FGF2 gene with a crRNA sequence selected from SEQ ID NOs: 673-720. In some embodiments, the crRNA sequence is selected from SEQ ID NOs: 673-697. In some embodiments, the crRNA sequence is selected from 673-682. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the FGF2 gene is delivered to a mammalian cell via viral vector. In some embodiments, the CRISPR gene-editing system targeting the FGF2 gene is delivered to a mammalian cell via an AAV vector. In some embodiments, the CRISPR gene-editing system targeting the FGF2 gene is delivered to a mammalian cell via a lentiviral vector. In some embodiments, the CRISPR gene-editing system targeting the FGF2 gene is delivered to a mammalian cell via a lipid nanoparticle. In some embodiments, the CRISPR gene-editing system targeting the FGF2 gene is delivered to a mammalian cell via a virus-like particle. In some embodiments, the CRISPR gene-editing system targeting the FGF2 gene is delivered to a mammalian cell via a liposome. In some embodiments, the CRISPR gene-editing system targeting the FGF2 gene is delivered to a mammalian cell via a lipid nanocrystal.

In some embodiments, the pharmaceutical composition comprises an sgRNA having a crRNA sequence selected from SEQ ID NOs: 673-720 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 673-720 and a S. pyogenes Cas9 protein encapsulated in a lipid nanoparticle.

In various embodiments, the pharmaceutical composition comprising a CRISPR gene-editing system targeting the FGF2 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the FGF2 gene, as described herein, to a subject in need thereof. In some embodiments, the pharmaceutical composition comprises an LNP encapsuling an sgRNA having a crRNA sequence selected from SEQ ID NOs: 673-720 and a S. pyogenes Cas9 protein. In some embodiments, the crRNA sequence is selected from SEQ ID NOs: 673-697. In some embodiments, the crRNA sequence is selected from SEQ ID NOs: 673-682. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the FGF2 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the FGF2 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an FGF2 gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the FGF2 gene targeted by an RNA-guided nuclease is from a mammal. In some embodiments, the FGF2 gene targeted by an RNA-guided nuclease is from a human (hFGF2). In some embodiments, the FGF2 gene targeted by an RNA-guided nuclease is from a dog (cFGF2). In some embodiments, the FGF2 gene targeted by an RNA-guided nuclease is from a horse (eFGF2). In some embodiments, the FGF2 gene targeted by an RNA-guided nuclease is from a cat (fFGF2).

### B. FGFR1

In one aspect, methods and pharmaceutical compositions are provided for treating a back or spine condition or disorder by administering a therapeutically effective amount of a CRISPR pharmaceutical composition that targets an FGFR1 gene to a subject in need thereof. In some embodiments, the CRISPR pharmaceutical composition comprises an RNA-guided nuclease and an sgRNA targeting the FGFR1 gene with a crRNA sequence selected from SEQ ID NOs: 721-768. In some embodiments, the crRNA sequence is selected from SEQ ID NOs: 721-745. In some embodiments, the crRNA sequence is selected from 721-730. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the FGFR1 gene is delivered to a mammalian cell via viral vector. In some embodiments, the CRISPR gene-editing system targeting the FGFR1 gene is delivered to a mammalian cell via an AAV vector. In some embodiments, the CRISPR gene-editing system targeting the FGFR1 gene is delivered to a mammalian cell via a lentiviral vector. In some embodiments, the CRISPR gene-editing system targeting the FGFR1 gene is delivered to a mammalian cell via a lipid nanoparticle. In some embodiments, the CRISPR gene-editing system targeting the FGFR1 gene is delivered to a mammalian cell via a virus-like particle. In some embodiments, the CRISPR gene-editing system targeting the FGFR1 gene is delivered to a mammalian cell via a liposome. In some embodiments, the CRISPR gene-editing system targeting the FGFR1 gene is delivered to a mammalian cell via a lipid nanocrystal.

In some embodiments, the pharmaceutical composition comprises an sgRNA having a crRNA sequence selected from SEQ ID NOs: 721-768 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 721-768 and a S. pyogenes Cas9 protein encapsulated in a lipid nanoparticle.

In various embodiments, the pharmaceutical composition comprising a CRISPR gene-editing system targeting the FGFR1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the FGFR1 gene, as described herein, to a subject in need thereof. In some embodiments, the pharmaceutical composition comprises an LNP encapsuling an sgRNA having a crRNA sequence selected from SEQ ID NOs: 721-768 and a S. pyogenes Cas9 protein. In some embodiments, the crRNA sequence is selected from SEQ ID NOs: 721-745. In some embodiments, the crRNA sequence is selected from SEQ ID NOs: 721-730. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the FGFR1 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the FGFR1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an FGFR1 gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18 exon 19, exon 20, exon 21, exon 22, exon 23, exon 24 any intervening intronic regions, intron/exon junctions, the 3′ UTR, or polyadenylation signal) is targeted by an RNA-guided nuclease to alter the gene. In some embodiments, the FGFR1 gene targeted by an RNA-guided nuclease is from a mammal. In some embodiments, the FGFR1 gene targeted by an RNA-guided nuclease is from a human (hFGFR1). In some embodiments, the FGR1 gene targeted by an RNA-guided nuclease is from a dog (cFGFR1). In some embodiments, the FGFR1 gene targeted by an RNA-guided nuclease is from a horse (eFGFR1). In some embodiments, the FGFR1 gene targeted by an RNA-guided nuclease is from a cat (fFGFR1).

### C. CCN2

In one aspect, methods and pharmaceutical compositions are provided for treating a back or spine condition or disorder by administering a therapeutically effective amount of a CRISPR pharmaceutical composition that targets an CCN2 gene to a subject in need thereof. In some embodiments, the CRISPR pharmaceutical composition comprises an RNA-guided nuclease and an sgRNA targeting the CCN2 gene with a crRNA sequence selected from SEQ ID NOs: 426-473. In some embodiments, the crRNA sequence is selected from SEQ ID NOs: 426-450. In some embodiments, the crRNA sequence is selected from 426-435. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CCN2 gene is delivered to a mammalian cell via viral vector. In some embodiments, the CRISPR gene-editing system targeting the CCN2 gene is delivered to a mammalian cell via an AAV vector. In some embodiments, the CRISPR gene-editing system targeting the CCN2 gene is delivered to a mammalian cell via a lentiviral vector. In some embodiments, the CRISPR gene-editing system targeting the CCN2 gene is delivered to a mammalian cell via a lipid nanoparticle. In some embodiments, the CRISPR gene-editing system targeting the CCN2 gene is delivered to a mammalian cell via a virus-like particle. In some embodiments, the CRISPR gene-editing system targeting the CCN2 gene is delivered to a mammalian cell via a liposome. In some embodiments, the CRISPR gene-editing system targeting the CCN2 gene is delivered to a mammalian cell via a lipid nanocrystal.

In some embodiments, the pharmaceutical composition comprises an sgRNA having a crRNA sequence selected from SEQ ID NOs: 426-473 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 426-473 and a S. pyogenes Cas9 protein encapsulated in a lipid nanoparticle.

In various embodiments, the pharmaceutical composition comprising a CRISPR gene-editing system targeting the CCN2 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CCN2 gene, as described herein, to a subject in need thereof. In some embodiments, the pharmaceutical composition comprises an LNP encapsuling an sgRNA having a crRNA sequence selected from SEQ ID NOs: 426-473 and a S. pyogenes Cas9 protein. In some embodiments, the crRNA sequence is selected from SEQ ID NOs: 426-450. In some embodiments, the crRNA sequence is selected from SEQ ID NOs: 426-475. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CCN2 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CCN2 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of a CCN2 gene (e.g., 5′ untranslated region [UTR], exon 1, exon 2, exon 3, exon 4, exon 5, any intervening intronic regions, intron/exon junctions, the 3′ UTR, or polyadenylation signal) is targeted by an RNA-guided nuclease to alter the gene. In some embodiments, the CCN2 gene targeted by an RNA-guided nuclease is from a mammal. In some embodiments, the CCN2 gene targeted by an RNA-guided nuclease is from a human (hCCN2). In some embodiments, the CCN2 gene targeted by an RNA-guided nuclease is from a dog (cCCN2). In some embodiments, the CCN2 gene targeted by an RNA-guided nuclease is from a horse (eCCN2). In some embodiments, the CCN2 gene targeted by an RNA-guided nuclease is from a cat (fCCN2).

### D. ADAMTS5

In one aspect, methods and pharmaceutical compositions are provided for treating a back or spine condition or disorder by administering a therapeutically effective amount of a CRISPR pharmaceutical composition that targets an ADAMTS5 gene to a subject in need thereof. In some embodiments, the CRISPR pharmaceutical composition comprises an RNA-guided nuclease and an sgRNA targeting the ADAMTS5 gene with a crRNA sequence selected from SEQ ID NOs: 97-144. In some embodiments, the crRNA sequence is selected from SEQ ID NOs: 97-121. In some embodiments, the crRNA sequence is selected from 97-106. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the ADAMTS5 gene is delivered to a mammalian cell via viral vector. In some embodiments, the CRISPR gene-editing system targeting the ADAMTS5 gene is delivered to a mammalian cell via an AAV vector. In some embodiments, the CRISPR gene-editing system targeting the ADAMTS5 gene is delivered to a mammalian cell via a lentiviral vector. In some embodiments, the CRISPR gene-editing system targeting the ADAMTS5 gene is delivered to a mammalian cell via a lipid nanoparticle. In some embodiments, the CRISPR gene-editing system targeting the ADAMTS5 gene is delivered to a mammalian cell via a virus-like particle. In some embodiments, the CRISPR gene-editing system targeting the ADAMTS5 gene is delivered to a mammalian cell via a liposome. In some embodiments, the CRISPR gene-editing system targeting the ADAMTS5 gene is delivered to a mammalian cell via a lipid nanocrystal.

In some embodiments, the pharmaceutical composition comprises an sgRNA having a crRNA sequence selected from SEQ ID NOs: 97-144 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 97-144 and a S. pyogenes Cas9 protein encapsulated in a lipid nanoparticle.

In various embodiments, the pharmaceutical composition comprising a CRISPR gene-editing system targeting the ADAMTS5 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the ADAMTS5 gene, as described herein, to a subject in need thereof. In some embodiments, the pharmaceutical composition comprises an LNP encapsuling an sgRNA having a crRNA sequence selected from SEQ ID NOs: 97-144 and a S. pyogenes Cas9 protein. In some embodiments, the crRNA sequence is selected from SEQ ID NOs: 97-121. In some embodiments, the crRNA sequence is selected from SEQ ID NOs: 97-106. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the ADAMTS5 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the ADAMTS5 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an ADAMTS5 gene (e.g., 5′ untranslated region [UTR], exon 1, exon 2, exon 3, exon 4, exon 5, exon 6, exon 7, exon 8, exon 9, any intervening intronic regions, intron/exon junctions, the 3′ UTR, or polyadenylation signal) is targeted by an RNA-guided nuclease to alter the gene. In some embodiments, the ADAMTS5 gene targeted by an RNA-guided nuclease is from a mammal. In some embodiments, the ADAMTS5 gene targeted by an RNA-guided nuclease is from a human (hADAMTS5). In some embodiments, the ADAMTS5 gene targeted by an RNA-guided nuclease is from a dog (cADAMTS5). In some embodiments, the ADAMTS5 gene targeted by an RNA-guided nuclease is from a horse (eADAMTS5). In some embodiments, the ADAMTS5 gene targeted by an RNA-guided nuclease is from a cat (fADAMTS5).

### E. ADAMTS1

In one aspect, methods and pharmaceutical compositions are provided for treating a back or spine condition or disorder by administering a therapeutically effective amount of a CRISPR pharmaceutical composition that targets an ADAMTS1 gene to a subject in need thereof. In some embodiments, the CRISPR pharmaceutical composition comprises an RNA-guided nuclease and an sgRNA targeting the ADAMTS1 gene with a crRNA sequence selected from SEQ ID NOs: 49-96. In some embodiments, the crRNA sequence is selected from SEQ ID NOs: 49-73. In some embodiments, the crRNA sequence is selected from 49-58. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the ADAMTS1 gene is delivered to a mammalian cell via viral vector. In some embodiments, the CRISPR gene-editing system targeting the ADAMTS1 gene is delivered to a mammalian cell via an AAV vector. In some embodiments, the CRISPR gene-editing system targeting the ADAMTS1 gene is delivered to a mammalian cell via a lentiviral vector. In some embodiments, the CRISPR gene-editing system targeting the ADAMTS1 gene is delivered to a mammalian cell via a lipid nanoparticle. In some embodiments, the CRISPR gene-editing system targeting the ADAMTS1 gene is delivered to a mammalian cell via a virus-like particle. In some embodiments, the CRISPR gene-editing system targeting the ADAMTS1 gene is delivered to a mammalian cell via a liposome. In some embodiments, the CRISPR gene-editing system targeting the ADAMTS1 gene is delivered to a mammalian cell via a lipid nanocrystal.

In some embodiments, the pharmaceutical composition comprises an sgRNA having a crRNA sequence selected from SEQ ID NOs: 49-96 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 49-96 and a S. pyogenes Cas9 protein encapsulated in a lipid nanoparticle.

In various embodiments, the pharmaceutical composition comprising a CRISPR gene-editing system targeting the ADAMTS1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the ADAMTS1 gene, as described herein, to a subject in need thereof. In some embodiments, the pharmaceutical composition comprises an LNP encapsuling an sgRNA having a crRNA sequence selected from SEQ ID NOs: 49-96and a S. pyogenes Cas9 protein. In some embodiments, the crRNA sequence is selected from SEQ ID NOs: 49-73. In some embodiments, the crRNA sequence is selected from SEQ ID NOs: 49-58. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the ADAMTS1 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the ADAMTS1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an ADAMTS1 gene (e.g., 5′ untranslated region [UTR], exon 1, exon 2, exon 3, exon 4, exon 5, exon 6, exon 7, exon 8, exon 9, any intervening intronic regions, intron/exon junctions, the 3′ UTR, or polyadenylation signal) is targeted by an RNA-guided nuclease to alter the gene. In some embodiments, the ADAMTS1 gene targeted by an RNA-guided nuclease is from a mammal. In some embodiments, the ADAMTS1 gene targeted by an RNA-guided nuclease is from a human (hADAMTS1). In some embodiments, the ADAMTS1 gene targeted by an RNA-guided nuclease is from a dog (cADAMTS1). In some embodiments, the ADAMTS1 gene targeted by an RNA-guided nuclease is from a horse (eADAMTS1). In some embodiments, the ADAMTS1 gene targeted by an RNA-guided nuclease is from a cat (fADAMTS1).

### F. MMP1

In one aspect, methods and pharmaceutical compositions are provided for treating a back or spine condition or disorder by administering a therapeutically effective amount of a CRISPR pharmaceutical composition that targets an MMP1 gene to a subject in need thereof. In some embodiments, the CRISPR pharmaceutical composition comprises an RNA-guided nuclease and an sgRNA targeting the MMP1 gene with a crRNA sequence selected from SEQ ID NOs: 1311-1343. In some embodiments, the crRNA sequence is selected from SEQ ID NOs: 1311-1335. In some embodiments, the crRNA sequence is selected from 1311-1320. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the MMP1 gene is delivered to a mammalian cell via viral vector. In some embodiments, the CRISPR gene-editing system targeting the MMP1 gene is delivered to a mammalian cell via an AAV vector. In some embodiments, the CRISPR gene-editing system targeting the MMP1 gene is delivered to a mammalian cell via a lentiviral vector. In some embodiments, the CRISPR gene-editing system targeting the MMP1 gene is delivered to a mammalian cell via a lipid nanoparticle. In some embodiments, the CRISPR gene-editing system targeting the MMP1 gene is delivered to a mammalian cell via a virus-like particle. In some embodiments, the CRISPR gene-editing system targeting the MMP1 gene is delivered to a mammalian cell via a liposome. In some embodiments, the CRISPR gene-editing system targeting the MMP1 gene is delivered to a mammalian cell via a lipid nanocrystal.

In some embodiments, the pharmaceutical composition comprises an sgRNA having a crRNA sequence selected from SEQ ID NOs: 1311-1343 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1311-1343 and a S. pyogenes Cas9 protein encapsulated in a lipid nanoparticle.

In various embodiments, the pharmaceutical composition comprising a CRISPR gene-editing system targeting the MMP1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the MMP1 gene, as described herein, to a subject in need thereof. In some embodiments, the pharmaceutical composition comprises an LNP encapsuling an sgRNA having a crRNA sequence selected from SEQ ID NOs: 1311-1343 and a S. pyogenes Cas9 protein. In some embodiments, the crRNA sequence is selected from SEQ ID NOs: 1311-1335. In some embodiments, the crRNA sequence is selected from SEQ ID NOs: 1311-1320. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the MMP1 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the MMP1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an MMP1 gene (e.g., 5′ untranslated region [UTR], exon 1, exon 2, exon 3, exon 4, exon 5, exon 6, exon 7, exon 8, exon 9, exon 10, any intervening intronic regions, intron/exon junctions, the 3′ UTR, or polyadenylation signal) is targeted by an RNA-guided nuclease to alter the gene. In some embodiments, the MMP1 gene targeted by an RNA-guided nuclease is from a mammal. In some embodiments, the MMP1 gene targeted by an RNA-guided nuclease is from a human (hMMP1). In some embodiments, the MMP1 gene targeted by an RNA-guided nuclease is from a dog (cMMP1). In some embodiments, the MMP1 gene targeted by an RNA-guided nuclease is from a horse (eMMP1). In some embodiments, the MMP1 gene targeted by an RNA-guided nuclease is from a cat (fMMP1).

### G. MMP2

In one aspect, methods and pharmaceutical compositions are provided for treating a back or spine condition or disorder by administering a therapeutically effective amount of a CRISPR pharmaceutical composition that targets an MMP2 gene to a subject in need thereof. In some embodiments, the CRISPR pharmaceutical composition comprises an RNA-guided nuclease and an sgRNA targeting the MMP2 gene with a crRNA sequence selected from SEQ ID NOs: 1344-1391. In some embodiments, the crRNA sequence is selected from SEQ ID NOs: 1344-1368. In some embodiments, the crRNA sequence is selected from 1344-1353. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the MMP2 gene is delivered to a mammalian cell via viral vector. In some embodiments, the CRISPR gene-editing system targeting the MMP2 gene is delivered to a mammalian cell via an AAV vector. In some embodiments, the CRISPR gene-editing system targeting the MMP2 gene is delivered to a mammalian cell via a lentiviral vector. In some embodiments, the CRISPR gene-editing system targeting the MMP2 gene is delivered to a mammalian cell via a lipid nanoparticle. In some embodiments, the CRISPR gene-editing system targeting the MMP2 gene is delivered to a mammalian cell via a virus-like particle. In some embodiments, the CRISPR gene-editing system targeting the MMP2 gene is delivered to a mammalian cell via a liposome. In some embodiments, the CRISPR gene-editing system targeting the MMP2 gene is delivered to a mammalian cell via a lipid nanocrystal.

In some embodiments, the pharmaceutical composition comprises an sgRNA having a crRNA sequence selected from SEQ ID NOs: 1344-1391 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1344-1391 and a S. pyogenes Cas9 protein encapsulated in a lipid nanoparticle.

In various embodiments, the pharmaceutical composition comprising a CRISPR gene-editing system targeting the MMP2 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the MMP2 gene, as described herein, to a subject in need thereof. In some embodiments, the pharmaceutical composition comprises an LNP encapsuling an sgRNA having a crRNA sequence selected from SEQ ID NOs: 1344-1391 and a S. pyogenes Cas9 protein. In some embodiments, the crRNA sequence is selected from SEQ ID NOs: 1344-1368. In some embodiments, the crRNA sequence is selected from SEQ ID NOs: 1344-1353. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the MMP2 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the MMP2 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an MMP2 gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, any intervening intronic regions, intron/exon junctions, the 3′ UTR, or polyadenylation signal) is targeted by an RNA-guided nuclease to alter the gene. In some embodiments, the MMP2 gene targeted by an RNA-guided nuclease is from a mammal. In some embodiments, the MMP2 gene targeted by an RNA-guided nuclease is from a human (hMMP2). In some embodiments, the MMP2 gene targeted by an RNA-guided nuclease is from a dog (cMMP2). In some embodiments, the MMP2 gene targeted by an RNA-guided nuclease is from a horse (eMMP2). In some embodiments, the MMP2 gene targeted by an RNA-guided nuclease is from a cat (fMMP2).

### H. MMP3

In one aspect, methods and pharmaceutical compositions are provided for treating a back or spine condition or disorder by administering a therapeutically effective amount of a CRISPR pharmaceutical composition that targets an MMP3 gene to a subject in need thereof. In some embodiments, the CRISPR pharmaceutical composition comprises an RNA-guided nuclease and an sgRNA targeting the MMP3 gene with a crRNA sequence selected from SEQ ID NOs: 1392-1417. In some embodiments, the crRNA sequence is selected from SEQ ID NOs: 1392-1416. In some embodiments, the crRNA sequence is selected from 1392-1401. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the MMP3 gene is delivered to a mammalian cell via viral vector. In some embodiments, the CRISPR gene-editing system targeting the MMP3 gene is delivered to a mammalian cell via an AAV vector. In some embodiments, the CRISPR gene-editing system targeting the MMP3 gene is delivered to a mammalian cell via a lentiviral vector. In some embodiments, the CRISPR gene-editing system targeting the MMP3 gene is delivered to a mammalian cell via a lipid nanoparticle. In some embodiments, the CRISPR gene-editing system targeting the MMP3 gene is delivered to a mammalian cell via a virus-like particle. In some embodiments, the CRISPR gene-editing system targeting the MMP3 gene is delivered to a mammalian cell via a liposome. In some embodiments, the CRISPR gene-editing system targeting the MMP3 gene is delivered to a mammalian cell via a lipid nanocrystal.

In some embodiments, the pharmaceutical composition comprises an sgRNA having a crRNA sequence selected from SEQ ID NOs: 1392-1417 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1392-1417 and a S. pyogenes Cas9 protein encapsulated in a lipid nanoparticle.

In various embodiments, the pharmaceutical composition comprising a CRISPR gene-editing system targeting the MMP3 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the MMP3 gene, as described herein, to a subject in need thereof. In some embodiments, the pharmaceutical composition comprises an LNP encapsuling an sgRNA having a crRNA sequence selected from SEQ ID NOs: 1392-1417 and a S. pyogenes Cas9 protein. In some embodiments, the crRNA sequence is selected from SEQ ID NOs: 1392-1416. In some embodiments, the crRNA sequence is selected from SEQ ID NOs: 1392-1401. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the MMP3 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the MMP3 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an MMP3 gene (e.g., 5′ untranslated region [UTR], exon 1, exon 2, exon 3, exon 4, exon 5, exon 6, exon 7, exon 8, exon 9, exon 10, any intervening intronic regions, intron/exon junctions, the 3′ UTR, or polyadenylation signal) is targeted by an RNA-guided nuclease to alter the gene. In some embodiments, the MMP3 gene targeted by an RNA-guided nuclease is from a mammal. In some embodiments, the MMP3 gene targeted by an RNA-guided nuclease is from a human (hMMP3). In some embodiments, the MMP3 gene targeted by an RNA-guided nuclease is from a dog (cMMP3). In some embodiments, the MMP3 gene targeted by an RNA-guided nuclease is from a horse (eMMP3). In some embodiments, the MMP3 gene targeted by an RNA-guided nuclease is from a cat (fMMP3).

### I. MMP7

In one aspect, methods and pharmaceutical compositions are provided for treating a back or spine condition or disorder by administering a therapeutically effective amount of a CRISPR pharmaceutical composition that targets an MMP7 gene to a subject in need thereof. In some embodiments, the CRISPR pharmaceutical composition comprises an RNA-guided nuclease and an sgRNA targeting the MMP7 gene with a crRNA sequence selected from SEQ ID NOs: 1418-1436. In some embodiments, the crRNA sequence is selected from 1418-1427. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the MMP7 gene is delivered to a mammalian cell via viral vector. In some embodiments, the CRISPR gene-editing system targeting the MMP7 gene is delivered to a mammalian cell via an AAV vector. In some embodiments, the CRISPR gene-editing system targeting the MMP7 gene is delivered to a mammalian cell via a lentiviral vector. In some embodiments, the CRISPR gene-editing system targeting the MMP7 gene is delivered to a mammalian cell via a lipid nanoparticle. In some embodiments, the CRISPR gene-editing system targeting the MMP7 gene is delivered to a mammalian cell via a virus-like particle. In some embodiments, the CRISPR gene-editing system targeting the MMP7 gene is delivered to a mammalian cell via a liposome. In some embodiments, the CRISPR gene-editing system targeting the MMP7 gene is delivered to a mammalian cell via a lipid nanocrystal.

In some embodiments, the pharmaceutical composition comprises an sgRNA having a crRNA sequence selected from SEQ ID NOs: 1418-1436 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1418-1436 and a S. pyogenes Cas9 protein encapsulated in a lipid nanoparticle.

In various embodiments, the pharmaceutical composition comprising a CRISPR gene-editing system targeting the MMP7 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the MMP7 gene, as described herein, to a subject in need thereof. In some embodiments, the pharmaceutical composition comprises an LNP encapsuling an sgRNA having a crRNA sequence selected from SEQ ID NOs: 1418-1436 and a S. pyogenes Cas9 protein. In some embodiments, the crRNA sequence is selected from SEQ ID NOs: 1418-1427. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the MMP7 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the MMP7 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an MMP7 gene (e.g., 5′ untranslated region [UTR], exon 1, exon 2, exon 3, exon 4, exon 5, exon 6, any intervening intronic regions, intron/exon junctions, the 3′ UTR, or polyadenylation signal) is targeted by an RNA-guided nuclease to alter the gene. In some embodiments, the MMP7 gene targeted by an RNA-guided nuclease is from a mammal. In some embodiments, the MMP7 gene targeted by an RNA-guided nuclease is from a human (hMMP7). In some embodiments, the MMP7 gene targeted by an RNA-guided nuclease is from a dog (cMMP7). In some embodiments, the MMP7 gene targeted by an RNA-guided nuclease is from ahorse (eMMP7). In some embodiments, the MMP7 gene targeted by an RNA-guided nuclease is from a cat (fMMP7).

### J. MMP8

In one aspect, methods and pharmaceutical compositions are provided for treating a back or spine condition or disorder by administering a therapeutically effective amount of a CRISPR pharmaceutical composition that targets an MMP8 gene to a subject in need thereof. In some embodiments, the CRISPR pharmaceutical composition comprises an RNA-guided nuclease and an sgRNA targeting the MMP8 gene with a crRNA sequence selected from SEQ ID NOs: 1437-1474. In some embodiments, the crRNA sequence is selected from SEQ ID NOs: 1437-1461. In some embodiments, the crRNA sequence is selected from 1437-1446. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the MMP8 gene is delivered to a mammalian cell via viral vector. In some embodiments, the CRISPR gene-editing system targeting the MMP8 gene is delivered to a mammalian cell via an AAV vector. In some embodiments, the CRISPR gene-editing system targeting the MMP8 gene is delivered to a mammalian cell via a lentiviral vector. In some embodiments, the CRISPR gene-editing system targeting the MMP8 gene is delivered to a mammalian cell via a lipid nanoparticle. In some embodiments, the CRISPR gene-editing system targeting the MMP8 gene is delivered to a mammalian cell via a virus-like particle. In some embodiments, the CRISPR gene-editing system targeting the MMP8 gene is delivered to a mammalian cell via a liposome. In some embodiments, the CRISPR gene-editing system targeting the MMP8 gene is delivered to a mammalian cell via a lipid nanocrystal.

In some embodiments, the pharmaceutical composition comprises an sgRNA having a crRNA sequence selected from SEQ ID NOs: 1437-1474 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1437-1474 and a S. pyogenes Cas9 protein encapsulated in a lipid nanoparticle.

In various embodiments, the pharmaceutical composition comprising a CRISPR gene-editing system targeting the MMP8 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the MMP8 gene, as described herein, to a subject in need thereof. In some embodiments, the pharmaceutical composition comprises an LNP encapsuling an sgRNA having a crRNA sequence selected from SEQ ID NOs: 1437-1474 and a S. pyogenes Cas9 protein. In some embodiments, the crRNA sequence is selected from SEQ ID NOs: 1437-1461. In some embodiments, the crRNA sequence is selected from SEQ ID NOs: 1437-1446. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the MMP8 gene, as described herein, to a subject in need thereof. In some embodiments, the musculoskeletal disease or disorder is Arthritis. Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the MMP8 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an MMP8 gene (e.g., 5′ untranslated region [UTR], exon 1, exon 2, exon 3, exon 4, exon 5, exon 6, any intervening intronic regions, intron/exon junctions, the 3′ UTR, or polyadenylation signal) is targeted by an RNA-guided nuclease to alter the gene. In some embodiments, the MMP8 gene targeted by an RNA-guided nuclease is from a mammal. In some embodiments, the MMP8 gene targeted by an RNA-guided nuclease is from a human (hMMP8). In some embodiments, the MMP8 gene targeted by an RNA-guided nuclease is from a dog (cMMP8). In some embodiments, the MMP8 gene targeted by an RNA-guided nuclease is from a horse (eMMP8). In some embodiments, the MMP8 gene targeted by an RNA-guided nuclease is from a cat (fMMP8).

### K. MMP10

In one aspect, methods and pharmaceutical compositions are provided for treating a back or spine condition or disorder by administering a therapeutically effective amount of a CRISPR pharmaceutical composition that targets an MMP10 gene to a subject in need thereof. In some embodiments, the CRISPR pharmaceutical composition comprises an RNA-guided nuclease and an sgRNA targeting the MMP10 gene with a crRNA sequence selected from SEQ ID NOs: 1475-1497. In some embodiments, the crRNA sequence is selected from 1475-1484. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the MMP10 gene is delivered to a mammalian cell via viral vector. In some embodiments, the CRISPR gene-editing system targeting the MMP10 gene is delivered to a mammalian cell via an AAV vector. In some embodiments, the CRISPR gene-editing system targeting the MMP10 gene is delivered to a mammalian cell via a lentiviral vector. In some embodiments, the CRISPR gene-editing system targeting the MMP10 gene is delivered to a mammalian cell via a lipid nanoparticle. In some embodiments, the CRISPR gene-editing system targeting the MMP10 gene is delivered to a mammalian cell via a virus-like particle. In some embodiments, the CRISPR gene-editing system targeting the MMP10 gene is delivered to a mammalian cell via a liposome. In some embodiments, the CRISPR gene-editing system targeting the MMP10 gene is delivered to a mammalian cell via a lipid nanocrystal.

In some embodiments, the pharmaceutical composition comprises an sgRNA having a crRNA sequence selected from SEQ ID NOs: 1475-1497 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1475-1497 and a S. pyogenes Cas9 protein encapsulated in a lipid nanoparticle.

In various embodiments, the pharmaceutical composition comprising a CRISPR gene-editing system targeting the MMP10 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the MMP10 gene, as described herein, to a subject in need thereof. In some embodiments, the pharmaceutical composition comprises an LNP encapsuling an sgRNA having a crRNA sequence selected from SEQ ID NOs: 1475-1497 and a S. pyogenes Cas9 protein. In some embodiments, the crRNA sequence is selected from SEQ ID NOs: 1475-1484. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the MMP10 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the MMP10 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an MMP10 gene (e.g., 5′ untranslated region [UTR], exon 1, exon 2, exon 3, exon 4, exon 5, exon 6, exon 7, exon 8, exon 9, exon 10, any intervening intronic regions, intron/exon junctions, the 3′ UTR, or polyadenylation signal) is targeted by an RNA-guided nuclease to alter the gene. In some embodiments, the MMP10 gene targeted by an RNA-guided nuclease is from a mammal. In some embodiments, the MMP10 gene targeted by an RNA-guided nuclease is from a human (hMMP10). In some embodiments, the MMP10 gene targeted by an RNA-guided nuclease is from a dog (cMMP10). In some embodiments, the MMP10 gene targeted by an RNA-guided nuclease is from a horse (eMMP10). In some embodiments, the MMP10 gene targeted by an RNA-guided nuclease is from a cat (fMMP10).

### L. MMP12

In one aspect, methods and pharmaceutical compositions are provided for treating a back or spine condition or disorder by administering a therapeutically effective amount of a CRISPR pharmaceutical composition that targets an MMP12 gene to a subject in need thereof. In some embodiments, the CRISPR pharmaceutical composition comprises an RNA-guided nuclease and an sgRNA targeting the MMP12 gene with a crRNA sequence selected from SEQ ID NOs: 1498-1541. In some embodiments, the crRNA sequence is selected from SEQ ID NOs: 1498-1522. In some embodiments, the crRNA sequence is selected from 1498-1507. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the MMP12 gene is delivered to a mammalian cell via viral vector. In some embodiments, the CRISPR gene-editing system targeting the MMP12 gene is delivered to a mammalian cell via an AAV vector. In some embodiments, the CRISPR gene-editing system targeting the MMP12 gene is delivered to a mammalian cell via a lentiviral vector. In some embodiments, the CRISPR gene-editing system targeting the MMP12 gene is delivered to a mammalian cell via a lipid nanoparticle. In some embodiments, the CRISPR gene-editing system targeting the MMP12 gene is delivered to a mammalian cell via a virus-like particle. In some embodiments, the CRISPR gene-editing system targeting the MMP12 gene is delivered to a mammalian cell via a liposome. In some embodiments, the CRISPR gene-editing system targeting the MMP12 gene is delivered to a mammalian cell via a lipid nanocrystal.

In some embodiments, the pharmaceutical composition comprises an sgRNA having a crRNA sequence selected from SEQ ID NOs: 1498-1541 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1498-1541 and a S. pyogenes Cas9 protein encapsulated in a lipid nanoparticle.

In various embodiments, the pharmaceutical composition comprising a CRISPR gene-editing system targeting the MMP12 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the MMP12 gene, as described herein, to a subject in need thereof. In some embodiments, the pharmaceutical composition comprises an LNP encapsuling an sgRNA having a crRNA sequence selected from SEQ ID NOs: 1498-1541 and a S. pyogenes Cas9 protein. In some embodiments, the crRNA sequence is selected from SEQ ID NOs: 1498-1522. In some embodiments, the crRNA sequence is selected from SEQ ID NOs: 1498-1507. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the MMP12 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the MMP12 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an MMP12 gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, any intervening intronic regions, intron/exon junctions, the 3′ UTR, or polyadenylation signal) is targeted by an RNA-guided nuclease to alter the gene. In some embodiments, the MMP12 gene targeted by an RNA-guided nuclease is from a mammal. In some embodiments, the MMP12 gene targeted by an RNA-guided nuclease is from a human (hMMP12). In some embodiments, the MMP12 gene targeted by an RNA-guided nuclease is from a dog (cMMP12). In some embodiments, the MMP12 gene targeted by an RNA-guided nuclease is from ahorse (eMMP12). In some embodiments, the MMP12 gene targeted by an RNA-guided nuclease is from a cat (fMMP12).

### M. MMP13

In one aspect, methods and pharmaceutical compositions are provided for treating a back or spine condition or disorder by administering a therapeutically effective amount of a CRISPR pharmaceutical composition that targets an MMP13 gene to a subject in need thereof. In some embodiments, the CRISPR pharmaceutical composition comprises an RNA-guided nuclease and an sgRNA targeting the MMP13 gene with a crRNA sequence selected from SEQ ID NOs: 1542-1568. In some embodiments, the crRNA sequence is selected from 1542-1566. In some embodiments, the crRNA sequence is selected from 1542-1551. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the MMP13 gene is delivered to a mammalian cell via viral vector. In some embodiments, the CRISPR gene-editing system targeting the MMP13 gene is delivered to a mammalian cell via an AAV vector. In some embodiments, the CRISPR gene-editing system targeting the MMP13 gene is delivered to a mammalian cell via a lentiviral vector. In some embodiments, the CRISPR gene-editing system targeting the MMP13 gene is delivered to a mammalian cell via a lipid nanoparticle. In some embodiments, the CRISPR gene-editing system targeting the MMP13 gene is delivered to a mammalian cell via a virus-like particle. In some embodiments, the CRISPR gene-editing system targeting the MMP13 gene is delivered to a mammalian cell via a liposome. In some embodiments, the CRISPR gene-editing system targeting the MMP13 gene is delivered to a mammalian cell via a lipid nanocrystal.

In some embodiments, the pharmaceutical composition comprises an sgRNA having a crRNA sequence selected from SEQ ID NOs: 1542-1568 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1542-1568 and a S. pyogenes Cas9 protein encapsulated in a lipid nanoparticle.

In various embodiments, the pharmaceutical composition comprising a CRISPR gene-editing system targeting the MMP13 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the MMP13 gene, as described herein, to a subject in need thereof. In some embodiments, the pharmaceutical composition comprises an LNP encapsuling an sgRNA having a crRNA sequence selected from SEQ ID NOs: 1542-1568 and a S. pyogenes Cas9 protein. In some embodiments, the crRNA sequence is selected from SEQ ID NOs: 1542-1566. In some embodiments, the crRNA sequence is selected from SEQ ID NOs: 1542-1551. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the MMP13 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the MMP13 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an MMP13 gene (e.g., 5′ untranslated region [UTR], exon 1, exon 2, exon 3, exon 4, exon 5, exon 6, exon 7, exon 8, exon 9, exon 10, any intervening intronic regions, intron/exon junctions, the 3′ UTR, or polyadenylation signal) is targeted by an RNA-guided nuclease to alter the gene. In some embodiments, the MMP13 gene targeted by an RNA-guided nuclease is from a mammal. In some embodiments, the MMP13 gene targeted by an RNA-guided nuclease is from a human (hMMP13). In some embodiments, the MMP13 gene targeted by an RNA-guided nuclease is from a dog (cMMP13). In some embodiments, the MMP13 gene targeted by an RNA-guided nuclease is from a horse (eMMP13). In some embodiments, the MMP13 gene targeted by an RNA-guided nuclease is from a cat (fMMP13).

### N. TIMP1

In one aspect, methods and pharmaceutical compositions are provided for treating a back or spine condition or disorder by administering a therapeutically effective amount of a CRISPR pharmaceutical composition that targets an TIMP1 gene to a subject in need thereof. In some embodiments, the CRISPR pharmaceutical composition comprises an RNA-guided nuclease and an sgRNA targeting the TIMP1 gene with a crRNA sequence selected from SEQ ID NOs: 2470-2509. In some embodiments, the crRNA sequence is selected from SEQ ID NOs: 2470-2494. In some embodiments, the crRNA sequence is selected from 2470-2479. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the TIMP1 gene is delivered to a mammalian cell via viral vector. In some embodiments, the CRISPR gene-editing system targeting the TIMP1 gene is delivered to a mammalian cell via an AAV vector. In some embodiments, the CRISPR gene-editing system targeting the TIMP1 gene is delivered to a mammalian cell via a lentiviral vector. In some embodiments, the CRISPR gene-editing system targeting the TIMP1 gene is delivered to a mammalian cell via a lipid nanoparticle. In some embodiments, the CRISPR gene-editing system targeting the TIMP1 gene is delivered to a mammalian cell via a virus-like particle. In some embodiments, the CRISPR gene-editing system targeting the TIMP1 gene is delivered to a mammalian cell via a liposome. In some embodiments, the CRISPR gene-editing system targeting the TIMP1 gene is delivered to a mammalian cell via a lipid nanocrystal.

In some embodiments, the pharmaceutical composition comprises an sgRNA having a crRNA sequence selected from SEQ ID NOs: 2470-2509 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2470-2509 and a S. pyogenes Cas9 protein encapsulated in a lipid nanoparticle.

In various embodiments, the pharmaceutical composition comprising a CRISPR gene-editing system targeting the TIMP1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the TIMP1 gene, as described herein, to a subject in need thereof. In some embodiments, the pharmaceutical composition comprises an LNP encapsuling an sgRNA having a crRNA sequence selected from SEQ ID NOs: 2470-2509 and a S. pyogenes Cas9 protein. In some embodiments, the crRNA sequence is selected from SEQ ID NOs: 2470-2494. In some embodiments, the crRNA sequence is selected from SEQ ID NOs: 2470-2479. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the TIMP1 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the TIMP1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an TIMP1 gene (e.g., 5′ untranslated region [UTR], exon 1, exon 2, exon 3, exon 4, exon 5, exon 6, any intervening intronic regions, intron/exon junctions, the 3′ UTR, or polyadenylation signal) is targeted by an RNA-guided nuclease to alter the gene. In some embodiments, the TIMP1 gene targeted by an RNA-guided nuclease is from a mammal. In some embodiments, the TIMP1 gene targeted by an RNA-guided nuclease is from a human (hTIMP1). In some embodiments, the TIMP1 gene targeted by an RNA-guided nuclease is from a dog (cTIMP1). In some embodiments, the TIMP1 gene targeted by an RNA-guided nuclease is from a horse (eTIMP1). In some embodiments, the TIMP1 gene targeted by an RNA-guided nuclease is from a cat (fTIMP1).

### O. TIMP3

In one aspect, methods and pharmaceutical compositions are provided for treating a back or spine condition or disorder by administering a therapeutically effective amount of a CRISPR pharmaceutical composition that targets an TIMP3 gene to a subject in need thereof. In some embodiments, the CRISPR pharmaceutical composition comprises an RNA-guided nuclease and an sgRNA targeting the TIMP3 gene with a crRNA sequence selected from SEQ ID NOs: 2510-2557. In some embodiments, the crRNA sequence is selected from SEQ ID NOs: 2510-2534. In some embodiments, the crRNA sequence is selected from 2510-2519. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the TIMP3 gene is delivered to a mammalian cell via viral vector. In some embodiments, the CRISPR gene-editing system targeting the TIMP3 gene is delivered to a mammalian cell via an AAV vector. In some embodiments, the CRISPR gene-editing system targeting the TIMP3 gene is delivered to a mammalian cell via a lentiviral vector. In some embodiments, the CRISPR gene-editing system targeting the TIMP3 gene is delivered to a mammalian cell via a lipid nanoparticle. In some embodiments, the CRISPR gene-editing system targeting the TIMP3 gene is delivered to a mammalian cell via a virus-like particle. In some embodiments, the CRISPR gene-editing system targeting the TIMP3 gene is delivered to a mammalian cell via a liposome. In some embodiments, the CRISPR gene-editing system targeting the TIMP3 gene is delivered to a mammalian cell via a lipid nanocrystal.

In some embodiments, the pharmaceutical composition comprises an sgRNA having a crRNA sequence selected from SEQ ID NOs: 2510-2557 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2510-2557 and a S. pyogenes Cas9 protein encapsulated in a lipid nanoparticle.

In various embodiments, the pharmaceutical composition comprising a CRISPR gene-editing system targeting the TIMP3 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the TIMP3 gene, as described herein, to a subject in need thereof. In some embodiments, the pharmaceutical composition comprises an LNP encapsuling an sgRNA having a crRNA sequence selected from SEQ ID NOs: 2510-2557 and a S. pyogenes Cas9 protein. In some embodiments, the crRNA sequence is selected from SEQ ID NOs: 2510-2534. In some embodiments, the crRNA sequence is selected from SEQ ID NOs: 2510-2519. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the TIMP3 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the TIMP3 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an TIMP3 gene (e.g., 5′ untranslated region [UTR], exon 1, exon 2, exon 3, exon 4, exon 5, exon 6, exon 7, exon 8, exon 9, exon 10, any intervening intronic regions, intron/exon junctions, the 3′ UTR, or polyadenylation signal) is targeted by an RNA-guided nuclease to alter the gene. In some embodiments, the TIMP3 gene targeted by an RNA-guided nuclease is from a mammal. In some embodiments, the TIMP3 gene targeted by an RNA-guided nuclease is from a human (hTIMP3). In some embodiments, the TIMP3 gene targeted by an RNA-guided nuclease is from a dog (cTIMP3). In some embodiments, the TIMP3 gene targeted by an RNA-guided nuclease is from a horse (eTIMP3). In some embodiments, the TIMP3 gene targeted by an RNA-guided nuclease is from a cat (fTIMP3).

### P. CXCL1

In one aspect, methods and pharmaceutical compositions are provided for treating a back or spine condition or disorder by administering a therapeutically effective amount of a CRISPR pharmaceutical composition that targets an CXCL1 gene to a subject in need thereof. In some embodiments, the CRISPR pharmaceutical composition comprises an RNA-guided nuclease and an sgRNA targeting the CXCL1 gene with a crRNA sequence selected from SEQ ID NOs: 535-551. In some embodiments, the crRNA sequence is selected from 535-544. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CXCL1 gene is delivered to a mammalian cell via viral vector. In some embodiments, the CRISPR gene-editing system targeting the CXCL1 gene is delivered to a mammalian cell via an AAV vector. In some embodiments, the CRISPR gene-editing system targeting the CXCL1 gene is delivered to a mammalian cell via a lentiviral vector. In some embodiments, the CRISPR gene-editing system targeting the CXCL1 gene is delivered to a mammalian cell via a lipid nanoparticle. In some embodiments, the CRISPR gene-editing system targeting the CXCL1 gene is delivered to a mammalian cell via a virus-like particle. In some embodiments, the CRISPR gene-editing system targeting the CXCL1 gene is delivered to a mammalian cell via a liposome. In some embodiments, the CRISPR gene-editing system targeting the CXCL1 gene is delivered to a mammalian cell via a lipid nanocrystal.

In some embodiments, the pharmaceutical composition comprises an sgRNA having a crRNA sequence selected from SEQ ID NOs: 535-551 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 535-551 and a S. pyogenes Cas9 protein encapsulated in a lipid nanoparticle.

In various embodiments, the pharmaceutical composition comprising a CRISPR gene-editing system targeting the CXCL1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CXCL1 gene, as described herein, to a subject in need thereof. In some embodiments, the pharmaceutical composition comprises an LNP encapsuling an sgRNA having a crRNA sequence selected from SEQ ID NOs: 535-551 and a S. pyogenes Cas9 protein. In some embodiments, the crRNA sequence is selected from SEQ ID NOs: 535-544. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CXCL1 gene, as described herein, to a subject in need thereof. In some embodiments, the musculoskeletal disease or disorder is Arthritis. Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CXCL1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CXCL1 gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the CXCL1 gene targeted by an RNA-guided nuclease is from a mammal. In some embodiments, the CXCL1 gene targeted by an RNA-guided nuclease is from a human (hCXCL1). In some embodiments, the CXCL1 gene targeted by an RNA-guided nuclease is from a dog (cCXCL1). In some embodiments, the CXCL1 gene targeted by an RNA-guided nuclease is from a horse (eCXCL1). In some embodiments, the CXCL1 gene targeted by an RNA-guided nuclease is from a cat (fCXCL1).

### Q. CXCL2

In one aspect, methods and pharmaceutical compositions are provided for treating a back or spine condition or disorder by administering a therapeutically effective amount of a CRISPR pharmaceutical composition that targets an CXCL2 gene to a subject in need thereof. In some embodiments, the CRISPR pharmaceutical composition comprises an RNA-guided nuclease and an sgRNA targeting the CXCL2 gene with a crRNA sequence selected from SEQ ID NOs: 552-568. In some embodiments, the crRNA sequence is selected from 552-561. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CXCL2 gene is delivered to a mammalian cell via viral vector. In some embodiments, the CRISPR gene-editing system targeting the CXCL2 gene is delivered to a mammalian cell via an AAV vector. In some embodiments, the CRISPR gene-editing system targeting the CXCL2 gene is delivered to a mammalian cell via a lentiviral vector. In some embodiments, the CRISPR gene-editing system targeting the CXCL2 gene is delivered to a mammalian cell via a lipid nanoparticle. In some embodiments, the CRISPR gene-editing system targeting the CXCL2 gene is delivered to a mammalian cell via a virus-like particle. In some embodiments, the CRISPR gene-editing system targeting the CXCL2 gene is delivered to a mammalian cell via a liposome. In some embodiments, the CRISPR gene-editing system targeting the CXCL2 gene is delivered to a mammalian cell via a lipid nanocrystal.

In some embodiments, the pharmaceutical composition comprises an sgRNA having a crRNA sequence selected from SEQ ID NOs: 552-568 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 552-568 and a S. pyogenes Cas9 protein encapsulated in a lipid nanoparticle.

In various embodiments, the pharmaceutical composition comprising a CRISPR gene-editing system targeting the CXCL2 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CXCL2 gene, as described herein, to a subject in need thereof. In some embodiments, the pharmaceutical composition comprises an LNP encapsuling an sgRNA having a crRNA sequence selected from SEQ ID NOs: 552-568 and a S. pyogenes Cas9 protein. In some embodiments, the crRNA sequence is selected from SEQ ID NOs: 552-561. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CXCL2 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CXCL2 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CXCL2 gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the CXCL2 gene targeted by an RNA-guided nuclease is from a mammal. In some embodiments, the CXCL2 gene targeted by an RNA-guided nuclease is from a human (hCXCL2). In some embodiments, the CXCL2 gene targeted by an RNA-guided nuclease is from a dog (cCXCL2). In some embodiments, the CXCL2 gene targeted by an RNA-guided nuclease is from a horse (eCXCL2). In some embodiments, the CXCL2 gene targeted by an RNA-guided nuclease is from a cat (fCXCL2).

### R. CXCL3

In one aspect, methods and pharmaceutical compositions are provided for treating a back or spine condition or disorder by administering a therapeutically effective amount of a CRISPR pharmaceutical composition that targets an CXCL3 gene to a subject in need thereof. In some embodiments, the CRISPR pharmaceutical composition comprises an RNA-guided nuclease and an sgRNA targeting the CXCL3 gene with a crRNA sequence selected from SEQ ID NOs: 569-585. In some embodiments, the crRNA sequence is selected from 569-578. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CXCL3 gene is delivered to a mammalian cell via viral vector. In some embodiments, the CRISPR gene-editing system targeting the CXCL3 gene is delivered to a mammalian cell via an AAV vector. In some embodiments, the CRISPR gene-editing system targeting the CXCL3 gene is delivered to a mammalian cell via a lentiviral vector. In some embodiments, the CRISPR gene-editing system targeting the CXCL3 gene is delivered to a mammalian cell via a lipid nanoparticle. In some embodiments, the CRISPR gene-editing system targeting the CXCL3 gene is delivered to a mammalian cell via a virus-like particle. In some embodiments, the CRISPR gene-editing system targeting the CXCL3 gene is delivered to a mammalian cell via a liposome. In some embodiments, the CRISPR gene-editing system targeting the CXCL3 gene is delivered to a mammalian cell via a lipid nanocrystal.

In some embodiments, the pharmaceutical composition comprises an sgRNA having a crRNA sequence selected from SEQ ID NOs: 569-585 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 569-585 and a S. pyogenes Cas9 protein encapsulated in a lipid nanoparticle.

In various embodiments, the pharmaceutical composition comprising a CRISPR gene-editing system targeting the CXCL3 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CXCL3 gene, as described herein, to a subject in need thereof. In some embodiments, the pharmaceutical composition comprises an LNP encapsuling an sgRNA having a crRNA sequence selected from SEQ ID NOs: 569-585 and a S. pyogenes Cas9 protein. In some embodiments, the crRNA sequence is selected from SEQ ID NOs: 569-578. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CXCL3 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CXCL3 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CXCL3 gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the CXCL3 gene targeted by an RNA-guided nuclease is from a mammal. In some embodiments, the CXCL3 gene targeted by an RNA-guided nuclease is from a human (hCXCL3). In some embodiments, the CXCL3 gene targeted by an RNA-guided nuclease is from a dog (cCXCL3). In some embodiments, the CXCL3 gene targeted by an RNA-guided nuclease is from a horse (eCXCL3). In some embodiments, the CXCL3 gene targeted by an RNA-guided nuclease is from a cat (fCXCL3).

### S. CXCL5

In one aspect, methods and pharmaceutical compositions are provided for treating a back or spine condition or disorder by administering a therapeutically effective amount of a CRISPR pharmaceutical composition that targets an CXCL5 gene to a subject in need thereof. In some embodiments, the CRISPR pharmaceutical composition comprises an RNA-guided nuclease and an sgRNA targeting the CXCL5 gene with a crRNA sequence selected from SEQ ID NOs: 586-602. In some embodiments, the crRNA sequence is selected from 586-595. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CXCL5 gene is delivered to a mammalian cell via viral vector. In some embodiments, the CRISPR gene-editing system targeting the CXCL5 gene is delivered to a mammalian cell via an AAV vector. In some embodiments, the CRISPR gene-editing system targeting the CXCL5 gene is delivered to a mammalian cell via a lentiviral vector. In some embodiments, the CRISPR gene-editing system targeting the CXCL5 gene is delivered to a mammalian cell via a lipid nanoparticle. In some embodiments, the CRISPR gene-editing system targeting the CXCL5 gene is delivered to a mammalian cell via a virus-like particle. In some embodiments, the CRISPR gene-editing system targeting the CXCL5 gene is delivered to a mammalian cell via a liposome. In some embodiments, the CRISPR gene-editing system targeting the CXCL5 gene is delivered to a mammalian cell via a lipid nanocrystal.

In some embodiments, the pharmaceutical composition comprises an sgRNA having a crRNA sequence selected from SEQ ID NOs: 586-602 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 586-602 and a S. pyogenes Cas9 protein encapsulated in a lipid nanoparticle.

In various embodiments, the pharmaceutical composition comprising a CRISPR gene-editing system targeting the CXCL5 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CXCL5 gene, as described herein, to a subject in need thereof. In some embodiments, the pharmaceutical composition comprises an LNP encapsuling an sgRNA having a crRNA sequence selected from SEQ ID NOs: 586-602 and a S. pyogenes Cas9 protein. In some embodiments, the crRNA sequence is selected from SEQ ID NOs: 586-595. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CXCL5 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CXCL5 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CXCL5 gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the CXCL5 gene targeted by an RNA-guided nuclease is from a mammal. In some embodiments, the CXCL5 gene targeted by an RNA-guided nuclease is from a human (hCXCL5). In some embodiments, the CXCL5 gene targeted by an RNA-guided nuclease is from a dog (cCXCL5). In some embodiments, the CXCL5 gene targeted by an RNA-guided nuclease is from a horse (eCXCL5). In some embodiments, the CXCL5 gene targeted by an RNA-guided nuclease is from a cat (fCXCL5).

### T. CXCL6

In one aspect, methods and pharmaceutical compositions are provided for treating a back or spine condition or disorder by administering a therapeutically effective amount of a CRISPR pharmaceutical composition that targets an CXCL6 gene to a subject in need thereof. In some embodiments, the CRISPR pharmaceutical composition comprises an RNA-guided nuclease and an sgRNA targeting the CXCL6 gene with a crRNA sequence selected from SEQ ID NOs: 603-619. In some embodiments, the crRNA sequence is selected from 603-612. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CXCL6 gene is delivered to a mammalian cell via viral vector. In some embodiments, the CRISPR gene-editing system targeting the CXCL6 gene is delivered to a mammalian cell via an AAV vector. In some embodiments, the CRISPR gene-editing system targeting the CXCL6 gene is delivered to a mammalian cell via a lentiviral vector. In some embodiments, the CRISPR gene-editing system targeting the CXCL6 gene is delivered to a mammalian cell via a lipid nanoparticle. In some embodiments, the CRISPR gene-editing system targeting the CXCL6 gene is delivered to a mammalian cell via a virus-like particle. In some embodiments, the CRISPR gene-editing system targeting the CXCL6 gene is delivered to a mammalian cell via a liposome. In some embodiments, the CRISPR gene-editing system targeting the CXCL6 gene is delivered to a mammalian cell via a lipid nanocrystal.

In some embodiments, the pharmaceutical composition comprises an sgRNA having a crRNA sequence selected from SEQ ID NOs: 603-619 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 603-619 and a S. pyogenes Cas9 protein encapsulated in a lipid nanoparticle.

In various embodiments, the pharmaceutical composition comprising a CRISPR gene-editing system targeting the CXCL6 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CXCL6 gene, as described herein, to a subject in need thereof. In some embodiments, the pharmaceutical composition comprises an LNP encapsuling an sgRNA having a crRNA sequence selected from SEQ ID NOs: 603-619 and a S. pyogenes Cas9 protein. In some embodiments, the crRNA sequence is selected from SEQ ID NOs: 603-612. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CXCL6 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CXCL6 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CXCL6 gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the CXCL6 gene targeted by an RNA-guided nuclease is from a mammal. In some embodiments, the CXCL6 gene targeted by an RNA-guided nuclease is from a human (hCXCL6). In some embodiments, the CXCL6 gene targeted by an RNA-guided nuclease is from a dog (cCXCL6). In some embodiments, the CXCL6 gene targeted by an RNA-guided nuclease is from a horse (eCXCL6). In some embodiments, the CXCL6 gene targeted by an RNA-guided nuclease is from a cat (fCXCL6).

### U. CXCL8

In one aspect, methods and pharmaceutical compositions are provided for treating a back or spine condition or disorder by administering a therapeutically effective amount of a CRISPR pharmaceutical composition that targets an CXCL8 gene to a subject in need thereof. In some embodiments, the CRISPR pharmaceutical composition comprises an RNA-guided nuclease and an sgRNA targeting the CXCL8 gene with a crRNA sequence selected from SEQ ID NOs: 620-636. In some embodiments, the crRNA sequence is selected from 620-629. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CXCL8 gene is delivered to a mammalian cell via viral vector. In some embodiments, the CRISPR gene-editing system targeting the CXCL8 gene is delivered to a mammalian cell via an AAV vector. In some embodiments, the CRISPR gene-editing system targeting the CXCL8 gene is delivered to a mammalian cell via a lentiviral vector. In some embodiments, the CRISPR gene-editing system targeting the CXCL8 gene is delivered to a mammalian cell via a lipid nanoparticle. In some embodiments, the CRISPR gene-editing system targeting the CXCL8 gene is delivered to a mammalian cell via a virus-like particle. In some embodiments, the CRISPR gene-editing system targeting the CXCL8 gene is delivered to a mammalian cell via a liposome. In some embodiments, the CRISPR gene-editing system targeting the CXCL8 gene is delivered to a mammalian cell via a lipid nanocrystal.

In some embodiments, the pharmaceutical composition comprises an sgRNA having a crRNA sequence selected from SEQ ID NOs: 620-636 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 620-636 and a S. pyogenes Cas9 protein encapsulated in a lipid nanoparticle.

In various embodiments, the pharmaceutical composition comprising a CRISPR gene-editing system targeting the CXCL8 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CXCL8 gene, as described herein, to a subject in need thereof. In some embodiments, the pharmaceutical composition comprises an LNP encapsuling an sgRNA having a crRNA sequence selected from SEQ ID NOs: 620-636 and a S. pyogenes Cas9 protein. In some embodiments, the crRNA sequence is selected from SEQ ID NOs: 620-629. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CXCL8 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CXCL8 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CXCL8 gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the CXCL8 gene targeted by an RNA-guided nuclease is from a mammal. In some embodiments, the CXCL8 gene targeted by an RNA-guided nuclease is from a human (hCXCL8). In some embodiments, the CXCL8 gene targeted by an RNA-guided nuclease is from a dog (cCXCL8). In some embodiments, the CXCL8 gene targeted by an RNA-guided nuclease is from a horse (eCXCL8). In some embodiments, the CXCL8 gene targeted by an RNA-guided nuclease is from a cat (fCXCL8).

### V. CCL2

In one aspect, methods and pharmaceutical compositions are provided for treating a back or spine condition or disorder by administering a therapeutically effective amount of a CRISPR pharmaceutical composition that targets an CCL2 gene to a subject in need thereof. In some embodiments, the CRISPR pharmaceutical composition comprises an RNA-guided nuclease and an sgRNA targeting the CCL2 gene with a crRNA sequence selected from SEQ ID NOs: 341-357. In some embodiments, the crRNA sequence is selected from 341-350. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CCL2 gene is delivered to a mammalian cell via viral vector. In some embodiments, the CRISPR gene-editing system targeting the CCL2 gene is delivered to a mammalian cell via an AAV vector. In some embodiments, the CRISPR gene-editing system targeting the CCL2 gene is delivered to a mammalian cell via a lentiviral vector. In some embodiments, the CRISPR gene-editing system targeting the CCL2 gene is delivered to a mammalian cell via a lipid nanoparticle. In some embodiments, the CRISPR gene-editing system targeting the CCL2 gene is delivered to a mammalian cell via a virus-like particle. In some embodiments, the CRISPR gene-editing system targeting the CCL2 gene is delivered to a mammalian cell via a liposome. In some embodiments, the CRISPR gene-editing system targeting the CCL2 gene is delivered to a mammalian cell via a lipid nanocrystal.

In some embodiments, the pharmaceutical composition comprises an sgRNA having a crRNA sequence selected from SEQ ID NOs: 341-357 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 341-357 and a S. pyogenes Cas9 protein encapsulated in a lipid nanoparticle.

In various embodiments, the pharmaceutical composition comprising a CRISPR gene-editing system targeting the CCL2 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CCL2 gene, as described herein, to a subject in need thereof. In some embodiments, the pharmaceutical composition comprises an LNP encapsuling an sgRNA having a crRNA sequence selected from SEQ ID NOs: 341-357 and a S. pyogenes Cas9 protein. In some embodiments, the crRNA sequence is selected from SEQ ID NOs: 341-350. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CCL2 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CCL2 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CCL2 gene (e.g., 5′ untranslated region [UTR], exon 1, exon 2, exon 3, any intervening intronic regions, intron/exon junctions, the 3′ UTR, or polyadenylation signal) is targeted by an RNA-guided nuclease to alter the gene. In some embodiments, the CCL2 gene targeted by an RNA-guided nuclease is from a mammal. In some embodiments, the CCL2 gene targeted by an RNA-guided nuclease is from a human (hCCL2). In some embodiments, the CCL2 gene targeted by an RNA-guided nuclease is from a dog (cCCL2). In some embodiments, the CCL2 gene targeted by an RNA-guided nuclease is from a horse (eCCL2). In some embodiments, the CCL2 gene targeted by an RNA-guided nuclease is from a cat (fCCL2).

### W. CCL3

In one aspect, methods and pharmaceutical compositions are provided for treating a back or spine condition or disorder by administering a therapeutically effective amount of a CRISPR pharmaceutical composition that targets an CCL3 gene to a subject in need thereof. In some embodiments, the CRISPR pharmaceutical composition comprises an RNA-guided nuclease and an sgRNA targeting the CCL3 gene with a crRNA sequence selected from SEQ ID NOs: 358-374. In some embodiments, the crRNA sequence is selected from 358-367. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7537_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CCL3 gene is delivered to a mammalian cell via viral vector. In some embodiments, the CRISPR gene-editing system targeting the CCL3 gene is delivered to a mammalian cell via an AAV vector. In some embodiments, the CRISPR gene-editing system targeting the CCL3 gene is delivered to a mammalian cell via a lentiviral vector. In some embodiments, the CRISPR gene-editing system targeting the CCL3 gene is delivered to a mammalian cell via a lipid nanoparticle. In some embodiments, the CRISPR gene-editing system targeting the CCL3 gene is delivered to a mammalian cell via a virus-like particle. In some embodiments, the CRISPR gene-editing system targeting the CCL3 gene is delivered to a mammalian cell via a liposome. In some embodiments, the CRISPR gene-editing system targeting the CCL3 gene is delivered to a mammalian cell via a lipid nanocrystal.

In some embodiments, the pharmaceutical composition comprises an sgRNA having a crRNA sequence selected from SEQ ID NOs: 358-374 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 358-374 and a S. pyogenes Cas9 protein encapsulated in a lipid nanoparticle.

In various embodiments, the pharmaceutical composition comprising a CRISPR gene-editing system targeting the CCL3 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CCL3 gene, as described herein, to a subject in need thereof. In some embodiments, the pharmaceutical composition comprises an LNP encapsuling an sgRNA having a crRNA sequence selected from SEQ ID NOs: 358-374 and a S. pyogenes Cas9 protein. In some embodiments, the crRNA sequence is selected from SEQ ID NOs: 358-367. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CCL3 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CCL3 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CCL3 gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the CCL3 gene targeted by an RNA-guided nuclease is from a mammal. In some embodiments, the CCL3 gene targeted by an RNA-guided nuclease is from a human (hCCL3). In some embodiments, the CCL3 gene targeted by an RNA-guided nuclease is from a dog (cCCL3). In some embodiments, the CCL3 gene targeted by an RNA-guided nuclease is from a horse (eCCL3). In some embodiments, the CCL3 gene targeted by an RNA-guided nuclease is from a cat (fCCL3).

### X. CCL5

In one aspect, methods and pharmaceutical compositions are provided for treating a back or spine condition or disorder by administering a therapeutically effective amount of a CRISPR pharmaceutical composition that targets an CCL5 gene to a subject in need thereof. In some embodiments, the CRISPR pharmaceutical composition comprises an RNA-guided nuclease and an sgRNA targeting the CCL5 gene with a crRNA sequence selected from SEQ ID NOs: 375-391. In some embodiments, the crRNA sequence is selected from 375-384. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CCL5 gene is delivered to a mammalian cell via viral vector. In some embodiments, the CRISPR gene-editing system targeting the CCL5 gene is delivered to a mammalian cell via an AAV vector. In some embodiments, the CRISPR gene-editing system targeting the CCL5 gene is delivered to a mammalian cell via a lentiviral vector. In some embodiments, the CRISPR gene-editing system targeting the CCL5 gene is delivered to a mammalian cell via a lipid nanoparticle. In some embodiments, the CRISPR gene-editing system targeting the CCL5 gene is delivered to a mammalian cell via a virus-like particle. In some embodiments, the CRISPR gene-editing system targeting the CCL5 gene is delivered to a mammalian cell via a liposome. In some embodiments, the CRISPR gene-editing system targeting the CCL5 gene is delivered to a mammalian cell via a lipid nanocrystal.

In some embodiments, the pharmaceutical composition comprises an sgRNA having a crRNA sequence selected from SEQ ID NOs: 375-391 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 375-391 and a S. pyogenes Cas9 protein encapsulated in a lipid nanoparticle.

In various embodiments, the pharmaceutical composition comprising a CRISPR gene-editing system targeting the CCL5 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CCL5 gene, as described herein, to a subject in need thereof. In some embodiments, the pharmaceutical composition comprises an LNP encapsuling an sgRNA having a crRNA sequence selected from SEQ ID NOs: 375-391 and a S. pyogenes Cas9 protein. In some embodiments, the crRNA sequence is selected from SEQ ID NOs: 375-384. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CCL5 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CCL5 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CCL5 gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the CCL5 gene targeted by an RNA-guided nuclease is from a mammal. In some embodiments, the CCL5 gene targeted by an RNA-guided nuclease is from a human (hCCL5). In some embodiments, the CCL5 gene targeted by an RNA-guided nuclease is from a dog (cCCL5). In some embodiments, the CCL5 gene targeted by an RNA-guided nuclease is from a horse (eCCL5). In some embodiments, the CCL5 gene targeted by an RNA-guided nuclease is from a cat (fCCL5).

### Y. CCL7

In one aspect, methods and pharmaceutical compositions are provided for treating a back or spine condition or disorder by administering a therapeutically effective amount of a CRISPR pharmaceutical composition that targets an CCL7 gene to a subject in need thereof. In some embodiments, the CRISPR pharmaceutical composition comprises an RNA-guided nuclease and an sgRNA targeting the CCL7 gene with a crRNA sequence selected from SEQ ID NOs: 392-408. In some embodiments, the crRNA sequence is selected from 392-401. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CCL7 gene is delivered to a mammalian cell via viral vector. In some embodiments, the CRISPR gene-editing system targeting the CCL7 gene is delivered to a mammalian cell via an AAV vector. In some embodiments, the CRISPR gene-editing system targeting the CCL7 gene is delivered to a mammalian cell via a lentiviral vector. In some embodiments, the CRISPR gene-editing system targeting the CCL7 gene is delivered to a mammalian cell via a lipid nanoparticle. In some embodiments, the CRISPR gene-editing system targeting the CCL7 gene is delivered to a mammalian cell via a virus-like particle. In some embodiments, the CRISPR gene-editing system targeting the CCL7 gene is delivered to a mammalian cell via a liposome. In some embodiments, the CRISPR gene-editing system targeting the CCL7 gene is delivered to a mammalian cell via a lipid nanocrystal.

In some embodiments, the pharmaceutical composition comprises an sgRNA having a crRNA sequence selected from SEQ ID NOs: 392-408 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 392-408 and a S. pyogenes Cas9 protein encapsulated in a lipid nanoparticle.

In various embodiments, the pharmaceutical composition comprising a CRISPR gene-editing system targeting the CCL7 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CCL7 gene, as described herein, to a subject in need thereof. In some embodiments, the pharmaceutical composition comprises an LNP encapsuling an sgRNA having a crRNA sequence selected from SEQ ID NOs: 392-408 and a S. pyogenes Cas9 protein. In some embodiments, the crRNA sequence is selected from SEQ ID NOs: 392-401. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CCL7 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CCL7 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CCL7 gene (e.g., 5′ untranslated region [UTR], exon 1, exon 2, exon 3, any intervening intronic regions, intron/exon junctions, the 3′ UTR, or polyadenylation signal) is targeted by an RNA-guided nuclease to alter the gene. In some embodiments, the CCL7 gene targeted by an RNA-guided nuclease is from a mammal. In some embodiments, the CCL7 gene targeted by an RNA-guided nuclease is from a human (hCCL7). In some embodiments, the CCL7 gene targeted by an RNA-guided nuclease is from a dog (cCCL7). In some embodiments, the CCL7 gene targeted by an RNA-guided nuclease is from a horse (eCCL7). In some embodiments, the CCL7 gene targeted by an RNA-guided nuclease is from a cat (fCCL7).

### Z. CCL20

In one aspect, methods and pharmaceutical compositions are provided for treating a back or spine condition or disorder by administering a therapeutically effective amount of a CRISPR pharmaceutical composition that targets an CCL20 gene to a subject in need thereof. In some embodiments, the CRISPR pharmaceutical composition comprises an RNA-guided nuclease and an sgRNA targeting the CCL20 gene with a crRNA sequence selected from SEQ ID NOs: 409-425. In some embodiments, the crRNA sequence is selected from 409-418. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CCL20 gene is delivered to a mammalian cell via viral vector. In some embodiments, the CRISPR gene-editing system targeting the CCL20 gene is delivered to a mammalian cell via an AAV vector. In some embodiments, the CRISPR gene-editing system targeting the CCL20 gene is delivered to a mammalian cell via a lentiviral vector. In some embodiments, the CRISPR gene-editing system targeting the CCL20 gene is delivered to a mammalian cell via a lipid nanoparticle. In some embodiments, the CRISPR gene-editing system targeting the CCL20 gene is delivered to a mammalian cell via a virus-like particle. In some embodiments, the CRISPR gene-editing system targeting the CCL20 gene is delivered to a mammalian cell via a liposome. In some embodiments, the CRISPR gene-editing system targeting the CCL20 gene is delivered to a mammalian cell via a lipid nanocrystal.

In some embodiments, the pharmaceutical composition comprises an sgRNA having a crRNA sequence selected from SEQ ID NOs: 409-425 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 409-425 and a S. pyogenes Cas9 protein encapsulated in a lipid nanoparticle.

In various embodiments, the pharmaceutical composition comprising a CRISPR gene-editing system targeting the CCL20 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CCL20 gene, as described herein, to a subject in need thereof. In some embodiments, the pharmaceutical composition comprises an LNP encapsuling an sgRNA having a crRNA sequence selected from SEQ ID NOs: 409-425 and a S. pyogenes Cas9 protein. In some embodiments, the crRNA sequence is selected from SEQ ID NOs: 409-418. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CCL20 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CCL20 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CCL20 gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the CCL20 gene targeted by an RNA-guided nuclease is from a mammal. In some embodiments, the CCL20 gene targeted by an RNA-guided nuclease is from a human (hCCL20). In some embodiments, the CCL20 gene targeted by an RNA-guided nuclease is from a dog (cCCL20). In some embodiments, the CCL20 gene targeted by an RNA-guided nuclease is from a horse (eCCL20). In some embodiments, the CCL20 gene targeted by an RNA-guided nuclease is from a cat (fCCL20).

### AA. CXCR1

In one aspect, methods and pharmaceutical compositions are provided for treating a back or spine condition or disorder by administering a therapeutically effective amount of a CRISPR pharmaceutical composition that targets an CXCR1 gene to a subject in need thereof. In some embodiments, the CRISPR pharmaceutical composition comprises an RNA-guided nuclease and an sgRNA targeting the CXCR1 gene with a crRNA sequence selected from SEQ ID NOs: 637-655. In some embodiments, the crRNA sequence is selected from 637-646. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CXCR1 gene is delivered to a mammalian cell via viral vector. In some embodiments, the CRISPR gene-editing system targeting the CXCR1 gene is delivered to a mammalian cell via an AAV vector. In some embodiments, the CRISPR gene-editing system targeting the CXCR1 gene is delivered to a mammalian cell via a lentiviral vector. In some embodiments, the CRISPR gene-editing system targeting the CXCR1 gene is delivered to a mammalian cell via a lipid nanoparticle. In some embodiments, the CRISPR gene-editing system targeting the CXCR1 gene is delivered to a mammalian cell via a virus-like particle. In some embodiments, the CRISPR gene-editing system targeting the CXCR1 gene is delivered to a mammalian cell via a liposome. In some embodiments, the CRISPR gene-editing system targeting the CXCR1 gene is delivered to a mammalian cell via a lipid nanocrystal.

In some embodiments, the pharmaceutical composition comprises an sgRNA having a crRNA sequence selected from SEQ ID NOs: 637-655 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 637-655 and a S. pyogenes Cas9 protein encapsulated in a lipid nanoparticle.

In various embodiments, the pharmaceutical composition comprising a CRISPR gene-editing system targeting the CXCR1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CXCR1 gene, as described herein, to a subject in need thereof. In some embodiments, the pharmaceutical composition comprises an LNP encapsuling an sgRNA having a crRNA sequence selected from SEQ ID NOs: 637-655 and a S. pyogenes Cas9 protein. In some embodiments, the crRNA sequence is selected from SEQ ID NOs: 637-646. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CXCR1 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CXCR1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CXCR1 gene (e.g., 5′ untranslated region [UTR], exon 1, exon 2, any intervening intronic regions, intron/exon junctions, the 3′ UTR, or polyadenylation signal) is targeted by an RNA-guided nuclease to alter the gene. In some embodiments, the CXCR1 gene targeted by an RNA-guided nuclease is from a mammal. In some embodiments, the CXCR1 gene targeted by an RNA-guided nuclease is from a human (hCXCR1). In some embodiments, the CXCR1 gene targeted by an RNA-guided nuclease is from a dog (cCXCR1). In some embodiments, the CXCR1 gene targeted by an RNA-guided nuclease is from a horse (eCXCR1). In some embodiments, the CXCR1 gene targeted by an RNA-guided nuclease is from a cat (fCXCR1).

### BB. CXCR2

In one aspect, methods and pharmaceutical compositions are provided for treating a back or spine condition or disorder by administering a therapeutically effective amount of a CRISPR pharmaceutical composition that targets an CXCR2 gene to a subject in need thereof. In some embodiments, the CRISPR pharmaceutical composition comprises an RNA-guided nuclease and an sgRNA targeting the CXCR2 gene with a crRNA sequence selected from SEQ ID NOs: 656-672. In some embodiments, the crRNA sequence is selected from 656-665. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CXCR2 gene is delivered to a mammalian cell via viral vector. In some embodiments, the CRISPR gene-editing system targeting the CXCR2 gene is delivered to a mammalian cell via an AAV vector. In some embodiments, the CRISPR gene-editing system targeting the CXCR2 gene is delivered to a mammalian cell via a lentiviral vector. In some embodiments, the CRISPR gene-editing system targeting the CXCR2 gene is delivered to a mammalian cell via a lipid nanoparticle. In some embodiments, the CRISPR gene-editing system targeting the CXCR2 gene is delivered to a mammalian cell via a virus-like particle. In some embodiments, the CRISPR gene-editing system targeting the CXCR2 gene is delivered to a mammalian cell via a liposome. In some embodiments, the CRISPR gene-editing system targeting the CXCR2 gene is delivered to a mammalian cell via a lipid nanocrystal.

In some embodiments, the pharmaceutical composition comprises an sgRNA having a crRNA sequence selected from SEQ ID NOs: 656-672 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 656-672 and a S. pyogenes Cas9 protein encapsulated in a lipid nanoparticle.

In various embodiments, the pharmaceutical composition comprising a CRISPR gene-editing system targeting the CXCR2 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CXCR2 gene, as described herein, to a subject in need thereof. In some embodiments, the pharmaceutical composition comprises an LNP encapsuling an sgRNA having a crRNA sequence selected from SEQ ID NOs: 656-672 and a S. pyogenes Cas9 protein. In some embodiments, the crRNA sequence is selected from SEQ ID NOs: 656-665. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CXCR2 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CXCR2 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CXCR2 gene (e.g., 5′ untranslated region [UTR], exon 1, exon 2, exon 3, exon 4, exon 5, exon 6, any intervening intronic regions, intron/exon junctions, the 3′ UTR, or polyadenylation signal) is targeted by an RNA-guided nuclease to alter the gene. In some embodiments, the CXCR2 gene targeted by an RNA-guided nuclease is from a mammal. In some embodiments, the CXCR2 gene targeted by an RNA-guided nuclease is from a human (hCXCR2). In some embodiments, the CXCR2 gene targeted by an RNA-guided nuclease is from a dog (cCXCR2). In some embodiments, the CXCR2 gene targeted by an RNA-guided nuclease is from a horse (eCXCR2). In some embodiments, the CXCR2 gene targeted by an RNA-guided nuclease is from a cat (fCXCR2).

### CC. CCR7

In one aspect, methods and pharmaceutical compositions are provided for treating a back or spine condition or disorder by administering a therapeutically effective amount of a CRISPR pharmaceutical composition that targets an CCR7 gene to a subject in need thereof. In some embodiments, the CRISPR pharmaceutical composition comprises an RNA-guided nuclease and an sgRNA targeting the CCR7 gene with a crRNA sequence selected from SEQ ID NOs: 474-517. In some embodiments, the crRNA sequence is selected from SEQ ID NOs: 474-498. In some embodiments, the crRNA sequence is selected from 474-483. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CCR7 gene is delivered to a mammalian cell via viral vector. In some embodiments, the CRISPR gene-editing system targeting the CCR7 gene is delivered to a mammalian cell via an AAV vector. In some embodiments, the CRISPR gene-editing system targeting the CCR7 gene is delivered to a mammalian cell via a lentiviral vector. In some embodiments, the CRISPR gene-editing system targeting the CCR7 gene is delivered to a mammalian cell via a lipid nanoparticle. In some embodiments, the CRISPR gene-editing system targeting the CCR7 gene is delivered to a mammalian cell via a virus-like particle. In some embodiments, the CRISPR gene-editing system targeting the CCR7 gene is delivered to a mammalian cell via a liposome. In some embodiments, the CRISPR gene-editing system targeting the CCR7 gene is delivered to a mammalian cell via a lipid nanocrystal.

In some embodiments, the pharmaceutical composition comprises an sgRNA having a crRNA sequence selected from SEQ ID NOs: 474-517 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 474-517 and a S. pyogenes Cas9 protein encapsulated in a lipid nanoparticle.

In various embodiments, the pharmaceutical composition comprising a CRISPR gene-editing system targeting the CCR7 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CCR7 gene, as described herein, to a subject in need thereof. In some embodiments, the pharmaceutical composition comprises an LNP encapsuling an sgRNA having a crRNA sequence selected from SEQ ID NOs: 474-517 and a S. pyogenes Cas9 protein. In some embodiments, the crRNA sequence is selected from SEQ ID NOs: 474-498. In some embodiments, the crRNA sequence is selected from SEQ ID NOs: 474-483. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CCR7 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CCR7 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CCR7 gene (e.g., 5′ untranslated region [UTR], exon 1, exon 2, exon 3, exon 4, exon 5, any intervening intronic regions, intron/exon junctions, the 3′ UTR, or polyadenylation signal) is targeted by an RNA-guided nuclease to alter the gene. In some embodiments, the CCR7 gene targeted by an RNA-guided nuclease is from a mammal. In some embodiments, the CCR7 gene targeted by an RNA-guided nuclease is from a human (hCCR7). In some embodiments, the CCR7 gene targeted by an RNA-guided nuclease is from a dog (cCCR7). In some embodiments, the CCR7 gene targeted by an RNA-guided nuclease is from a horse (eCCR7). In some embodiments, the CCR7 gene targeted by an RNA-guided nuclease is from a cat (fCCR7).

### DD. ADAM17

In one aspect, methods and pharmaceutical compositions are provided for treating a back or spine condition or disorder by administering a therapeutically effective amount of a CRISPR pharmaceutical composition that targets an ADAM17 gene to a subject in need thereof. In some embodiments, the CRISPR pharmaceutical composition comprises an RNA-guided nuclease and an sgRNA targeting the ADAM17 gene with a crRNA sequence selected from SEQ ID NOs: 1-48. In some embodiments, the crRNA sequence is selected from SEQ ID NOs: 1-25. In some embodiments, the crRNA sequence is selected from 1-10. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the ADAM17 gene is delivered to a mammalian cell via viral vector. In some embodiments, the CRISPR gene-editing system targeting the ADAM17 gene is delivered to a mammalian cell via an AAV vector. In some embodiments, the CRISPR gene-editing system targeting the ADAM17 gene is delivered to a mammalian cell via a lentiviral vector. In some embodiments, the CRISPR gene-editing system targeting the ADAM17 gene is delivered to a mammalian cell via a lipid nanoparticle. In some embodiments, the CRISPR gene-editing system targeting the ADAM17 gene is delivered to a mammalian cell via a virus-like particle. In some embodiments, the CRISPR gene-editing system targeting the ADAM17 gene is delivered to a mammalian cell via a liposome. In some embodiments, the CRISPR gene-editing system targeting the ADAM17 gene is delivered to a mammalian cell via a lipid nanocrystal.

In some embodiments, the pharmaceutical composition comprises an sgRNA having a crRNA sequence selected from SEQ ID NOs: 1-48 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1-48 and a S. pyogenes Cas9 protein encapsulated in a lipid nanoparticle.

In various embodiments, the pharmaceutical composition comprising a CRISPR gene-editing system targeting the ADAM17 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the ADAM17 gene, as described herein, to a subject in need thereof. In some embodiments, the pharmaceutical composition comprises an LNP encapsuling an sgRNA having a crRNA sequence selected from SEQ ID NOs: 1-48 and a S. pyogenes Cas9 protein. In some embodiments, the crRNA sequence is selected from SEQ ID NOs: 1-25. In some embodiments, the crRNA sequence is selected from SEQ ID NOs: 1-10. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the ADAM17 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the ADAM17 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an ADAM17 gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, any intervening intronic regions, intron/exon junctions, the 3′ UTR, or polyadenylation signal) is targeted by an RNA-guided nuclease to alter the gene. In some embodiments, the ADAM17 gene targeted by an RNA-guided nuclease is from a mammal. In some embodiments, the ADAM17 gene targeted by an RNA-guided nuclease is from a human (hADAM17). In some embodiments, the ADAM17 gene targeted by an RNA-guided nuclease is from a dog (cADAM17). In some embodiments, the ADAM17 gene targeted by an RNA-guided nuclease is from a horse (eADAM17). In some embodiments, the ADAM17 gene targeted by an RNA-guided nuclease is from a cat (fADAM17).

### EE. TNF

In one aspect, methods and pharmaceutical compositions are provided for treating a back or spine condition or disorder by administering a therapeutically effective amount of a CRISPR pharmaceutical composition that targets an TNF gene to a subject in need thereof. In some embodiments, the CRISPR pharmaceutical composition comprises an RNA-guided nuclease and an sgRNA targeting the TNF gene with a crRNA sequence selected from SEQ ID NOs: 2558-2574. In some embodiments, the crRNA sequence is selected from 2558-2567. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7537_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the TNF gene is delivered to a mammalian cell via viral vector. In some embodiments, the CRISPR gene-editing system targeting the TNF gene is delivered to a mammalian cell via an AAV vector. In some embodiments, the CRISPR gene-editing system targeting the TNF gene is delivered to a mammalian cell via a lentiviral vector. In some embodiments, the CRISPR gene-editing system targeting the TNF gene is delivered to a mammalian cell via a lipid nanoparticle. In some embodiments, the CRISPR gene-editing system targeting the TNF gene is delivered to a mammalian cell via a virus-like particle. In some embodiments, the CRISPR gene-editing system targeting the TNF gene is delivered to a mammalian cell via a liposome. In some embodiments, the CRISPR gene-editing system targeting the TNF gene is delivered to a mammalian cell via a lipid nanocrystal.

In some embodiments, the pharmaceutical composition comprises an sgRNA having a crRNA sequence selected from SEQ ID NOs: 2558-2574 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2558-2574 and a S. pyogenes Cas9 protein encapsulated in a lipid nanoparticle.

In various embodiments, the pharmaceutical composition comprising a CRISPR gene-editing system targeting the TNF gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the TNF gene, as described herein, to a subject in need thereof. In some embodiments, the pharmaceutical composition comprises an LNP encapsuling an sgRNA having a crRNA sequence selected from SEQ ID NOs: 2558-2574 and a S. pyogenes Cas9 protein. In some embodiments, the crRNA sequence is selected from SEQ ID NOs: 2558-2567. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the TNF gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the TNF gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an TNF gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the TNF gene targeted by an RNA-guided nuclease is from a mammal. In some embodiments, the TNF gene targeted by an RNA-guided nuclease is from a human (hTNF). In some embodiments, the TNF gene targeted by an RNA-guided nuclease is from a dog (cTNF). In some embodiments, the TNF gene targeted by an RNA-guided nuclease is from a horse (eTNF). In some embodiments, the TNF gene targeted by an RNA-guided nuclease is from a cat (fFNF).

### FF. TNFRSF1A

In one aspect, methods and pharmaceutical compositions are provided for treating a back or spine condition or disorder by administering a therapeutically effective amount of a CRISPR pharmaceutical composition that targets an TNFRSF1A gene to a subject in need thereof. In some embodiments, the CRISPR pharmaceutical composition comprises an RNA-guided nuclease and an sgRNA targeting the TNFRSF1A gene with a crRNA sequence selected from SEQ ID NOs: 2575-2622. In some embodiments, the crRNA sequence is selected from SEQ ID NOs: 2575-2599. In some embodiments, the crRNA sequence is selected from 2575-2584. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the TNFRSF1A gene is delivered to a mammalian cell via viral vector. In some embodiments, the CRISPR gene-editing system targeting the TNFRSF1A gene is delivered to a mammalian cell via an AAV vector. In some embodiments, the CRISPR gene-editing system targeting the TNFRSF1A gene is delivered to a mammalian cell via a lentiviral vector. In some embodiments, the CRISPR gene-editing system targeting the TNFRSF1A gene is delivered to a mammalian cell via a lipid nanoparticle. In some embodiments, the CRISPR gene-editing system targeting the TNFRSF1A gene is delivered to a mammalian cell via a virus-like particle. In some embodiments, the CRISPR gene-editing system targeting the TNFRSF1A gene is delivered to a mammalian cell via a liposome. In some embodiments, the CRISPR gene-editing system targeting the TNFRSF1A gene is delivered to a mammalian cell via a lipid nanocrystal.

In some embodiments, the pharmaceutical composition comprises an sgRNA having a crRNA sequence selected from SEQ ID NOs: 2575-2622 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2575-2622 and a S. pyogenes Cas9 protein encapsulated in a lipid nanoparticle.

In various embodiments, the pharmaceutical composition comprising a CRISPR gene-editing system targeting the TNFRSF1A gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the TNFRSF1A gene, as described herein, to a subject in need thereof. In some embodiments, the pharmaceutical composition comprises an LNP encapsuling an sgRNA having a crRNA sequence selected from SEQ ID NOs: 2575-2622 and a S. pyogenes Cas9 protein. In some embodiments, the crRNA sequence is selected from SEQ ID NOs: 2575-2599. In some embodiments, the crRNA sequence is selected from SEQ ID NOs: 2575-2584. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the TNFRSF1A gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the TNFRSF1A gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an TNFRSF1A gene (e.g., 5′ untranslated region [UTR], exon 1, exon 2, exon 3, exon 4, exon 5, exon 6, exon 7, exon 8, exon 9, exon 10, exon 11, any intervening intronic regions, intron/exon junctions, the 3′ UTR, or polyadenylation signal) is targeted by an RNA-guided nuclease to alter the gene. In some embodiments, the TNFRSF1A gene targeted by an RNA-guided nuclease is from a mammal. In some embodiments, the TNFRSF1A gene targeted by an RNA-guided nuclease is from a human (hTNFRSF1A). In some embodiments, the TNFRSF1A gene targeted by an RNA-guided nuclease is from a dog (cTNFRSF1A). In some embodiments, the TNFRSF1A gene targeted by an RNA-guided nuclease is from a horse (eTNFRSF1A). In some embodiments, the TNFRSF1A gene targeted by an RNA-guided nuclease is from a cat (fTNFRSF1A).

### GG. TNFRSF1B

In one aspect, methods and pharmaceutical compositions are provided for treating a back or spine condition or disorder by administering a therapeutically effective amount of a CRISPR pharmaceutical composition that targets an TNFRSF1B gene to a subject in need thereof. In some embodiments, the CRISPR pharmaceutical composition comprises an RNA-guided nuclease and an sgRNA targeting the TNFRSF1B gene with a crRNA sequence selected from SEQ ID NOs: 2623-2670. In some embodiments, the crRNA sequence is selected from SEQ ID NOs: 2623-2647. In some embodiments, the crRNA sequence is selected from 2623-2632. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the TNFRSF1B gene is delivered to a mammalian cell via viral vector. In some embodiments, the CRISPR gene-editing system targeting the TNFRSF1B gene is delivered to a mammalian cell via an AAV vector. In some embodiments, the CRISPR gene-editing system targeting the TNFRSF1B gene is delivered to a mammalian cell via a lentiviral vector. In some embodiments, the CRISPR gene-editing system targeting the TNFRSF1B gene is delivered to a mammalian cell via a lipid nanoparticle. In some embodiments, the CRISPR gene-editing system targeting the TNFRSF1B gene is delivered to a mammalian cell via a virus-like particle. In some embodiments, the CRISPR gene-editing system targeting the TNFRSF1B gene is delivered to a mammalian cell via a liposome. In some embodiments, the CRISPR gene-editing system targeting the TNFRSF1B gene is delivered to a mammalian cell via a lipid nanocrystal.

In some embodiments, the pharmaceutical composition comprises an sgRNA having a crRNA sequence selected from SEQ ID NOs: 2623-2670 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2623-2670 and a S. pyogenes Cas9 protein encapsulated in a lipid nanoparticle.

In various embodiments, the pharmaceutical composition comprising a CRISPR gene-editing system targeting the TNFRSF1B gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the TNFRSF1B gene, as described herein, to a subject in need thereof. In some embodiments, the pharmaceutical composition comprises an LNP encapsuling an sgRNA having a crRNA sequence selected from SEQ ID NOs: 2623-2670 and a S. pyogenes Cas9 protein. In some embodiments, the crRNA sequence is selected from SEQ ID NOs: 2623-2647. In some embodiments, the crRNA sequence is selected from SEQ ID NOs: 2623-2632. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the TNFRSF1B gene, as described herein, to a subject in need thereof. In some embodiments, the musculoskeletal disease or disorder is Arthritis. Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the TNFRSF1B gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an TNFRSF1B gene (e.g., 5′ untranslated region [UTR], exon 1, exon 2, exon 3, exon 4, exon 5, exon 6, exon 7, exon 8, exon 9, exon 10, exon 11, exon 12, exon 13, any intervening intronic regions, intron/exon junctions, the 3′ UTR, or polyadenylation signal) is targeted by an RNA-guided nuclease to alter the gene. In some embodiments, the TNFRSF1B gene targeted by an RNA-guided nuclease is from a mammal. In some embodiments, the TNFRSF1B gene targeted by an RNA-guided nuclease is from a human (hTNFRSF1B). In some embodiments, the TNFRSF1B gene targeted by an RNA-guided nuclease is from a dog (cTNFRSF1B). In some embodiments, the TNFRSF1B gene targeted by an RNA-guided nuclease is from a horse (eTNFRSF1B). In some embodiments, the TNFRSF1B gene targeted by an RNA-guided nuclease is from a cat (fTNFRSF1B).

### HH. IL4

In one aspect, methods and pharmaceutical compositions are provided for treating a back or spine condition or disorder by administering a therapeutically effective amount of a CRISPR pharmaceutical composition that targets an IL4 gene to a subject in need thereof. In some embodiments, the CRISPR pharmaceutical composition comprises an RNA-guided nuclease and an sgRNA targeting the IL4 gene with a crRNA sequence selected from SEQ ID NOs: 888-911. In some embodiments, the crRNA sequence is selected from 888-897. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL4 gene is delivered to a mammalian cell via viral vector. In some embodiments, the CRISPR gene-editing system targeting the IL4 gene is delivered to a mammalian cell via an AAV vector. In some embodiments, the CRISPR gene-editing system targeting the IL4 gene is delivered to a mammalian cell via a lentiviral vector. In some embodiments, the CRISPR gene-editing system targeting the IL4 gene is delivered to a mammalian cell via a lipid nanoparticle. In some embodiments, the CRISPR gene-editing system targeting the IL4 gene is delivered to a mammalian cell via a virus-like particle. In some embodiments, the CRISPR gene-editing system targeting the IL4 gene is delivered to a mammalian cell via a liposome. In some embodiments, the CRISPR gene-editing system targeting the IL4 gene is delivered to a mammalian cell via a lipid nanocrystal.

In some embodiments, the pharmaceutical composition comprises an sgRNA having a crRNA sequence selected from SEQ ID NOs: 888-911 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 888-911 and a S. pyogenes Cas9 protein encapsulated in a lipid nanoparticle.

In various embodiments, the pharmaceutical composition comprising a CRISPR gene-editing system targeting the IL4 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL4 gene, as described herein, to a subject in need thereof. In some embodiments, the pharmaceutical composition comprises an LNP encapsuling an sgRNA having a crRNA sequence selected from SEQ ID NOs: 888-911 and a S. pyogenes Cas9 protein. In some embodiments, the crRNA sequence is selected from SEQ ID NOs: 888-897. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL4 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL4 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL4 gene (e.g., 5′ untranslated region [UTR], exon 1, exon 2, exon 3, exon 4, exon 5, any intervening intronic regions, intron/exon junctions, the 3′ UTR, or polyadenylation signal) is targeted by an RNA-guided nuclease to alter the gene. In some embodiments, the IL4 gene targeted by an RNA-guided nuclease is from a mammal. In some embodiments, the IL4 gene targeted by an RNA-guided nuclease is from a human (hIL4). In some embodiments, the IL4 gene targeted by an RNA-guided nuclease is from a dog (cIL4). In some embodiments, the IL4 gene targeted by an RNA-guided nuclease is from a horse (eIL4). In some embodiments, the IL4 gene targeted by an RNA-guided nuclease is from a cat (fIL4).

### II. IL4R

In one aspect, methods and pharmaceutical compositions are provided for treating a back or spine condition or disorder by administering a therapeutically effective amount of a CRISPR pharmaceutical composition that targets an IL4R gene to a subject in need thereof. In some embodiments, the CRISPR pharmaceutical composition comprises an RNA-guided nuclease and an sgRNA targeting the IL4R gene with a crRNA sequence selected from SEQ ID NOs: XXX-XXXX. In some embodiments, the crRNA sequence is selected from SEQ ID NOs: YYY-YYYY. In some embodiments, the crRNA sequence is selected from ZZZ-ZZZZ. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL4R gene is delivered to a mammalian cell via viral vector. In some embodiments, the CRISPR gene-editing system targeting the IL4R gene is delivered to a mammalian cell via an AAV vector. In some embodiments, the CRISPR gene-editing system targeting the IL4R gene is delivered to a mammalian cell via a lentiviral vector. In some embodiments, the CRISPR gene-editing system targeting the IL4R gene is delivered to a mammalian cell via a lipid nanoparticle. In some embodiments, the CRISPR gene-editing system targeting the IL4R gene is delivered to a mammalian cell via a virus-like particle. In some embodiments, the CRISPR gene-editing system targeting the IL4R gene is delivered to a mammalian cell via a liposome. In some embodiments, the CRISPR gene-editing system targeting the IL4R gene is delivered to a mammalian cell via a lipid nanocrystal.

In some embodiments, the pharmaceutical composition comprises an sgRNA having a crRNA sequence selected from SEQ ID NOs: XXX-XXXX and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: XXX-XXXX and a S. pyogenes Cas9 protein encapsulated in a lipid nanoparticle.

In various embodiments, the pharmaceutical composition comprising a CRISPR gene-editing system targeting the IL4R gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL4R gene, as described herein, to a subject in need thereof. In some embodiments, the pharmaceutical composition comprises an LNP encapsuling an sgRNA having a crRNA sequence selected from SEQ ID NOs: XXX-XXXX and a S. pyogenes Cas9 protein. In some embodiments, the crRNA sequence is selected from SEQ ID NOs: YYY-YYYY. In some embodiments, the crRNA sequence is selected from SEQ ID NOs: ZZZ-ZZZZ. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL4R gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL4R gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL4R gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, any intervening intronic regions, intron/exon junctions, the 3′ UTR, or polyadenylation signal) is targeted by an RNA-guided nuclease to alter the gene. In some embodiments, the IL4R gene targeted by an RNA-guided nuclease is from a mammal. In some embodiments, the IL4R gene targeted by an RNA-guided nuclease is from a human (hIL4R). In some embodiments, the IL4R gene targeted by an RNA-guided nuclease is from a dog (cIL4R). In some embodiments, the IL4R gene targeted by an RNA-guided nuclease is from a horse (eIL4R). In some embodiments, the IL4R gene targeted by an RNA-guided nuclease is from a cat (fIL4R).

### JJ. IL6

In one aspect, methods and pharmaceutical compositions are provided for treating a back or spine condition or disorder by administering a therapeutically effective amount of a CRISPR pharmaceutical composition that targets an IL6 gene to a subject in need thereof. In some embodiments, the CRISPR pharmaceutical composition comprises an RNA-guided nuclease and an sgRNA targeting the IL6 gene with a crRNA sequence selected from SEQ ID NOs: 912-928. In some embodiments, the crRNA sequence is selected from 912-921. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL6 gene is delivered to a mammalian cell via viral vector. In some embodiments, the CRISPR gene-editing system targeting the IL6 gene is delivered to a mammalian cell via an AAV vector. In some embodiments, the CRISPR gene-editing system targeting the IL6 gene is delivered to a mammalian cell via a lentiviral vector. In some embodiments, the CRISPR gene-editing system targeting the IL6 gene is delivered to a mammalian cell via a lipid nanoparticle. In some embodiments, the CRISPR gene-editing system targeting the IL6 gene is delivered to a mammalian cell via a virus-like particle. In some embodiments, the CRISPR gene-editing system targeting the IL6 gene is delivered to a mammalian cell via a liposome. In some embodiments, the CRISPR gene-editing system targeting the IL6 gene is delivered to a mammalian cell via a lipid nanocrystal.

In some embodiments, the pharmaceutical composition comprises an sgRNA having a crRNA sequence selected from SEQ ID NOs: 912-928 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 912-928 and a S. pyogenes Cas9 protein encapsulated in a lipid nanoparticle.

In various embodiments, the pharmaceutical composition comprising a CRISPR gene-editing system targeting the IL6 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL6 gene, as described herein, to a subject in need thereof. In some embodiments, the pharmaceutical composition comprises an LNP encapsuling an sgRNA having a crRNA sequence selected from SEQ ID NOs: 912-928 and a S. pyogenes Cas9 protein. In some embodiments, the crRNA sequence is selected from SEQ ID NOs: 912-921. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL6 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL6 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL6 gene (e.g., 5′ untranslated region [UTR], exon 1, exon 2, exon 3, exon 4, exon 5, exon 6, any intervening intronic regions, intron/exon junctions, the 3′ UTR, or polyadenylation signal) is targeted by an RNA-guided nuclease to alter the gene. In some embodiments, the IL6 gene targeted by an RNA-guided nuclease is from a mammal. In some embodiments, the IL6 gene targeted by an RNA-guided nuclease is from a human (hIL6). In some embodiments, the IL6 gene targeted by an RNA-guided nuclease is from a dog (cIL6). In some embodiments, the IL6 gene targeted by an RNA-guided nuclease is from a horse (eIL6). In some embodiments, the IL6 gene targeted by an RNA-guided nuclease is from a cat (fIL6).

### KK. IL6R

In one aspect, methods and pharmaceutical compositions are provided for treating a back or spine condition or disorder by administering a therapeutically effective amount of a CRISPR pharmaceutical composition that targets an IL6R gene to a subject in need thereof. In some embodiments, the CRISPR pharmaceutical composition comprises an RNA-guided nuclease and an sgRNA targeting the IL6R gene with a crRNA sequence selected from SEQ ID NOs: 929-963. In some embodiments, the crRNA sequence is selected from 929-953. In some embodiments, the crRNA sequence is selected from 929-938. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL6R gene is delivered to a mammalian cell via viral vector. In some embodiments, the CRISPR gene-editing system targeting the IL6R gene is delivered to a mammalian cell via an AAV vector. In some embodiments, the CRISPR gene-editing system targeting the IL6R gene is delivered to a mammalian cell via a lentiviral vector. In some embodiments, the CRISPR gene-editing system targeting the IL6R gene is delivered to a mammalian cell via a lipid nanoparticle. In some embodiments, the CRISPR gene-editing system targeting the IL6R gene is delivered to a mammalian cell via a virus-like particle. In some embodiments, the CRISPR gene-editing system targeting the IL6R gene is delivered to a mammalian cell via a liposome. In some embodiments, the CRISPR gene-editing system targeting the IL6R gene is delivered to a mammalian cell via a lipid nanocrystal.

In some embodiments, the pharmaceutical composition comprises an sgRNA having a crRNA sequence selected from SEQ ID NOs: 929-963 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 929-963 and a S. pyogenes Cas9 protein encapsulated in a lipid nanoparticle.

In various embodiments, the pharmaceutical composition comprising a CRISPR gene-editing system targeting the IL6R gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL6R gene, as described herein, to a subject in need thereof. In some embodiments, the pharmaceutical composition comprises an LNP encapsuling an sgRNA having a crRNA sequence selected from SEQ ID NOs: 929-963 and a S. pyogenes Cas9 protein. In some embodiments, the crRNA sequence is selected from SEQ ID NOs: 929-953. In some embodiments, the crRNA sequence is selected from SEQ ID NOs: 929-938. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL6R gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL6R gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL6R gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, any intervening intronic regions, intron/exon junctions, the 3′ UTR, or polyadenylation signal) is targeted by an RNA-guided nuclease to alter the gene. In some embodiments, the IL6R gene targeted by an RNA-guided nuclease is from a mammal. In some embodiments, the IL6R gene targeted by an RNA-guided nuclease is from a human (hIL6R). In some embodiments, the IL6R gene targeted by an RNA-guided nuclease is from a dog (cIL6R). In some embodiments, the IL6R gene targeted by an RNA-guided nuclease is from a horse (eIL6R). In some embodiments, the IL6R gene targeted by an RNA-guided nuclease is from a cat (fIL6R).

### LL. IL6ST

In one aspect, methods and pharmaceutical compositions are provided for treating a back or spine condition or disorder by administering a therapeutically effective amount of a CRISPR pharmaceutical composition that targets an IL6ST gene to a subject in need thereof. In some embodiments, the CRISPR pharmaceutical composition comprises an RNA-guided nuclease and an sgRNA targeting the IL6ST gene with a crRNA sequence selected from SEQ ID NOs: 964-990. In some embodiments, the crRNA sequence is selected from 964-988. In some embodiments, the crRNA sequence is selected from 964-973. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL6ST gene is delivered to a mammalian cell via viral vector. In some embodiments, the CRISPR gene-editing system targeting the IL6ST gene is delivered to a mammalian cell via an AAV vector. In some embodiments, the CRISPR gene-editing system targeting the IL6ST gene is delivered to a mammalian cell via a lentiviral vector. In some embodiments, the CRISPR gene-editing system targeting the IL6ST gene is delivered to a mammalian cell via a lipid nanoparticle. In some embodiments, the CRISPR gene-editing system targeting the IL6ST gene is delivered to a mammalian cell via a virus-like particle. In some embodiments, the CRISPR gene-editing system targeting the IL6ST gene is delivered to a mammalian cell via a liposome. In some embodiments, the CRISPR gene-editing system targeting the IL6ST gene is delivered to a mammalian cell via a lipid nanocrystal.

In some embodiments, the pharmaceutical composition comprises an sgRNA having a crRNA sequence selected from SEQ ID NOs: 964-990 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 964-990 and a S. pyogenes Cas9 protein encapsulated in a lipid nanoparticle.

In various embodiments, the pharmaceutical composition comprising a CRISPR gene-editing system targeting the IL6ST gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL6ST gene, as described herein, to a subject in need thereof. In some embodiments, the pharmaceutical composition comprises an LNP encapsuling an sgRNA having a crRNA sequence selected from SEQ ID NOs: 964-990 and a S. pyogenes Cas9 protein. In some embodiments, the crRNA sequence is selected from SEQ ID NOs: 964-988. In some embodiments, the crRNA sequence is selected from SEQ ID NOs: 964-973. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL6ST gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL6ST gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL6ST gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, any intervening intronic regions, intron/exon junctions, the 3′ UTR, or polyadenylation signal) is targeted by an RNA-guided nuclease to alter the gene. In some embodiments, the IL6ST gene targeted by an RNA-guided nuclease is from a mammal. In some embodiments, the IL6ST gene targeted by an RNA-guided nuclease is from a human (hIL6ST). In some embodiments, the IL6ST gene targeted by an RNA-guided nuclease is from a dog (cIL6ST). In some embodiments, the IL6ST gene targeted by an RNA-guided nuclease is from a horse (eIL6ST). In some embodiments, the IL6ST gene targeted by an RNA-guided nuclease is from a cat (fIL6ST).

### MM. IL10

In one aspect, methods and pharmaceutical compositions are provided for treating a back or spine condition or disorder by administering a therapeutically effective amount of a CRISPR pharmaceutical composition that targets an IL10 gene to a subject in need thereof. In some embodiments, the CRISPR pharmaceutical composition comprises an RNA-guided nuclease and an sgRNA targeting the IL10 gene with a crRNA sequence selected from SEQ ID NOs: 964-990. In some embodiments, the crRNA sequence is selected from SEQ ID NOs: 964-988. In some embodiments, the crRNA sequence is selected from 964-973. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL10 gene is delivered to a mammalian cell via viral vector. In some embodiments, the CRISPR gene-editing system targeting the IL10 gene is delivered to a mammalian cell via an AAV vector. In some embodiments, the CRISPR gene-editing system targeting the IL10 gene is delivered to a mammalian cell via a lentiviral vector. In some embodiments, the CRISPR gene-editing system targeting the IL10 gene is delivered to a mammalian cell via a lipid nanoparticle. In some embodiments, the CRISPR gene-editing system targeting the IL10 gene is delivered to a mammalian cell via a virus-like particle. In some embodiments, the CRISPR gene-editing system targeting the IL10 gene is delivered to a mammalian cell via a liposome. In some embodiments, the CRISPR gene-editing system targeting the IL10 gene is delivered to a mammalian cell via a lipid nanocrystal.

In some embodiments, the pharmaceutical composition comprises an sgRNA having a crRNA sequence selected from SEQ ID NOs: 964-990 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 964-990 and a S. pyogenes Cas9 protein encapsulated in a lipid nanoparticle.

In various embodiments, the pharmaceutical composition comprising a CRISPR gene-editing system targeting the IL10 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL10 gene, as described herein, to a subject in need thereof. In some embodiments, the pharmaceutical composition comprises an LNP encapsuling an sgRNA having a crRNA sequence selected from SEQ ID NOs: 964-990 and a S. pyogenes Cas9 protein. In some embodiments, the crRNA sequence is selected from SEQ ID NOs: 964-988. In some embodiments, the crRNA sequence is selected from SEQ ID NOs: 964-973. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL10 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL10 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL10 gene (e.g., 5′ untranslated region [UTR], exon 1, exon 2, exon 3, exon 4, exon 5, exon 6, exon 7, any intervening intronic regions, intron/exon junctions, the 3′ UTR, or polyadenylation signal) is targeted by an RNA-guided nuclease to alter the gene. In some embodiments, the IL10 gene targeted by an RNA-guided nuclease is from a mammal. In some embodiments, the IL10 gene targeted by an RNA-guided nuclease is from a human (hIL10). In some embodiments, the IL10 gene targeted by an RNA-guided nuclease is from a dog (cIL10). In some embodiments, the IL10 gene targeted by an RNA-guided nuclease is from ahorse (eIL10). In some embodiments, the IL10 gene targeted by an RNA-guided nuclease is from a cat (fIL10).

### NN. IL10RA

In one aspect, methods and pharmaceutical compositions are provided for treating a back or spine condition or disorder by administering a therapeutically effective amount of a CRISPR pharmaceutical composition that targets an IL10RA gene to a subject in need thereof. In some embodiments, the CRISPR pharmaceutical composition comprises an RNA-guided nuclease and an sgRNA targeting the IL10RA gene with a crRNA sequence selected from SEQ ID NOs: 1008-1055. In some embodiments, the crRNA sequence is selected from SEQ ID NOs: 1008-1032. In some embodiments, the crRNA sequence is selected from 1008-1017. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL10RA gene is delivered to a mammalian cell via viral vector. In some embodiments, the CRISPR gene-editing system targeting the IL10RA gene is delivered to a mammalian cell via an AAV vector. In some embodiments, the CRISPR gene-editing system targeting the IL10RA gene is delivered to a mammalian cell via a lentiviral vector. In some embodiments, the CRISPR gene-editing system targeting the IL10RA gene is delivered to a mammalian cell via a lipid nanoparticle. In some embodiments, the CRISPR gene-editing system targeting the IL10RA gene is delivered to a mammalian cell via a virus-like particle. In some embodiments, the CRISPR gene-editing system targeting the IL10RA gene is delivered to a mammalian cell via a liposome. In some embodiments, the CRISPR gene-editing system targeting the IL10RA gene is delivered to a mammalian cell via a lipid nanocrystal.

In some embodiments, the pharmaceutical composition comprises an sgRNA having a crRNA sequence selected from SEQ ID NOs: 1008-1055 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1008-1055 and a S. pyogenes Cas9 protein encapsulated in a lipid nanoparticle.

In various embodiments, the pharmaceutical composition comprising a CRISPR gene-editing system targeting the IL10RA gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL10RA gene, as described herein, to a subject in need thereof. In some embodiments, the pharmaceutical composition comprises an LNP encapsuling an sgRNA having a crRNA sequence selected from SEQ ID NOs: 1008-1055 and a S. pyogenes Cas9 protein. In some embodiments, the crRNA sequence is selected from SEQ ID NOs: 1008-1032. In some embodiments, the crRNA sequence is selected from SEQ ID NOs: 1008-1017. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL10RA gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL10RA gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL10RA gene (e.g., 5′ untranslated region [UTR], exon 1, exon 2, exon 3, exon 4, exon 5, exon 6, exon 7, exon 8, exon 9, any intervening intronic regions, intron/exon junctions, the 3′ UTR, or polyadenylation signal) is targeted by an RNA-guided nuclease to alter the gene. In some embodiments, the IL10RA gene targeted by an RNA-guided nuclease is from a mammal. In some embodiments, the IL10RA gene targeted by an RNA-guided nuclease is from a human (hIL10RA). In some embodiments, the IL10RA gene targeted by an RNA-guided nuclease is from a dog (cIL10RA). In some embodiments, the IL10RA gene targeted by an RNA-guided nuclease is from a horse (eIL10RA). In some embodiments, the IL10RA gene targeted by an RNA-guided nuclease is from a cat (fIL10RA).

### OO. IL10RB

In one aspect, methods and pharmaceutical compositions are provided for treating a back or spine condition or disorder by administering a therapeutically effective amount of a CRISPR pharmaceutical composition that targets an IL10RB gene to a subject in need thereof. In some embodiments, the CRISPR pharmaceutical composition comprises an RNA-guided nuclease and an sgRNA targeting the IL10RB gene with a crRNA sequence selected from SEQ ID NOs: 1056-1082. In some embodiments, the crRNA sequence is selected from SEQ ID NOs: 1056-1080. In some embodiments, the crRNA sequence is selected from 1056-1065. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL10RB gene is delivered to a mammalian cell via viral vector. In some embodiments, the CRISPR gene-editing system targeting the IL10RB gene is delivered to a mammalian cell via an AAV vector. In some embodiments, the CRISPR gene-editing system targeting the IL10RB gene is delivered to a mammalian cell via a lentiviral vector. In some embodiments, the CRISPR gene-editing system targeting the IL10RB gene is delivered to a mammalian cell via a lipid nanoparticle. In some embodiments, the CRISPR gene-editing system targeting the IL10RB gene is delivered to a mammalian cell via a virus-like particle. In some embodiments, the CRISPR gene-editing system targeting the IL10RB gene is delivered to a mammalian cell via a liposome. In some embodiments, the CRISPR gene-editing system targeting the IL10RB gene is delivered to a mammalian cell via a lipid nanocrystal.

In some embodiments, the pharmaceutical composition comprises an sgRNA having a crRNA sequence selected from SEQ ID NOs: 1056-1082 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1056-1082 and a S. pyogenes Cas9 protein encapsulated in a lipid nanoparticle.

In various embodiments, the pharmaceutical composition comprising a CRISPR gene-editing system targeting the IL10RB gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL10RB gene, as described herein, to a subject in need thereof. In some embodiments, the pharmaceutical composition comprises an LNP encapsuling an sgRNA having a crRNA sequence selected from SEQ ID NOs: 1056-1082 and a S. pyogenes Cas9 protein. In some embodiments, the crRNA sequence is selected from SEQ ID NOs: 1056-1080. In some embodiments, the crRNA sequence is selected from SEQ ID NOs: 1056-1065. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL10RB gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL10RB gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL10RB gene (e.g., 5′ untranslated region [UTR], exon 1, exon 2, exon 3, exon 4, exon 5, exon 6, exon 7, exon 8, exon 9, any intervening intronic regions, intron/exon junctions, the 3′ UTR, or polyadenylation signal) is targeted by an RNA-guided nuclease to alter the gene. In some embodiments, the IL10RB gene targeted by an RNA-guided nuclease is from a mammal. In some embodiments, the IL10RB gene targeted by an RNA-guided nuclease is from a human (hIL10RB). In some embodiments, the IL10RB gene targeted by an RNA-guided nuclease is from a dog (cIL10RB). In some embodiments, the IL10RB gene targeted by an RNA-guided nuclease is from a horse (eIL10RB). In some embodiments, the IL10RB gene targeted by an RNA-guided nuclease is from a cat (fIL10RB).

### PP. IL13

In one aspect, methods and pharmaceutical compositions are provided for treating a back or spine condition or disorder by administering a therapeutically effective amount of a CRISPR pharmaceutical composition that targets an IL13 gene to a subject in need thereof. In some embodiments, the CRISPR pharmaceutical composition comprises an RNA-guided nuclease and an sgRNA targeting the IL13 gene with a crRNA sequence selected from SEQ ID NOs: 1083-1104. In some embodiments, the crRNA sequence is selected from 1083-1092. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL13 gene is delivered to a mammalian cell via viral vector. In some embodiments, the CRISPR gene-editing system targeting the IL13 gene is delivered to a mammalian cell via an AAV vector. In some embodiments, the CRISPR gene-editing system targeting the IL13 gene is delivered to a mammalian cell via a lentiviral vector. In some embodiments, the CRISPR gene-editing system targeting the IL13 gene is delivered to a mammalian cell via a lipid nanoparticle. In some embodiments, the CRISPR gene-editing system targeting the IL13 gene is delivered to a mammalian cell via a virus-like particle. In some embodiments, the CRISPR gene-editing system targeting the IL13 gene is delivered to a mammalian cell via a liposome. In some embodiments, the CRISPR gene-editing system targeting the IL13 gene is delivered to a mammalian cell via a lipid nanocrystal.

In some embodiments, the pharmaceutical composition comprises an sgRNA having a crRNA sequence selected from SEQ ID NOs: 1083-1104 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1083-1104 and a S. pyogenes Cas9 protein encapsulated in a lipid nanoparticle.

In various embodiments, the pharmaceutical composition comprising a CRISPR gene-editing system targeting the IL13 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL13 gene, as described herein, to a subject in need thereof. In some embodiments, the pharmaceutical composition comprises an LNP encapsuling an sgRNA having a crRNA sequence selected from SEQ ID NOs: 1083-1104 and a S. pyogenes Cas9 protein. In some embodiments, the crRNA sequence is selected from SEQ ID NOs: 1083-1092. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL13 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL13 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL13 gene (e.g., 5′ untranslated region [UTR], exon 1, exon 2, exon 3, exon 4, exon 5, exon 6, any intervening intronic regions, intron/exon junctions, the 3′ UTR, or polyadenylation signal) is targeted by an RNA-guided nuclease to alter the gene. In some embodiments, the IL13 gene targeted by an RNA-guided nuclease is from a mammal. In some embodiments, the IL13 gene targeted by an RNA-guided nuclease is from a human (hIL13). In some embodiments, the IL13 gene targeted by an RNA-guided nuclease is from a dog (cIL13). In some embodiments, the IL13 gene targeted by an RNA-guided nuclease is from a horse (eIL13). In some embodiments, the IL13 gene targeted by an RNA-guided nuclease is from a cat (fIL13).

### QQ. IL13RA1

In one aspect, methods and pharmaceutical compositions are provided for treating a back or spine condition or disorder by administering a therapeutically effective amount of a CRISPR pharmaceutical composition that targets an IL13RA1 gene to a subject in need thereof. In some embodiments, the CRISPR pharmaceutical composition comprises an RNA-guided nuclease and an sgRNA targeting the IL13RA1 gene with a crRNA sequence selected from SEQ ID NOs: 1105-1130. In some embodiments, the crRNA sequence is selected from SEQ ID NOs: 1105-1129. In some embodiments, the crRNA sequence is selected from 1105-1114. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL13RA1 gene is delivered to a mammalian cell via viral vector. In some embodiments, the CRISPR gene-editing system targeting the IL13RA1 gene is delivered to a mammalian cell via an AAV vector. In some embodiments, the CRISPR gene-editing system targeting the IL13RA1 gene is delivered to a mammalian cell via a lentiviral vector. In some embodiments, the CRISPR gene-editing system targeting the IL13RA1 gene is delivered to a mammalian cell via a lipid nanoparticle. In some embodiments, the CRISPR gene-editing system targeting the IL13RA1 gene is delivered to a mammalian cell via a virus-like particle. In some embodiments, the CRISPR gene-editing system targeting the IL13RA1 gene is delivered to a mammalian cell via a liposome. In some embodiments, the CRISPR gene-editing system targeting the IL13RA1 gene is delivered to a mammalian cell via a lipid nanocrystal.

In some embodiments, the pharmaceutical composition comprises an sgRNA having a crRNA sequence selected from SEQ ID NOs: 1105-1130 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1105-1130 and a S. pyogenes Cas9 protein encapsulated in a lipid nanoparticle.

In various embodiments, the pharmaceutical composition comprising a CRISPR gene-editing system targeting the IL13RA1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL13RA1 gene, as described herein, to a subject in need thereof. In some embodiments, the pharmaceutical composition comprises an LNP encapsuling an sgRNA having a crRNA sequence selected from SEQ ID NOs: 1105-1130 and a S. pyogenes Cas9 protein. In some embodiments, the crRNA sequence is selected from SEQ ID NOs: 1105-1129. In some embodiments, the crRNA sequence is selected from SEQ ID NOs: 1105-1114. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL13RA1 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL13RA1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL13RA1 gene (e.g., 5′ untranslated region [UTR], exon 1, exon 2, exon 3, exon 4, exon 5, exon 6, exon 7, exon 8, exon 9, exon 10, exon 11, exon 12, any intervening intronic regions, intron/exon junctions, the 3′ UTR, or polyadenylation signal) is targeted by an RNA-guided nuclease to alter the gene. In some embodiments, the IL13RA1 gene targeted by an RNA-guided nuclease is from a mammal. In some embodiments, the IL13RA1 gene targeted by an RNA-guided nuclease is from a human (hIL13RA1). In some embodiments, the IL13RA1 gene targeted by an RNA-guided nuclease is from a dog (cIL13RA1). In some embodiments, the IL13RA1 gene targeted by an RNA-guided nuclease is from a horse (eIL13RA1). In some embodiments, the IL13RA1 gene targeted by an RNA-guided nuclease is from a cat (fIL13RA1).

### RR. IL13RA2

In one aspect, methods and pharmaceutical compositions are provided for treating a back or spine condition or disorder by administering a therapeutically effective amount of a CRISPR pharmaceutical composition that targets an IL13RA2 gene to a subject in need thereof. In some embodiments, the CRISPR pharmaceutical composition comprises an RNA-guided nuclease and an sgRNA targeting the IL13RA2 gene with a crRNA sequence selected from SEQ ID NOs: 1131-1147. In some embodiments, the crRNA sequence is selected from 1131-1140. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL13RA2 gene is delivered to a mammalian cell via viral vector. In some embodiments, the CRISPR gene-editing system targeting the IL13RA2 gene is delivered to a mammalian cell via an AAV vector. In some embodiments, the CRISPR gene-editing system targeting the IL13RA2 gene is delivered to a mammalian cell via a lentiviral vector. In some embodiments, the CRISPR gene-editing system targeting the IL13RA2 gene is delivered to a mammalian cell via a lipid nanoparticle. In some embodiments, the CRISPR gene-editing system targeting the IL13RA2 gene is delivered to a mammalian cell via a virus-like particle. In some embodiments, the CRISPR gene-editing system targeting the IL13RA2 gene is delivered to a mammalian cell via a liposome. In some embodiments, the CRISPR gene-editing system targeting the IL13RA2 gene is delivered to a mammalian cell via a lipid nanocrystal.

In some embodiments, the pharmaceutical composition comprises an sgRNA having a crRNA sequence selected from SEQ ID NOs: 1131-1147 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1131-1147 and a S. pyogenes Cas9 protein encapsulated in a lipid nanoparticle.

In various embodiments, the pharmaceutical composition comprising a CRISPR gene-editing system targeting the IL13RA2 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL13RA2 gene, as described herein, to a subject in need thereof. In some embodiments, the pharmaceutical composition comprises an LNP encapsuling an sgRNA having a crRNA sequence selected from SEQ ID NOs: 1131-1147 and a S. pyogenes Cas9 protein. In some embodiments, the crRNA sequence is selected from SEQ ID NOs: 1131-1140. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL13RA2 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL13RA2 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL13RA2 gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, any intervening intronic regions, intron/exon junctions, the 3′ UTR, or polyadenylation signal) is targeted by an RNA-guided nuclease to alter the gene. In some embodiments, the IL13RA2 gene targeted by an RNA-guided nuclease is from a mammal. In some embodiments, the IL13RA2 gene targeted by an RNA-guided nuclease is from a human (hIL13RA2). In some embodiments, the IL13RA2 gene targeted by an RNA-guided nuclease is from a dog (cIL13RA2). In some embodiments, the IL13RA2 gene targeted by an RNA-guided nuclease is from a horse (eIL13RA2). In some embodiments, the IL13RA2 gene targeted by an RNA-guided nuclease is from a cat (fIL13RA2).

### SS. IL17A

In one aspect, methods and pharmaceutical compositions are provided for treating a back or spine condition or disorder by administering a therapeutically effective amount of a CRISPR pharmaceutical composition that targets an IL17A gene to a subject in need thereof. In some embodiments, the CRISPR pharmaceutical composition comprises an RNA-guided nuclease and an sgRNA targeting the IL17A gene with a crRNA sequence selected from SEQ ID NOs: 1148-1173. In some embodiments, the crRNA sequence is selected from SEQ ID NOs: 1148-1172. In some embodiments, the crRNA sequence is selected from 1148-1157. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7537_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL17A gene is delivered to a mammalian cell via viral vector. In some embodiments, the CRISPR gene-editing system targeting the IL17A gene is delivered to a mammalian cell via an AAV vector. In some embodiments, the CRISPR gene-editing system targeting the IL17A gene is delivered to a mammalian cell via a lentiviral vector. In some embodiments, the CRISPR gene-editing system targeting the IL17A gene is delivered to a mammalian cell via a lipid nanoparticle. In some embodiments, the CRISPR gene-editing system targeting the IL17A gene is delivered to a mammalian cell via a virus-like particle. In some embodiments, the CRISPR gene-editing system targeting the IL17A gene is delivered to a mammalian cell via a liposome. In some embodiments, the CRISPR gene-editing system targeting the IL17A gene is delivered to a mammalian cell via a lipid nanocrystal.

In some embodiments, the pharmaceutical composition comprises an sgRNA having a crRNA sequence selected from SEQ ID NOs: 1148-1173 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1148-1173 and a S. pyogenes Cas9 protein encapsulated in a lipid nanoparticle.

In various embodiments, the pharmaceutical composition comprising a CRISPR gene-editing system targeting the IL17A gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL17A gene, as described herein, to a subject in need thereof. In some embodiments, the pharmaceutical composition comprises an LNP encapsuling an sgRNA having a crRNA sequence selected from SEQ ID NOs: 1148-1173 and a S. pyogenes Cas9 protein. In some embodiments, the crRNA sequence is selected from SEQ ID NOs: 1148-1172. In some embodiments, the crRNA sequence is selected from SEQ ID NOs: 1148-1157. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL17A gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL17A gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL17A gene (e.g., 5′ untranslated region [UTR], exon 1, exon 2, exon 3, any intervening intronic regions, intron/exon junctions, the 3′ UTR, or polyadenylation signal) is targeted by an RNA-guided nuclease to alter the gene. In some embodiments, the IL17A gene targeted by an RNA-guided nuclease is from a mammal. In some embodiments, the IL17A gene targeted by an RNA-guided nuclease is from a human (hIL17A). In some embodiments, the IL17A gene targeted by an RNA-guided nuclease is from a dog (cIL17A). In some embodiments, the IL17A gene targeted by an RNA-guided nuclease is from a horse (eIL17A). In some embodiments, the IL17A gene targeted by an RNA-guided nuclease is from a cat (fIL17A).

### TT. IL17RA

In one aspect, methods and pharmaceutical compositions are provided for treating a back or spine condition or disorder by administering a therapeutically effective amount of a CRISPR pharmaceutical composition that targets an IL17RA gene to a subject in need thereof. In some embodiments, the CRISPR pharmaceutical composition comprises an RNA-guided nuclease and an sgRNA targeting the IL17RA gene with a crRNA sequence selected from SEQ ID NOs: 1174-1221. In some embodiments, the crRNA sequence is selected from SEQ ID NOs: 1174-1198. In some embodiments, the crRNA sequence is selected from 1174-1183. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL17RA gene is delivered to a mammalian cell via viral vector. In some embodiments, the CRISPR gene-editing system targeting the IL17RA gene is delivered to a mammalian cell via an AAV vector. In some embodiments, the CRISPR gene-editing system targeting the IL17RA gene is delivered to a mammalian cell via a lentiviral vector. In some embodiments, the CRISPR gene-editing system targeting the IL17RA gene is delivered to a mammalian cell via a lipid nanoparticle. In some embodiments, the CRISPR gene-editing system targeting the IL17RA gene is delivered to a mammalian cell via a virus-like particle. In some embodiments, the CRISPR gene-editing system targeting the IL17RA gene is delivered to a mammalian cell via a liposome. In some embodiments, the CRISPR gene-editing system targeting the IL17RA gene is delivered to a mammalian cell via a lipid nanocrystal.

In some embodiments, the pharmaceutical composition comprises an sgRNA having a crRNA sequence selected from SEQ ID NOs: 1174-1221 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1174-1221 and a S. pyogenes Cas9 protein encapsulated in a lipid nanoparticle.

In various embodiments, the pharmaceutical composition comprising a CRISPR gene-editing system targeting the IL17RA gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL17RA gene, as described herein, to a subject in need thereof. In some embodiments, the pharmaceutical composition comprises an LNP encapsuling an sgRNA having a crRNA sequence selected from SEQ ID NOs: 1174-1221 and a S. pyogenes Cas9 protein. In some embodiments, the crRNA sequence is selected from SEQ ID NOs: 1174-1198. In some embodiments, the crRNA sequence is selected from SEQ ID NOs: 1174-1183. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL17RA gene, as described herein, to a subject in need thereof. In some embodiments, the musculoskeletal disease or disorder is Arthritis. Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL17RA gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL17RA gene (e.g., 5′ untranslated region [UTR], exon 1, exon 2, exon 3, exon 4, exon 5, exon 6, exon 7, exon 8, exon 9, exon 10, exon 11, exon 12, exon 13, any intervening intronic regions, intron/exon junctions, the 3′ UTR, or polyadenylation signal) is targeted by an RNA-guided nuclease to alter the gene. In some embodiments, the IL17RA gene targeted by an RNA-guided nuclease is from a mammal. In some embodiments, the IL17RA gene targeted by an RNA-guided nuclease is from a human (hIL17RA). In some embodiments, the IL17RA gene targeted by an RNA-guided nuclease is from a dog (cIL17RA). In some embodiments, the IL17RA gene targeted by an RNA-guided nuclease is from a horse (eIL17RA). In some embodiments, the IL17RA gene targeted by an RNA-guided nuclease is from a cat (fIL17RA).

### UU. IL18

In one aspect, methods and pharmaceutical compositions are provided for treating a back or spine condition or disorder by administering a therapeutically effective amount of a CRISPR pharmaceutical composition that targets an IL18 gene to a subject in need thereof. In some embodiments, the CRISPR pharmaceutical composition comprises an RNA-guided nuclease and an sgRNA targeting the IL18 gene with a crRNA sequence selected from SEQ ID NOs: 1222-1238. In some embodiments, the crRNA sequence is selected from 1222-1231. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL18 gene is delivered to a mammalian cell via viral vector. In some embodiments, the CRISPR gene-editing system targeting the IL18 gene is delivered to a mammalian cell via an AAV vector. In some embodiments, the CRISPR gene-editing system targeting the IL18 gene is delivered to a mammalian cell via a lentiviral vector. In some embodiments, the CRISPR gene-editing system targeting the IL18 gene is delivered to a mammalian cell via a lipid nanoparticle. In some embodiments, the CRISPR gene-editing system targeting the IL18 gene is delivered to a mammalian cell via a virus-like particle. In some embodiments, the CRISPR gene-editing system targeting the IL18 gene is delivered to a mammalian cell via a liposome. In some embodiments, the CRISPR gene-editing system targeting the IL18 gene is delivered to a mammalian cell via a lipid nanocrystal.

In some embodiments, the pharmaceutical composition comprises an sgRNA having a crRNA sequence selected from SEQ ID NOs: 1222-1238 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1222-1238 and a S. pyogenes Cas9 protein encapsulated in a lipid nanoparticle.

In various embodiments, the pharmaceutical composition comprising a CRISPR gene-editing system targeting the IL18 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL18 gene, as described herein, to a subject in need thereof. In some embodiments, the pharmaceutical composition comprises an LNP encapsuling an sgRNA having a crRNA sequence selected from SEQ ID NOs: 1222-1238 and a S. pyogenes Cas9 protein. In some embodiments, the crRNA sequence is selected from SEQ ID NOs: 1222-1231. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL18 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL18 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL18 gene (e.g., 5′ untranslated region [UTR], exon 1, exon 2, exon 3, exon 4, exon 5, exon 6, exon 7, exon 8, exon 9, any intervening intronic regions, intron/exon junctions, the 3′ UTR, or polyadenylation signal) is targeted by an RNA-guided nuclease to alter the gene. In some embodiments, the IL18 gene targeted by an RNA-guided nuclease is from a mammal. In some embodiments, the IL18 gene targeted by an RNA-guided nuclease is from a human (hIL18). In some embodiments, the IL18 gene targeted by an RNA-guided nuclease is from a dog (cIL18). In some embodiments, the IL18 gene targeted by an RNA-guided nuclease is from a horse (eIL18). In some embodiments, the IL18 gene targeted by an RNA-guided nuclease is from a cat (fIL18).

### VV. IL18R1

In one aspect, methods and pharmaceutical compositions are provided for treating a back or spine condition or disorder by administering a therapeutically effective amount of a CRISPR pharmaceutical composition that targets an IL18R1 gene to a subject in need thereof. In some embodiments, the CRISPR pharmaceutical composition comprises an RNA-guided nuclease and an sgRNA targeting the IL18R1 gene with a crRNA sequence selected from SEQ ID NOs: 1239-1262. In some embodiments, the crRNA sequence is selected from 1239-1248. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL18R1 gene is delivered to a mammalian cell via viral vector. In some embodiments, the CRISPR gene-editing system targeting the IL18R1 gene is delivered to a mammalian cell via an AAV vector. In some embodiments, the CRISPR gene-editing system targeting the IL18R1 gene is delivered to a mammalian cell via a lentiviral vector. In some embodiments, the CRISPR gene-editing system targeting the IL18R1 gene is delivered to a mammalian cell via a lipid nanoparticle. In some embodiments, the CRISPR gene-editing system targeting the IL18R1 gene is delivered to a mammalian cell via a virus-like particle. In some embodiments, the CRISPR gene-editing system targeting the IL18R1 gene is delivered to a mammalian cell via a liposome. In some embodiments, the CRISPR gene-editing system targeting the IL18R1 gene is delivered to a mammalian cell via a lipid nanocrystal.

In some embodiments, the pharmaceutical composition comprises an sgRNA having a crRNA sequence selected from SEQ ID NOs: 1239-1262 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1239-1262 and a S. pyogenes Cas9 protein encapsulated in a lipid nanoparticle.

In various embodiments, the pharmaceutical composition comprising a CRISPR gene-editing system targeting the IL18R1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL18R1 gene, as described herein, to a subject in need thereof. In some embodiments, the pharmaceutical composition comprises an LNP encapsuling an sgRNA having a crRNA sequence selected from SEQ ID NOs: 1239-1262 and a S. pyogenes Cas9 protein. In some embodiments, the crRNA sequence is selected from SEQ ID NOs: 1239-1248. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL18R1 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL18R1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL18R1 gene (e.g., 5′ untranslated region [UTR], exon 1, exon 2, exon 3, exon 4, exon 5, exon 6, exon 7, exon 8, exon 9, exon 10, exon 11, exon 12, exon 13, any intervening intronic regions, intron/exon junctions, the 3′ UTR, or polyadenylation signal) is targeted by an RNA-guided nuclease to alter the gene. In some embodiments, the IL18R1 gene targeted by an RNA-guided nuclease is from a mammal. In some embodiments, the IL18R1 gene targeted by an RNA-guided nuclease is from a human (hIL18R1). In some embodiments, the IL18R1 gene targeted by an RNA-guided nuclease is from a dog (cIL18R1). In some embodiments, the IL18R1 gene targeted by an RNA-guided nuclease is from a horse (eIL18R1). In some embodiments, the IL18R1 gene targeted by an RNA-guided nuclease is from a cat (fIL18R1).

### WW. IL18RAP

In one aspect, methods and pharmaceutical compositions are provided for treating a back or spine condition or disorder by administering a therapeutically effective amount of a CRISPR pharmaceutical composition that targets an IL18RAP gene to a subject in need thereof. In some embodiments, the CRISPR pharmaceutical composition comprises an RNA-guided nuclease and an sgRNA targeting the IL18RAP gene with a crRNA sequence selected from SEQ ID NOs: 1263-1310. In some embodiments, the crRNA sequence is selected from SEQ ID NOs: 1263-1287. In some embodiments, the crRNA sequence is selected from 1263-1272. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL18RAP gene is delivered to a mammalian cell via viral vector. In some embodiments, the CRISPR gene-editing system targeting the IL18RAP gene is delivered to a mammalian cell via an AAV vector. In some embodiments, the CRISPR gene-editing system targeting the IL18RAP gene is delivered to a mammalian cell via a lentiviral vector. In some embodiments, the CRISPR gene-editing system targeting the IL18RAP gene is delivered to a mammalian cell via a lipid nanoparticle. In some embodiments, the CRISPR gene-editing system targeting the IL18RAP gene is delivered to a mammalian cell via a virus-like particle. In some embodiments, the CRISPR gene-editing system targeting the IL18RAP gene is delivered to a mammalian cell via a liposome. In some embodiments, the CRISPR gene-editing system targeting the IL18RAP gene is delivered to a mammalian cell via a lipid nanocrystal.

In some embodiments, the pharmaceutical composition comprises an sgRNA having a crRNA sequence selected from SEQ ID NOs: 1263-1310 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1263-1310 and a S. pyogenes Cas9 protein encapsulated in a lipid nanoparticle.

In various embodiments, the pharmaceutical composition comprising a CRISPR gene-editing system targeting the IL18RAP gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL18RAP gene, as described herein, to a subject in need thereof. In some embodiments, the pharmaceutical composition comprises an LNP encapsuling an sgRNA having a crRNA sequence selected from SEQ ID NOs: 1263-1310 and a S. pyogenes Cas9 protein. In some embodiments, the crRNA sequence is selected from SEQ ID NOs: 1263-1287. In some embodiments, the crRNA sequence is selected from SEQ ID NOs: 1263-1272. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL18RAP gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL18RAP gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL18RAP gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, any intervening intronic regions, intron/exon junctions, the 3′ UTR, or polyadenylation signal) is targeted by an RNA-guided nuclease to alter the gene. In some embodiments, the IL18RAP gene targeted by an RNA-guided nuclease is from a mammal. In some embodiments, the IL18RAP gene targeted by an RNA-guided nuclease is from a human (hIL18RAP). In some embodiments, the IL18RAP gene targeted by an RNA-guided nuclease is from a dog (cIL18RAP). In some embodiments, the IL18RAP gene targeted by an RNA-guided nuclease is from a horse (eIL18RAP). In some embodiments, the IL18RAP gene targeted by an RNA-guided nuclease is from a cat (fIL18RAP).

### XX. SCN1A

In one aspect, methods and pharmaceutical compositions are provided for treating a back or spine condition or disorder by administering a therapeutically effective amount of a CRISPR pharmaceutical composition that targets an SCN1A gene to a subject in need thereof. In some embodiments, the CRISPR pharmaceutical composition comprises an RNA-guided nuclease and an sgRNA targeting the SCN1A gene with a crRNA sequence selected from SEQ ID NOs: 1860-1907. In some embodiments, the crRNA sequence is selected from SEQ ID NOs: 1860-1884. In some embodiments, the crRNA sequence is selected from 1860-1869. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the SCN1A gene is delivered to a mammalian cell via viral vector. In some embodiments, the CRISPR gene-editing system targeting the SCN1A gene is delivered to a mammalian cell via an AAV vector. In some embodiments, the CRISPR gene-editing system targeting the SCN1A gene is delivered to a mammalian cell via a lentiviral vector. In some embodiments, the CRISPR gene-editing system targeting the SCN1A gene is delivered to a mammalian cell via a lipid nanoparticle. In some embodiments, the CRISPR gene-editing system targeting the SCN1A gene is delivered to a mammalian cell via a virus-like particle. In some embodiments, the CRISPR gene-editing system targeting the SCN1A gene is delivered to a mammalian cell via a liposome. In some embodiments, the CRISPR gene-editing system targeting the SCN1A gene is delivered to a mammalian cell via a lipid nanocrystal.

In some embodiments, the pharmaceutical composition comprises an sgRNA having a crRNA sequence selected from SEQ ID NOs: 1860-1907 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1860-1907 and a S. pyogenes Cas9 protein encapsulated in a lipid nanoparticle.

In various embodiments, the pharmaceutical composition comprising a CRISPR gene-editing system targeting the SCN1A gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the SCN1A gene, as described herein, to a subject in need thereof. In some embodiments, the pharmaceutical composition comprises an LNP encapsuling an sgRNA having a crRNA sequence selected from SEQ ID NOs: 1860-1907 and a S. pyogenes Cas9 protein. In some embodiments, the crRNA sequence is selected from SEQ ID NOs: 1860-1884. In some embodiments, the crRNA sequence is selected from SEQ ID NOs: 1860-1869. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the SCN1A gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the SCN1A gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an SCN1A gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, any intervening intronic regions, intron/exon junctions, the 3′ UTR, or polyadenylation signal) is targeted by an RNA-guided nuclease to alter the gene. In some embodiments, the SCN1A gene targeted by an RNA-guided nuclease is from a mammal. In some embodiments, the SCN1A gene targeted by an RNA-guided nuclease is from a human (hSCN1A). In some embodiments, the SCN1A gene targeted by an RNA-guided nuclease is from a dog (cSCN1A). In some embodiments, the SCN1A gene targeted by an RNA-guided nuclease is from a horse (eSCN1A). In some embodiments, the SCN1A gene targeted by an RNA-guided nuclease is from a cat (fSCN1A).

### YY. SCN2A

In one aspect, methods and pharmaceutical compositions are provided for treating a back or spine condition or disorder by administering a therapeutically effective amount of a CRISPR pharmaceutical composition that targets an SCN2A gene to a subject in need thereof. In some embodiments, the CRISPR pharmaceutical composition comprises an RNA-guided nuclease and an sgRNA targeting the SCN2A gene with a crRNA sequence selected from SEQ ID NOs: 1908-1955. In some embodiments, the crRNA sequence is selected from SEQ ID NOs: 1908-1932. In some embodiments, the crRNA sequence is selected from 1908-1917. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the SCN2A gene is delivered to a mammalian cell via viral vector. In some embodiments, the CRISPR gene-editing system targeting the SCN2A gene is delivered to a mammalian cell via an AAV vector. In some embodiments, the CRISPR gene-editing system targeting the SCN2A gene is delivered to a mammalian cell via a lentiviral vector. In some embodiments, the CRISPR gene-editing system targeting the SCN2A gene is delivered to a mammalian cell via a lipid nanoparticle. In some embodiments, the CRISPR gene-editing system targeting the SCN2A gene is delivered to a mammalian cell via a virus-like particle. In some embodiments, the CRISPR gene-editing system targeting the SCN2A gene is delivered to a mammalian cell via a liposome. In some embodiments, the CRISPR gene-editing system targeting the SCN2A gene is delivered to a mammalian cell via a lipid nanocrystal.

In some embodiments, the pharmaceutical composition comprises an sgRNA having a crRNA sequence selected from SEQ ID NOs: 1908-1955 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1908-1955 and a S. pyogenes Cas9 protein encapsulated in a lipid nanoparticle.

In various embodiments, the pharmaceutical composition comprising a CRISPR gene-editing system targeting the SCN2A gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the SCN2A gene, as described herein, to a subject in need thereof. In some embodiments, the pharmaceutical composition comprises an LNP encapsuling an sgRNA having a crRNA sequence selected from SEQ ID NOs: 1908-1955 and a S. pyogenes Cas9 protein. In some embodiments, the crRNA sequence is selected from SEQ ID NOs: 1908-1932. In some embodiments, the crRNA sequence is selected from SEQ ID NOs: 1908-1917. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the SCN2A gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the SCN2A gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an SCN2A gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, any intervening intronic regions, intron/exon junctions, the 3′ UTR, or polyadenylation signal) is targeted by an RNA-guided nuclease to alter the gene. In some embodiments, the SCN2A gene targeted by an RNA-guided nuclease is from a mammal. In some embodiments, the SCN2A gene targeted by an RNA-guided nuclease is from a human (hSCN2A). In some embodiments, the SCN2A gene targeted by an RNA-guided nuclease is from a dog (cSCN2A). In some embodiments, the SCN2A gene targeted by an RNA-guided nuclease is from a horse (eSCN2A). In some embodiments, the SCN2A gene targeted by an RNA-guided nuclease is from a cat (fSCN2A).

### ZZ. SCN3A

In one aspect, methods and pharmaceutical compositions are provided for treating a back or spine condition or disorder by administering a therapeutically effective amount of a CRISPR pharmaceutical composition that targets an SCN3A gene to a subject in need thereof. In some embodiments, the CRISPR pharmaceutical composition comprises an RNA-guided nuclease and an sgRNA targeting the SCN3A gene with a crRNA sequence selected from SEQ ID NOs: 1956-2003. In some embodiments, the crRNA sequence is selected from SEQ ID NOs: 1956-1980. In some embodiments, the crRNA sequence is selected from 1956-1965. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the SCN3A gene is delivered to a mammalian cell via viral vector. In some embodiments, the CRISPR gene-editing system targeting the SCN3A gene is delivered to a mammalian cell via an AAV vector. In some embodiments, the CRISPR gene-editing system targeting the SCN3A gene is delivered to a mammalian cell via a lentiviral vector. In some embodiments, the CRISPR gene-editing system targeting the SCN3A gene is delivered to a mammalian cell via a lipid nanoparticle. In some embodiments, the CRISPR gene-editing system targeting the SCN3A gene is delivered to a mammalian cell via a virus-like particle. In some embodiments, the CRISPR gene-editing system targeting the SCN3A gene is delivered to a mammalian cell via a liposome. In some embodiments, the CRISPR gene-editing system targeting the SCN3A gene is delivered to a mammalian cell via a lipid nanocrystal.

In some embodiments, the pharmaceutical composition comprises an sgRNA having a crRNA sequence selected from SEQ ID NOs: 1956-2003 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1956-2003 and a S. pyogenes Cas9 protein encapsulated in a lipid nanoparticle.

In various embodiments, the pharmaceutical composition comprising a CRISPR gene-editing system targeting the SCN3A gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the SCN3A gene, as described herein, to a subject in need thereof. In some embodiments, the pharmaceutical composition comprises an LNP encapsuling an sgRNA having a crRNA sequence selected from SEQ ID NOs: 1956-2003 and a S. pyogenes Cas9 protein. In some embodiments, the crRNA sequence is selected from SEQ ID NOs: 1956-1980. In some embodiments, the crRNA sequence is selected from SEQ ID NOs: 1956-1965. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the SCN3A gene, as described herein, to a subject in need thereof. In some embodiments, the musculoskeletal disease or disorder is Arthritis. Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the SCN3A gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an SCN3A gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, any intervening intronic regions, intron/exon junctions, the 3′ UTR, or polyadenylation signal) is targeted by an RNA-guided nuclease to alter the gene. In some embodiments, the SCN3A gene targeted by an RNA-guided nuclease is from a mammal. In some embodiments, the SCN3A gene targeted by an RNA-guided nuclease is from a human (hSCN3A). In some embodiments, the SCN3A gene targeted by an RNA-guided nuclease is from a dog (cSCN3A). In some embodiments, the SCN3A gene targeted by an RNA-guided nuclease is from a horse (eSCN3A). In some embodiments, the SCN3A gene targeted by an RNA-guided nuclease is from a cat (fSCN3A).

### AAA. SCN4A

In one aspect, methods and pharmaceutical compositions are provided for treating a back or spine condition or disorder by administering a therapeutically effective amount of a CRISPR pharmaceutical composition that targets an SCN4A gene to a subject in need thereof. In some embodiments, the CRISPR pharmaceutical composition comprises an RNA-guided nuclease and an sgRNA targeting the SCN4A gene with a crRNA sequence selected from SEQ ID NOs: 2004-2051. In some embodiments, the crRNA sequence is selected from SEQ ID NOs: 2004-2028. In some embodiments, the crRNA sequence is selected from 2004-2013. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the SCN4A gene is delivered to a mammalian cell via viral vector. In some embodiments, the CRISPR gene-editing system targeting the SCN4A gene is delivered to a mammalian cell via an AAV vector. In some embodiments, the CRISPR gene-editing system targeting the SCN4A gene is delivered to a mammalian cell via a lentiviral vector. In some embodiments, the CRISPR gene-editing system targeting the SCN4A gene is delivered to a mammalian cell via a lipid nanoparticle. In some embodiments, the CRISPR gene-editing system targeting the SCN4A gene is delivered to a mammalian cell via a virus-like particle. In some embodiments, the CRISPR gene-editing system targeting the SCN4A gene is delivered to a mammalian cell via a liposome. In some embodiments, the CRISPR gene-editing system targeting the SCN4A gene is delivered to a mammalian cell via a lipid nanocrystal.

In some embodiments, the pharmaceutical composition comprises an sgRNA having a crRNA sequence selected from SEQ ID NOs: 2004-2051 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2004-2051 and a S. pyogenes Cas9 protein encapsulated in a lipid nanoparticle.

In various embodiments, the pharmaceutical composition comprising a CRISPR gene-editing system targeting the SCN4A gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the SCN4A gene, as described herein, to a subject in need thereof. In some embodiments, the pharmaceutical composition comprises an LNP encapsuling an sgRNA having a crRNA sequence selected from SEQ ID NOs: 2004-2051 and a S. pyogenes Cas9 protein. In some embodiments, the crRNA sequence is selected from SEQ ID NOs: 2004-2028. In some embodiments, the crRNA sequence is selected from SEQ ID NOs: 2004-2013. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the SCN4A gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the SCN4A gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an SCN4A gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, any intervening intronic regions, intron/exon junctions, the 3′ UTR, or polyadenylation signal) is targeted by an RNA-guided nuclease to alter the gene. In some embodiments, the SCN4A gene targeted by an RNA-guided nuclease is from a mammal. In some embodiments, the SCN4A gene targeted by an RNA-guided nuclease is from a human (hSCN4A). In some embodiments, the SCN4A gene targeted by an RNA-guided nuclease is from a dog (cSCN4A). In some embodiments, the SCN4A gene targeted by an RNA-guided nuclease is from a horse (eSCN4A). In some embodiments, the SCN4A gene targeted by an RNA-guided nuclease is from a cat (fSCN4A).

### BBB. SCN5A

In one aspect, methods and pharmaceutical compositions are provided for treating a back or spine condition or disorder by administering a therapeutically effective amount of a CRISPR pharmaceutical composition that targets an SCN5A gene to a subject in need thereof. In some embodiments, the CRISPR pharmaceutical composition comprises an RNA-guided nuclease and an sgRNA targeting the SCN5A gene with a crRNA sequence selected from SEQ ID NOs: 2052-2099. In some embodiments, the crRNA sequence is selected from SEQ ID NOs: 2052-2076. In some embodiments, the crRNA sequence is selected from 2052-2061. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the SCN5A gene is delivered to a mammalian cell via viral vector. In some embodiments, the CRISPR gene-editing system targeting the SCN5A gene is delivered to a mammalian cell via an AAV vector. In some embodiments, the CRISPR gene-editing system targeting the SCN5A gene is delivered to a mammalian cell via a lentiviral vector. In some embodiments, the CRISPR gene-editing system targeting the SCN5A gene is delivered to a mammalian cell via a lipid nanoparticle. In some embodiments, the CRISPR gene-editing system targeting the SCN5A gene is delivered to a mammalian cell via a virus-like particle. In some embodiments, the CRISPR gene-editing system targeting the SCN5A gene is delivered to a mammalian cell via a liposome. In some embodiments, the CRISPR gene-editing system targeting the SCN5A gene is delivered to a mammalian cell via a lipid nanocrystal.

In some embodiments, the pharmaceutical composition comprises an sgRNA having a crRNA sequence selected from SEQ ID NOs: 2052-2099 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2052-2099 and a S. pyogenes Cas9 protein encapsulated in a lipid nanoparticle.

In various embodiments, the pharmaceutical composition comprising a CRISPR gene-editing system targeting the SCN5A gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the SCN5A gene, as described herein, to a subject in need thereof. In some embodiments, the pharmaceutical composition comprises an LNP encapsuling an sgRNA having a crRNA sequence selected from SEQ ID NOs: 2052-2099 and a S. pyogenes Cas9 protein. In some embodiments, the crRNA sequence is selected from SEQ ID NOs: 2052-2076. In some embodiments, the crRNA sequence is selected from SEQ ID NOs: 2052-2061. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the SCN5A gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the SCN5A gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an SCN5A gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, any intervening intronic regions, intron/exon junctions, the 3′ UTR, or polyadenylation signal) is targeted by an RNA-guided nuclease to alter the gene. In some embodiments, the SCN5A gene targeted by an RNA-guided nuclease is from a mammal. In some embodiments, the SCN5A gene targeted by an RNA-guided nuclease is from a human (hSCN5A). In some embodiments, the SCN5A gene targeted by an RNA-guided nuclease is from a dog (cSCN5A). In some embodiments, the SCN5A gene targeted by an RNA-guided nuclease is from a horse (eSCN5A). In some embodiments, the SCN5A gene targeted by an RNA-guided nuclease is from a cat (fSCN5A).

### CCC. SCN8A

In one aspect, methods and pharmaceutical compositions are provided for treating a back or spine condition or disorder by administering a therapeutically effective amount of a CRISPR pharmaceutical composition that targets an SCN8A gene to a subject in need thereof. In some embodiments, the CRISPR pharmaceutical composition comprises an RNA-guided nuclease and an sgRNA targeting the SCN8A gene with a crRNA sequence selected from SEQ ID NOs: 2100-2147. In some embodiments, the crRNA sequence is selected from SEQ ID NOs: 2100-2124. In some embodiments, the crRNA sequence is selected from 2100-2109. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the SCN8A gene is delivered to a mammalian cell via viral vector. In some embodiments, the CRISPR gene-editing system targeting the SCN8A gene is delivered to a mammalian cell via an AAV vector. In some embodiments, the CRISPR gene-editing system targeting the SCN8A gene is delivered to a mammalian cell via a lentiviral vector. In some embodiments, the CRISPR gene-editing system targeting the SCN8A gene is delivered to a mammalian cell via a lipid nanoparticle. In some embodiments, the CRISPR gene-editing system targeting the SCN8A gene is delivered to a mammalian cell via a virus-like particle. In some embodiments, the CRISPR gene-editing system targeting the SCN8A gene is delivered to a mammalian cell via a liposome. In some embodiments, the CRISPR gene-editing system targeting the SCN8A gene is delivered to a mammalian cell via a lipid nanocrystal.

In some embodiments, the pharmaceutical composition comprises an sgRNA having a crRNA sequence selected from SEQ ID NOs: 2100-2147 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2100-2147 and a S. pyogenes Cas9 protein encapsulated in a lipid nanoparticle.

In various embodiments, the pharmaceutical composition comprising a CRISPR gene-editing system targeting the SCN8A gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the SCN8A gene, as described herein, to a subject in need thereof. In some embodiments, the pharmaceutical composition comprises an LNP encapsuling an sgRNA having a crRNA sequence selected from SEQ ID NOs: 2100-2147 and a S. pyogenes Cas9 protein. In some embodiments, the crRNA sequence is selected from SEQ ID NOs: 2100-2124. In some embodiments, the crRNA sequence is selected from SEQ ID NOs: 2100-2109. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the SCN8A gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the SCN8A gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an SCN8A gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, any intervening intronic regions, intron/exon junctions, the 3′ UTR, or polyadenylation signal) is targeted by an RNA-guided nuclease to alter the gene. In some embodiments, the SCN8A gene targeted by an RNA-guided nuclease is from a mammal. In some embodiments, the SCN8A gene targeted by an RNA-guided nuclease is from a human (hSCN8A). In some embodiments, the SCN8A gene targeted by an RNA-guided nuclease is from a dog (cSCN8A). In some embodiments, the SCN8A gene targeted by an RNA-guided nuclease is from a horse (eSCN8A). In some embodiments, the SCN8A gene targeted by an RNA-guided nuclease is from a cat (fSCN8A).

### DDD. SCN9A

In one aspect, methods and pharmaceutical compositions are provided for treating a back or spine condition or disorder by administering a therapeutically effective amount of a CRISPR pharmaceutical composition that targets an SCN9A gene to a subject in need thereof. In some embodiments, the CRISPR pharmaceutical composition comprises an RNA-guided nuclease and an sgRNA targeting the SCN9A gene with a crRNA sequence selected from SEQ ID NOs: 2148-2195. In some embodiments, the crRNA sequence is selected from SEQ ID NOs: 2148-2172. In some embodiments, the crRNA sequence is selected from 2148-2157. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the SCN9A gene is delivered to a mammalian cell via viral vector. In some embodiments, the CRISPR gene-editing system targeting the SCN9A gene is delivered to a mammalian cell via an AAV vector. In some embodiments, the CRISPR gene-editing system targeting the SCN9A gene is delivered to a mammalian cell via a lentiviral vector. In some embodiments, the CRISPR gene-editing system targeting the SCN9A gene is delivered to a mammalian cell via a lipid nanoparticle. In some embodiments, the CRISPR gene-editing system targeting the SCN9A gene is delivered to a mammalian cell via a virus-like particle. In some embodiments, the CRISPR gene-editing system targeting the SCN9A gene is delivered to a mammalian cell via a liposome. In some embodiments, the CRISPR gene-editing system targeting the SCN9A gene is delivered to a mammalian cell via a lipid nanocrystal.

In some embodiments, the pharmaceutical composition comprises an sgRNA having a crRNA sequence selected from SEQ ID NOs: 2148-2195 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2148-2195 and a S. pyogenes Cas9 protein encapsulated in a lipid nanoparticle.

In various embodiments, the pharmaceutical composition comprising a CRISPR gene-editing system targeting the SCN9A gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the SCN9A gene, as described herein, to a subject in need thereof. In some embodiments, the pharmaceutical composition comprises an LNP encapsuling an sgRNA having a crRNA sequence selected from SEQ ID NOs: 2148-2195 and a S. pyogenes Cas9 protein. In some embodiments, the crRNA sequence is selected from SEQ ID NOs: 2148-2172. In some embodiments, the crRNA sequence is selected from SEQ ID NOs: 2148-2157. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the SCN9A gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the SCN9A gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an SCN9A gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, any intervening intronic regions, intron/exon junctions, the 3′ UTR, or polyadenylation signal) is targeted by an RNA-guided nuclease to alter the gene. In some embodiments, the SCN9A gene targeted by an RNA-guided nuclease is from a mammal. In some embodiments, the SCN9A gene targeted by an RNA-guided nuclease is from a human (hSCN9A). In some embodiments, the SCN9A gene targeted by an RNA-guided nuclease is from a dog (cSCN9A). In some embodiments, the SCN9A gene targeted by an RNA-guided nuclease is from a horse (eSCN9A). In some embodiments, the SCN9A gene targeted by an RNA-guided nuclease is from a cat (fSCN9A).

### EEE. SCN10A

In one aspect, methods and pharmaceutical compositions are provided for treating a back or spine condition or disorder by administering a therapeutically effective amount of a CRISPR pharmaceutical composition that targets an SCN10A gene to a subject in need thereof. In some embodiments, the CRISPR pharmaceutical composition comprises an RNA-guided nuclease and an sgRNA targeting the SCN10A gene with a crRNA sequence selected from SEQ ID NOs: 2196-2243. In some embodiments, the crRNA sequence is selected from SEQ ID NOs: 2196-2220. In some embodiments, the crRNA sequence is selected from 2196-2205. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the SCN10A gene is delivered to a mammalian cell via viral vector. In some embodiments, the CRISPR gene-editing system targeting the SCN10A gene is delivered to a mammalian cell via an AAV vector. In some embodiments, the CRISPR gene-editing system targeting the SCN10A gene is delivered to a mammalian cell via a lentiviral vector. In some embodiments, the CRISPR gene-editing system targeting the SCN10A gene is delivered to a mammalian cell via a lipid nanoparticle. In some embodiments, the CRISPR gene-editing system targeting the SCN10A gene is delivered to a mammalian cell via a virus-like particle. In some embodiments, the CRISPR gene-editing system targeting the SCN10A gene is delivered to a mammalian cell via a liposome. In some embodiments, the CRISPR gene-editing system targeting the SCN10A gene is delivered to a mammalian cell via a lipid nanocrystal.

In some embodiments, the pharmaceutical composition comprises an sgRNA having a crRNA sequence selected from SEQ ID NOs: 2196-2243 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2196-2243 and a S. pyogenes Cas9 protein encapsulated in a lipid nanoparticle.

In various embodiments, the pharmaceutical composition comprising a CRISPR gene-editing system targeting the SCN10A gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the SCN10A gene, as described herein, to a subject in need thereof. In some embodiments, the pharmaceutical composition comprises an LNP encapsuling an sgRNA having a crRNA sequence selected from SEQ ID NOs: 2196-2243 and a S. pyogenes Cas9 protein. In some embodiments, the crRNA sequence is selected from SEQ ID NOs: 2196-2220. In some embodiments, the crRNA sequence is selected from SEQ ID NOs: 2196-2205. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the SCN10A gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the SCN10A gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an SCN10A gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, any intervening intronic regions, intron/exon junctions, the 3′ UTR, or polyadenylation signal) is targeted by an RNA-guided nuclease to alter the gene. In some embodiments, the SCN10A gene targeted by an RNA-guided nuclease is from a mammal. In some embodiments, the SCN10A gene targeted by an RNA-guided nuclease is from a human (hSCN10A). In some embodiments, the SCN10A gene targeted by an RNA-guided nuclease is from a dog (cSCN10A). In some embodiments, the SCN10A gene targeted by an RNA-guided nuclease is from a horse (eSCN10A). In some embodiments, the SCN10A gene targeted by an RNA-guided nuclease is from a cat (fSCN10A).

### FFF. SCN11A

In one aspect, methods and pharmaceutical compositions are provided for treating a back or spine condition or disorder by administering a therapeutically effective amount of a CRISPR pharmaceutical composition that targets an SCN11A gene to a subject in need thereof. In some embodiments, the CRISPR pharmaceutical composition comprises an RNA-guided nuclease and an sgRNA targeting the SCN11A gene with a crRNA sequence selected from SEQ ID NOs: 2244-2291. In some embodiments, the crRNA sequence is selected from SEQ ID NOs: 2244-2268. In some embodiments, the crRNA sequence is selected from 2244-2253. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the SCN11A gene is delivered to a mammalian cell via viral vector. In some embodiments, the CRISPR gene-editing system targeting the SCN11A gene is delivered to a mammalian cell via an AAV vector. In some embodiments, the CRISPR gene-editing system targeting the SCN11A gene is delivered to a mammalian cell via a lentiviral vector. In some embodiments, the CRISPR gene-editing system targeting the SCN11A gene is delivered to a mammalian cell via a lipid nanoparticle. In some embodiments, the CRISPR gene-editing system targeting the SCN11A gene is delivered to a mammalian cell via a virus-like particle. In some embodiments, the CRISPR gene-editing system targeting the SCN11A gene is delivered to a mammalian cell via a liposome. In some embodiments, the CRISPR gene-editing system targeting the SCN1 TA gene is delivered to a mammalian cell via a lipid nanocrystal.

In some embodiments, the pharmaceutical composition comprises an sgRNA having a crRNA sequence selected from SEQ ID NOs: 2244-2291 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2244-2291 and a S. pyogenes Cas9 protein encapsulated in a lipid nanoparticle.

In various embodiments, the pharmaceutical composition comprising a CRISPR gene-editing system targeting the SCN11A gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the SCN11A gene, as described herein, to a subject in need thereof. In some embodiments, the pharmaceutical composition comprises an LNP encapsuling an sgRNA having a crRNA sequence selected from SEQ ID NOs: 2244-2291 and a S. pyogenes Cas9 protein. In some embodiments, the crRNA sequence is selected from SEQ ID NOs: 2244-2268. In some embodiments, the crRNA sequence is selected from SEQ ID NOs: 2244-2253. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the SCN11A gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the SCN11A gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an SCN11A gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, any intervening intronic regions, intron/exon junctions, the 3′ UTR, or polyadenylation signal) is targeted by an RNA-guided nuclease to alter the gene. In some embodiments, the SCN11A gene targeted by an RNA-guided nuclease is from a mammal. In some embodiments, the SCN11A gene targeted by an RNA-guided nuclease is from a human (hSCN11A). In some embodiments, the SCN11A gene targeted by an RNA-guided nuclease is from a dog (cSCN11A). In some embodiments, the SCN11A gene targeted by an RNA-guided nuclease is from a horse (eSCN11A). In some embodiments, the SCN11A gene targeted by an RNA-guided nuclease is from a cat (fSCN11A).

### GGG. NGF

In one aspect, methods and pharmaceutical compositions are provided for treating a back or spine condition or disorder by administering a therapeutically effective amount of a CRISPR pharmaceutical composition that targets an NGF gene to a subject in need thereof. In some embodiments, the CRISPR pharmaceutical composition comprises an RNA-guided nuclease and an sgRNA targeting the NGF gene with a crRNA sequence selected from SEQ ID NOs: 1586-1628. In some embodiments, the crRNA sequence is selected from SEQ ID NOs: 1586-1610. In some embodiments, the crRNA sequence is selected from 1586-1595. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the NGF gene is delivered to a mammalian cell via viral vector. In some embodiments, the CRISPR gene-editing system targeting the NGF gene is delivered to a mammalian cell via an AAV vector. In some embodiments, the CRISPR gene-editing system targeting the NGF gene is delivered to a mammalian cell via a lentiviral vector. In some embodiments, the CRISPR gene-editing system targeting the NGF gene is delivered to a mammalian cell via a lipid nanoparticle. In some embodiments, the CRISPR gene-editing system targeting the NGF gene is delivered to a mammalian cell via a virus-like particle. In some embodiments, the CRISPR gene-editing system targeting the NGF gene is delivered to a mammalian cell via a liposome. In some embodiments, the CRISPR gene-editing system targeting the NGF gene is delivered to a mammalian cell via a lipid nanocrystal.

In some embodiments, the pharmaceutical composition comprises an sgRNA having a crRNA sequence selected from SEQ ID NOs: 1586-1628 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1586-1628 and a S. pyogenes Cas9 protein encapsulated in a lipid nanoparticle.

In various embodiments, the pharmaceutical composition comprising a CRISPR gene-editing system targeting the NGF gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the NGF gene, as described herein, to a subject in need thereof. In some embodiments, the pharmaceutical composition comprises an LNP encapsuling an sgRNA having a crRNA sequence selected from SEQ ID NOs: 1586-1628 and a S. pyogenes Cas9 protein. In some embodiments, the crRNA sequence is selected from SEQ ID NOs: 1586-1610. In some embodiments, the crRNA sequence is selected from SEQ ID NOs: 1586-1595. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the NGF gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the NGF gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an NGF gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the NGF gene targeted by an RNA-guided nuclease is from a mammal. In some embodiments, the NGF gene targeted by an RNA-guided nuclease is from a human (hNGF). In some embodiments, the NGF gene targeted by an RNA-guided nuclease is from a dog (cNGF). In some embodiments, the NGF gene targeted by an RNA-guided nuclease is from a horse (eNGF). In some embodiments, the NGF gene targeted by an RNA-guided nuclease is from a cat (fNGF).

### HHH. NGFR

In one aspect, methods and pharmaceutical compositions are provided for treating a back or spine condition or disorder by administering a therapeutically effective amount of a CRISPR pharmaceutical composition that targets an NGFR gene to a subject in need thereof. In some embodiments, the CRISPR pharmaceutical composition comprises an RNA-guided nuclease and an sgRNA targeting the NGFR gene with a crRNA sequence selected from SEQ ID NOs: 1629-1676. In some embodiments, the crRNA sequence is selected from SEQ ID NOs: 1629-1653. In some embodiments, the crRNA sequence is selected from 1629-1638. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the NGFR gene is delivered to a mammalian cell via viral vector. In some embodiments, the CRISPR gene-editing system targeting the NGFR gene is delivered to a mammalian cell via an AAV vector. In some embodiments, the CRISPR gene-editing system targeting the NGFR gene is delivered to a mammalian cell via a lentiviral vector. In some embodiments, the CRISPR gene-editing system targeting the NGFR gene is delivered to a mammalian cell via a lipid nanoparticle. In some embodiments, the CRISPR gene-editing system targeting the NGFR gene is delivered to a mammalian cell via a virus-like particle. In some embodiments, the CRISPR gene-editing system targeting the NGFR gene is delivered to a mammalian cell via a liposome. In some embodiments, the CRISPR gene-editing system targeting the NGFR gene is delivered to a mammalian cell via a lipid nanocrystal.

In some embodiments, the pharmaceutical composition comprises an sgRNA having a crRNA sequence selected from SEQ ID NOs: 1629-1676 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1629-1676 and a S. pyogenes Cas9 protein encapsulated in a lipid nanoparticle.

In various embodiments, the pharmaceutical composition comprising a CRISPR gene-editing system targeting the NGFR gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the NGFR gene, as described herein, to a subject in need thereof. In some embodiments, the pharmaceutical composition comprises an LNP encapsuling an sgRNA having a crRNA sequence selected from SEQ ID NOs: 1629-1676 and a S. pyogenes Cas9 protein. In some embodiments, the crRNA sequence is selected from SEQ ID NOs: 1629-1653. In some embodiments, the crRNA sequence is selected from SEQ ID NOs: 1629-1638. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the NGFR gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the NGFR gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an NGFR gene (e.g., 5′ untranslated region [UTR], exon 1, exon 2, exon 3, exon 4, exon 5, exon 6, any intervening intronic regions, intron/exon junctions, the 3′ UTR, or polyadenylation signal) is targeted by an RNA-guided nuclease to alter the gene. In some embodiments, the NGFR gene targeted by an RNA-guided nuclease is from a mammal. In some embodiments, the NGFR gene targeted by an RNA-guided nuclease is from a human (hNGFR). In some embodiments, the NGFR gene targeted by an RNA-guided nuclease is from a dog (cNGFR). In some embodiments, the NGFR gene targeted by an RNA-guided nuclease is from a horse (eNGFR). In some embodiments, the NGFR gene targeted by an RNA-guided nuclease is from a cat (fNGFR).

### III. NTF3

In one aspect, methods and pharmaceutical compositions are provided for treating a back or spine condition or disorder by administering a therapeutically effective amount of a CRISPR pharmaceutical composition that targets an NTF3 gene to a subject in need thereof. In some embodiments, the CRISPR pharmaceutical composition comprises an RNA-guided nuclease and an sgRNA targeting the NTF3 gene with a crRNA sequence selected from SEQ ID NOs: 1677-1724. In some embodiments, the crRNA sequence is selected from SEQ ID NOs: 1677-1701. In some embodiments, the crRNA sequence is selected from 1677-1686. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the NTF3 gene is delivered to a mammalian cell via viral vector. In some embodiments, the CRISPR gene-editing system targeting the NTF3 gene is delivered to a mammalian cell via an AAV vector. In some embodiments, the CRISPR gene-editing system targeting the NTF3 gene is delivered to a mammalian cell via a lentiviral vector. In some embodiments, the CRISPR gene-editing system targeting the NTF3 gene is delivered to a mammalian cell via a lipid nanoparticle. In some embodiments, the CRISPR gene-editing system targeting the NTF3 gene is delivered to a mammalian cell via a virus-like particle. In some embodiments, the CRISPR gene-editing system targeting the NTF3 gene is delivered to a mammalian cell via a liposome. In some embodiments, the CRISPR gene-editing system targeting the NTF3 gene is delivered to a mammalian cell via a lipid nanocrystal.

In some embodiments, the pharmaceutical composition comprises an sgRNA having a crRNA sequence selected from SEQ ID NOs: 1677-1724 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1677-1724 and a S. pyogenes Cas9 protein encapsulated in a lipid nanoparticle.

In various embodiments, the pharmaceutical composition comprising a CRISPR gene-editing system targeting the NTF3 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the NTF3 gene, as described herein, to a subject in need thereof. In some embodiments, the pharmaceutical composition comprises an LNP encapsuling an sgRNA having a crRNA sequence selected from SEQ ID NOs: 1677-1724 and a S. pyogenes Cas9 protein. In some embodiments, the crRNA sequence is selected from SEQ ID NOs: 1677-1701. In some embodiments, the crRNA sequence is selected from SEQ ID NOs: 1677-1686. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the NTF3 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the NTF3 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an NTF3 gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the NTF3 gene targeted by an RNA-guided nuclease is from a mammal. In some embodiments, the NTF3 gene targeted by an RNA-guided nuclease is from a human (hNTF3). In some embodiments, the NTF3 gene targeted by an RNA-guided nuclease is from a dog (cNTF3). In some embodiments, the NTF3 gene targeted by an RNA-guided nuclease is from a horse (eNTF3). In some embodiments, the NTF3 gene targeted by an RNA-guided nuclease is from a cat (fNTF3).

### JJJ. NTF4

In one aspect, methods and pharmaceutical compositions are provided for treating a back or spine condition or disorder by administering a therapeutically effective amount of a CRISPR pharmaceutical composition that targets an NTF4 gene to a subject in need thereof. In some embodiments, the CRISPR pharmaceutical composition comprises an RNA-guided nuclease and an sgRNA targeting the NTF4 gene with a crRNA sequence selected from SEQ ID NOs: 1725-1746. In some embodiments, the crRNA sequence is selected from 1725-1734. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the NTF4 gene is delivered to a mammalian cell via viral vector. In some embodiments, the CRISPR gene-editing system targeting the NTF4 gene is delivered to a mammalian cell via an AAV vector. In some embodiments, the CRISPR gene-editing system targeting the NTF4 gene is delivered to a mammalian cell via a lentiviral vector. In some embodiments, the CRISPR gene-editing system targeting the NTF4 gene is delivered to a mammalian cell via a lipid nanoparticle. In some embodiments, the CRISPR gene-editing system targeting the NTF4 gene is delivered to a mammalian cell via a virus-like particle. In some embodiments, the CRISPR gene-editing system targeting the NTF4 gene is delivered to a mammalian cell via a liposome. In some embodiments, the CRISPR gene-editing system targeting the NTF4 gene is delivered to a mammalian cell via a lipid nanocrystal.

In some embodiments, the pharmaceutical composition comprises an sgRNA having a crRNA sequence selected from SEQ ID NOs: 1725-1746 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1725-1746 and a S. pyogenes Cas9 protein encapsulated in a lipid nanoparticle.

In various embodiments, the pharmaceutical composition comprising a CRISPR gene-editing system targeting the NTF4 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the NTF4 gene, as described herein, to a subject in need thereof. In some embodiments, the pharmaceutical composition comprises an LNP encapsuling an sgRNA having a crRNA sequence selected from SEQ ID NOs: 1725-1746 and a S. pyogenes Cas9 protein. In some embodiments, the crRNA sequence is selected from SEQ ID NOs: 1725-1734. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the NTF4 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the NTF4 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an NTF4 gene (e.g., 5′ untranslated region [UTR], exon 1, exon 2, exon 3, exon 4, exon 5, exon 6, exon 7, any intervening intronic regions, intron/exon junctions, the 3′ UTR, or polyadenylation signal) is targeted by an RNA-guided nuclease to alter the gene. In some embodiments, the NTF4 gene targeted by an RNA-guided nuclease is from a mammal. In some embodiments, the NTF4 gene targeted by an RNA-guided nuclease is from a human (hNTF4). In some embodiments, the NTF4 gene targeted by an RNA-guided nuclease is from a dog (cNTF4). In some embodiments, the NTF4 gene targeted by an RNA-guided nuclease is from a horse (eNTF4). In some embodiments, the NTF4 gene targeted by an RNA-guided nuclease is from a cat (fNTF4).

### KKK. NTRK1

In one aspect, methods and pharmaceutical compositions are provided for treating a back or spine condition or disorder by administering a therapeutically effective amount of a CRISPR pharmaceutical composition that targets an NTRK1 gene to a subject in need thereof. In some embodiments, the CRISPR pharmaceutical composition comprises an RNA-guided nuclease and an sgRNA targeting the NTRK1 gene with a crRNA sequence selected from SEQ ID NOs: 1747-1794. In some embodiments, the crRNA sequence is selected from SEQ ID NOs: 1747-1771. In some embodiments, the crRNA sequence is selected from 1747-1756. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the NTRK1 gene is delivered to a mammalian cell via viral vector. In some embodiments, the CRISPR gene-editing system targeting the NTRK1 gene is delivered to a mammalian cell via an AAV vector. In some embodiments, the CRISPR gene-editing system targeting the NTRK1 gene is delivered to a mammalian cell via a lentiviral vector. In some embodiments, the CRISPR gene-editing system targeting the NTRK11 gene is delivered to a mammalian cell via a lipid nanoparticle. In some embodiments, the CRISPR gene-editing system targeting the NTRK1 gene is delivered to a mammalian cell via a virus-like particle. In some embodiments, the CRISPR gene-editing system targeting the NTRK1 gene is delivered to a mammalian cell via a liposome. In some embodiments, the CRISPR gene-editing system targeting the NTRK1 gene is delivered to a mammalian cell via a lipid nanocrystal.

In some embodiments, the pharmaceutical composition comprises an sgRNA having a crRNA sequence selected from SEQ ID NOs: 1747-1794 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1747-1794 and a S. pyogenes Cas9 protein encapsulated in a lipid nanoparticle.

In various embodiments, the pharmaceutical composition comprising a CRISPR gene-editing system targeting the NTRK1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the NTRK1 gene, as described herein, to a subject in need thereof. In some embodiments, the pharmaceutical composition comprises an LNP encapsuling an sgRNA having a crRNA sequence selected from SEQ ID NOs: 1747-1794 and a S. pyogenes Cas9 protein. In some embodiments, the crRNA sequence is selected from SEQ ID NOs: 1747-1771. In some embodiments, the crRNA sequence is selected from SEQ ID NOs: 1747-1756. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the NTRK11 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the NTRK11 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an NTRK1 gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, any intervening intronic regions, intron/exon junctions, the 3′ UTR, or polyadenylation signal) is targeted by an RNA-guided nuclease to alter the gene. In some embodiments, the NTRK1 gene targeted by an RNA-guided nuclease is from a mammal. In some embodiments, the NTRK1 gene targeted by an RNA-guided nuclease is from a human (hNTRK1). In some embodiments, the NTRK11 gene targeted by an RNA-guided nuclease is from a dog (cNTRK1). In some embodiments, the NTRK1 gene targeted by an RNA-guided nuclease is from a horse (eNTRK1). In some embodiments, the NTRK1 gene targeted by an RNA-guided nuclease is from a cat (fNTRK1).

### LLL. NTRK2

In one aspect, methods and pharmaceutical compositions are provided for treating a back or spine condition or disorder by administering a therapeutically effective amount of a CRISPR pharmaceutical composition that targets an NTRK2 gene to a subject in need thereof. In some embodiments, the CRISPR pharmaceutical composition comprises an RNA-guided nuclease and an sgRNA targeting the NTRK2 gene with a crRNA sequence selected from SEQ ID NOs: 1795-1842. In some embodiments, the crRNA sequence is selected from SEQ ID NOs: 1795-1819. In some embodiments, the crRNA sequence is selected from 1795-1804. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the NTRK2 gene is delivered to a mammalian cell via viral vector. In some embodiments, the CRISPR gene-editing system targeting the NTRK2 gene is delivered to a mammalian cell via an AAV vector. In some embodiments, the CRISPR gene-editing system targeting the NTRK2 gene is delivered to a mammalian cell via a lentiviral vector. In some embodiments, the CRISPR gene-editing system targeting the NTRK2 gene is delivered to a mammalian cell via a lipid nanoparticle. In some embodiments, the CRISPR gene-editing system targeting the NTRK2 gene is delivered to a mammalian cell via a virus-like particle. In some embodiments, the CRISPR gene-editing system targeting the NTRK2 gene is delivered to a mammalian cell via a liposome. In some embodiments, the CRISPR gene-editing system targeting the NTRK2 gene is delivered to a mammalian cell via a lipid nanocrystal.

In some embodiments, the pharmaceutical composition comprises an sgRNA having a crRNA sequence selected from SEQ ID NOs: 1795-1842 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1795-1842 and a S. pyogenes Cas9 protein encapsulated in a lipid nanoparticle.

In various embodiments, the pharmaceutical composition comprising a CRISPR gene-editing system targeting the NTRK2 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the NTRK2 gene, as described herein, to a subject in need thereof. In some embodiments, the pharmaceutical composition comprises an LNP encapsuling an sgRNA having a crRNA sequence selected from SEQ ID NOs: 1795-1842 and a S. pyogenes Cas9 protein. In some embodiments, the crRNA sequence is selected from SEQ ID NOs: 1795-1819. In some embodiments, the crRNA sequence is selected from SEQ ID NOs: 1795-1804. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the NTRK2 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the NTRK2 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an NTRK2 gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, any intervening intronic regions, intron/exon junctions, the 3′ UTR, or polyadenylation signal) is targeted by an RNA-guided nuclease to alter the gene. In some embodiments, the NTRK2 gene targeted by an RNA-guided nuclease is from a mammal. In some embodiments, the NTRK2 gene targeted by an RNA-guided nuclease is from a human (hNTRK2). In some embodiments, the NTRK2 gene targeted by an RNA-guided nuclease is from a dog (cNTRK2). In some embodiments, the NTRK2 gene targeted by an RNA-guided nuclease is from a horse (eNTRK2). In some embodiments, the NTRK2 gene targeted by an RNA-guided nuclease is from a cat (fNTRK2).

### MMM. BDNF

In one aspect, methods and pharmaceutical compositions are provided for treating a back or spine condition or disorder by administering a therapeutically effective amount of a CRISPR pharmaceutical composition that targets an BDNF gene to a subject in need thereof. In some embodiments, the CRISPR pharmaceutical composition comprises an RNA-guided nuclease and an sgRNA targeting the BDNF gene with a crRNA sequence selected from SEQ ID NOs: 240-287. In some embodiments, the crRNA sequence is selected from SEQ ID NOs: 240-264. In some embodiments, the crRNA sequence is selected from 240-249. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the BDNF gene is delivered to a mammalian cell via viral vector. In some embodiments, the CRISPR gene-editing system targeting the BDNF gene is delivered to a mammalian cell via an AAV vector. In some embodiments, the CRISPR gene-editing system targeting the BDNF gene is delivered to a mammalian cell via a lentiviral vector. In some embodiments, the CRISPR gene-editing system targeting the BDNF gene is delivered to a mammalian cell via a lipid nanoparticle. In some embodiments, the CRISPR gene-editing system targeting the BDNF gene is delivered to a mammalian cell via a virus-like particle. In some embodiments, the CRISPR gene-editing system targeting the BDNF gene is delivered to a mammalian cell via a liposome. In some embodiments, the CRISPR gene-editing system targeting the BDNF gene is delivered to a mammalian cell via a lipid nanocrystal.

In some embodiments, the pharmaceutical composition comprises an sgRNA having a crRNA sequence selected from SEQ ID NOs: 241-281 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 241-281 and a S. pyogenes Cas9 protein encapsulated in a lipid nanoparticle.

In various embodiments, the pharmaceutical composition comprising a CRISPR gene-editing system targeting the BDNF gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the BDNF gene, as described herein, to a subject in need thereof. In some embodiments, the pharmaceutical composition comprises an LNP encapsuling an sgRNA having a crRNA sequence selected from SEQ ID NOs: 241-281 and a S. pyogenes Cas9 protein. In some embodiments, the crRNA sequence is selected from SEQ ID NOs: 241-265. In some embodiments, the crRNA sequence is selected from SEQ ID NOs: 241-250. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the BDNF gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the BDNF gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an BDNF gene (e.g., 5′ untranslated region [UTR], exon 1, exon 2, exon 3, exon 4, exon 5, exon 6, exon 7, exon 8, exon 9, exon 10, exon 11, exon 12, any intervening intronic regions, intron/exon junctions, the 3′ UTR, or polyadenylation signal) is targeted by an RNA-guided nuclease to alter the gene. In some embodiments, the BDNF gene targeted by an RNA-guided nuclease is from a mammal. In some embodiments, the BDNF gene targeted by an RNA-guided nuclease is from a human (hBDNF). In some embodiments, the BDNF gene targeted by an RNA-guided nuclease is from a dog (cBDNF). In some embodiments, the BDNF gene targeted by an RNA-guided nuclease is from a horse (eBDNF). In some embodiments, the BDNF gene targeted by an RNA-guided nuclease is from a cat (fBDNF).

### NNN. TAC1

In one aspect, methods and pharmaceutical compositions are provided for treating a back or spine condition or disorder by administering a therapeutically effective amount of a CRISPR pharmaceutical composition that targets an TACT gene to a subject in need thereof. In some embodiments, the CRISPR pharmaceutical composition comprises an RNA-guided nuclease and an sgRNA targeting the TACT gene with a crRNA sequence selected from SEQ ID NOs: 2292-2308. In some embodiments, the crRNA sequence is selected from 2292-2301. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the TAC1 gene is delivered to a mammalian cell via viral vector. In some embodiments, the CRISPR gene-editing system targeting the TACT gene is delivered to a mammalian cell via an AAV vector. In some embodiments, the CRISPR gene-editing system targeting the TACT gene is delivered to a mammalian cell via a lentiviral vector. In some embodiments, the CRISPR gene-editing system targeting the TACT gene is delivered to a mammalian cell via a lipid nanoparticle. In some embodiments, the CRISPR gene-editing system targeting the TACT gene is delivered to a mammalian cell via a virus-like particle. In some embodiments, the CRISPR gene-editing system targeting the TACT gene is delivered to a mammalian cell via a liposome. In some embodiments, the CRISPR gene-editing system targeting the TACT gene is delivered to a mammalian cell via a lipid nanocrystal.

In some embodiments, the pharmaceutical composition comprises an sgRNA having a crRNA sequence selected from SEQ ID NOs: 2292-2308 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2292-2308 and a S. pyogenes Cas9 protein encapsulated in a lipid nanoparticle.

In various embodiments, the pharmaceutical composition comprising a CRISPR gene-editing system targeting the TACT gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the TAC1 gene, as described herein, to a subject in need thereof. In some embodiments, the pharmaceutical composition comprises an LNP encapsuling an sgRNA having a crRNA sequence selected from SEQ ID NOs: 2292-2308 and a S. pyogenes Cas9 protein. In some embodiments, the crRNA sequence is selected from SEQ ID NOs: 2292-2301. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the TAC1 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the TAC1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an TAC1 gene (e.g., 5′ untranslated region [UTR], exon 1, exon 2, exon 3, exon 4, exon 5, exon 6, exon 7, exon 8, any intervening intronic regions, intron/exon junctions, the 3′ UTR, or polyadenylation signal) is targeted by an RNA-guided nuclease to alter the gene. In some embodiments, the TAC1 gene targeted by an RNA-guided nuclease is from a mammal. In some embodiments, the TAC1 gene targeted by an RNA-guided nuclease is from a human (hTAC1). In some embodiments, the TAC1 gene targeted by an RNA-guided nuclease is from a dog (cTAC1). In some embodiments, the TAC1 gene targeted by an RNA-guided nuclease is from a horse (eTAC1). In some embodiments, the TAC1 gene targeted by an RNA-guided nuclease is from a cat (fTAC1).

### OOO. TAC3

In one aspect, methods and pharmaceutical compositions are provided for treating a back or spine condition or disorder by administering a therapeutically effective amount of a CRISPR pharmaceutical composition that targets an TAC3 gene to a subject in need thereof. In some embodiments, the CRISPR pharmaceutical composition comprises an RNA-guided nuclease and an sgRNA targeting the TAC3 gene with a crRNA sequence selected from SEQ ID NOs: 2309-2325. In some embodiments, the crRNA sequence is selected from 2309-2318. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the TAC3 gene is delivered to a mammalian cell via viral vector. In some embodiments, the CRISPR gene-editing system targeting the TAC3 gene is delivered to a mammalian cell via an AAV vector. In some embodiments, the CRISPR gene-editing system targeting the TAC3 gene is delivered to a mammalian cell via a lentiviral vector. In some embodiments, the CRISPR gene-editing system targeting the TAC3 gene is delivered to a mammalian cell via a lipid nanoparticle. In some embodiments, the CRISPR gene-editing system targeting the TAC3 gene is delivered to a mammalian cell via a virus-like particle. In some embodiments, the CRISPR gene-editing system targeting the TAC3 gene is delivered to a mammalian cell via a liposome. In some embodiments, the CRISPR gene-editing system targeting the TAC3 gene is delivered to a mammalian cell via a lipid nanocrystal.

In some embodiments, the pharmaceutical composition comprises an sgRNA having a crRNA sequence selected from SEQ ID NOs: 2309-2325 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2309-2325 and a S. pyogenes Cas9 protein encapsulated in a lipid nanoparticle.

In various embodiments, the pharmaceutical composition comprising a CRISPR gene-editing system targeting the TAC3 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the TAC3 gene, as described herein, to a subject in need thereof. In some embodiments, the pharmaceutical composition comprises an LNP encapsuling an sgRNA having a crRNA sequence selected from SEQ ID NOs: 2309-2325 and a S. pyogenes Cas9 protein. In some embodiments, the crRNA sequence is selected from SEQ ID NOs: 2309-2318. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the TAC3 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the TAC3 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an TAC3 gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, any intervening intronic regions, intron/exon junctions, the 3′ UTR, or polyadenylation signal) is targeted by an RNA-guided nuclease to alter the gene. In some embodiments, the TAC3 gene targeted by an RNA-guided nuclease is from a mammal. In some embodiments, the TAC3 gene targeted by an RNA-guided nuclease is from a human (hTAC3). In some embodiments, the TAC3 gene targeted by an RNA-guided nuclease is from a dog (cTAC3). In some embodiments, the TAC3 gene targeted by an RNA-guided nuclease is from a horse (eTAC3). In some embodiments, the TAC3 gene targeted by an RNA-guided nuclease is from a cat (fTAC3).

### PPP. TACR1

In one aspect, methods and pharmaceutical compositions are provided for treating a back or spine condition or disorder by administering a therapeutically effective amount of a CRISPR pharmaceutical composition that targets an TACR1 gene to a subject in need thereof. In some embodiments, the CRISPR pharmaceutical composition comprises an RNA-guided nuclease and an sgRNA targeting the TACR1 gene with a crRNA sequence selected from SEQ ID NOs: 2326-2373. In some embodiments, the crRNA sequence is selected from SEQ ID NOs: 2326-2350. In some embodiments, the crRNA sequence is selected from 2326-2335. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the TACR1 gene is delivered to a mammalian cell via viral vector. In some embodiments, the CRISPR gene-editing system targeting the TACR1 gene is delivered to a mammalian cell via an AAV vector. In some embodiments, the CRISPR gene-editing system targeting the TACR1 gene is delivered to a mammalian cell via a lentiviral vector. In some embodiments, the CRISPR gene-editing system targeting the TACR1 gene is delivered to a mammalian cell via a lipid nanoparticle. In some embodiments, the CRISPR gene-editing system targeting the TACR1 gene is delivered to a mammalian cell via a virus-like particle. In some embodiments, the CRISPR gene-editing system targeting the TACR1 gene is delivered to a mammalian cell via a liposome. In some embodiments, the CRISPR gene-editing system targeting the TACR1 gene is delivered to a mammalian cell via a lipid nanocrystal.

In some embodiments, the pharmaceutical composition comprises an sgRNA having a crRNA sequence selected from SEQ ID NOs: 2326-2373 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2326-2373 and a S. pyogenes Cas9 protein encapsulated in a lipid nanoparticle.

In various embodiments, the pharmaceutical composition comprising a CRISPR gene-editing system targeting the TACR1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the TACR1 gene, as described herein, to a subject in need thereof. In some embodiments, the pharmaceutical composition comprises an LNP encapsuling an sgRNA having a crRNA sequence selected from SEQ ID NOs: 2326-2373 and a S. pyogenes Cas9 protein. In some embodiments, the crRNA sequence is selected from SEQ ID NOs: 2326-2350. In some embodiments, the crRNA sequence is selected from SEQ ID NOs: 2326-2335. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the TACR1 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the TACR1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an TACR1 gene (e.g., 5′ untranslated region [UTR], exon 1, exon 2, exon 3, exon 4, exon 5, any intervening intronic regions, intron/exon junctions, the 3′ UTR, or polyadenylation signal) is targeted by an RNA-guided nuclease to alter the gene. In some embodiments, the TACR1 gene targeted by an RNA-guided nuclease is from a mammal. In some embodiments, the TACR1 gene targeted by an RNA-guided nuclease is from a human (hTACR1). In some embodiments, the TACR1 gene targeted by an RNA-guided nuclease is from a dog (cTACR1). In some embodiments, the TACR1 gene targeted by an RNA-guided nuclease is from a horse (eTACR1). In some embodiments, the TACR1 gene targeted by an RNA-guided nuclease is from a cat (fTACR1).

### QQQ. TACR2

In one aspect, methods and pharmaceutical compositions are provided for treating a back or spine condition or disorder by administering a therapeutically effective amount of a CRISPR pharmaceutical composition that targets an TACR2 gene to a subject in need thereof. In some embodiments, the CRISPR pharmaceutical composition comprises an RNA-guided nuclease and an sgRNA targeting the TACR2 gene with a crRNA sequence selected from SEQ ID NOs: 2374-2421. In some embodiments, the crRNA sequence is selected from SEQ ID NOs: 2374-2398. In some embodiments, the crRNA sequence is selected from 2374-2383. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the TACR2 gene is delivered to a mammalian cell via viral vector. In some embodiments, the CRISPR gene-editing system targeting the TACR2 gene is delivered to a mammalian cell via an AAV vector. In some embodiments, the CRISPR gene-editing system targeting the TACR2 gene is delivered to a mammalian cell via a lentiviral vector. In some embodiments, the CRISPR gene-editing system targeting the TACR2 gene is delivered to a mammalian cell via a lipid nanoparticle. In some embodiments, the CRISPR gene-editing system targeting the TACR2 gene is delivered to a mammalian cell via a virus-like particle. In some embodiments, the CRISPR gene-editing system targeting the TACR2 gene is delivered to a mammalian cell via a liposome. In some embodiments, the CRISPR gene-editing system targeting the TACR2 gene is delivered to a mammalian cell via a lipid nanocrystal.

In some embodiments, the pharmaceutical composition comprises an sgRNA having a crRNA sequence selected from SEQ ID NOs: 2374-2421 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2374-2421 and a S. pyogenes Cas9 protein encapsulated in a lipid nanoparticle.

In various embodiments, the pharmaceutical composition comprising a CRISPR gene-editing system targeting the TACR2 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the TACR2 gene, as described herein, to a subject in need thereof. In some embodiments, the pharmaceutical composition comprises an LNP encapsuling an sgRNA having a crRNA sequence selected from SEQ ID NOs: 2374-2421 and a S. pyogenes Cas9 protein. In some embodiments, the crRNA sequence is selected from SEQ ID NOs: 2374-2398. In some embodiments, the crRNA sequence is selected from SEQ ID NOs: 2374-2483. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the TACR2 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the TACR2 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an TACR2 gene (e.g., 5′ untranslated region [UTR], exon 1, exon 2, exon 3, exon 4, exon 5, exon 6, any intervening intronic regions, intron/exon junctions, the 3′ UTR, or polyadenylation signal) is targeted by an RNA-guided nuclease to alter the gene. In some embodiments, the TACR2 gene targeted by an RNA-guided nuclease is from a mammal. In some embodiments, the TACR2 gene targeted by an RNA-guided nuclease is from a human (hTACR2). In some embodiments, the TACR2 gene targeted by an RNA-guided nuclease is from a dog (cTACR2). In some embodiments, the TACR2 gene targeted by an RNA-guided nuclease is from a horse (eTACR2). In some embodiments, the TACR2 gene targeted by an RNA-guided nuclease is from a cat (fTACR2).

### RRR. TACR3

In one aspect, methods and pharmaceutical compositions are provided for treating a back or spine condition or disorder by administering a therapeutically effective amount of a CRISPR pharmaceutical composition that targets an TACR3 gene to a subject in need thereof. In some embodiments, the CRISPR pharmaceutical composition comprises an RNA-guided nuclease and an sgRNA targeting the TACR3 gene with a crRNA sequence selected from SEQ ID NOs: 2422-2469. In some embodiments, the crRNA sequence is selected from SEQ ID NOs: 2422-2446. In some embodiments, the crRNA sequence is selected from 2422-2431. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the TACR3 gene is delivered to a mammalian cell via viral vector. In some embodiments, the CRISPR gene-editing system targeting the TACR3 gene is delivered to a mammalian cell via an AAV vector. In some embodiments, the CRISPR gene-editing system targeting the TACR3 gene is delivered to a mammalian cell via a lentiviral vector. In some embodiments, the CRISPR gene-editing system targeting the TACR3 gene is delivered to a mammalian cell via a lipid nanoparticle. In some embodiments, the CRISPR gene-editing system targeting the TACR3 gene is delivered to a mammalian cell via a virus-like particle. In some embodiments, the CRISPR gene-editing system targeting the TACR3 gene is delivered to a mammalian cell via a liposome. In some embodiments, the CRISPR gene-editing system targeting the TACR3 gene is delivered to a mammalian cell via a lipid nanocrystal.

In some embodiments, the pharmaceutical composition comprises an sgRNA having a crRNA sequence selected from SEQ ID NOs: 2422-2469 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2422-2469 and a S. pyogenes Cas9 protein encapsulated in a lipid nanoparticle.

In various embodiments, the pharmaceutical composition comprising a CRISPR gene-editing system targeting the TACR3 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the TACR3 gene, as described herein, to a subject in need thereof. In some embodiments, the pharmaceutical composition comprises an LNP encapsuling an sgRNA having a crRNA sequence selected from SEQ ID NOs: 2422-2469 and a S. pyogenes Cas9 protein. In some embodiments, the crRNA sequence is selected from SEQ ID NOs: 2422-2446. In some embodiments, the crRNA sequence is selected from SEQ ID NOs: 2422-2431. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the TACR3 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the TACR3 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an TACR3 gene (e.g., 5′ untranslated region [UTR], exon 1, exon 2, exon 3, exon 4, exon 5, any intervening intronic regions, intron/exon junctions, the 3′ UTR, or polyadenylation signal) is targeted by an RNA-guided nuclease to alter the gene. In some embodiments, the TACR3 gene targeted by an RNA-guided nuclease is from a mammal. In some embodiments, the TACR3 gene targeted by an RNA-guided nuclease is from a human (hTACR3). In some embodiments, the TACR3 gene targeted by an RNA-guided nuclease is from a dog (cTACR3). In some embodiments, the TACR3 gene targeted by an RNA-guided nuclease is from a horse (eTACR3). In some embodiments, the TACR3 gene targeted by an RNA-guided nuclease is from a cat (fTACR3).

### SSS. MRGPRX2

In one aspect, methods and pharmaceutical compositions are provided for treating a back or spine condition or disorder by administering a therapeutically effective amount of a CRISPR pharmaceutical composition that targets an MRGPRX2 gene to a subject in need thereof. In some embodiments, the CRISPR pharmaceutical composition comprises an RNA-guided nuclease and an sgRNA targeting the MRGPRX2 gene with a crRNA sequence selected from SEQ ID NOs: 1569-1585. In some embodiments, the crRNA sequence is selected from 1569-1578. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the MRGPRX2 gene is delivered to a mammalian cell via viral vector. In some embodiments, the CRISPR gene-editing system targeting the MRGPRX2 gene is delivered to a mammalian cell via an AAV vector. In some embodiments, the CRISPR gene-editing system targeting the MRGPRX2 gene is delivered to a mammalian cell via a lentiviral vector. In some embodiments, the CRISPR gene-editing system targeting the MRGPRX2 gene is delivered to a mammalian cell via a lipid nanoparticle. In some embodiments, the CRISPR gene-editing system targeting the MRGPRX2 gene is delivered to a mammalian cell via a virus-like particle. In some embodiments, the CRISPR gene-editing system targeting the MRGPRX2 gene is delivered to a mammalian cell via a liposome. In some embodiments, the CRISPR gene-editing system targeting the MRGPRX2 gene is delivered to a mammalian cell via a lipid nanocrystal.

In some embodiments, the pharmaceutical composition comprises an sgRNA having a crRNA sequence selected from SEQ ID NOs: 1569-1585 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1569-1585 and a S. pyogenes Cas9 protein encapsulated in a lipid nanoparticle.

In various embodiments, the pharmaceutical composition comprising a CRISPR gene-editing system targeting the MRGPRX2 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the MRGPRX2 gene, as described herein, to a subject in need thereof. In some embodiments, the pharmaceutical composition comprises an LNP encapsuling an sgRNA having a crRNA sequence selected from SEQ ID NOs: 1569-1585 and a S. pyogenes Cas9 protein. In some embodiments, the crRNA sequence is selected from SEQ ID NOs: 1569-1578. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the MRGPRX2 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the MRGPRX2 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an MRGPRX2 gene (e.g., 5′ untranslated region [UTR], exon 1, exon 2, exon 3, exon 4, exon 5, any intervening intronic regions, intron/exon junctions, the 3′ UTR, or polyadenylation signal) is targeted by an RNA-guided nuclease to alter the gene. In some embodiments, the MRGPRX2 gene targeted by an RNA-guided nuclease is from a mammal. In some embodiments, the MRGPRX2 gene targeted by an RNA-guided nuclease is from a human (hMRGPRX2). In some embodiments, the MRGPRX2 gene targeted by an RNA-guided nuclease is from a dog (cMRGPRX2). In some embodiments, the MRGPRX2 gene targeted by an RNA-guided nuclease is from a horse (eMRGPRX2). In some embodiments, the MRGPRX2 gene targeted by an RNA-guided nuclease is from a cat (fMRGPRX2).

### TTT. ATP1A1

In one aspect, methods and pharmaceutical compositions are provided for treating a back or spine condition or disorder by administering a therapeutically effective amount of a CRISPR pharmaceutical composition that targets an ATP1A1 gene to a subject in need thereof. In some embodiments, the CRISPR pharmaceutical composition comprises an RNA-guided nuclease and an sgRNA targeting the ATP1A1 gene with a crRNA sequence selected from SEQ ID NOs: 193-240. In some embodiments, the crRNA sequence is selected from SEQ ID NOs: 193-217. In some embodiments, the crRNA sequence is selected from 193-202. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the ATP1A1 gene is delivered to a mammalian cell via viral vector. In some embodiments, the CRISPR gene-editing system targeting the ATP1A1 gene is delivered to a mammalian cell via an AAV vector. In some embodiments, the CRISPR gene-editing system targeting the ATP1A1 gene is delivered to a mammalian cell via a lentiviral vector. In some embodiments, the CRISPR gene-editing system targeting the ATP1A1 gene is delivered to a mammalian cell via a lipid nanoparticle. In some embodiments, the CRISPR gene-editing system targeting the ATP1A1 gene is delivered to a mammalian cell via a virus-like particle. In some embodiments, the CRISPR gene-editing system targeting the ATP1A1 gene is delivered to a mammalian cell via a liposome. In some embodiments, the CRISPR gene-editing system targeting the ATP1A1 gene is delivered to a mammalian cell via a lipid nanocrystal.

In some embodiments, the pharmaceutical composition comprises an sgRNA having a crRNA sequence selected from SEQ ID NOs: 193-239 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 193-239 and a S. pyogenes Cas9 protein encapsulated in a lipid nanoparticle.

In various embodiments, the pharmaceutical composition comprising a CRISPR gene-editing system targeting the ATP1A1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the ATP1 A1 gene, as described herein, to a subject in need thereof. In some embodiments, the pharmaceutical composition comprises an LNP encapsuling an sgRNA having a crRNA sequence selected from SEQ ID NOs: 193-239 and a S. pyogenes Cas9 protein. In some embodiments, the crRNA sequence is selected from SEQ ID NOs: 193-217. In some embodiments, the crRNA sequence is selected from SEQ ID NOs: 193-202. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the ATP1A1 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the ATP1A1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an ATP1A1 gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, any intervening intronic regions, intron/exon junctions, the 3′ UTR, or polyadenylation signal) is targeted by an RNA-guided nuclease to alter the gene. In some embodiments, the ATP1A1 gene targeted by an RNA-guided nuclease is from a mammal. In some embodiments, the ATP1A1 gene targeted by an RNA-guided nuclease is from a human (hATP1A1). In some embodiments, the ATP1A1 gene targeted by an RNA-guided nuclease is from a dog (cATP1A1). In some embodiments, the ATP1A1 gene targeted by an RNA-guided nuclease is from a horse (eATP1A1). In some embodiments, the ATP1A1 gene targeted by an RNA-guided nuclease is from a cat (fATP1A1).

### UUU. CALCA

In one aspect, methods and pharmaceutical compositions are provided for treating a back or spine condition or disorder by administering a therapeutically effective amount of a CRISPR pharmaceutical composition that targets an CALCA gene to a subject in need thereof. In some embodiments, the CRISPR pharmaceutical composition comprises an RNA-guided nuclease and an sgRNA targeting the CALCA gene with a crRNA sequence selected from SEQ ID NOs: 282-301. In some embodiments, the crRNA sequence is selected from 282-291. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CALCA gene is delivered to a mammalian cell via viral vector. In some embodiments, the CRISPR gene-editing system targeting the CALCA gene is delivered to a mammalian cell via an AAV vector. In some embodiments, the CRISPR gene-editing system targeting the CALCA gene is delivered to a mammalian cell via a lentiviral vector. In some embodiments, the CRISPR gene-editing system targeting the CALCA gene is delivered to a mammalian cell via a lipid nanoparticle. In some embodiments, the CRISPR gene-editing system targeting the CALCA gene is delivered to a mammalian cell via a virus-like particle. In some embodiments, the CRISPR gene-editing system targeting the CALCA gene is delivered to a mammalian cell via a liposome. In some embodiments, the CRISPR gene-editing system targeting the CALCA gene is delivered to a mammalian cell via a lipid nanocrystal.

In some embodiments, the pharmaceutical composition comprises an sgRNA having a crRNA sequence selected from SEQ ID NOs: 282-301 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 282-301 and a S. pyogenes Cas9 protein encapsulated in a lipid nanoparticle.

In various embodiments, the pharmaceutical composition comprising a CRISPR gene-editing system targeting the CALCA gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CALCA gene, as described herein, to a subject in need thereof. In some embodiments, the pharmaceutical composition comprises an LNP encapsuling an sgRNA having a crRNA sequence selected from SEQ ID NOs: 282-301 and a S. pyogenes Cas9 protein. In some embodiments, the crRNA sequence is selected from SEQ ID NOs: 282-291. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CALCA gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CALCA gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CALCA gene (e.g., 5′ untranslated region [UTR], exon 1, exon 2, exon 3, exon 4, exon 5, exon 6, exon 7, any intervening intronic regions, intron/exon junctions, the 3′ UTR, or polyadenylation signal) is targeted by an RNA-guided nuclease to alter the gene. In some embodiments, the CALCA gene targeted by an RNA-guided nuclease is from a mammal. In some embodiments, the CALCA gene targeted by an RNA-guided nuclease is from a human (hCALCA). In some embodiments, the CALCA gene targeted by an RNA-guided nuclease is from a dog (cCALCA). In some embodiments, the CALCA gene targeted by an RNA-guided nuclease is from a horse (eCALCA). In some embodiments, the CALCA gene targeted by an RNA-guided nuclease is from a cat (fCALCA).

### VVV. CALCB

In one aspect, methods and pharmaceutical compositions are provided for treating a back or spine condition or disorder by administering a therapeutically effective amount of a CRISPR pharmaceutical composition that targets an CALCB gene to a subject in need thereof. In some embodiments, the CRISPR pharmaceutical composition comprises an RNA-guided nuclease and an sgRNA targeting the CALCB gene with a crRNA sequence selected from SEQ ID NOs: 302-318. In some embodiments, the crRNA sequence is selected from 302-311. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CALCB gene is delivered to a mammalian cell via viral vector. In some embodiments, the CRISPR gene-editing system targeting the CALCB gene is delivered to a mammalian cell via an AAV vector. In some embodiments, the CRISPR gene-editing system targeting the CALCB gene is delivered to a mammalian cell via a lentiviral vector. In some embodiments, the CRISPR gene-editing system targeting the CALCB gene is delivered to a mammalian cell via a lipid nanoparticle. In some embodiments, the CRISPR gene-editing system targeting the CALCB gene is delivered to a mammalian cell via a virus-like particle. In some embodiments, the CRISPR gene-editing system targeting the CALCB gene is delivered to a mammalian cell via a liposome. In some embodiments, the CRISPR gene-editing system targeting the CALCB gene is delivered to a mammalian cell via a lipid nanocrystal.

In some embodiments, the pharmaceutical composition comprises an sgRNA having a crRNA sequence selected from SEQ ID NOs: 302-318 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 302-318 and a S. pyogenes Cas9 protein encapsulated in a lipid nanoparticle.

In various embodiments, the pharmaceutical composition comprising a CRISPR gene-editing system targeting the CALCB gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CALCB gene, as described herein, to a subject in need thereof. In some embodiments, the pharmaceutical composition comprises an LNP encapsuling an sgRNA having a crRNA sequence selected from SEQ ID NOs: 302-318 and a S. pyogenes Cas9 protein. In some embodiments, the crRNA sequence is selected from SEQ ID NOs: 302-311. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CALCB gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CALCB gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CALCB gene (e.g., 5′ untranslated region [UTR], exon 1, exon 2, exon 3, exon 4, exon 5, exon 6, exon 7, any intervening intronic regions, intron/exon junctions, the 3′ UTR, or polyadenylation signal) is targeted by an RNA-guided nuclease to alter the gene. In some embodiments, the CALCB gene targeted by an RNA-guided nuclease is from a mammal. In some embodiments, the CALCB gene targeted by an RNA-guided nuclease is from a human (hCALCB). In some embodiments, the CALCB gene targeted by an RNA-guided nuclease is from a dog (cCALCB). In some embodiments, the CALCB gene targeted by an RNA-guided nuclease is from a horse (eCALCB). In some embodiments, the CALCB gene targeted by an RNA-guided nuclease is from a cat (fCALCB).

### WWW. CALCRL

In one aspect, methods and pharmaceutical compositions are provided for treating a back or spine condition or disorder by administering a therapeutically effective amount of a CRISPR pharmaceutical composition that targets an CALCRL gene to a subject in need thereof. In some embodiments, the CRISPR pharmaceutical composition comprises an RNA-guided nuclease and an sgRNA targeting the CALCRL gene with a crRNA sequence selected from SEQ ID NOs: 319-340. In some embodiments, the crRNA sequence is selected from 319-328. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CALCRL gene is delivered to a mammalian cell via viral vector. In some embodiments, the CRISPR gene-editing system targeting the CALCRL gene is delivered to a mammalian cell via an AAV vector. In some embodiments, the CRISPR gene-editing system targeting the CALCRL gene is delivered to a mammalian cell via a lentiviral vector. In some embodiments, the CRISPR gene-editing system targeting the CALCRL gene is delivered to a mammalian cell via a lipid nanoparticle. In some embodiments, the CRISPR gene-editing system targeting the CALCRL gene is delivered to a mammalian cell via a virus-like particle. In some embodiments, the CRISPR gene-editing system targeting the CALCRL gene is delivered to a mammalian cell via a liposome. In some embodiments, the CRISPR gene-editing system targeting the CALCRL gene is delivered to a mammalian cell via a lipid nanocrystal.

In some embodiments, the pharmaceutical composition comprises an sgRNA having a crRNA sequence selected from SEQ ID NOs: 319-340 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 319-340 and a S. pyogenes Cas9 protein encapsulated in a lipid nanoparticle.

In various embodiments, the pharmaceutical composition comprising a CRISPR gene-editing system targeting the CALCRL gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CALCRL gene, as described herein, to a subject in need thereof. In some embodiments, the pharmaceutical composition comprises an LNP encapsuling an sgRNA having a crRNA sequence selected from SEQ ID NOs: 319-340 and a S. pyogenes Cas9 protein. In some embodiments, the crRNA sequence is selected from SEQ ID NOs: 319-328. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CALCRL gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CALCRL gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CALCRL gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, any intervening intronic regions, intron/exon junctions, the 3′ UTR, or polyadenylation signal) is targeted by an RNA-guided nuclease to alter the gene. In some embodiments, the CALCRL gene targeted by an RNA-guided nuclease is from a mammal. In some embodiments, the CALCRL gene targeted by an RNA-guided nuclease is from a human (hCALCRL). In some embodiments, the CALCRL gene targeted by an RNA-guided nuclease is from a dog (cCALCRL). In some embodiments, the CALCRL gene targeted by an RNA-guided nuclease is from a horse (eCALCRL). In some embodiments, the CALCRL gene targeted by an RNA-guided nuclease is from a cat (fCALCRL).

### XXX. RAMP1

In one aspect, methods and pharmaceutical compositions are provided for treating a back or spine condition or disorder by administering a therapeutically effective amount of a CRISPR pharmaceutical composition that targets an RAMP1 gene to a subject in need thereof. In some embodiments, the CRISPR pharmaceutical composition comprises an RNA-guided nuclease and an sgRNA targeting the RAMP1 gene with a crRNA sequence selected from SEQ ID NOs: 1843-1859. In some embodiments, the crRNA sequence is selected from 1843-1852. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the RAMP1 gene is delivered to a mammalian cell via viral vector. In some embodiments, the CRISPR gene-editing system targeting the RAMP1 gene is delivered to a mammalian cell via an AAV vector. In some embodiments, the CRISPR gene-editing system targeting the RAMP1 gene is delivered to a mammalian cell via a lentiviral vector. In some embodiments, the CRISPR gene-editing system targeting the RAMP1 gene is delivered to a mammalian cell via a lipid nanoparticle. In some embodiments, the CRISPR gene-editing system targeting the RAMP1 gene is delivered to a mammalian cell via a virus-like particle. In some embodiments, the CRISPR gene-editing system targeting the RAMP1 gene is delivered to a mammalian cell via a liposome. In some embodiments, the CRISPR gene-editing system targeting the RAMP1 gene is delivered to a mammalian cell via a lipid nanocrystal.

In some embodiments, the pharmaceutical composition comprises an sgRNA having a crRNA sequence selected from SEQ ID NOs: 1843-1859 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 1843-1859 and a S. pyogenes Cas9 protein encapsulated in a lipid nanoparticle.

In various embodiments, the pharmaceutical composition comprising a CRISPR gene-editing system targeting the RAMP1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the RAMP1 gene, as described herein, to a subject in need thereof. In some embodiments, the pharmaceutical composition comprises an LNP encapsuling an sgRNA having a crRNA sequence selected from SEQ ID NOs: 1843-1859 and a S. pyogenes Cas9 protein. In some embodiments, the crRNA sequence is selected from SEQ ID NOs: 1843-1852. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the RAMP1 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the RAMP1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an RAMP1 gene (e.g., 5′ untranslated region [UTR], exon 1, exon 2, exon 3, exon 4, exon 5, exon 6, any intervening intronic regions, intron/exon junctions, the 3′ UTR, or polyadenylation signal) is targeted by an RNA-guided nuclease to alter the gene. In some embodiments, the RAMP1 gene targeted by an RNA-guided nuclease is from a mammal. In some embodiments, the RAMP1 gene targeted by an RNA-guided nuclease is from a human (hRAMP1). In some embodiments, the RAMP1 gene targeted by an RNA-guided nuclease is from a dog (cRAMP1). In some embodiments, the RAMP1 gene targeted by an RNA-guided nuclease is from a horse (eRAMP1). In some embodiments, the RAMP1 gene targeted by an RNA-guided nuclease is from a cat (fRAMP1).

### YYY. ADM

In one aspect, methods and pharmaceutical compositions are provided for treating a back or spine condition or disorder by administering a therapeutically effective amount of a CRISPR pharmaceutical composition that targets an ADM gene to a subject in need thereof. In some embodiments, the CRISPR pharmaceutical composition comprises an RNA-guided nuclease and an sgRNA targeting the ADM gene with a crRNA sequence selected from SEQ ID NOs: 145-192. In some embodiments, the crRNA sequence is selected from SEQ ID NOs: 145-169. In some embodiments, the crRNA sequence is selected from 145-154. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the ADM gene is delivered to a mammalian cell via viral vector. In some embodiments, the CRISPR gene-editing system targeting the ADM gene is delivered to a mammalian cell via an AAV vector. In some embodiments, the CRISPR gene-editing system targeting the ADM gene is delivered to a mammalian cell via a lentiviral vector. In some embodiments, the CRISPR gene-editing system targeting the ADM gene is delivered to a mammalian cell via a lipid nanoparticle. In some embodiments, the CRISPR gene-editing system targeting the ADM gene is delivered to a mammalian cell via a virus-like particle. In some embodiments, the CRISPR gene-editing system targeting the ADM gene is delivered to a mammalian cell via a liposome. In some embodiments, the CRISPR gene-editing system targeting the ADM gene is delivered to a mammalian cell via a lipid nanocrystal.

In some embodiments, the pharmaceutical composition comprises an sgRNA having a crRNA sequence selected from SEQ ID NOs: 145-192 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 145-192 and a S. pyogenes Cas9 protein encapsulated in a lipid nanoparticle.

In various embodiments, the pharmaceutical composition comprising a CRISPR gene-editing system targeting the ADM gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the ADM gene, as described herein, to a subject in need thereof. In some embodiments, the pharmaceutical composition comprises an LNP encapsuling an sgRNA having a crRNA sequence selected from SEQ ID NOs: 145-192 and a S. pyogenes Cas9 protein. In some embodiments, the crRNA sequence is selected from SEQ ID NOs: 145-169. In some embodiments, the crRNA sequence is selected from SEQ ID NOs: 145-154. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the ADM gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the ADM gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an ADM gene (e.g., 5′ untranslated region [UTR], exon 1, exon 2, exon 3, exon 4, any intervening intronic regions, intron/exon junctions, the 3′ UTR, or polyadenylation signal) is targeted by an RNA-guided nuclease to alter the gene. In some embodiments, the ADM gene targeted by an RNA-guided nuclease is from a mammal. In some embodiments, the ADM gene targeted by an RNA-guided nuclease is from a human (hADM). In some embodiments, the ADM gene targeted by an RNA-guided nuclease is from a dog (cADM). In some embodiments, the ADM gene targeted by an RNA-guided nuclease is from a horse (eADM). In some embodiments, the ADM gene targeted by an RNA-guided nuclease is from a cat (fADM).

### ZZZ. CRCP

In one aspect, methods and pharmaceutical compositions are provided for treating a back or spine condition or disorder by administering a therapeutically effective amount of a CRISPR pharmaceutical composition that targets an CRCP gene to a subject in need thereof. In some embodiments, the CRISPR pharmaceutical composition comprises an RNA-guided nuclease and an sgRNA targeting the CRCP gene with a crRNA sequence selected from SEQ ID NOs: 518-534. In some embodiments, the crRNA sequence is selected from 518-527. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the CRCP gene is delivered to a mammalian cell via viral vector. In some embodiments, the CRISPR gene-editing system targeting the CRCP gene is delivered to a mammalian cell via an AAV vector.

In some embodiments, the CRISPR gene-editing system targeting the CRCP gene is delivered to a mammalian cell via a lentiviral vector. In some embodiments, the CRISPR gene-editing system targeting the CRCP gene is delivered to a mammalian cell via a lipid nanoparticle. In some embodiments, the CRISPR gene-editing system targeting the CRCP gene is delivered to a mammalian cell via a virus-like particle. In some embodiments, the CRISPR gene-editing system targeting the CRCP gene is delivered to a mammalian cell via a liposome. In some embodiments, the CRISPR gene-editing system targeting the CRCP gene is delivered to a mammalian cell via a lipid nanocrystal.

In some embodiments, the pharmaceutical composition comprises an sgRNA having a crRNA sequence selected from SEQ ID NOs: 518-534 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 518-534 and a S. pyogenes Cas9 protein encapsulated in a lipid nanoparticle.

In various embodiments, the pharmaceutical composition comprising a CRISPR gene-editing system targeting the CRCP gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the CRCP gene, as described herein, to a subject in need thereof. In some embodiments, the pharmaceutical composition comprises an LNP encapsuling an sgRNA having a crRNA sequence selected from SEQ ID NOs: 518-534 and a S. pyogenes Cas9 protein. In some embodiments, the crRNA sequence is selected from SEQ ID NOs: 518-527. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the CRCP gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the CRCP gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an CRCP gene (e.g., 5′ untranslated region [UTR], exon 1, exon 2, exon 3, exon 4, exon 5, exon 6, exon 7, exon 8, exon 9, exon 10, exon 11, any intervening intronic regions, intron/exon junctions, the 3′ UTR, or polyadenylation signal) is targeted by an RNA-guided nuclease to alter the gene. In some embodiments, the CRCP gene targeted by an RNA-guided nuclease is from a mammal. In some embodiments, the CRCP gene targeted by an RNA-guided nuclease is from a human (hCRCP). In some embodiments, the CRCP gene targeted by an RNA-guided nuclease is from a dog (cCRCP). In some embodiments, the CRCP gene targeted by an RNA-guided nuclease is from a horse (eCRCP). In some embodiments, the CRCP gene targeted by an RNA-guided nuclease is from a cat (fCRCP).

### AAAA. YAP1

In one aspect, methods and pharmaceutical compositions are provided for treating a back or spine condition or disorder by administering a therapeutically effective amount of a CRISPR pharmaceutical composition that targets an YAP1 gene to a subject in need thereof. In some embodiments, the CRISPR pharmaceutical composition comprises an RNA-guided nuclease and an sgRNA targeting the YAP1 gene with a crRNA sequence selected from SEQ ID NOs: 2671-2718. In some embodiments, the crRNA sequence is selected from SEQ ID NOs: 2671-2695. In some embodiments, the crRNA sequence is selected from 2671-2680. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the YAP1 gene is delivered to a mammalian cell via viral vector. In some embodiments, the CRISPR gene-editing system targeting the YAP1 gene is delivered to a mammalian cell via an AAV vector. In some embodiments, the CRISPR gene-editing system targeting the YAP1 gene is delivered to a mammalian cell via a lentiviral vector. In some embodiments, the CRISPR gene-editing system targeting the YAP1 gene is delivered to a mammalian cell via a lipid nanoparticle. In some embodiments, the CRISPR gene-editing system targeting the YAP1 gene is delivered to a mammalian cell via a virus-like particle. In some embodiments, the CRISPR gene-editing system targeting the YAP1 gene is delivered to a mammalian cell via a liposome. In some embodiments, the CRISPR gene-editing system targeting the YAP1 gene is delivered to a mammalian cell via a lipid nanocrystal.

In some embodiments, the pharmaceutical composition comprises an sgRNA having a crRNA sequence selected from SEQ ID NOs: 2671-2718 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 2671-2718 and a S. pyogenes Cas9 protein encapsulated in a lipid nanoparticle.

In various embodiments, the pharmaceutical composition comprising a CRISPR gene-editing system targeting the YAP1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the YAP1 gene, as described herein, to a subject in need thereof. In some embodiments, the pharmaceutical composition comprises an LNP encapsuling an sgRNA having a crRNA sequence selected from SEQ ID NOs: 2671-2718 and a S. pyogenes Cas9 protein. In some embodiments, the crRNA sequence is selected from SEQ ID NOs: 2671-2695. In some embodiments, the crRNA sequence is selected from SEQ ID NOs: 2671-2680. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the YAP1 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the YAP1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an YAP1 gene (e.g., 5′ untranslated region [UTR], exon 1, exon 2, exon 3, exon 4, exon 5, exon 6, exon 7, exon 8, exon 9, exon 10, exon 11, any intervening intronic regions, intron/exon junctions, the 3′ UTR, or polyadenylation signal) is targeted by an RNA-guided nuclease to alter the gene. In some embodiments, the YAP1 gene targeted by an RNA-guided nuclease is from a mammal. In some embodiments, the YAP1 gene targeted by an RNA-guided nuclease is from a human (hYAP1). In some embodiments, the YAP1 gene targeted by an RNA-guided nuclease is from a dog (cYAP1). In some embodiments, the YAP1 gene targeted by an RNA-guided nuclease is from a horse (eYAP1). In some embodiments, the YAP1 gene targeted by an RNA-guided nuclease is from a cat (fYAP1).

### BBBB. IL1RAP

In one aspect, methods and pharmaceutical compositions are provided for treating a back or spine condition or disorder by administering a therapeutically effective amount of a CRISPR pharmaceutical composition that targets an IL1RAP gene to a subject in need thereof. In some embodiments, the CRISPR pharmaceutical composition comprises an RNA-guided nuclease and an sgRNA targeting the IL1RAP gene with a crRNA sequence selected from SEQ ID NOs: 840-887. In some embodiments, the crRNA sequence is selected from SEQ ID NOs: 840-864. In some embodiments, the crRNA sequence is selected from 840-849. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL1RAP gene is delivered to a mammalian cell via viral vector. In some embodiments, the CRISPR gene-editing system targeting the IL1RAP gene is delivered to a mammalian cell via an AAV vector. In some embodiments, the CRISPR gene-editing system targeting the IL1RAP gene is delivered to a mammalian cell via a lentiviral vector. In some embodiments, the CRISPR gene-editing system targeting the IL1RAP gene is delivered to a mammalian cell via a lipid nanoparticle. In some embodiments, the CRISPR gene-editing system targeting the IL1RAP gene is delivered to a mammalian cell via a virus-like particle. In some embodiments, the CRISPR gene-editing system targeting the IL1RAP gene is delivered to a mammalian cell via a liposome. In some embodiments, the CRISPR gene-editing system targeting the IL1RAP gene is delivered to a mammalian cell via a lipid nanocrystal.

In some embodiments, the pharmaceutical composition comprises an sgRNA having a crRNA sequence selected from SEQ ID NOs: 840-887 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 840-887 and a S. pyogenes Cas9 protein encapsulated in a lipid nanoparticle.

In various embodiments, the pharmaceutical composition comprising a CRISPR gene-editing system targeting the IL1RAP gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL1RAP gene, as described herein, to a subject in need thereof. In some embodiments, the pharmaceutical composition comprises an LNP encapsuling an sgRNA having a crRNA sequence selected from SEQ ID NOs: 840-887 and a S. pyogenes Cas9 protein. In some embodiments, the crRNA sequence is selected from SEQ ID NOs: 840-864. In some embodiments, the crRNA sequence is selected from SEQ ID NOs: 840-849. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL1RAP gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL1RAP gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

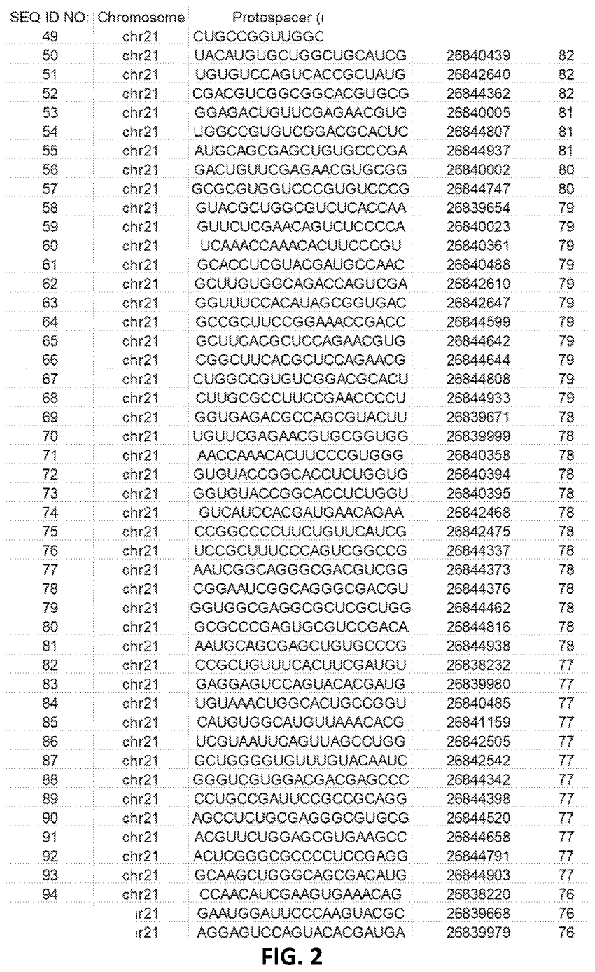

In certain embodiments, any region of an IL1RAP gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, any intervening intronic regions, intron/exon junctions, the 3′ UTR, or polyadenylation signal) is targeted by an RNA-guided nuclease to alter the gene (see, e.g., FIG. 2A). In some embodiments, the IL1RAP gene targeted by an RNA-guided nuclease is from a mammal. In some embodiments, the IL1RAP gene targeted by an RNA-guided nuclease is from a human (hIL1RAP). In some embodiments, the IL1RAP gene targeted by an RNA-guided nuclease is from a dog (cIL1RAP). In some embodiments, the IL1RAP gene targeted by an RNA-guided nuclease is from a horse (eIL1RAP). In some embodiments, the IL1RAP gene targeted by an RNA-guided nuclease is from a cat (fIL1RAP).

### CCCC. IL1R1

In one aspect, methods and pharmaceutical compositions are provided for treating a back or spine condition or disorder by administering a therapeutically effective amount of a CRISPR pharmaceutical composition that targets an IL1R1 gene to a subject in need thereof.

In some embodiments, the CRISPR pharmaceutical composition comprises an RNA-guided nuclease and an sgRNA targeting the IL1R1 gene with a crRNA sequence selected from SEQ ID NOs: 806-839. In some embodiments, the crRNA sequence is selected from SEQ ID NOs: 806-830. In some embodiments, the crRNA sequence is selected from 806-815. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL1R1 gene is delivered to a mammalian cell via viral vector. In some embodiments, the CRISPR gene-editing system targeting the IL1R1 gene is delivered to a mammalian cell via an AAV vector. In some embodiments, the CRISPR gene-editing system targeting the IL1R1 gene is delivered to a mammalian cell via a lentiviral vector. In some embodiments, the CRISPR gene-editing system targeting the IL1R1 gene is delivered to a mammalian cell via a lipid nanoparticle. In some embodiments, the CRISPR gene-editing system targeting the IL1R1 gene is delivered to a mammalian cell via a virus-like particle. In some embodiments, the CRISPR gene-editing system targeting the IL1R1 gene is delivered to a mammalian cell via a liposome. In some embodiments, the CRISPR gene-editing system targeting the IL1R1 gene is delivered to a mammalian cell via a lipid nanocrystal.

In some embodiments, the pharmaceutical composition comprises an sgRNA having a crRNA sequence selected from SEQ ID NOs: 806-839 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 806-839 and a S. pyogenes Cas9 protein encapsulated in a lipid nanoparticle.

In various embodiments, the pharmaceutical composition comprising a CRISPR gene-editing system targeting the IL1R1 gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

Tn some embodiments, a method is provided for treating a discogenic disease or disorder by administering a therapeutically affective amount of a pharmaceutical composition comprising a CRISPR gene-editing system targeting the IL1R1 gene, as described herein, to a subject in need thereof. In some embodiments, the pharmaceutical composition comprises an LNP encapsuling an sgRNA having a crRNA sequence selected from SEQ ID NOs: 806-839 and a S. pyogenes Cas9 protein. In some embodiments, the crRNA sequence is selected from SEQ ID NOs: 806-830. In some embodiments, the crRNA sequence is selected from SEQ ID NOs: 806-815. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL1R1 gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL1R1 gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL1R1 gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, any intervening intronic regions, intron/exon junctions, the 3′ UTR, or polyadenylation signal) is targeted by an RNA-guided nuclease to alter the gene. In some embodiments, the IL1R1 gene targeted by an RNA-guided nuclease is from a mammal. In some embodiments, the IL1R1 gene targeted by an RNA-guided nuclease is from a human (hIL1R1). In some embodiments, the IL1R1 gene targeted by an RNA-guided nuclease is from a dog (cIL1R1). In some embodiments, the IL1R1 gene targeted by an RNA-guided nuclease is from a horse (eIL1R1). In some embodiments, the IL1R1 gene targeted by an RNA-guided nuclease is from a cat (fIL1R1).

### DDDD. IL1A

In one aspect, methods and pharmaceutical compositions are provided for treating a back or spine condition or disorder by administering a therapeutically effective amount of a CRISPR pharmaceutical composition that targets an IL1A gene to a subject in need thereof. In some embodiments, the CRISPR pharmaceutical composition comprises an RNA-guided nuclease and an sgRNA targeting the IL1A gene with a crRNA sequence selected from SEQ ID NOs: 769-786. In some embodiments, the crRNA sequence is selected from 769-778. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL1A gene is delivered to a mammalian cell via viral vector. In some embodiments, the CRISPR gene-editing system targeting the IL1A gene is delivered to a mammalian cell via an AAV vector. In some embodiments, the CRISPR gene-editing system targeting the IL1A gene is delivered to a mammalian cell via a lentiviral vector. In some embodiments, the CRISPR gene-editing system targeting the IL1A gene is delivered to a mammalian cell via a lipid nanoparticle. In some embodiments, the CRISPR gene-editing system targeting the IL1A gene is delivered to a mammalian cell via a virus-like particle. In some embodiments, the CRISPR gene-editing system targeting the IL1A gene is delivered to a mammalian cell via a liposome. In some embodiments, the CRISPR gene-editing system targeting the IL1A gene is delivered to a mammalian cell via a lipid nanocrystal.

In some embodiments, the pharmaceutical composition comprises an sgRNA having a crRNA sequence selected from SEQ ID NOs: 769-786 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 769-786 and a S. pyogenes Cas9 protein encapsulated in a lipid nanoparticle.

In various embodiments, the pharmaceutical composition comprising a CRISPR gene-editing system targeting the IL1A gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL1A gene, as described herein, to a subject in need thereof. In some embodiments, the pharmaceutical composition comprises an LNP encapsuling an sgRNA having a crRNA sequence selected from SEQ ID NOs: 769-786 and a S. pyogenes Cas9 protein. In some embodiments, the crRNA sequence is selected from SEQ ID NOs: 769-778. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL1A gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL1A gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL1A gene (e.g., 5′ untranslated region [UTR], exon 1, exon 2, exon 3, exon 4, exon 5, exon 6, exon 7, exon 8, any intervening intronic regions, intron/exon junctions, the 3′ UTR, or polyadenylation signal) is targeted by an RNA-guided nuclease to alter the gene (see, e.g., FIG. 2A). In some embodiments, the IL1A gene targeted by an RNA-guided nuclease is from a mammal. In some embodiments, the IL1A gene targeted by an RNA-guided nuclease is from a human (hIL1A). In some embodiments, the IL1A gene targeted by an RNA-guided nuclease is from a dog (cIL1A). In some embodiments, the IL1A gene targeted by an RNA-guided nuclease is from a horse (eIL1A). In some embodiments, the IL1A gene targeted by an RNA-guided nuclease is from a cat (fIL1A).

### EEEE. IL1B

In one aspect, methods and pharmaceutical compositions are provided for treating a back or spine condition or disorder by administering a therapeutically effective amount of a CRISPR pharmaceutical composition that targets an IL1B gene to a subject in need thereof. In some embodiments, the CRISPR pharmaceutical composition comprises an RNA-guided nuclease and an sgRNA targeting the IL1B gene with a crRNA sequence selected from SEQ ID NOs: 787-805. In some embodiments, the crRNA sequence is selected from 787-796. In some embodiments, the RNA-guided nuclease is a Cas9 protein. In some embodiments, the Cas9 protein is an S. pyogenes Cas9 protein. In some embodiments, the S. pyogenes Cas9 protein comprises K848A, K1003A, and R1060A amino acid substitutions relative to the SpCas9 sequence of NCBI accession number 7S37_P. In some embodiments, the S. pyogenes Cas9 protein comprises an R691A amino acid substitution relative to the SpCas9 sequence of NCBI accession number 7S37_P.

In some embodiments, the CRISPR gene-editing system targeting the IL1B gene is delivered to a mammalian cell via viral vector. In some embodiments, the CRISPR gene-editing system targeting the IL1B gene is delivered to a mammalian cell via an AAV vector. In some embodiments, the CRISPR gene-editing system targeting the IL1B gene is delivered to a mammalian cell via a lentiviral vector. In some embodiments, the CRISPR gene-editing system targeting the IL1B gene is delivered to a mammalian cell via a lipid nanoparticle. In some embodiments, the CRISPR gene-editing system targeting the IL1B gene is delivered to a mammalian cell via a virus-like particle. In some embodiments, the CRISPR gene-editing system targeting the IL1B gene is delivered to a mammalian cell via a liposome. In some embodiments, the CRISPR gene-editing system targeting the IL1B gene is delivered to a mammalian cell via a lipid nanocrystal.

In some embodiments, the pharmaceutical composition comprises an sgRNA having a crRNA sequence selected from SEQ ID NOs: 787-805 and a S. pyogenes Cas9 protein, optionally comprising K848A, K1003A, and R1060A amino acid substitutions or an R691A amino acid substitution, encapsulated in i) a viral vector selected from an AAV vector and a lentiviral vector; ii) a lipid nanoparticle; iii) a virus-like particle; iv) a liposome; or v) a lipid nanocrystal. In some embodiments, the pharmaceutical composition comprises an sgRNA having a crRNA sequence selected from SEQ ID NOs: 787-805 and a S. pyogenes Cas9 protein encapsulated in a lipid nanoparticle.

In various embodiments, the pharmaceutical composition comprising a CRISPR gene-editing system targeting the IL1B gene is used in a method of treating a mammal in need thereof. In some embodiments, the method treats a discogenic disease or disorder. In some embodiments, the discogenic disease or disorder is low back pain, neck pain, lumbar pain, degenerative disc disease, discogenic pain annual ligament tear, herniated disc, spinal facet joint arthritis, intervertebral disc disease, spondylosis, painful scoliosis, or spinal stenosis. In some embodiments, the discogenic disease or disorder is discogenic pain.

In some embodiments, a method is provided for treating a discogenic disease or disorder by administering a therapeutically affective amount of a pharmaceutical composition comprising a CRISPR gene-editing system targeting the IL1B gene, as described herein, to a subject in need thereof. In some embodiments, the pharmaceutical composition comprises an LNP encapsuling an sgRNA having a crRNA sequence selected from SEQ ID NOs: 787-805 and a S. pyogenes Cas9 protein. In some embodiments, the crRNA sequence is selected from SEQ ID NOs: 787-796. In some embodiments, the pharmaceutical composition is administered by intradiscal injection.

In some embodiments, a method is provided for treating a musculoskeletal disease or disorder by administering a therapeutically affective amount of a pharmaceutical composition comprising a CRISPR gene-editing system targeting the IL1B gene, as described herein, to a subject in need thereof. In some embodiments, the musculoskeletal disease or disorder is Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, or Polymyositis.

In yet other embodiments, a method is provided for treating an inflammatory disease or disorder by administering a therapeutically affective amount of a pharmaceutical composition comprising a CRISPR gene-editing system targeting the IL1B gene, as described herein, to a subject in need thereof. In some embodiments, the inflammatory disease or disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general).

In certain embodiments, any region of an IL1B gene (e.g., 5′ untranslated region [UTR], exon 1, exon 2, exon 3, exon 4, exon 5, exon 6, exon 7, exon 8, any intervening intronic regions, intron/exon junctions, the 3′ UTR, or polyadenylation signal) is targeted by an RNA-guided nuclease to alter the gene (see, e.g., FIG. 2A). In some embodiments, the IL1B gene targeted by an RNA-guided nuclease is from a mammal. In some embodiments, the IL1B gene targeted by an RNA-guided nuclease is from a human (hIL1B). In some embodiments, the IL1B gene targeted by an RNA-guided nuclease is from a dog (cIL1B). In some embodiments, the IL1B gene targeted by an RNA-guided nuclease is from a horse (eIL1B). In some embodiments, the IL1B gene targeted by an RNA-guided nuclease is from a cat (fIL1B).

### VII. Administration Routes

The methods and compositions herein described encompass the use of pharmaceutical compositions comprising a CRISPR gene-editing system as an active ingredient.

Depending on the method/route of administration, pharmaceutical dosage forms come in several types. These include many kinds of liquid, solid, and semisolid dosage forms. Common pharmaceutical dosage forms include pill, tablet, or capsule, drink or syrup, and natural or herbal form such as plant or food of sorts, among many others. Notably, the route of administration (ROA) for drug delivery is dependent on the dosage form of the substance in question. A liquid pharmaceutical dosage form is the liquid form of a dose of a chemical compound used as a drug or medication intended for administration or consumption.

As described below, a composition of the present disclosure can be delivered to a subject subcutaneously (e.g., intra-articular or intradiscal injection), dermally (e.g., transdermally via patch), and/or via implant. Exemplary pharmaceutical dosage forms include, e.g., pills, osmotic delivery systems, elixirs, emulsions, hydrogels, suspensions, syrups, capsules, tablets, orally dissolving tablets (ODTs), gel capsules, thin films, adhesive topical patches, lollipops, lozenges, chewing gum, dry powder inhalers (DPIs), vaporizers, nebulizers, metered dose inhalers (MDIs), ointments, transdermal patches, intradermal implant.

As used herein, “dermal delivery” or “dermal administration” can refer to a route of administration wherein the pharmaceutical dosage form is taken to, or through, the dermis (i.e., layer of skin between the epidermis (with which it makes up the cutis) and subcutaneous tissues). “Subcutaneous delivery” can refer to a route of administration wherein the pharmaceutical dosage form is to or beneath the subcutaneous tissue layer.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy)propane-co-sebacic-acid] (PCPP-SA) matrix, fatty acid dimer-sebacic acid (FAD-SA) copolymer, poly(lactide-co-glycolide)), polyglycolic acid, collagen, polyorthoesters, polyethylene glycol-coated liposomes, hyaluronic acid and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006 December; 79(3-4): 141-152.

The pharmaceutical compositions described herein may be included in a container, kit, pack, or dispenser together with instructions for administration.

### A. Systemic Administration

In some embodiments, a pharmaceutical composition comprising a CRISPR gene-editing system is administered systemically to a mammal in need thereof. In some embodiments, the composition is formulated for intravenous injection. In some embodiments, the composition is formulated for oral administration. In some embodiments, the composition is formulated for parenteral administration.

### B. Local Administration

In some embodiments, a pharmaceutical composition comprising a CRISPR gene-editing system is administered locally to a mammal in need thereof. In some embodiments, the local administration is an intra-articular injection. In some embodiments, the composition is formulated for intradiscal injection as described in, for instance, Migliore, A., et al. (2020). Therapeutics and Clinical Risk Management, 953-968, which is hereby incorporated by reference in its entirety for all purposes. In some embodiments, the composition is formulated for epidural injection as described in, for instance, Manchikanti, L., et al. (2021). Pain Physician, 24(6), 425, which is hereby incorporated by reference in its entirety for all purposes. In some embodiments, the composition is formulated for peridiscal injection. In some embodiments, the composition is formulated for perivertebral injection. In some embodiments, composition is formulated for administration to the facet joints of the spine.

In some embodiments, a pharmaceutical composition comprising a CRISPR gene-editing system is administered locally to a mammal in need thereof during a surgical procedure. In some embodiments, a pharmaceutical composition comprising a CRISPR gene-editing system is administered locally to a mammal in need thereof 1, 2, 3, 4, 5, 6, 7, 8, 9, 10, 20, or 30 days after a surgical procedure.

In some embodiments the administration route is intradiscal injection of a pharmaceutical compositions comprising a CRISPR gene-editing system for treatment of a mammal in need thereof, wherein the CRISPR gene-editing system targets a gene selected from (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v). In some embodiments, the mammal is selected from a human, a dog, a horse, and a cat.

In some embodiments the administration route is epidural injection of a pharmaceutical compositions comprising a CRISPR gene-editing system for treatment of a mammal in need thereof, wherein the CRISPR gene-editing system targets a gene selected from (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6. CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v). In some embodiments, the mammal is selected from a human, a dog, a horse, and a cat.

In some embodiments the administration route is peridiscal injection of a pharmaceutical compositions comprising a CRISPR gene-editing system for treatment of a mammal in need thereof, wherein the CRISPR gene-editing system targets a gene selected from (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8. MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v). In some embodiments, the mammal is selected from a human, a dog, a horse, and a cat.

In some embodiments the administration route is perivertebral injection of a pharmaceutical compositions comprising a CRISPR gene-editing system for treatment of a mammal in need thereof, wherein the CRISPR gene-editing system targets a gene selected from (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8. MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL6ST, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v). In some embodiments, the mammal is selected from a human, a dog, a horse, and a cat.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Design and Selection of Guides for Editing the IL1 Receptor (IL1R1)

A pipeline of in-silico and in-vitro screening was developed to identify candidate CRISPR-Cas guides for gene editing. The pipeline was applied to identify guides that effectively edit the canine IL1R1 receptor gene (cIL1R1), thereby disrupting IL1α/IL1β signaling in vivo. The first step in the pipeline was to identify all possible crRNA sequences for a particular CRISPR-Cas protein in the coding portions of the cIL1R1. Many algorithms for identifying such sequences are known in the art. Generally, these algorithms function by identifying a protospacer adjacent motif (PAM) sequence for the particular CRISPR-Cas protein and then locate the sequence spaced according to the requirements of the particular Cas protein, typically directly 5′ of the PAM site. For example, the S. pyogenes Cas9 (SpCas9) protein, which was used in this Example, recognizes a 5′-NGG-3′ PAM sequence. Thus, all sequences directly 5′ of an NGG trinucleotide are possible crRNA sequences, crRNA sequences identified in this fashion as shown in, e.g., FIGS. 79-87, along with biographical information about the crRNA sequence.

Next, each identified crRNA sequence was evaluated across three different metrics: possible off-target editing at locations in the genome other than the target gene, on-target editing efficiency, and the likelihood of editing causing frameshift mutations, using multiple algorithms for each metric, as illustrated in FIG. 90A. The basis of the combinatorial approach used lies in the assumption that every model has blind spots that may skew the fitness of a particular guide RNA. Weighting these scores to obtain a consensus score for each of these properties allows for much better prediction of sgRNA fitness.

Off-target editing effects were predicted by averaging scores generated by the MIT, CFD and Elevation (human only) model. The MIT algorithm, also known as Hsu-Zhang score. Hsu, P. D. et al. (2013). Nature Biotechnology 31, 827-832. This model is based on a positional penalty matrix (1×20) generated from 15 EMX1 sgRNA libraries with mismatches against target at every position. The CFD algorithm (Cutting Frequency Determination) is based on threat matrix (12×20) considering both position and mismatch type and PAM integrity (27,897 ‘CD33’ sgRNAs+10,618 negative control sgRNAs). Doench, J. G. et al. (2016). Nature Biotechnology 34, 184-191. The Elevation score using machine learning algorithms trained by genome-wide (GUIDE-Seq) and other aggregated off-target profiling data. Listgarten, J. et al. (2018). Nature Biomedical Engineering 2, 38-47. The column labelled “OFF” shows the mean of the scores provided by the two or three models, respectively. The column labelled “OffTarget #” shows the number of potential off-targets with up to four mismatches as calculated by CRISPOR. See, Haeussler, M. et al. (2016). Genome Biology 17, 148.

On-target editing efficiencies were predicted by averaging scores generated by the Azimuth model, the DeepSpCas9 model, and the CRISPRScan model. The Azimuth model is a boosted regression tree model, trained with 881 sgRNAs (MOLM13/NB4/TF1 cells and additional unpublished data) delivered by lentivirus. Doench, J. G. et al. (2016). Nature Biotechnology 34, 184-191. DeepSpCas9 is a deep learning model trained using editing data from 12,832 sgRNA. Kim, H. K. et al. (2019). Science Advances 5(11). CRISPRscan is a linear regression model, trained using editing data from 1000 sgRNAs injected into zebrafish embryos targeting >100 genes. Moreno-Mateos, M. A. et al. (2015). Nature Methods 12, 982-988. The column labelled “ON” shows the mean of the scores provided by the three models.

The putative guides' potential to generate frameshift mutations were predicted by averaging scores generated by the Lindel model and the InDelphi model. Lindel is a machine learning model trained using profile data of 1.16 million independent mutational events triggered by CRISPR/Cas9-mediated cleavage and non-homologous end joining-mediated double strand break repair of 6872 synthetic target sequences, introduced into a human cell line via lentiviral infection. Chen, W. et al. (2019). Nucleic Acids Research 47, 7989-8003. InDelphi is machine learning model trained with indels generated by 1872 sgRNAs. Shen, M. W. et al. (2018). Nature 563, 646-651 (2018). The column labelled “Frameshift” shows the mean of the scores provided by the two models. The column labelled “Precision” shows the frequency distribution of indels estimated by inDelphi. High precision is closer to 100 and represents sgRNAs that are characterised by one or a very low number of repair outcomes.

The candidate crRNA sequences were then evaluated for the presence of Graf motifs, TT or GCC present in the 4 PAM proximal bases of the crRNA sequence, as indicated in, e.g., FIGS. 79-87, as either TT or GCC. Graf, R. et al. (2019). Cell Reports 26(5), 1098-103. Graf et al. reported that TT- and GCC-motifs are a hallmark of inefficient sgRNAs. If possible, crRNA with Graf motifs and in particular the GCC motif were avoided. In contrast to the TT motif, the GCC motif remains critical if sgRNAs are synthesized de novo rather than by transcription.

As shown in, e.g., FIGS. 79-87, three consensus scores were then calculated for each crRNA sequence. The “ON-OFF” score represents the mean of the “ON” and “OFF” score. The “OFF-FS” score represents the mean of the “OFF” and “Frameshift” score. The OVERALL score represents the mean of the “ON”, “OFF” and “Frameshift” score. Additionally, for human only, VBC's Bioscore was used to predict whether a possible in-frame mutation could disturb protein function. This is more likely to occur in conserved genomic DNA sequences coding for critical protein domains. Thus, Bioscore is based on protein domain annotations, phylogenetic conservation, amino acid identities and exon size. Michlits, G. et al. (2020). Nature Methods 17, 708-716. The final criteria for selecting candidates is mainly based on the OVERALL score (in most cases >70), the relative low counts of potential off-targets (in most cases <200 off-targets), the absence of Graf motifs (if possible) and the genomic cut position within the coding sequence to produce knockouts or truncated proteins with reduced functionality (e.g. decoy receptors),

Finally, the IL1R1 domain in which each crRNA guides editing was determined based on the nucleotide position in the gene. The domain location is used to predict whether the edit will result in a wild-type like protein (edits in the sequence encoding the extreme C-terminus of a receptor that are unlikely to disrupt receptor-mediated signaling), complete knock-out of any functional protein (edits in the sequence encoding the N-terminus and/or essential functional domains), a soluble decoy receptor (edits in the sequence encoding the transmembrane domain of the receptor), or a membrane-bound decoy receptor (edits in the sequence encoding the intracellular signaling domain of the receptor).

Indeed, this in silico analysis established that several candidate sgRNAs were predicted to yield genomic edits that would create a dominant negative (DN) IL1R1, thereby impacting intracellular pro-inflammatory signaling (FIG. 90A). In support of the robustness of these analyses, structural predictions also demonstrated that editing with these particular sgRNAs would yield an expressible form of IL1R1 protein (either soluble or membrane-bound) that cannot induce the ligand's signal (FIG. 90B). Taken together, these predictive studies demonstrated the feasibility of inducing a dominant negative canine IL1 receptor as a unique approach to tamping down inflammation in cells and provided a high-throughput method for validating candidates without the need for time- and resource-intensive brute force methods.

### Example 2: Validation of DN IL1R1 In Canine Cells

Having designed and analyzed numerous sgRNA candidates that were predicted to edit IL1 receptor to produce a dominant negative reception, the next step was to assess their ability to act in vitro. Of particular interest is whether the preferred (i.e., on-target) edit would be observed in edited cells.

To test this, 80 pmoles of sgRNA candidates were introduced into canine DH82 monocytes via electroporation as part of a ribonucleoprotein (i.e., pre-assembled with 25 pmoles of SpCas9 protein at room temperature for at least 5 min). In brief, around 600,000 cells resuspended in 20 μl PKM buffer (2.7 mM KCl, 137 mM NaCl, 10 mM Na2HPO4/1.8 mM NaH2PO4 (pH 7.4) and 50 mM D-Mannitol) were added to the pre-assembled SpCas9 ribonucleoprotein complex, transferred into 16-well nucleocuvettes and electroporated with pulse code CM-137 using the 4D nucleofector (Lonza). Electroporated cells were kept in PKM buffer for about 10 min before transferring them into 6-well tissue culture plates with complete culture media. After 2-5 days in culture, cells were dissociated for genomic DNA extraction using the DNeasy Blood & Tissue Kit (Qiagen). sgRNA target regions were amplified by PCR and editing efficacy was then deduced from Sanger sequencing traces using Inference of CRISPR Edits (ICE v1.2). See, Conant, D. et al. (2022). The CRISPR Journal 5(1), 123-130.

While multiple candidates were indeed able to induce mutation in the IL1R1 gene at high efficiency-including several exhibiting frequencies above 95%-two lead candidates exhibited the ability to consistently induce the same on-target edit in a high percentage of cells (FIG. 91). These results confirmed the robustness of the in silico analysis and suggested that at least OCR13 and OCR14 are able to produced DN IL1 receptors in canine cells.

### Example 3: Impact of Cas9 Mutants with Enhanced Specificity

With the ultimate aim to eliminate off-target editing without sacrificing on-target activity, the lead candidate sgRNAs were assembled with different high-fidelity SpCas9 variants (i.e. mutated SpCas9 nucleases with enhanced specificity; see generally, Slaymaker, I. M. et al (2016). Science 351(6268), 84-88; Vakulskas, C. A. et al. (2018). Nature Medicine 24(8), 1216-1224) and assessed for frameshift efficacy and precision (i.e. the primacy for single or low number of repair outcomes) in canine DH82 monocytes.

Among the high-fidelity SpCas9 nucleases, AR-Cas9 (IDT, Catalogue #1081060; Vakulskas, C. A. et al. (2018). Nature Medicine 24(8), 1216-1224) performed best overall with the lead candidate sgRNAs. In comparison to wild-type SpCas9 (WT-Cas9), AR-Cas9 reached similarly high frameshift efficacy (>97%) and precision (>83%) when paired with OCR13 (FIG. 92A) and OCR14 (FIG. 92B). The other high-fidelity Cas9 nucleases tested were ES-Cas9 (Growth Factory, eSpCas9 (1.1); Slaymaker, I. M. et al (2016). Science 351(6268), 84-88), HF-Cas9 (Invitrogen, Catalogue #A50576) and PE-Cas9 (Merck Life Science, Catalogue #PECAS9-250).

However, AR-Cas9 like all other high-fidelity SpCas9 nucleases (ES-Cas9, PE-Cas9 and HF-Cas9) showed decreased editing efficacy with OCR06, as compared to a combination with WT-Cas9 (FIG. 92C). Also, OCR10, which exhibits high precision with WT-Cas9, gives rise to a second frameshift edit at frequency 20-30% with all tested high-fidelity SpCas9 nucleases (FIG. 92D). These results, taken as a whole, suggested that pairings between high-fidelity RNA-dependent nuclease and sgRNA would likely need to be done on a case-by-case basis.

### Example 4: Design for IL1RAP/IL1RAP Decoy Proteins

IL1RAP is the co-receptor of all IL1 ligands, including IL33, which activates the IL1RL1/IL1RAP receptor dimer rather than the IL1A/B-specific IL1R1/IL1RAP. Thus, like other IL1R complex proteins, IL1RAP may be another potent mediator of inflammation that can be exploited for therapeutic purposes.

To test this, 10 candidate sgRNAs were designed to target different exons of canine IL1RAP with the objective to either ablate IL1RAP expressing (OCP01 to OCP06) or generate decoy IL1RAP proteins that will not transduce a pro-inflammatory signal (OCP07 to OCP10) (FIG. 93A). For example, OCP07 is predicted to predominantly produce an A duplication in exon 9 of IL1RAP, which shifts the open reading frame at position 355. At the protein level, this causes a Threonine (T) to Asparagine (N) amino acid change and prematurely terminates translation at position 395. Based on AlphaFold2-generated models, this +1 frameshift converts IL1RAP into a soluble IL1RAP decoy (IL1RAP_{T355NfsTer41}) missing the transmembrane and the cytoplasmic TIR domain (FIG. 93B).

Having done the predictive analysis, the next step was to assess performance in vitro. Candidate sgRNAs were paired with wild-typeSpCas9 (WT-Cas9) and introduced into canine DH82 monocytes via electroporation and editing efficacy and precision assessed by ICE. As predicted with an OVERALL score of 82, OCP02 performed best among the knockout candidates with virtually 100% KO rate due to a single C deletion introducing a premature stop codon at position 32 (M32*) (FIG. 94). All decoy-producing sgRNA candidates exhibited strong editing efficacy (greater than 95%). OCP09 and OCP10 demonstrated the strongest editing primacy with the top edit reaching 89% and 95%, respectively.

### Example 5: Editing of IL1RAP/IL1RAP in Various Canine Cell Types with High Fidelity Nucleases

Having designed and validated numerous guides targeting IL1RAP, the next step was to validate them with high-fidelity SpCas9 nuclease to mitigate potential off-target effects during the editing process. To do this, one or two sgRNAs with the strongest frameshift scores (FIG. 95A) for each type of interference (e.g., knockout or decoy receptor) were selected and paired with different high-fidelity SpCas9 nucleases to see which, if any, would show similar on-target activity to wild-type SpCas9 (WT-Cas9), while potentially reducing undesirable off-target effects.

Indeed, for each selected sgRNA, the editing efficacy in canine DH82 monocytes remained strongly dependent upon which high-fidelity SpCas9 nuclease was used (FIG. 95B). Notably, AR-Cas9 most consistently showed high frequency of the desired edit in canine IL1RAP. This was particularly true in the case of OCP02, OCP07, and OCP10. However, this effect was not universal, as the frameshift score for OCP09 was decreased when paired with AR-Cas9. OCP02, OCP07 and OCP10 also performed well in a head-to-head comparison of WT-Cas9 and AR-Cas9 in canine synovial fibroblasts (FIG. 95C). As such, high-fidelity RNA-dependent nucleases appeared to generally impact editing efficiency, though the magnitude of such effects will likely vary depending upon the nuclease used and to a lesser extent the cell type to be edited.

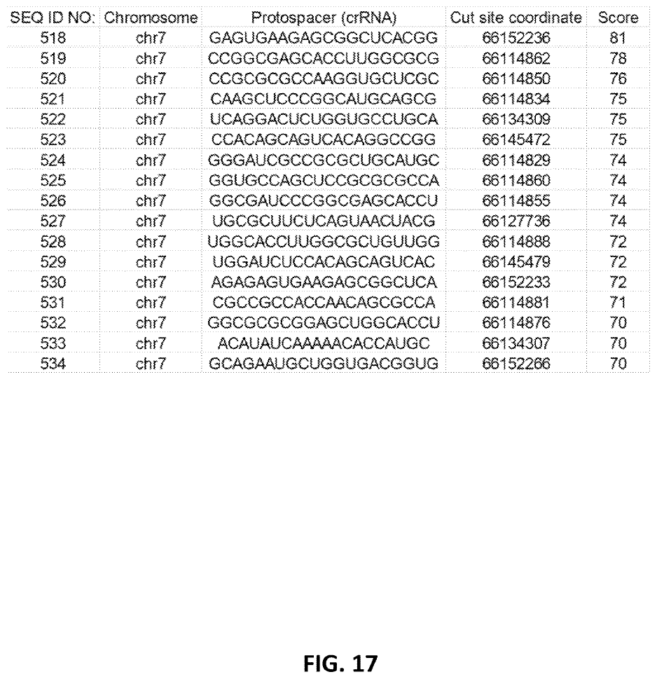

Having observed strong on-target activity when pairing sgRNA with high-fidelity SpCas9 nucleases, the next step involved expansion of the assay to include numerous relevant canine cell types, such as DH82 monocytes, chondrocytes, and synovial fibroblasts. Specifically, the same select sgRNAs were used with AR-Cas9 in DH82 monocytes, where a strong preference for each desired edit was again observed (FIG. 96A). Similar editing efficacies were also observed in chondrocytes (FIG. 96B) and synovial fibroblasts (FIG. 17C). This result demonstrated that consistently high editing efficacies and precision for a given sgRNA and high-fidelity RNA-dependent nuclease pairing was achieved across various cell types.

### Example 6: Editing in Human Cells

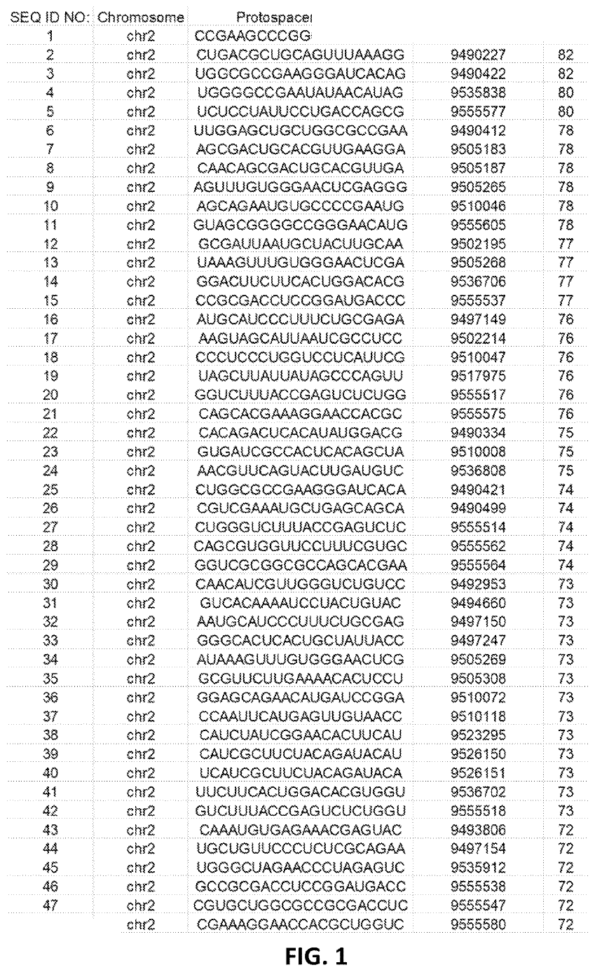

Having designed and validated numerous guides for multiple mammalian species (see FIGS. 1-89) and confirming the editing efficacy of these guides in canine cells, the next was to begin screening human-directed guides for their in vitro editing efficacy. As part of this testing, around 10,000 to 500,000 HEK cells were electroporated as described in detail above to introduce wild-type SpCas9 (WT-Cas9) paired with various synthetic sgRNA targeting human IL1RAP (hIL1RAP). Subsequent Sanger sequencing of the edited hIL1RAP gene, as previously described above, followed after two to three days of cell culture.

The inference of CRISPR edits from Sanger traces showed that multiple hIL1RAP sgRNAs caused about 50% or higher frameshifts. The top performer was sgRNA OHP06 showing >95% frameshift and high precision (i.e. only edit was detected). The results in this Example showed that multiple human guide RNAs were able to edit a targeted gene, thereby providing further support for the bioinformatic approach to guide RNA selection described herein.

### Example 7: Editing in Primary Human Disc Cells

Following the identification of numerous human guides with strong editing efficacy, next steps included observing editing in cell types relevant to intradiscal administration. As part of this testing, sgRNA OHP06 was selected from the initial screen described in Example 6 and paired with wild-type SpCas9 protein (WT-Cas9). These materials were electroporated into nucleus pulposus (primary disc) cells as described above, and after two days, the primary disc cells were then genotyped as previously described.

Sanger sequencing traces showed that the CRISPR-mediated frameshift occurred with 94% efficacy (R²>0.90) and that most detected edits (93%) being one single T duplication (FIG. 98A). Further interrogation of the edited cells found that the position of the sgRNA binding in exon 8 of human IL1RAP (hIL1RAP), which encodes Ig-like C2-type domain 3 (FIG. 98B, upper panel) carried the duplicated T as compared to control nucleus pulposus cells (FIG. 98B, lower panel). Finally, AlphaFold2-predictions of the 3D structures of wild-type and CRISPR-edited IL1RAP were rendered (FIG. 98C). The T duplication caused a frameshift at amino acid position 266 converting Cysteine (Cys) into Leucine (Leu) and prematurely terminating translation at codon position 6 as counted from the first changed amino acid to the premature stop codon (Cys266Leufs*6). As a result, the CRISPR-edited hIL1RAP lacks the last of three Ig-like C2-type domains as well as the transmembrane and TIR domains.

These results provided further support for the bioinformatic approach used in selecting guide RNA sequences. Moreover, they showed that at least one gene, IL1RAP, could be uniformly and efficaciously edited in human intervertebral disc cells by the methods herein disclosed. Finally, the results demonstrate that the editing has the desired effect (i.e., modifying the expression of an encoded human gene product). Taken together, the results in this Example provide a mechanism for use of CRISPR to edit cells of the human spine.

The examples set forth above are provided to give those of ordinary skill in the art a complete disclosure and description of how to make and use the embodiments of the compositions, systems and methods of the disclosure, and are not intended to limit the scope of what the inventors regard as their invention. Modifications of the above-described modes for carrying out the embodiments of the disclosure that are obvious to persons of skill in the art are intended to be within the scope of the following claims. All patents and publications mentioned in the specification are indicative of the levels of skill of those skilled in the art to which the disclosure pertains.

All headings and section designations are used for clarity and reference purposes only and are not to be considered limiting in any way. For example, those of skill in the art will appreciate the usefulness of combining various aspects from different headings and sections as appropriate according to the spirit and scope of the disclosure described herein.

It is to be understood that the methods described herein are not limited to the particular methodology, protocols, subjects, and sequencing techniques described herein and as such can vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

The examples set forth above are provided to give those of ordinary skill in the art a complete disclosure and description of how to make and use the embodiments of the compositions, systems and methods of the disclosure, and are not intended to limit the scope of what the inventors regard as their invention. Modifications of the above-described modes for carrying out the embodiments of the disclosure that are obvious to persons of skill in the art are intended to be within the scope of the following claims. All patents and publications mentioned in the specification are indicative of the levels of skill of those skilled in the art to which the disclosure pertains.

All headings and section designations are used for clarity and reference purposes only and are not to be considered limiting in any way. For example, those of skill in the art will appreciate the usefulness of combining various aspects from different headings and sections as appropriate according to the spirit and scope of the disclosure described herein.

It is to be understood that the methods described herein are not limited to the particular methodology, protocols, subjects, and sequencing techniques described herein and as such can vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

While some embodiments have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure.

Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein can be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

What is claimed is:

1. A pharmaceutical composition for treating a spinal disorder in a mammalian subject in need thereof, the composition comprising:
(i) an RNA-guided nuclease or a nucleic acid encoding an RNA-guided nuclease; and
(ii) at least one guide RNA or a nucleic acid encoding at least one guide RNA targeting a gene selected from the group consisting of FGF2, FGFR1, CCN2, ADAMTS1, ADAMTS5, MMP1, MMP2, MMP3, MMP7, MMP8, MMP10, MMP12, MMP13, TIMP1, TIMP3, CXCL1, CXCL2, CXCL3, CXCL5, CXCL6, CXCL8, CCL2, CCL3, CCL5, CCL7, CCL20, CXCR2, CCR7, YAP1, TNF, ADAM17, TNFRSF1A, TNFRSF1B, IL4, IL1R1, IL6, IL6R, CXCR1, IL10, IL10RB, IL10RA, IL13, IL13RA1, IL13RA2, IL17A, IL17RA, IL18, IL18RAP, IL18R1, SCN1A, SCN2A, SCN3A, SCN4A, SCN5A, SCN8A, SCN9A, SCN10A, SCN11A, TAC1, TACR1, MRGPRX2, ATP1A1, TACR2, TAC3, TACR3, CALCA, CALCB, RAMP1, CALCRL, ADM, CRCP, NGF, NGFR, NTRK1, NTF3, NTF4, NTRK2, IL1RAP, IL1α gene, IL1β and BDNF.

2. The pharmaceutical composition of claim 1, wherein the spinal disorder is intervertebral disc degeneration.

3. The pharmaceutical composition of claim 1, wherein the spinal disorder is disc herniation.

4. The pharmaceutical composition of claim 1, wherein the spinal disorder is spinal stenosis.

5. The pharmaceutical composition of claim 1, wherein the spinal disorder is spondylosis.

6. The pharmaceutical composition of claim 1, wherein the spinal disorder is spondylolisthesis.

7. The pharmaceutical composition of claim 1, wherein the spinal disorder is a spinal infection.

8. The pharmaceutical composition of claim 7, wherein the spinal infection is discospondylitis.

9. The pharmaceutical composition of claim 1, wherein the spinal disorder is a spinal neuropathy.

10. The pharmaceutical composition of claim 9, wherein the spinal neuropathy is discogenic pain, radiculopathy, sciatica, or post-herpetic neuralgia.

11. The pharmaceutical composition of any one of claims 1-10, wherein the pharmaceutical composition is for treating low back pain or neck pain associated with the spinal disorder.

12. The pharmaceutical composition of any one of claims 1-11, wherein the at least one guide RNA or a nucleic acid encoding at least one guide RNA targets a mammalian FGF2 gene.

13. The pharmaceutical composition of claim 12, wherein the mammalian FGF2 gene is a human FGF2 gene.

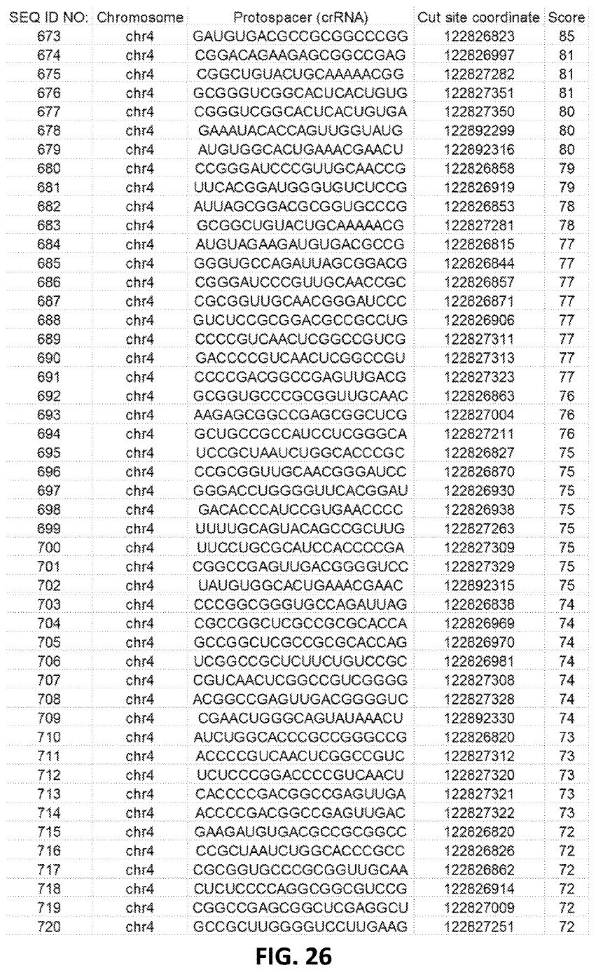

14. The pharmaceutical composition of claim 13, wherein the at least one guide RNA comprises a crRNA sequence selected from the group consisting of those sequences shown in FIG. 26 (SEQ ID NOs: 673-720).

15. The pharmaceutical composition of claim 13, wherein the at least one guide RNA comprises a crRNA sequence selected from the group consisting of SEQ ID NOs: 673-682.

16. The pharmaceutical composition of any one of claims 1-11, wherein the at least one guide RNA or a nucleic acid encoding at least one guide RNA targets a mammalian FGFR1 gene.

17. The pharmaceutical composition of claim 16, wherein the mammalian FGFR1 gene is a human FGFR1 gene.

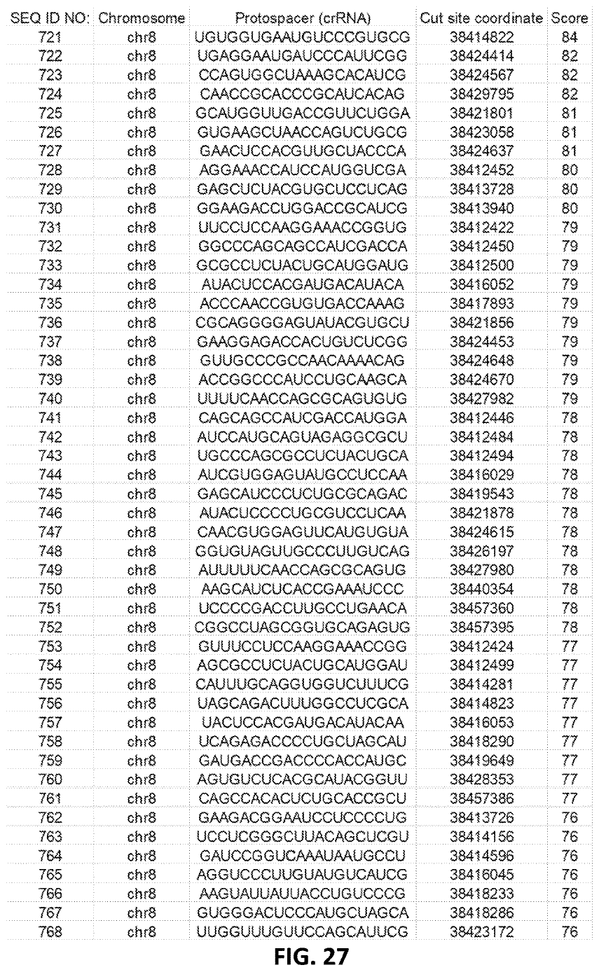

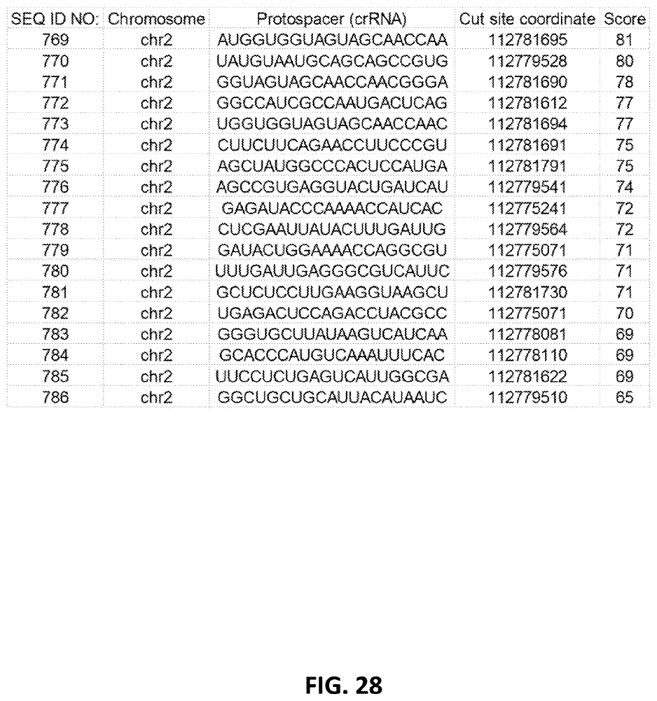

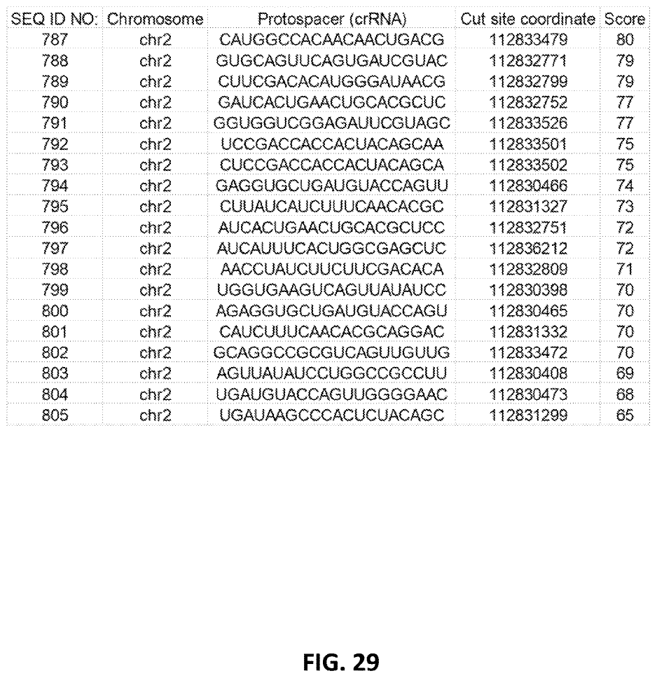

18. The pharmaceutical composition of claim 17, wherein the at least one guide RNA comprises a crRNA sequence selected from the group consisting of those sequences shown in FIG. 27 (SEQ ID NOs: 721-768).

19. The pharmaceutical composition of claim 17, wherein the at least one guide RNA comprises a crRNA sequence selected from the group consisting of SEQ ID NOs: 721-730.

20. The pharmaceutical composition of any one of claims 1-11, wherein the at least one guide RNA or a nucleic acid encoding at least one guide RNA targets a mammalian CCN2 gene.

21. The pharmaceutical composition of claim 20, wherein the mammalian CCN2 gene is a human CCN2 gene.

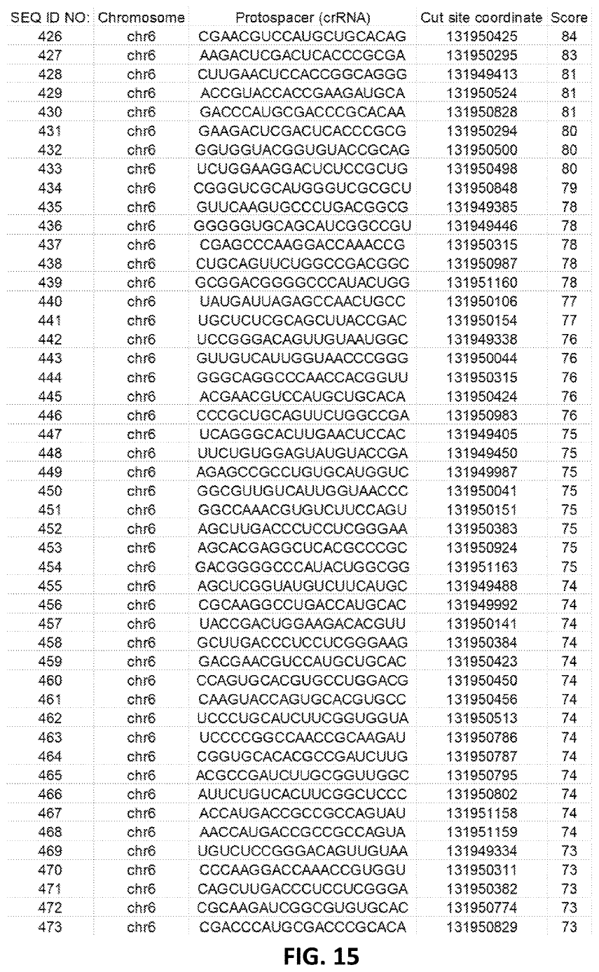

22. The pharmaceutical composition of claim 21, wherein the at least one guide RNA comprises a crRNA sequence selected from the group consisting of those sequences shown in FIG. 15 (SEQ ID NOs: 426-473).

23. The pharmaceutical composition of claim 21, wherein the at least one guide RNA comprises a crRNA sequence selected from the group consisting of SEQ ID NOs: 426-435.

24. The pharmaceutical composition of any one of claims 1-11, wherein the at least one guide RNA or a nucleic acid encoding at least one guide RNA targets a mammalian ADAMTS1 gene.

25. The pharmaceutical composition of claim 24, wherein the mammalian ADAMTS1 gene is a human ADAMTS1 gene.

26. The pharmaceutical composition of claim 25, wherein the at least one guide RNA comprises a crRNA sequence selected from the group consisting of those sequences shown in FIG. 2 (SEQ ID NOs: 49-96).

27. The pharmaceutical composition of claim 25, wherein the at least one guide RNA comprises a crRNA sequence selected from the group consisting of SEQ ID NOs:49-58.

28. The pharmaceutical composition of any one of claims 1-11, wherein the at least one guide RNA or a nucleic acid encoding at least one guide RNA targets a mammalian ADAMTS5 gene.

29. The pharmaceutical composition of claim 28, wherein the mammalian ADAMTS5 gene is a human ADAMTS5 gene.

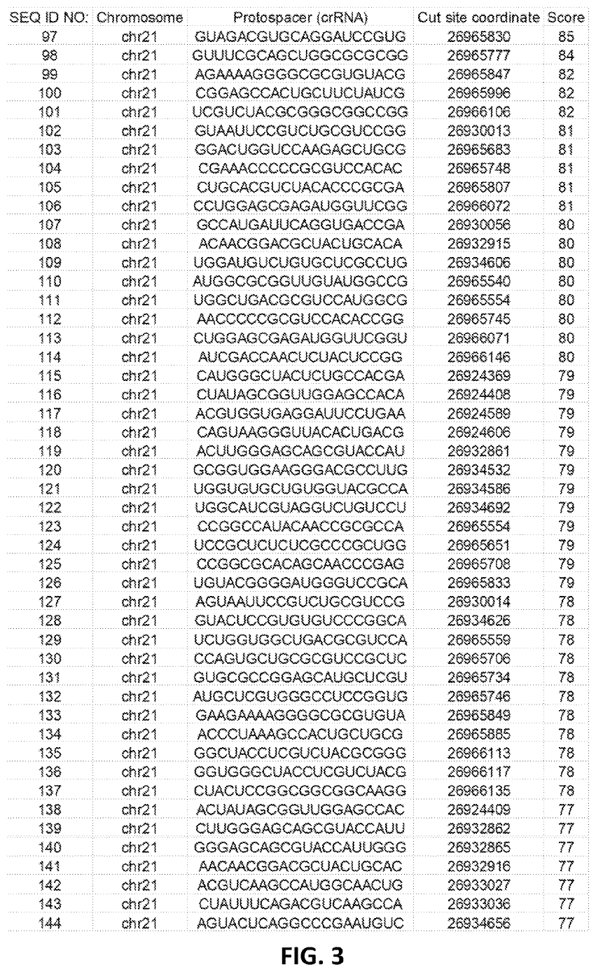

30. The pharmaceutical composition of claim 29, wherein the at least one guide RNA comprises a crRNA sequence selected from the group consisting of those sequences shown in FIG. 3 (SEQ ID NOs: 97-144).

31. The pharmaceutical composition of claim 29, wherein the at least one guide RNA comprises a crRNA sequence selected from the group consisting of SEQ ID NOs:97-106.

32. The pharmaceutical composition of any one of claims 1-11, wherein the at least one guide RNA or a nucleic acid encoding at least one guide RNA targets a mammalian MMP1 gene.

33. The pharmaceutical composition of claim 32, wherein the mammalian MMP1 gene is a human MMP1 gene.

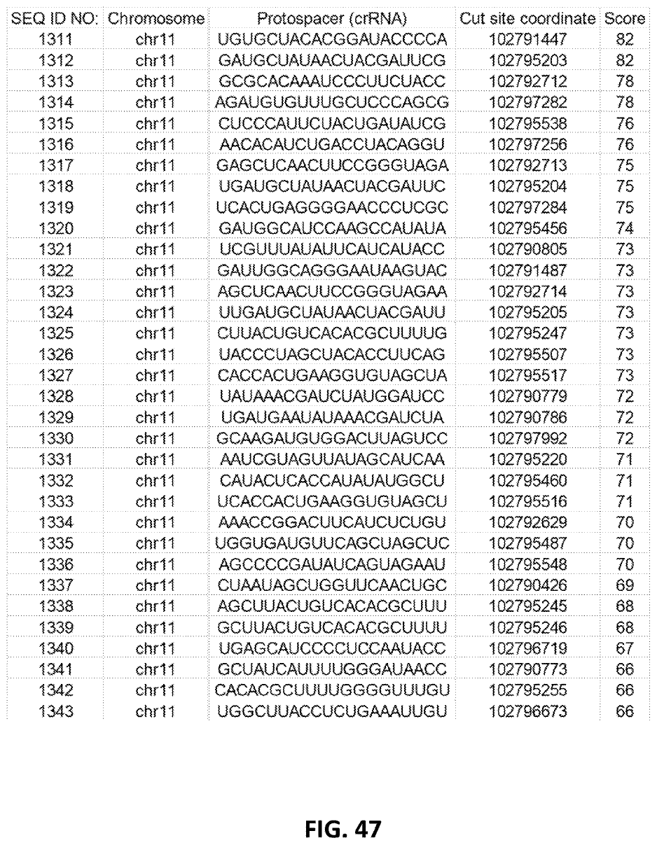

34. The pharmaceutical composition of claim 33, wherein the at least one guide RNA comprises a crRNA sequence selected from the group consisting of those sequences shown in FIG. 47 (SEQ ID NOs: 1311-1343).

35. The pharmaceutical composition of claim 33, wherein the at least one guide RNA comprises a crRNA sequence selected from the group consisting of SEQ ID NOs: 1311-1320.

36. The pharmaceutical composition of any one of claims 1-11, wherein the at least one guide RNA or a nucleic acid encoding at least one guide RNA targets a mammalian MMP2 gene.

37. The pharmaceutical composition of claim 36, wherein the mammalian MMP2 gene is a human MMP2 gene.

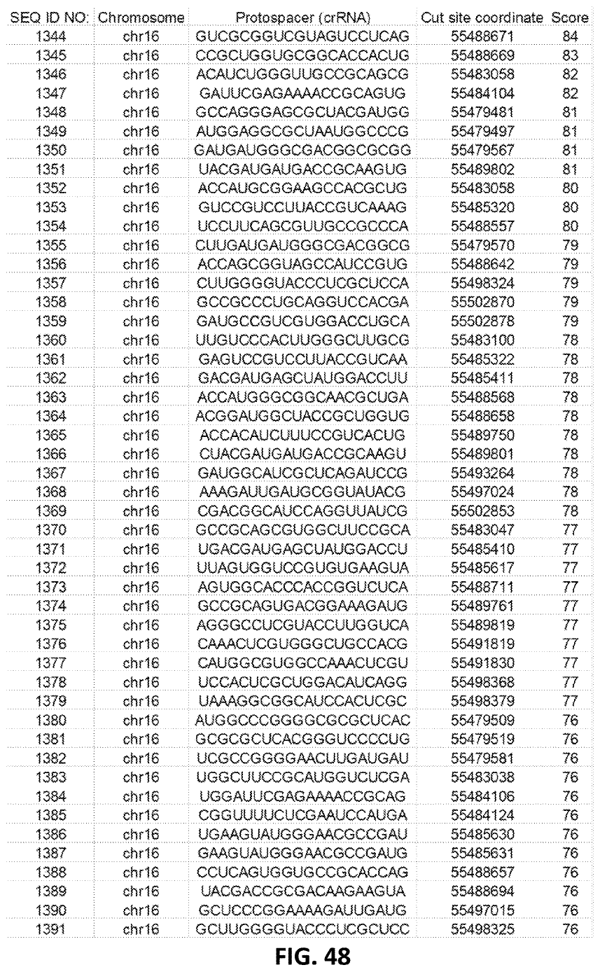

38. The pharmaceutical composition of claim 37, wherein the at least one guide RNA comprises a crRNA sequence selected from the group consisting of those sequences shown in FIG. 48 (SEQ ID NOs: 1344-1391).

39. The pharmaceutical composition of claim 37, wherein the at least one guide RNA comprises a crRNA sequence selected from the group consisting of SEQ ID NOs: 1615-1624.

40. The pharmaceutical composition of any one of claims 1-11, wherein the at least one guide RNA or a nucleic acid encoding at least one guide RNA targets a mammalian MMP3 gene.

41. The pharmaceutical composition of claim 40, wherein the mammalian MMP3 gene is a human MMP3 gene.

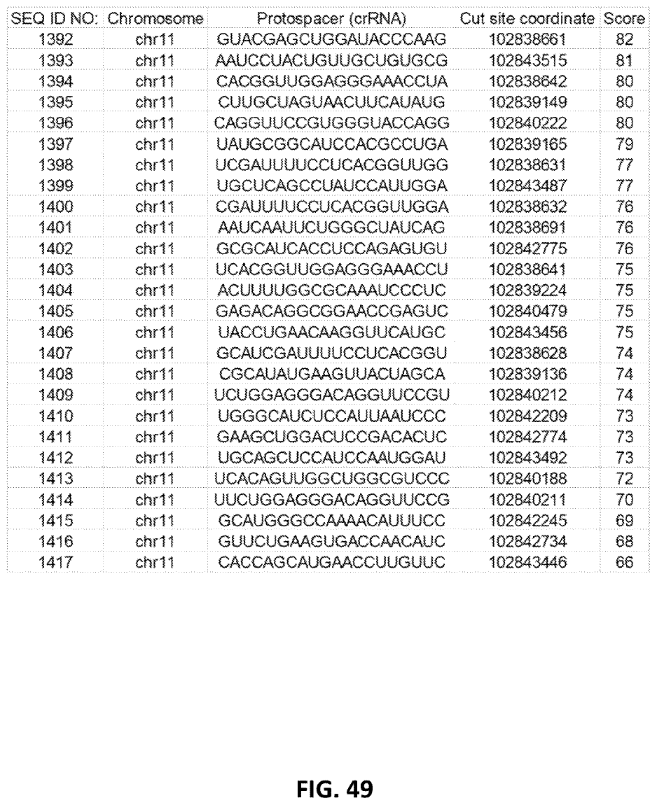

42. The pharmaceutical composition of claim 41, wherein the at least one guide RNA comprises a crRNA sequence selected from the group consisting of those sequences shown in FIG. 49 (SEQ ID NOs: 1392-1417).

43. The pharmaceutical composition of claim 41, wherein the at least one guide RNA comprises a crRNA sequence selected from the group consisting of SEQ ID NOs: 1392-1401.

44. The pharmaceutical composition of any one of claims 1-11, wherein the at least one guide RNA or a nucleic acid encoding at least one guide RNA targets a mammalian MMP7 gene.

45. The pharmaceutical composition of claim 44, wherein the mammalian MMP7 gene is a human MMP7 gene.

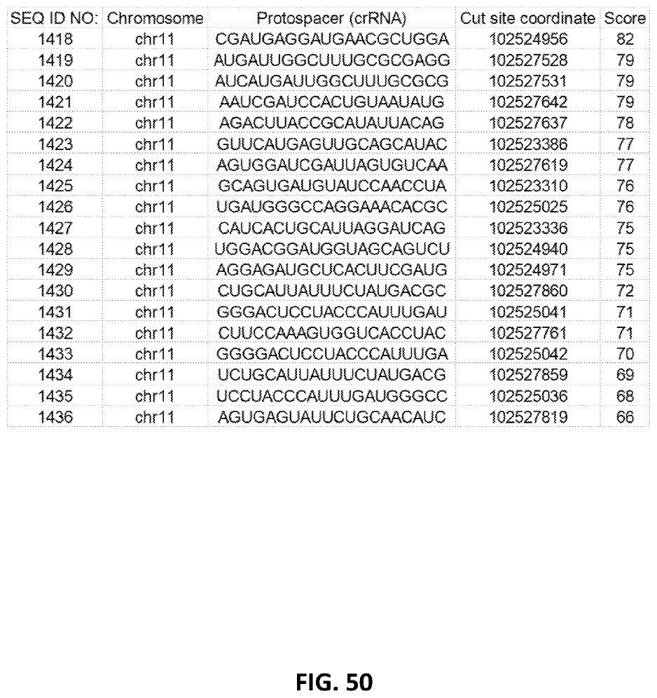

46. The pharmaceutical composition of claim 45, wherein the at least one guide RNA comprises a crRNA sequence selected from the group consisting of those sequences shown in FIG. 50 (SEQ ID NOs: 1418-1436).

47. The pharmaceutical composition of claim 45, wherein the at least one guide RNA comprises a crRNA sequence selected from the group consisting of SEQ ID NOs: 1418-1427.

48. The pharmaceutical composition of any one of claims 1-11, wherein the at least one guide RNA or a nucleic acid encoding at least one guide RNA targets a mammalian MMP8 gene.

49. The pharmaceutical composition of claim 48, wherein the mammalian MMP8 gene is a human MMP8 gene.

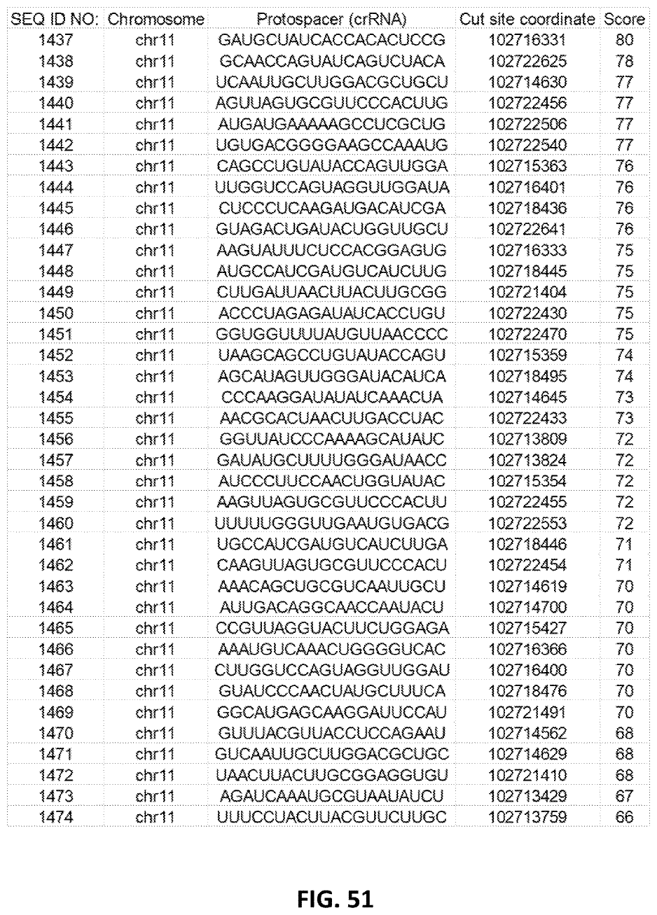

50. The pharmaceutical composition of claim 49, wherein the at least one guide RNA comprises a crRNA sequence selected from the group consisting of those sequences shown in FIG. 51 (SEQ ID NOs: 1437-1474).

51. The pharmaceutical composition of claim 49, wherein the at least one guide RNA comprises a crRNA sequence selected from the group consisting of SEQ ID NOs: 1437-1446.

52. The pharmaceutical composition of any one of claims 1-11, wherein the at least one guide RNA or a nucleic acid encoding at least one guide RNA targets a mammalian MMP10 gene.

53. The pharmaceutical composition of claim 52, wherein the mammalian MMP10 gene is a human MMP10 gene.

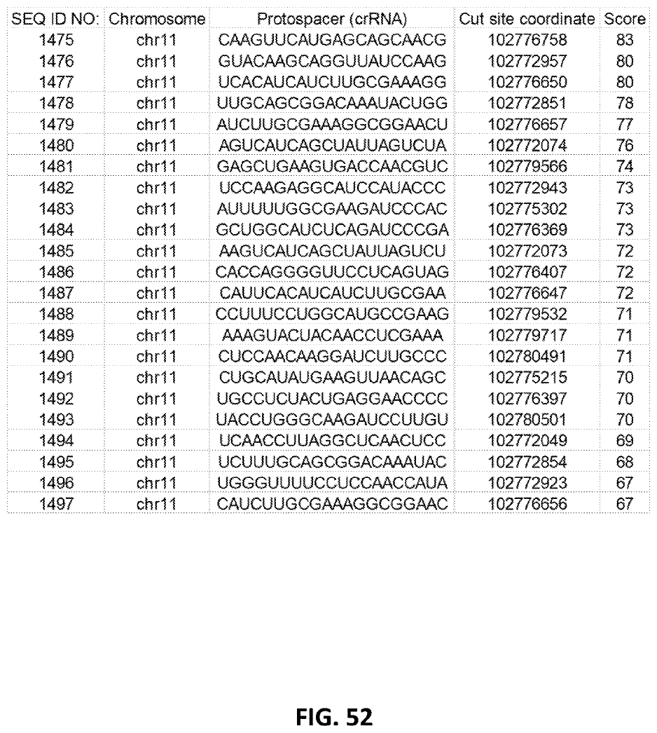

54. The pharmaceutical composition of claim 53, wherein the at least one guide RNA comprises a crRNA sequence selected from the group consisting of those sequences shown in FIG. 52 (SEQ ID NOs: 1475-1497).

55. The pharmaceutical composition of claim 53, wherein the at least one guide RNA comprises a crRNA sequence selected from the group consisting of SEQ ID NOs: 1475-1484.

56. The pharmaceutical composition of any one of claims 1-11, wherein the at least one guide RNA or a nucleic acid encoding at least one guide RNA targets a mammalian MMP12 gene.

57. The pharmaceutical composition of claim 56, wherein the mammalian MMP12 gene is a human MMP12 gene.

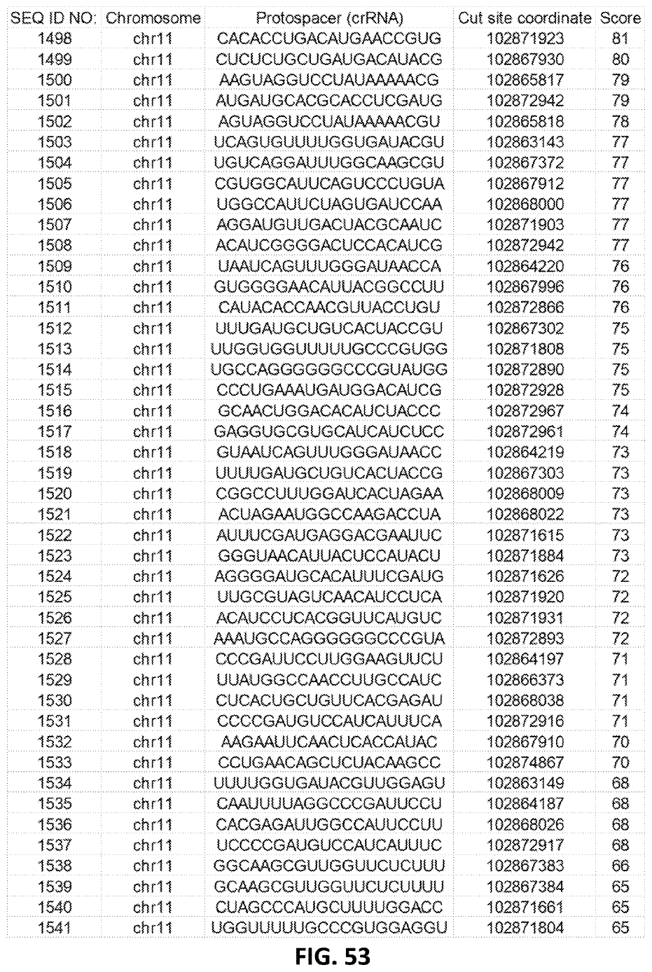

58. The pharmaceutical composition of claim 57, wherein the at least one guide RNA comprises a crRNA sequence selected from the group consisting of those sequences shown in FIG. 53 (SEQ ID NOs: 1498-1541).

59. The pharmaceutical composition of claim 57, wherein the at least one guide RNA comprises a crRNA sequence selected from the group consisting of SEQ ID NOs: 1498-1507.

60. The pharmaceutical composition of any one of claims 1-11, wherein the at least one guide RNA or a nucleic acid encoding at least one guide RNA targets a mammalian MMP13 gene.

61. The pharmaceutical composition of claim 60, wherein the mammalian MMP13 gene is a human MMP13 gene.

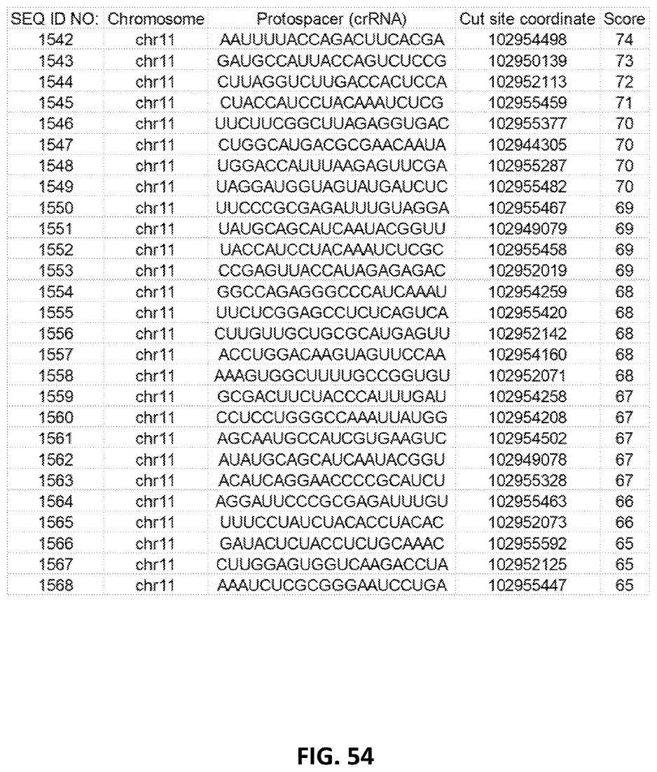

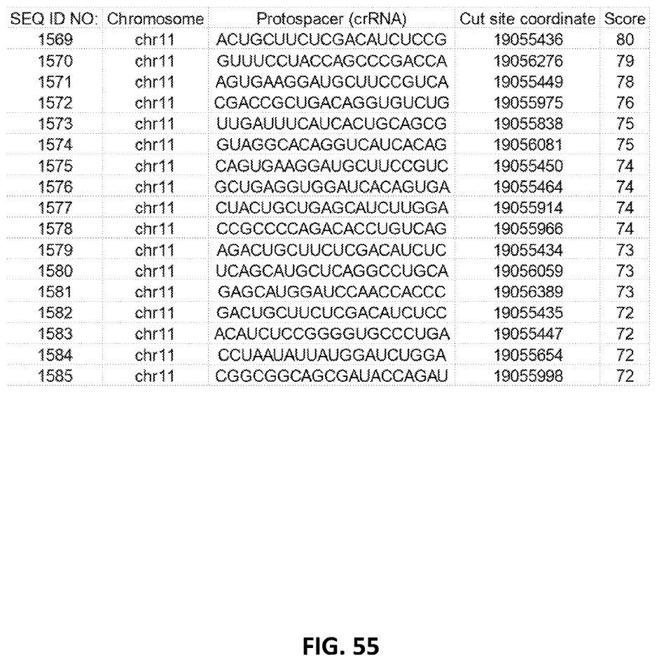

62. The pharmaceutical composition of claim 61, wherein the at least one guide RNA comprises a crRNA sequence selected from the group consisting of those sequences shown in FIG. 54 (SEQ ID NOs: 1542-1568).

63. The pharmaceutical composition of claim 61, wherein the at least one guide RNA comprises a crRNA sequence selected from the group consisting of SEQ ID NOs: 1542-1551.

64. The pharmaceutical composition of any one of claims 1-11, wherein the at least one guide RNA or a nucleic acid encoding at least one guide RNA targets a mammalian TIMP1 gene.

65. The pharmaceutical composition of claim 64, wherein the mammalian TIMP1 gene is a human TIMP1 gene.

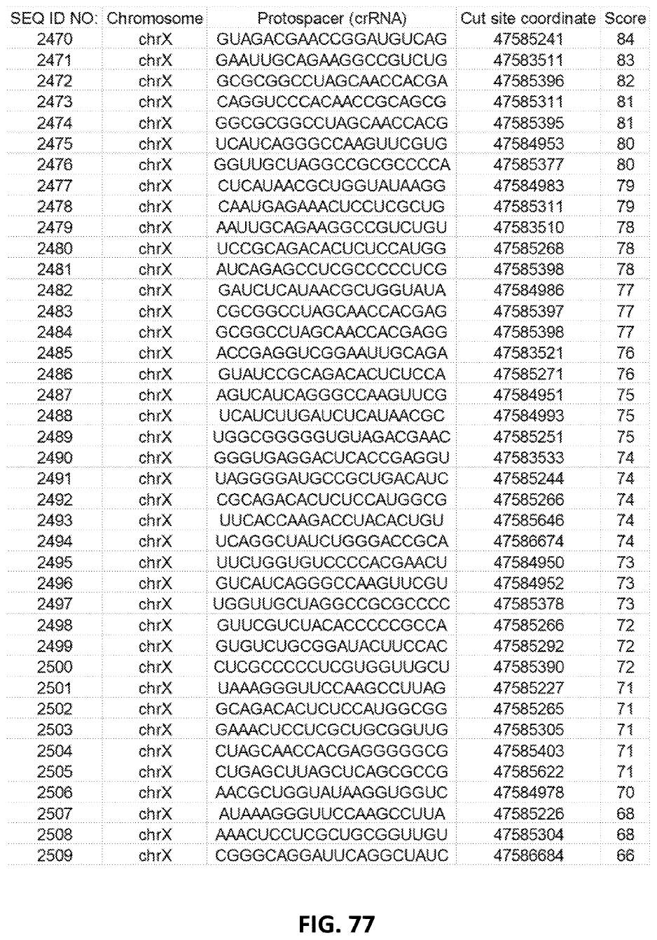

66. The pharmaceutical composition of claim 65, wherein the at least one guide RNA comprises a crRNA sequence selected from the group consisting of those sequences shown in FIG. 77 (SEQ ID NOs: 2470-2509).

67. The pharmaceutical composition of claim 65, wherein the at least one guide RNA comprises a crRNA sequence selected from the group consisting of SEQ ID NOs: 2470-2479.

68. The pharmaceutical composition of any one of claims 1-11, wherein the at least one guide RNA or a nucleic acid encoding at least one guide RNA targets a mammalian TIMP3 gene.

69. The pharmaceutical composition of claim 68, wherein the mammalian TIMP3 gene is a human TIMP3 gene.

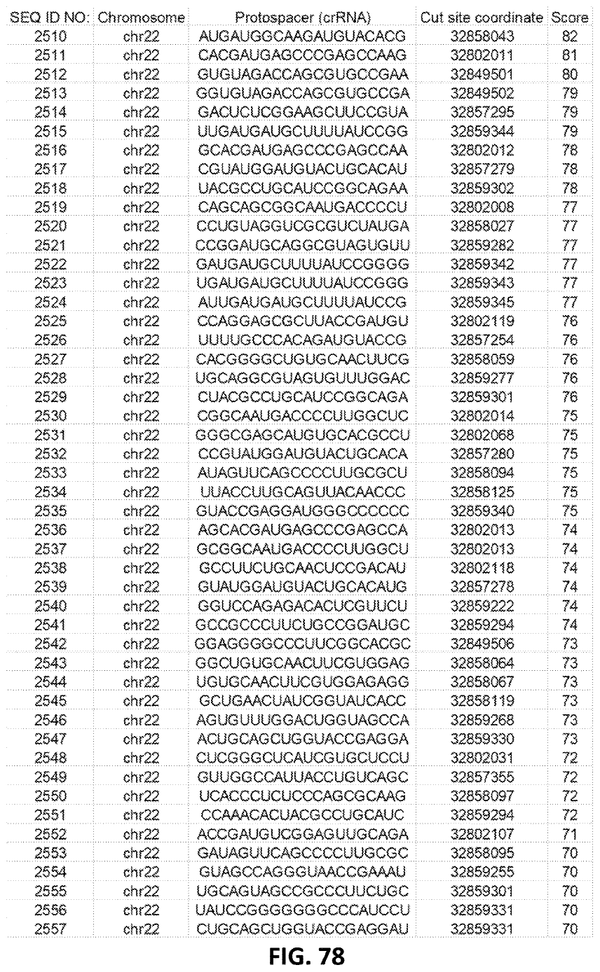

70. The pharmaceutical composition of claim 69, wherein the at least one guide RNA comprises a crRNA sequence selected from the group consisting of those sequences shown in FIG. 78 (SEQ ID NOs: 2510-2557).

71. The pharmaceutical composition of claim 69, wherein the at least one guide RNA comprises a crRNA sequence selected from the group consisting of SEQ ID NOs: 2510-2519.

72. The pharmaceutical composition of any one of claims 1-11, wherein the at least one guide RNA or a nucleic acid encoding at least one guide RNA targets a mammalian CXCL1 gene.

73. The pharmaceutical composition of claim 72, wherein the mammalian CXCL1 gene is a human CXCL1 gene.

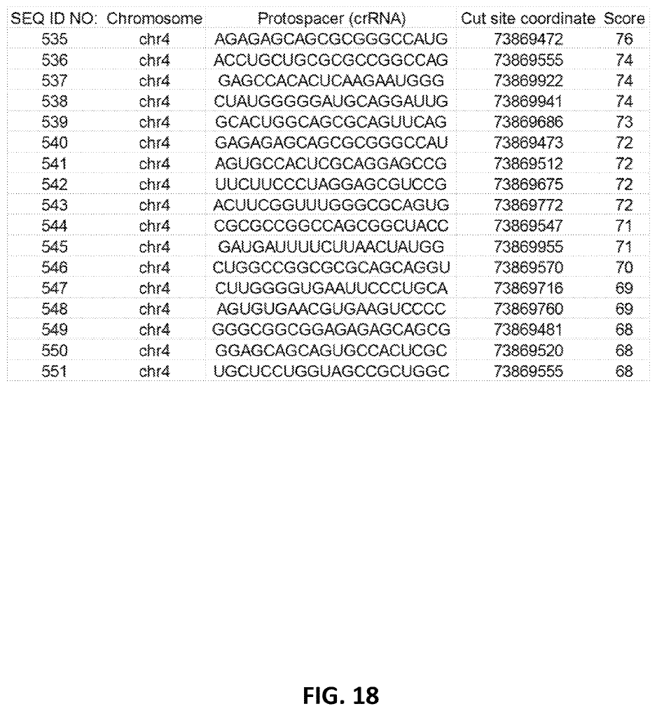

74. The pharmaceutical composition of claim 73, wherein the at least one guide RNA comprises a crRNA sequence selected from the group consisting of those sequences shown in FIG. 18 (SEQ ID NOs: 535-551).

75. The pharmaceutical composition of claim 73, wherein the at least one guide RNA comprises a crRNA sequence selected from the group consisting of SEQ ID NOs: 535-544.

76. The pharmaceutical composition of any one of claims 1-11, wherein the at least one guide RNA or a nucleic acid encoding at least one guide RNA targets a mammalian CXCL2 gene.

77. The pharmaceutical composition of claim 76, wherein the mammalian CXCL2 gene is a human CXCL2 gene.

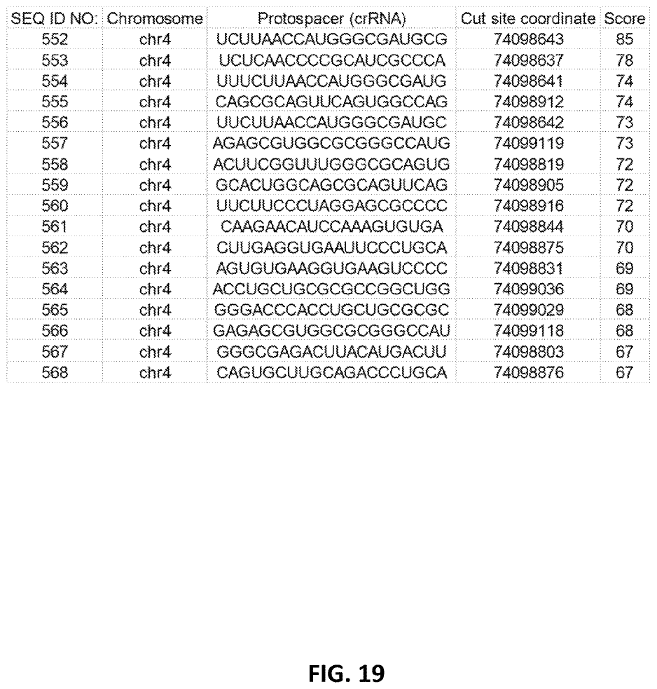

78. The pharmaceutical composition of claim 77, wherein the at least one guide RNA comprises a crRNA sequence selected from the group consisting of those sequences shown in FIG. 19 (SEQ ID NOs: 552-568).

79. The pharmaceutical composition of claim 77, wherein the at least one guide RNA comprises a crRNA sequence selected from the group consisting of SEQ ID NOs: 552-561.

80. The pharmaceutical composition of any one of claims 1-11, wherein the at least one guide RNA or a nucleic acid encoding at least one guide RNA targets a mammalian CXCL3 gene.

81. The pharmaceutical composition of claim 80, wherein the mammalian CXCL3 gene is a human CXCL3 gene.

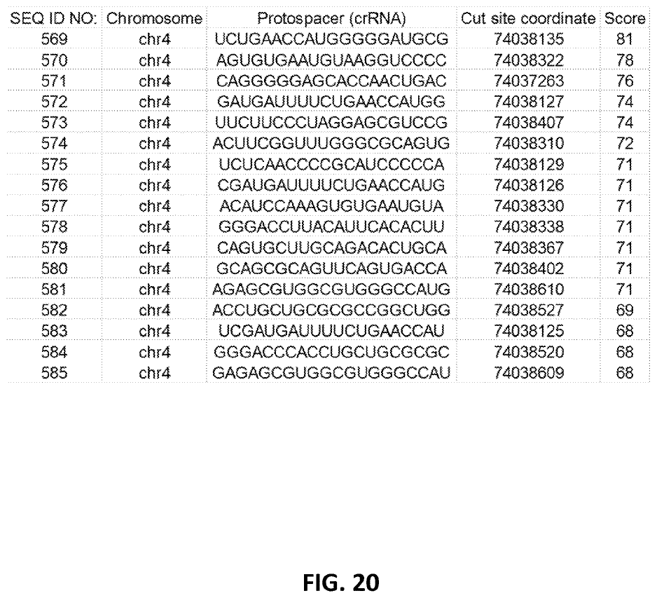

82. The pharmaceutical composition of claim 81, wherein the at least one guide RNA comprises a crRNA sequence selected from the group consisting of those sequences shown in FIG. 20 (SEQ ID NOs: 569-585).

83. The pharmaceutical composition of claim 81, wherein the at least one guide RNA comprises a crRNA sequence selected from the group consisting of SEQ ID NOs: 569-578.

84. The pharmaceutical composition of any one of claims 1-11, wherein the at least one guide RNA or a nucleic acid encoding at least one guide RNA targets a mammalian CXCL5 gene.

85. The pharmaceutical composition of claim 84, wherein the mammalian CXCL5 gene is a human CXCL5 gene.

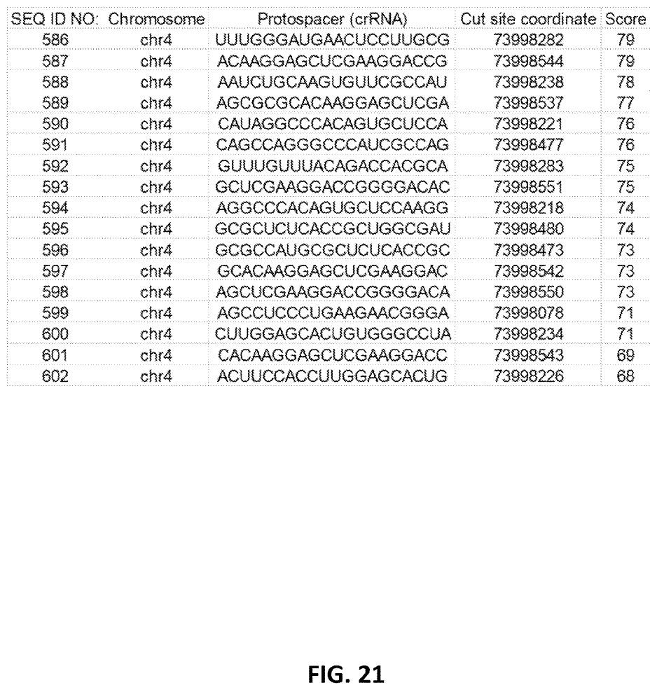

86. The pharmaceutical composition of claim 85, wherein the at least one guide RNA comprises a crRNA sequence selected from the group consisting of those sequences shown in FIG. 21 (SEQ ID NOs: 586-602).

87. The pharmaceutical composition of claim 85, wherein the at least one guide RNA comprises a crRNA sequence selected from the group consisting of SEQ ID NOs: 586-595.

88. The pharmaceutical composition of any one of claims 1-11, wherein the at least one guide RNA or a nucleic acid encoding at least one guide RNA targets a mammalian CXCL6 gene.

89. The pharmaceutical composition of claim 88, wherein the mammalian CXCL6 gene is a human CXCL6 gene.

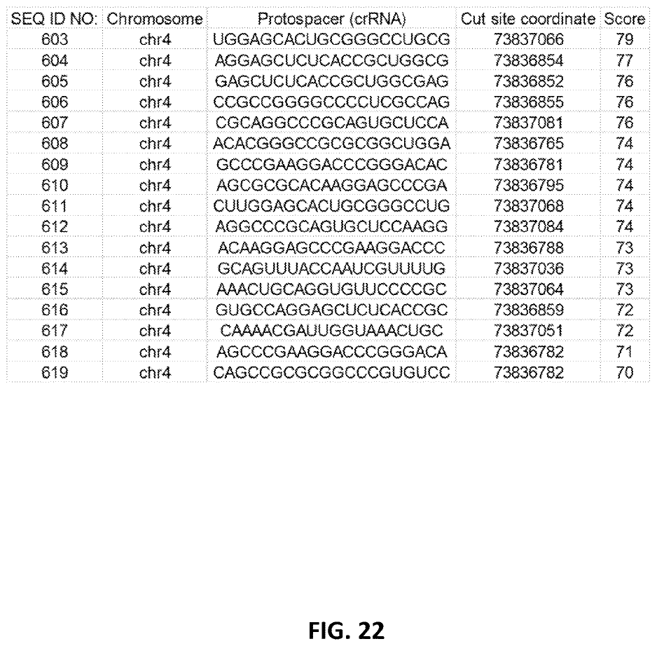

90. The pharmaceutical composition of claim 89, wherein the at least one guide RNA comprises a crRNA sequence selected from the group consisting of those sequences shown in FIG. 22 (SEQ ID NOs: 603-619).

91. The pharmaceutical composition of claim 89, wherein the at least one guide RNA comprises a crRNA sequence selected from the group consisting of SEQ ID NOs: 603-612.

92. The pharmaceutical composition of any one of claims 1-11, wherein the at least one guide RNA or a nucleic acid encoding at least one guide RNA targets a mammalian CXCL8 gene.

93. The pharmaceutical composition of claim 92, wherein the mammalian CXCL8 gene is a human CXCL8 gene.

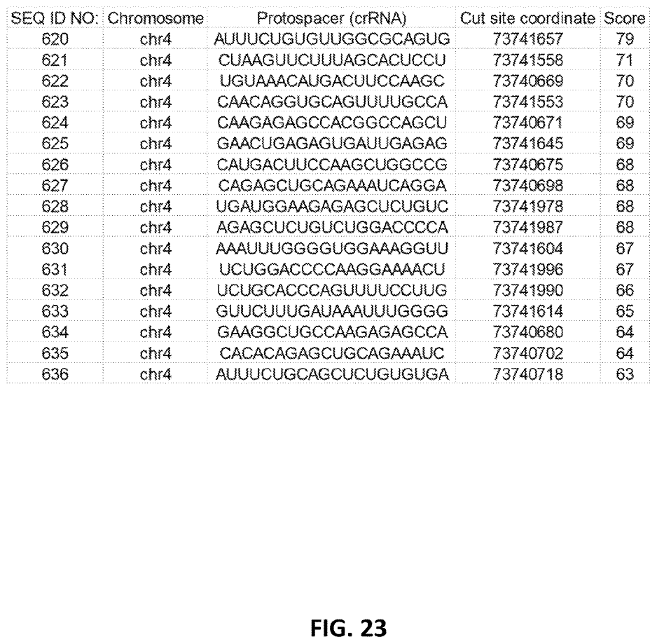

94. The pharmaceutical composition of claim 93, wherein the at least one guide RNA comprises a crRNA sequence selected from the group consisting of those sequences shown in FIG. 23 (SEQ ID NOs: 620-636).

95. The pharmaceutical composition of claim 93, wherein the at least one guide RNA comprises a crRNA sequence selected from the group consisting of SEQ ID NOs: 620-629.

96. The pharmaceutical composition of any one of claims 1-11, wherein the at least one guide RNA or a nucleic acid encoding at least one guide RNA targets a mammalian CCL2 gene.

97. The pharmaceutical composition of claim 96, wherein the mammalian CCL2 gene is a human CCL2 gene.

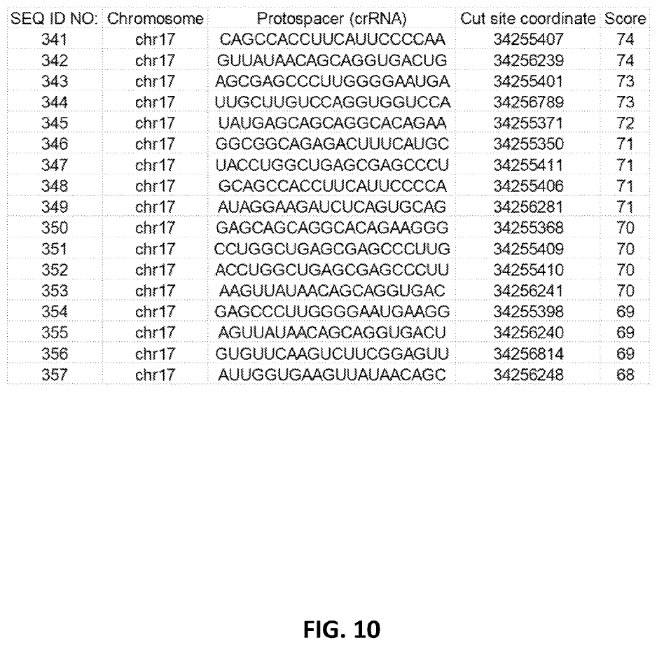

98. The pharmaceutical composition of claim 97, wherein the at least one guide RNA comprises a crRNA sequence selected from the group consisting of those sequences shown in FIG. 10 (SEQ ID NOs: 341-357).

99. The pharmaceutical composition of claim 97, wherein the at least one guide RNA comprises a crRNA sequence selected from the group consisting of SEQ ID NOs: 341-350.

100. The pharmaceutical composition of any one of claims 1-11, wherein the at least one guide RNA or a nucleic acid encoding at least one guide RNA targets a mammalian CCL3 gene.

101. The pharmaceutical composition of claim 100, wherein the mammalian CCL3 gene is a human CCL3 gene.

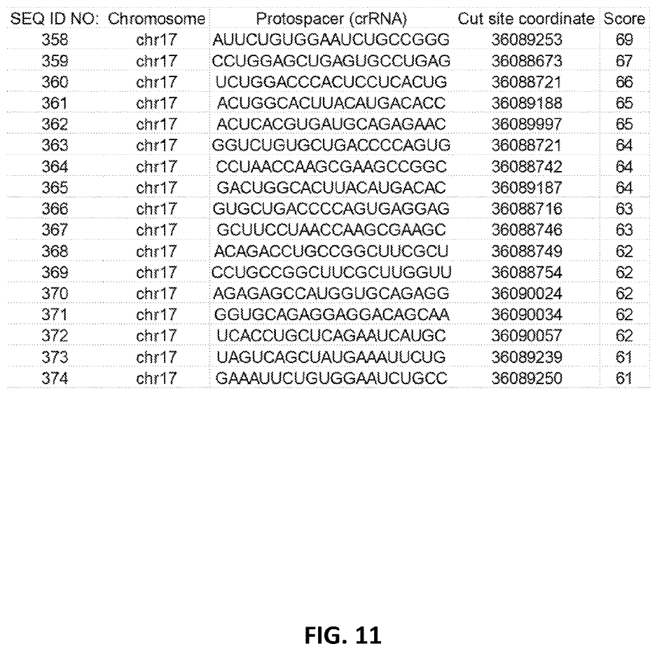

102. The pharmaceutical composition of claim 101, wherein the at least one guide RNA comprises a crRNA sequence selected from the group consisting of those sequences shown in FIG. 11 (SEQ ID NOs: 358-374).

103. The pharmaceutical composition of claim 101, wherein the at least one guide RNA comprises a crRNA sequence selected from the group consisting of SEQ ID NOs: 358-367.

104. The pharmaceutical composition of any one of claims 1-11, wherein the at least one guide RNA or a nucleic acid encoding at least one guide RNA targets a mammalian CCL5 gene.

105. The pharmaceutical composition of claim 104, wherein the mammalian CCL5 gene is a human CCL5 gene.

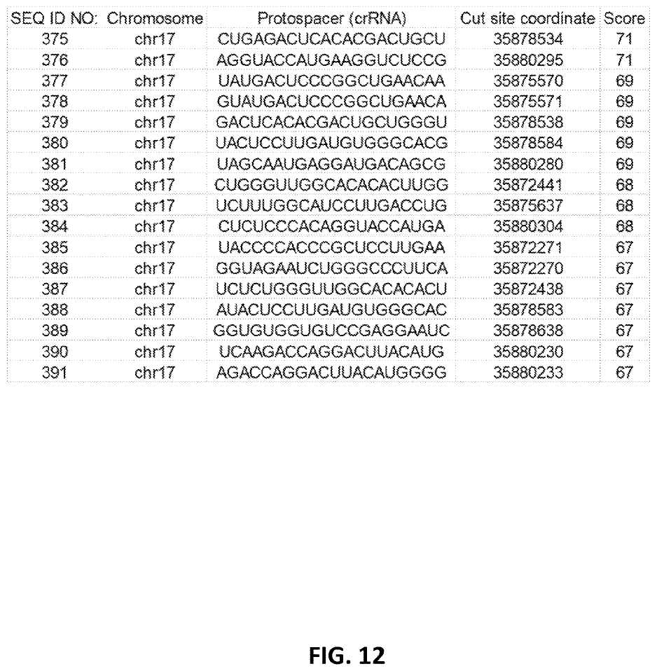

106. The pharmaceutical composition of claim 105, wherein the at least one guide RNA comprises a crRNA sequence selected from the group consisting of those sequences shown in FIG. 12 (SEQ ID NOs: 375-391).

107. The pharmaceutical composition of claim 105, wherein the at least one guide RNA comprises a crRNA sequence selected from the group consisting of SEQ ID NOs: 375-384.

108. The pharmaceutical composition of any one of claims 1-11, wherein the at least one guide RNA or a nucleic acid encoding at least one guide RNA targets a mammalian CCL7 gene.

109. The pharmaceutical composition of claim 108, wherein the mammalian CCL7 gene is a human CCL7 gene.

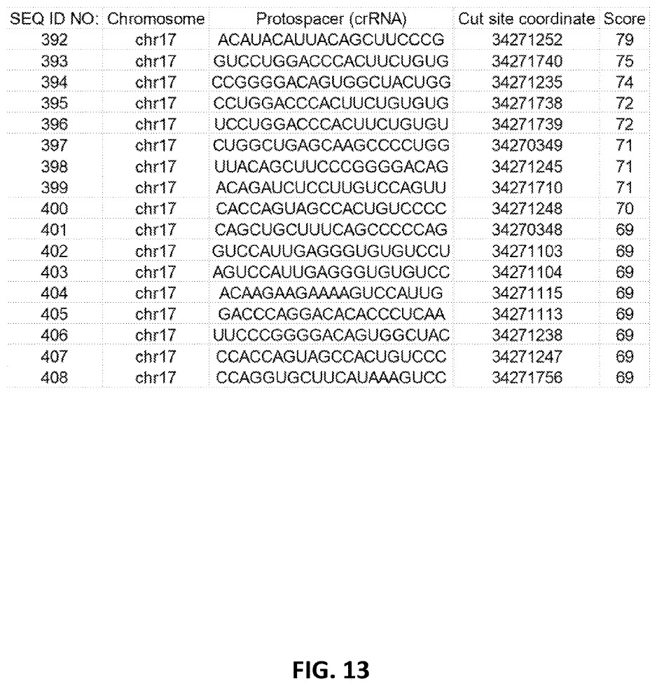

110. The pharmaceutical composition of claim 109, wherein the at least one guide RNA comprises a crRNA sequence selected from the group consisting of those sequences shown in FIG. 13 (SEQ ID NOs: 392-408).

111. The pharmaceutical composition of claim 109, wherein the at least one guide RNA comprises a crRNA sequence selected from the group consisting of SEQ ID NOs: 392-401.

112. The pharmaceutical composition of any one of claims 1-11, wherein the at least one guide RNA or a nucleic acid encoding at least one guide RNA targets a mammalian CCL20 gene.

113. The pharmaceutical composition of claim 112, wherein the mammalian CCL20 gene is a human CCL20 gene.

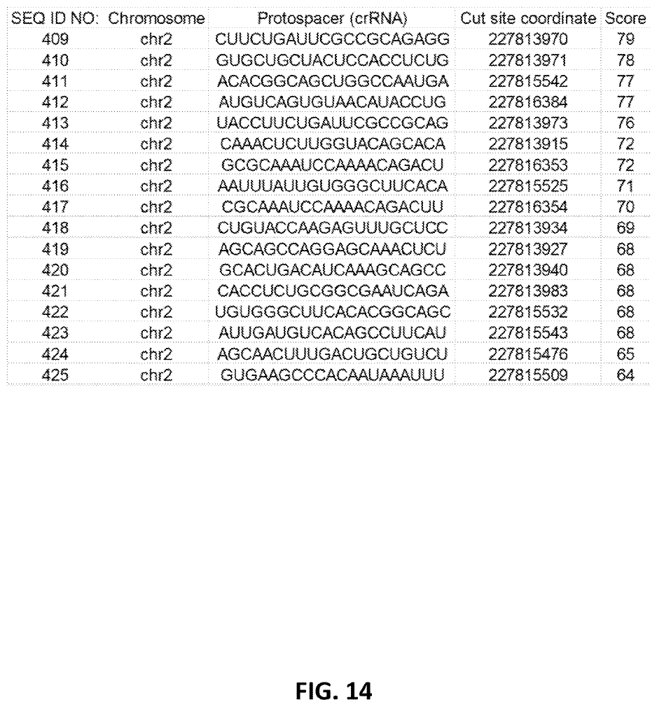

114. The pharmaceutical composition of claim 113, wherein the at least one guide RNA comprises a crRNA sequence selected from the group consisting of those sequences shown in FIG. 14 (SEQ ID NOs: 409-425).

115. The pharmaceutical composition of claim 113, wherein the at least one guide RNA comprises a crRNA sequence selected from the group consisting of SEQ ID NOs: 409-418.

116. The pharmaceutical composition of any one of claims 1-11, wherein the at least one guide RNA or a nucleic acid encoding at least one guide RNA targets a mammalian CXCR2 gene.

117. The pharmaceutical composition of claim 116, wherein the mammalian CXCR2 gene is a human CXCR2 gene.

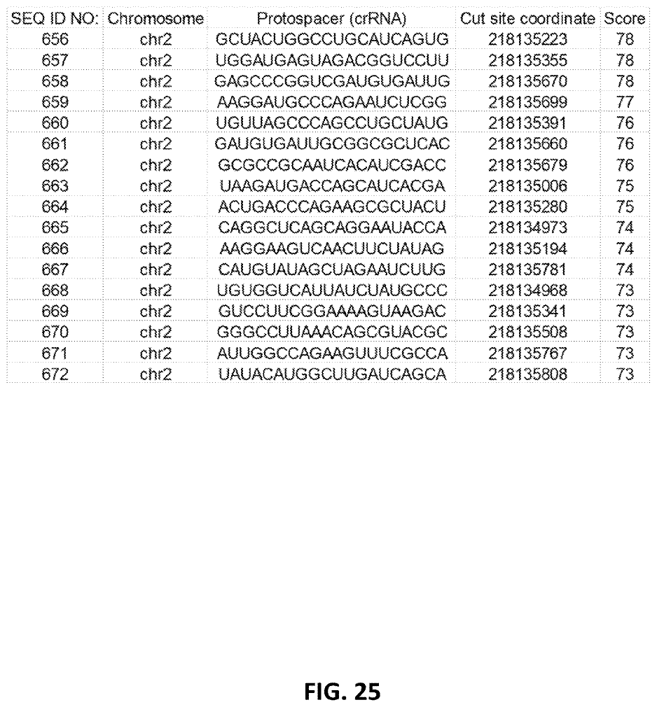

118. The pharmaceutical composition of claim 117, wherein the at least one guide RNA comprises a crRNA sequence selected from the group consisting of those sequences shown in FIG. 25 (SEQ ID NOs: 656-672).

119. The pharmaceutical composition of claim 117, wherein the at least one guide RNA comprises a crRNA sequence selected from the group consisting of SEQ ID NOs: 656-665.

120. The pharmaceutical composition of any one of claims 1-11, wherein the at least one guide RNA or a nucleic acid encoding at least one guide RNA targets a mammalian CCR7 gene.

121. The pharmaceutical composition of claim 120, wherein the mammalian CCR7 gene is a human CCR7 gene.

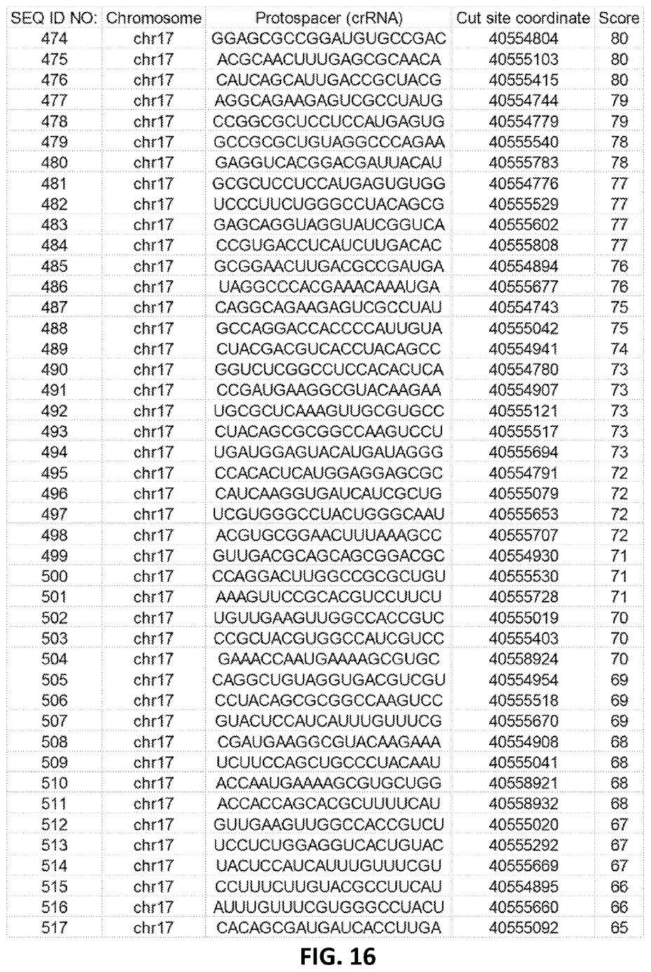

122. The pharmaceutical composition of claim 121, wherein the at least one guide RNA comprises a crRNA sequence selected from the group consisting of those sequences shown in FIG. 16 (SEQ ID NOs: 474-517).

123. The pharmaceutical composition of claim 121, wherein the at least one guide RNA comprises a crRNA sequence selected from the group consisting of SEQ ID NOs: 474-483.

124. The pharmaceutical composition of any one of claims 1-11, wherein the at least one guide RNA or a nucleic acid encoding at least one guide RNA targets a mammalian YAP1 gene.

125. The pharmaceutical composition of claim 124, wherein the mammalian YAP1 gene is a human YAP1 gene.

126. The pharmaceutical composition of claim 125, wherein the at least one guide RNA comprises a crRNA sequence selected from the group consisting of those sequences shown in FIG. 82 (SEQ ID NOs: 2671-2718).

127. The pharmaceutical composition of claim 125, wherein the at least one guide RNA comprises a crRNA sequence selected from the group consisting of SEQ ID NOS: 2671-2680.

128. The pharmaceutical composition of any one of claims 1-11, wherein the at least one guide RNA or a nucleic acid encoding at least one guide RNA targets a mammalian TNF gene.

129. The pharmaceutical composition of claim 128, wherein the mammalian TNF gene is a human TNF gene.

130. The pharmaceutical composition of claim 129, wherein the at least one guide RNA comprises a crRNA sequence selected from the group consisting of those sequences shown in FIG. 79 (SEQ ID NOs: 2558-2574).

131. The pharmaceutical composition of claim 129, wherein the at least one guide RNA comprises a crRNA sequence selected from the group consisting of SEQ ID NOs: 2558-2567.

132. The pharmaceutical composition of any one of claims 1-11, wherein the at least one guide RNA or a nucleic acid encoding at least one guide RNA targets a mammalian ADAM17 gene.

133. The pharmaceutical composition of claim 132, wherein the mammalian ADAM17 gene is a human ADAM17 gene.

134. The pharmaceutical composition of claim 133, wherein the at least one guide RNA comprises a crRNA sequence selected from the group consisting of those sequences shown in FIG. 1 (SEQ ID NOS:1-48).

135. The pharmaceutical composition of claim 133, wherein the at least one guide RNA comprises a crRNA sequence selected from the group consisting of SEQ ID NOS:1-10.

136. The pharmaceutical composition of any one of claims 1-11, wherein the at least one guide RNA or a nucleic acid encoding at least one guide RNA targets a mammalian TNFRSF1A gene.

137. The pharmaceutical composition of claim 136, wherein the mammalian TNFRSF1A gene is a human TNFRSF1A gene.

138. The pharmaceutical composition of claim 137, wherein the at least one guide RNA comprises a crRNA sequence selected from the group consisting of those sequences shown in FIG. 80 (SEQ ID NOS: 2575-2622).

139. The pharmaceutical composition of claim 137, wherein the at least one guide RNA comprises a crRNA sequence selected from the group consisting of SEQ ID NOS: 2575-2584.

140. The pharmaceutical composition of any one of claims 1-11, wherein the at least one guide RNA or a nucleic acid encoding at least one guide RNA targets a mammalian TNFRSF1B gene.

141. The pharmaceutical composition of claim 140, wherein the mammalian TNFRSF1B gene is a human TNFRSF1B gene.

142. The pharmaceutical composition of claim 141, wherein the at least one guide RNA comprises a crRNA sequence selected from the group consisting of those sequences shown in FIG. 81 (SEQ ID NOS: 2623-2670).

143. The pharmaceutical composition of claim 141, wherein the at least one guide RNA comprises a crRNA sequence selected from the group consisting of SEQ ID NOS: 2623-2632.

144. The pharmaceutical composition of any one of claims 1-11, wherein the at least one guide RNA or a nucleic acid encoding at least one guide RNA targets a mammalian IL4 gene.

145. The pharmaceutical composition of claim 144, wherein the mammalian IL4 gene is a human IL4 gene.

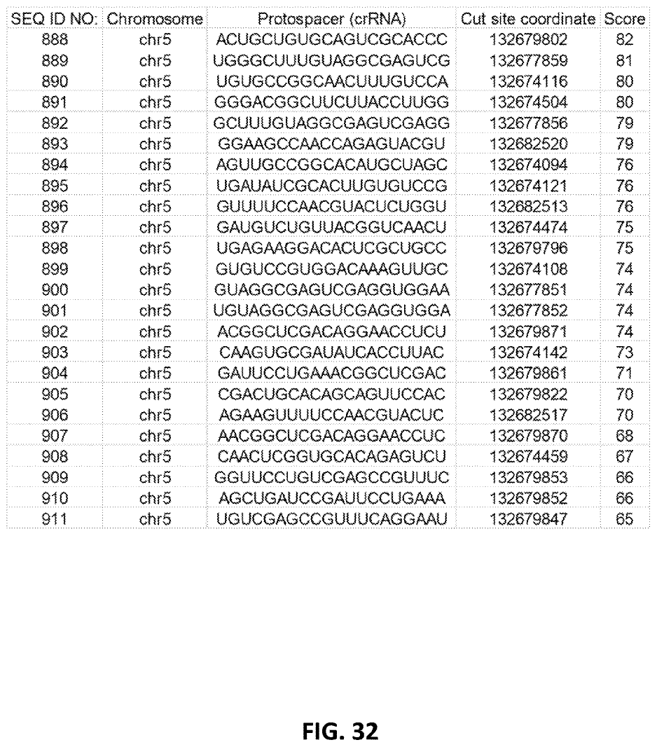

146. The pharmaceutical composition of claim 145, wherein the at least one guide RNA comprises a crRNA sequence selected from the group consisting of those sequences shown in FIG. 32 (SEQ ID NOS: 888-911).

147. The pharmaceutical composition of claim 145, wherein the at least one guide RNA comprises a crRNA sequence selected from the group consisting of SEQ ID NOS: 888-897.

148. The pharmaceutical composition of any one of claims 1-11, wherein the at least one guide RNA or a nucleic acid encoding at least one guide RNA targets a mammalian IL1R1 gene.

149. The pharmaceutical composition of claim 148, wherein the mammalian IL1R1 gene is a human IL1R1 gene.

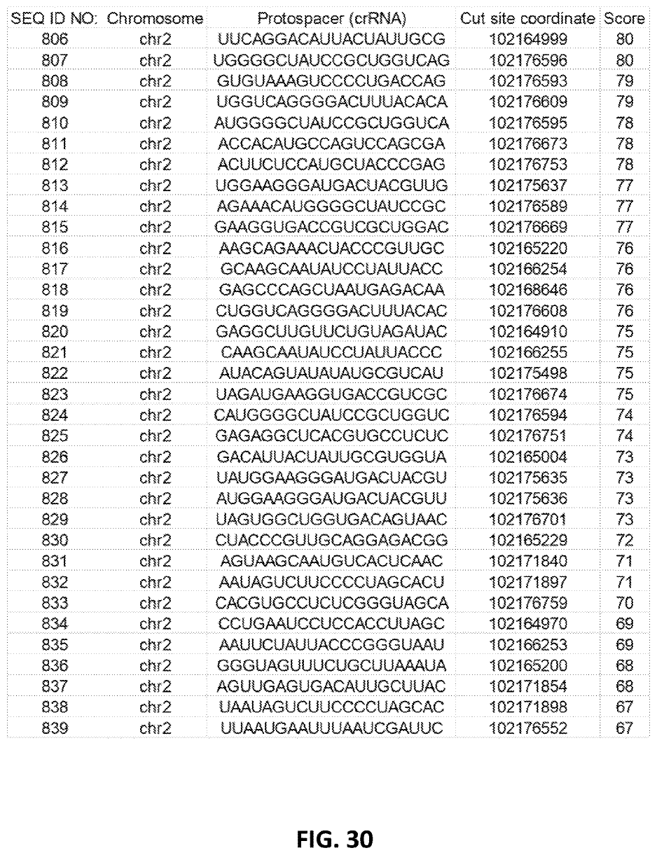

150. The pharmaceutical composition of claim 149, wherein the at least one guide RNA comprises a crRNA sequence selected from the group consisting of those sequences shown in FIG. 30 (SEQ ID NOS: 806-839).

151. The pharmaceutical composition of claim 149, wherein the at least one guide RNA comprises a crRNA sequence selected from the group consisting of SEQ ID NOS: 806-815.

152. The pharmaceutical composition of any one of claims 1-11, wherein the at least one guide RNA or a nucleic acid encoding at least one guide RNA targets a mammalian IL6 gene.

153. The pharmaceutical composition of claim 152, wherein the mammalian IL6 gene is a human IL6 gene.

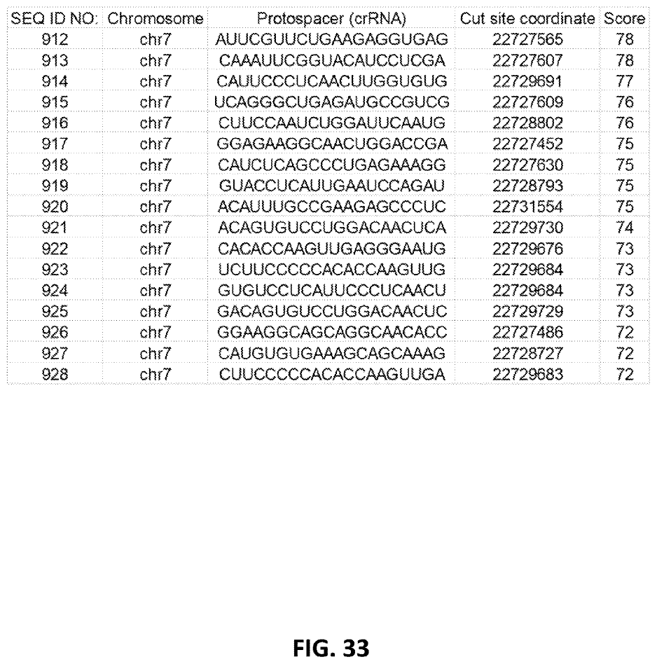

154. The pharmaceutical composition of claim 153, wherein the at least one guide RNA comprises a crRNA sequence selected from the group consisting of those sequences shown in FIG. 33 (SEQ ID NOS: 912-928).

155. The pharmaceutical composition of claim 153, wherein the at least one guide RNA comprises a crRNA sequence selected from the group consisting of SEQ ID NOS: 912-921.

156. The pharmaceutical composition of any one of claims 1-11, wherein the at least one guide RNA or a nucleic acid encoding at least one guide RNA targets a mammalian IL6R gene.

157. The pharmaceutical composition of claim 156, wherein the mammalian IL6R gene is a human IL6R gene.

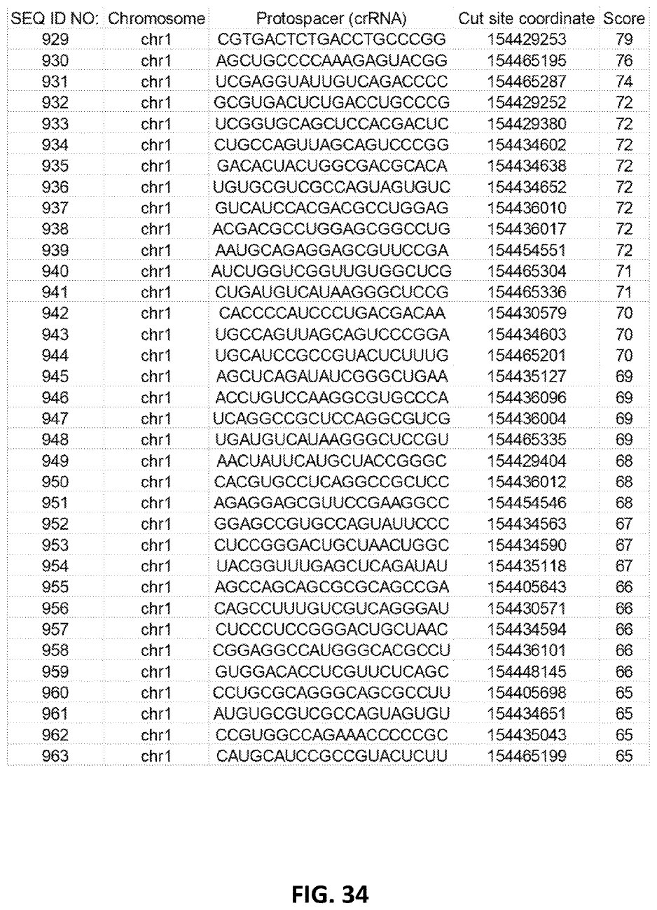

158. The pharmaceutical composition of claim 157, wherein the at least one guide RNA comprises a crRNA sequence selected from the group consisting of those sequences shown in FIG. 34 (SEQ ID NOS: 929-963).

159. The pharmaceutical composition of claim 157, wherein the at least one guide RNA comprises a crRNA sequence selected from the group consisting of SEQ ID NOS: 929-938.

160. The pharmaceutical composition of any one of claims 1-11, wherein the at least one guide RNA or a nucleic acid encoding at least one guide RNA targets a mammalian CXCR1 gene.

161. The pharmaceutical composition of claim 160, wherein the mammalian CXCR1 gene is a human CXCR1 gene.

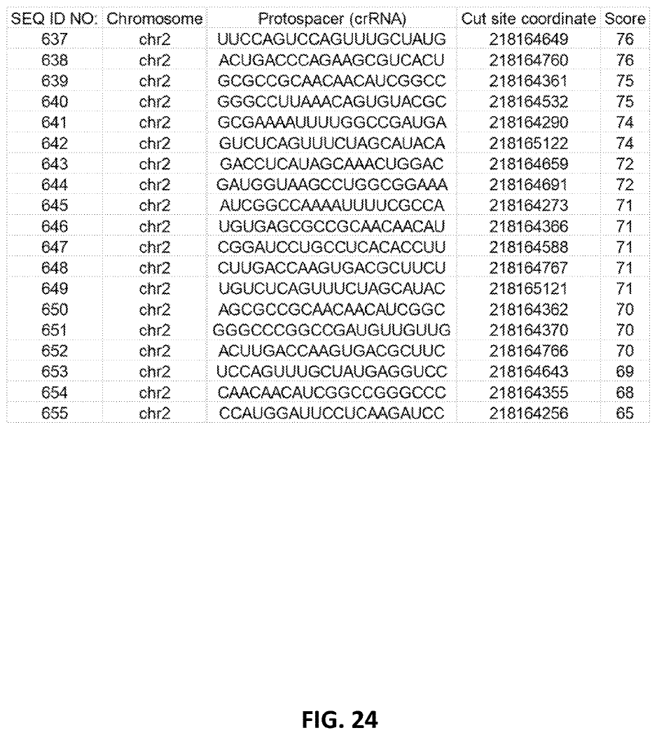

162. The pharmaceutical composition of claim 161, wherein the at least one guide RNA comprises a crRNA sequence selected from the group consisting of those sequences shown in FIG. 24 (SEQ ID NOS: 637-655).

163. The pharmaceutical composition of claim 161, wherein the at least one guide RNA comprises a crRNA sequence selected from the group consisting of SEQ ID NOS: 637-646.

164. The pharmaceutical composition of any one of claims 1-11, wherein the at least one guide RNA or a nucleic acid encoding at least one guide RNA targets a mammalian IL10 gene.

165. The pharmaceutical composition of claim 164, wherein the mammalian IL10 gene is a human IL10 gene.

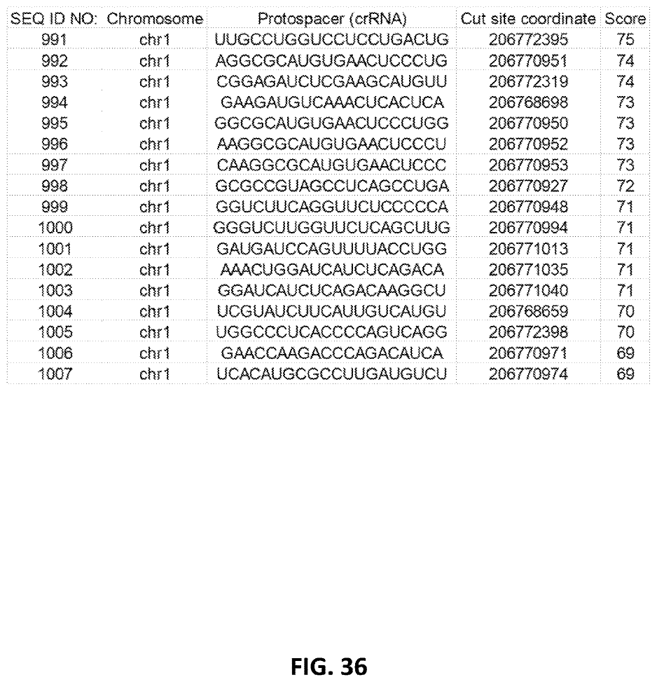

166. The pharmaceutical composition of claim 165, wherein the at least one guide RNA comprises a crRNA sequence selected from the group consisting of those sequences shown in FIG. 36 (SEQ ID NOS: 964-990).

167. The pharmaceutical composition of claim 165, wherein the at least one guide RNA comprises a crRNA sequence selected from the group consisting of SEQ ID NOS: 964-973.

168. The pharmaceutical composition of any one of claims 1-11, wherein the at least one guide RNA or a nucleic acid encoding at least one guide RNA targets a mammalian IL10RB gene.

169. The pharmaceutical composition of claim 168, wherein the mammalian IL10RB gene is a human IL10RB gene.

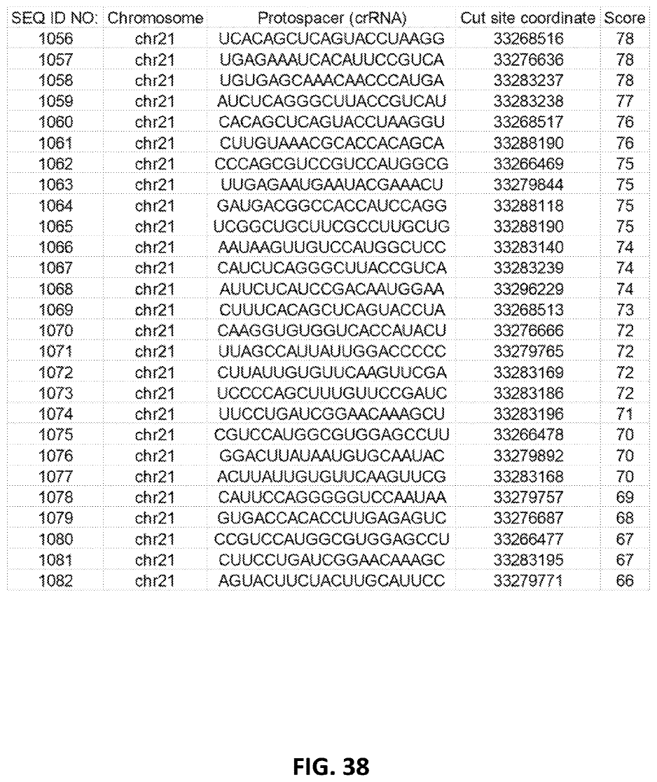

170. The pharmaceutical composition of claim 169, wherein the at least one guide RNA comprises a crRNA sequence selected from the group consisting of those sequences shown in FIG. 38 (SEQ ID NOS: 1056-1082).

171. The pharmaceutical composition of claim 169, wherein the at least one guide RNA comprises a crRNA sequence selected from the group consisting of SEQ ID NOS: 1056-1064.

172. The pharmaceutical composition of any one of claims 1-11, wherein the at least one guide RNA or a nucleic acid encoding at least one guide RNA targets a mammalian IL10RA gene.

173. The pharmaceutical composition of claim 172, wherein the mammalian IL10RA gene is a human IL10RA gene.

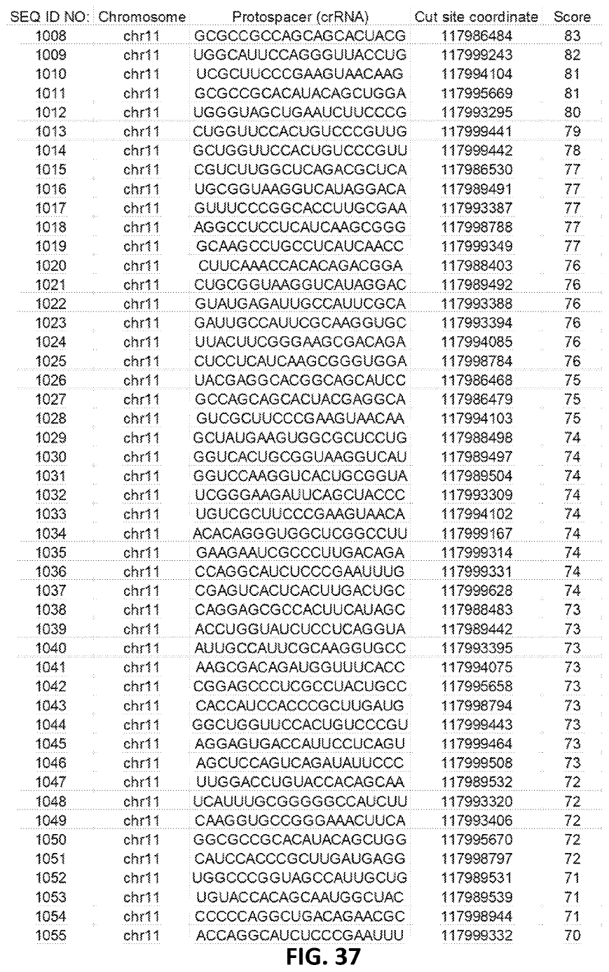

174. The pharmaceutical composition of claim 173, wherein the at least one guide RNA comprises a crRNA sequence selected from the group consisting of those sequences shown in FIG. 37 (SEQ ID NOS: 1008-1055).

175. The pharmaceutical composition of claim 173, wherein the at least one guide RNA comprises a crRNA sequence selected from the group consisting of SEQ ID NOS: 1008-1017.

176. The pharmaceutical composition of any one of claims 1-11, wherein the at least one guide RNA or a nucleic acid encoding at least one guide RNA targets a mammalian IL13 gene.

177. The pharmaceutical composition of claim 176, wherein the mammalian IL13 gene is a human IL13 gene.

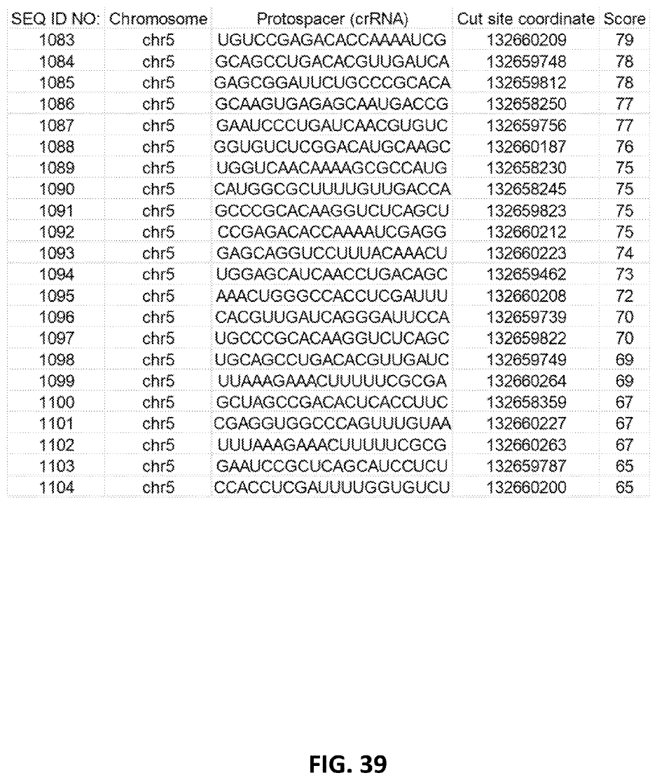

178. The pharmaceutical composition of claim 177, wherein the at least one guide RNA comprises a crRNA sequence selected from the group consisting of those sequences shown in FIG. 39 (SEQ ID NOS: 1083-1104).

179. The pharmaceutical composition of claim 177, wherein the at least one guide RNA comprises a crRNA sequence selected from the group consisting of SEQ ID NOS: 1083-1104.

180. The pharmaceutical composition of any one of claims 1-11, wherein the at least one guide RNA or a nucleic acid encoding at least one guide RNA targets a mammalian IL13RA1 gene.

181. The pharmaceutical composition of claim 180, wherein the mammalian IL13RA1 gene is a human IL13RA1 gene.

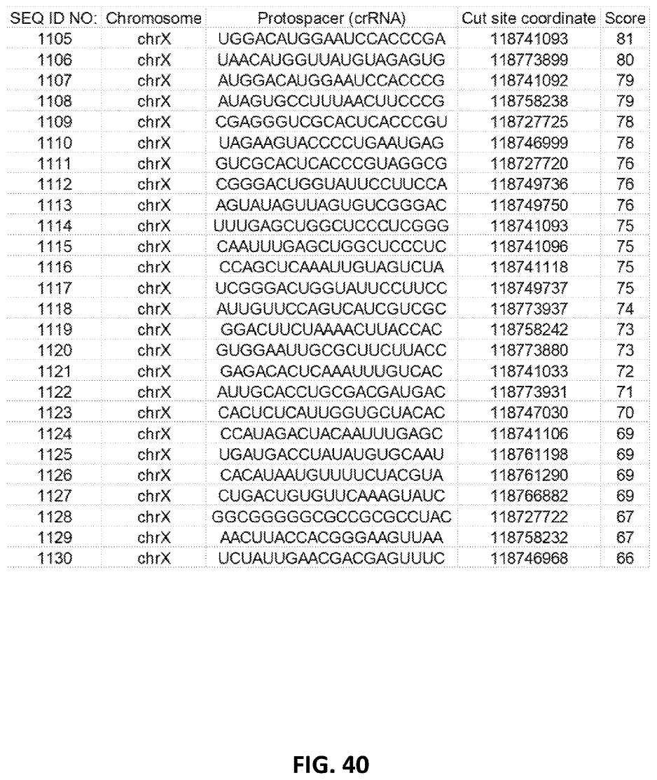

182. The pharmaceutical composition of claim 181, wherein the at least one guide RNA comprises a crRNA sequence selected from the group consisting of those sequences shown in FIG. 40 (SEQ ID NOS: 1105-1130).

183. The pharmaceutical composition of claim 181, wherein the at least one guide RNA comprises a crRNA sequence selected from the group consisting of SEQ ID NOS: 1105-1114.

184. The pharmaceutical composition of any one of claims 1-11, wherein the at least one guide RNA or a nucleic acid encoding at least one guide RNA targets a mammalian IL13RA2 gene.

185. The pharmaceutical composition of claim 184, wherein the mammalian IL13RA2 gene is a human IL13RA2 gene.

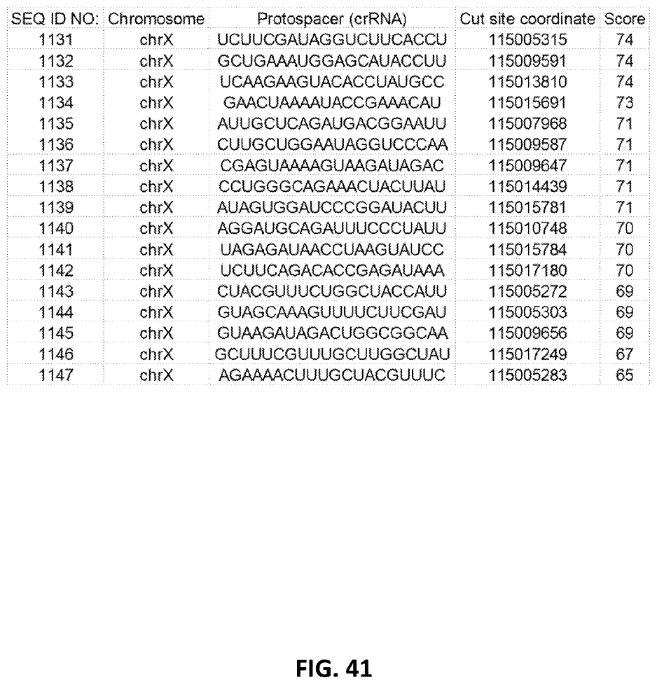

186. The pharmaceutical composition of claim 185, wherein the at least one guide RNA comprises a crRNA sequence selected from the group consisting of those sequences shown in FIG. 41 (SEQ ID NOS: 1131-1147).

187. The pharmaceutical composition of claim 185, wherein the at least one guide RNA comprises a crRNA sequence selected from the group consisting of SEQ ID NOS: 1131-1140.

188. The pharmaceutical composition of any one of claims 1-11, wherein the at least one guide RNA or a nucleic acid encoding at least one guide RNA targets a mammalian IL17A gene.

189. The pharmaceutical composition of claim 188, wherein the mammalian IL17A gene is a human IL17A gene.

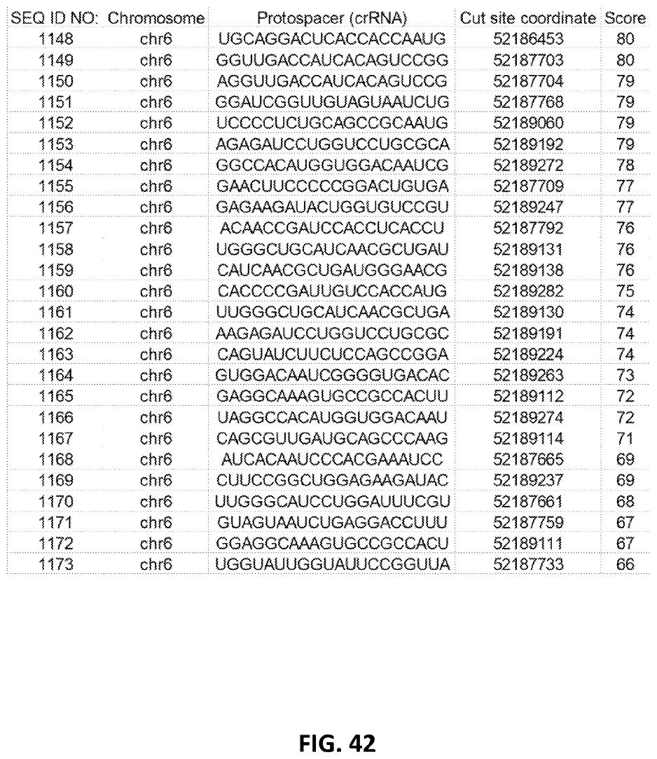

190. The pharmaceutical composition of claim 189, wherein the at least one guide RNA comprises a crRNA sequence selected from the group consisting of those sequences shown in FIG. 42 (1148-1173).

191. The pharmaceutical composition of claim 189, wherein the at least one guide RNA comprises a crRNA sequence selected from the group consisting of SEQ ID NOS: 1148-1157.

192. The pharmaceutical composition of any one of claims 1-11, wherein the at least one guide RNA or a nucleic acid encoding at least one guide RNA targets a mammalian IL17RA gene.

193. The pharmaceutical composition of claim 192, wherein the mammalian IL17RA gene is a human IL17RA gene.

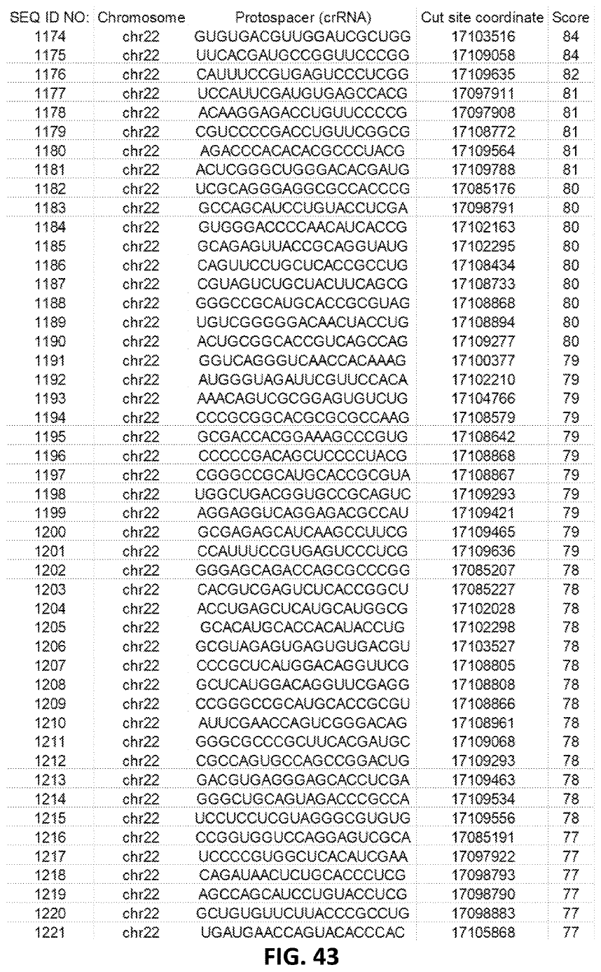

194. The pharmaceutical composition of claim 193, wherein the at least one guide RNA comprises a crRNA sequence selected from the group consisting of those sequences shown in FIG. 43 (1174-1221).

195. The pharmaceutical composition of claim 193, wherein the at least one guide RNA comprises a crRNA sequence selected from the group consisting of SEQ ID NOS: 1174-1183.

196. The pharmaceutical composition of any one of claims 1-11, wherein the at least one guide RNA or a nucleic acid encoding at least one guide RNA targets a mammalian IL18 gene.

197. The pharmaceutical composition of claim 196, wherein the mammalian IL18 gene is a human IL18 gene.

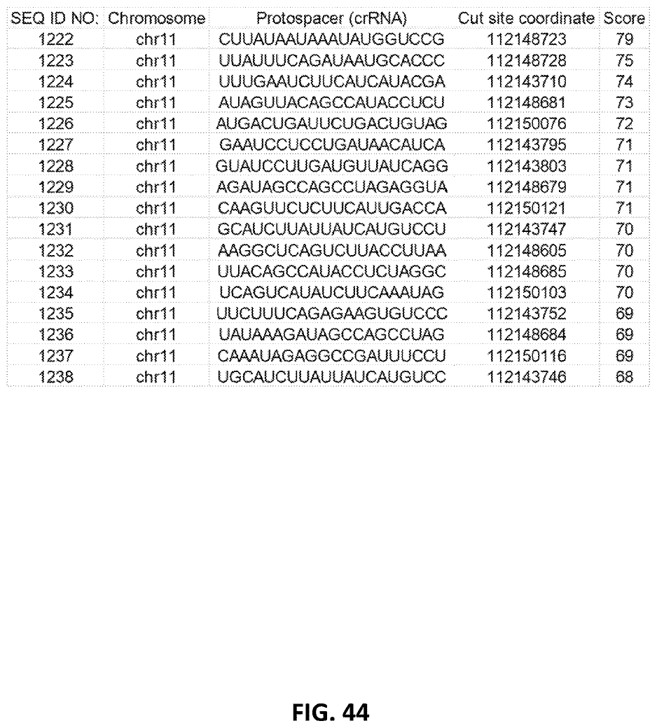

198. The pharmaceutical composition of claim 197, wherein the at least one guide RNA comprises a crRNA sequence selected from the group consisting of those sequences shown in FIG. 44 (SEQ ID NOS: 1222-1238).

199. The pharmaceutical composition of claim 197, wherein the at least one guide RNA comprises a crRNA sequence selected from the group consisting of SEQ ID NOS: 1222-1231.

200. The pharmaceutical composition of any one of claims 1-11, wherein the at least one guide RNA or a nucleic acid encoding at least one guide RNA targets a mammalian IL18RAP gene.

201. The pharmaceutical composition of claim 200, wherein the mammalian IL18RAP gene is a human IL18RAP gene.

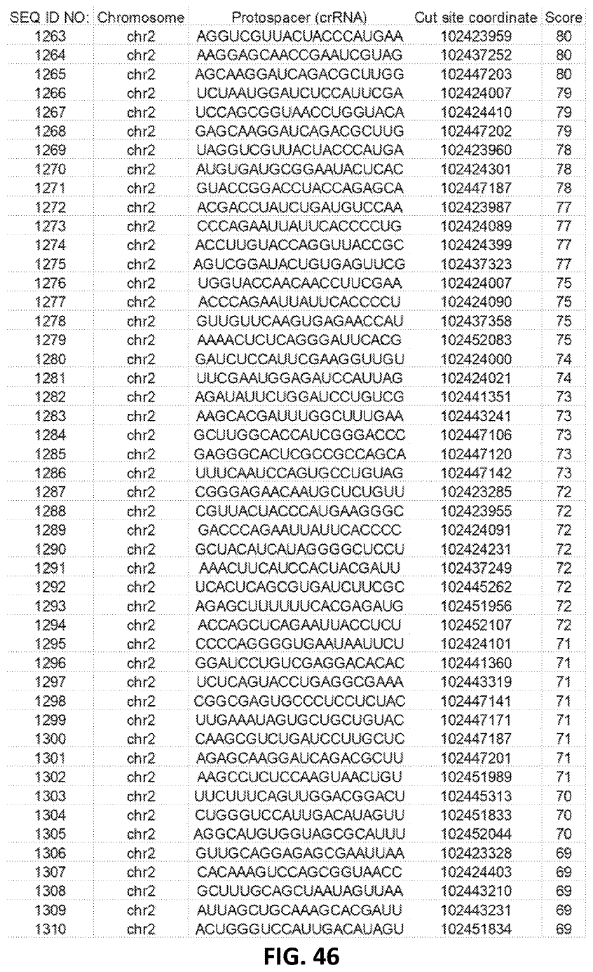

202. The pharmaceutical composition of claim 201, wherein the at least one guide RNA comprises a crRNA sequence selected from the group consisting of those sequences shown in FIG. 46 (SEQ ID NOS: 1263-1310).

203. The pharmaceutical composition of claim 201, wherein the at least one guide RNA comprises a crRNA sequence selected from the group consisting of SEQ ID NOS: 1263-1272.

204. The pharmaceutical composition of any one of claims 1-11, wherein the at least one guide RNA or a nucleic acid encoding at least one guide RNA targets a mammalian IL18R1 gene.

205. The pharmaceutical composition of claim 204, wherein the mammalian IL18R1 gene is a human IL18R1 gene.

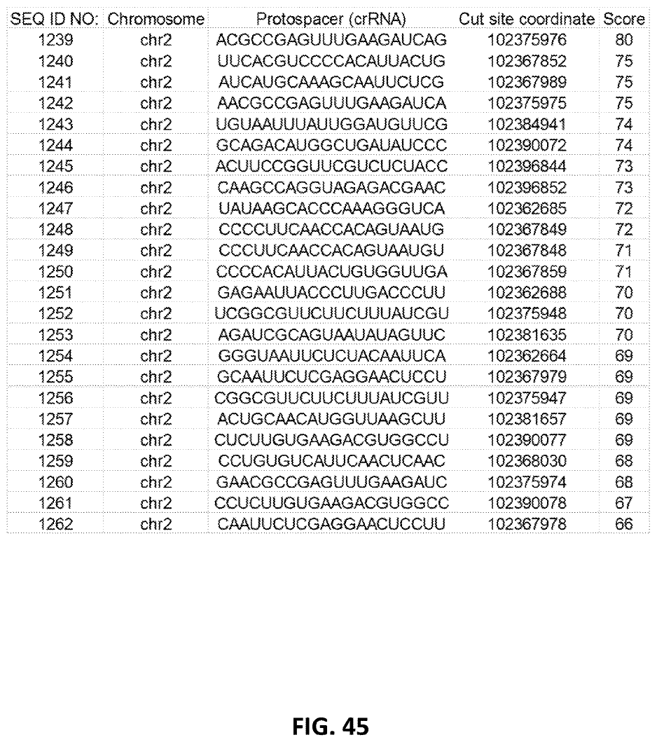

206. The pharmaceutical composition of claim 205, wherein the at least one guide RNA comprises a crRNA sequence selected from the group consisting of those sequences shown in FIG. 45 (SEQ ID NOS: 1239-1262).

207. The pharmaceutical composition of claim 205, wherein the at least one guide RNA comprises a crRNA sequence selected from the group consisting of SEQ ID NOS: 1239-1248.

208. The pharmaceutical composition of any one of claims 1-11, wherein the at least one guide RNA or a nucleic acid encoding at least one guide RNA targets a mammalian SCN1A gene.

209. The pharmaceutical composition of claim 208, wherein the mammalian SCN1A gene is a human SCN1A gene.

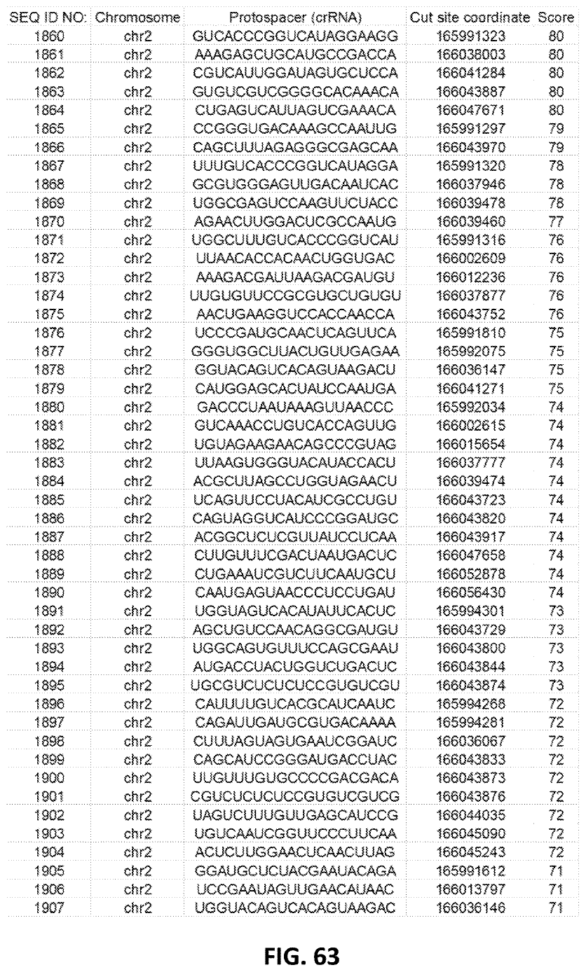

210. The pharmaceutical composition of claim 209, wherein the at least one guide RNA comprises a crRNA sequence selected from the group consisting of those sequences shown in FIG. 63 (SEQ ID NOS: 1860-1907).

211. The pharmaceutical composition of claim 209, wherein the at least one guide RNA comprises a crRNA sequence selected from the group consisting of SEQ ID NOS: 1860-1869.

212. The pharmaceutical composition of any one of claims 1-11, wherein the at least one guide RNA or a nucleic acid encoding at least one guide RNA targets a mammalian SCN2A gene.

213. The pharmaceutical composition of claim 212, wherein the mammalian SCN2A gene is a human SCN2A gene.

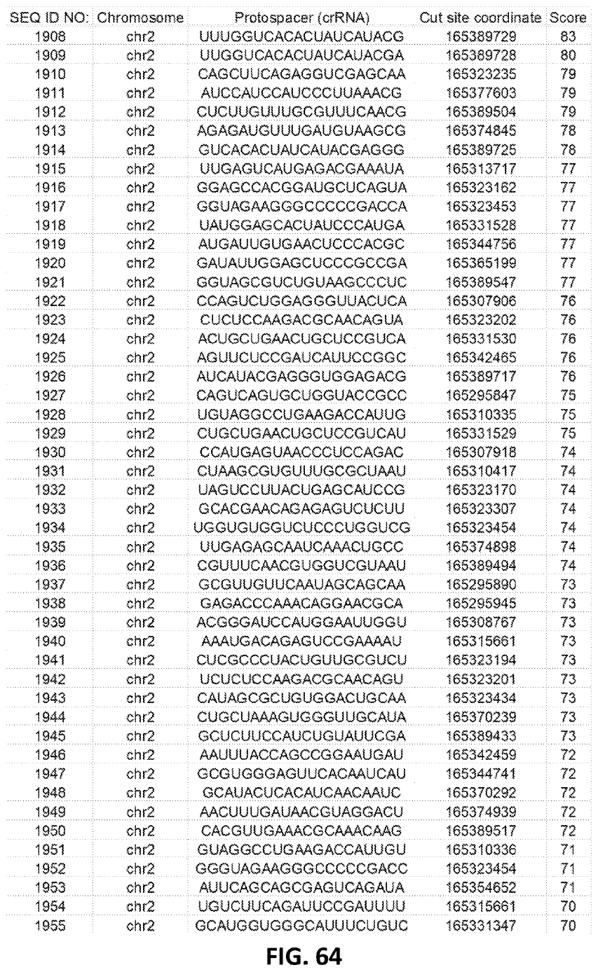

214. The pharmaceutical composition of claim 213, wherein the at least one guide RNA comprises a crRNA sequence selected from the group consisting of those sequences shown in FIG. 64 (SEQ ID NOS: 1908-1955).

215. The pharmaceutical composition of claim 213, wherein the at least one guide RNA comprises a crRNA sequence selected from the group consisting of SEQ ID NOS: 1908-1917.

216. The pharmaceutical composition of any one of claims 1-11, wherein the at least one guide RNA or a nucleic acid encoding at least one guide RNA targets a mammalian SCN3A gene.

217. The pharmaceutical composition of claim 216, wherein the mammalian SCN3A gene is a human SCN3A gene.

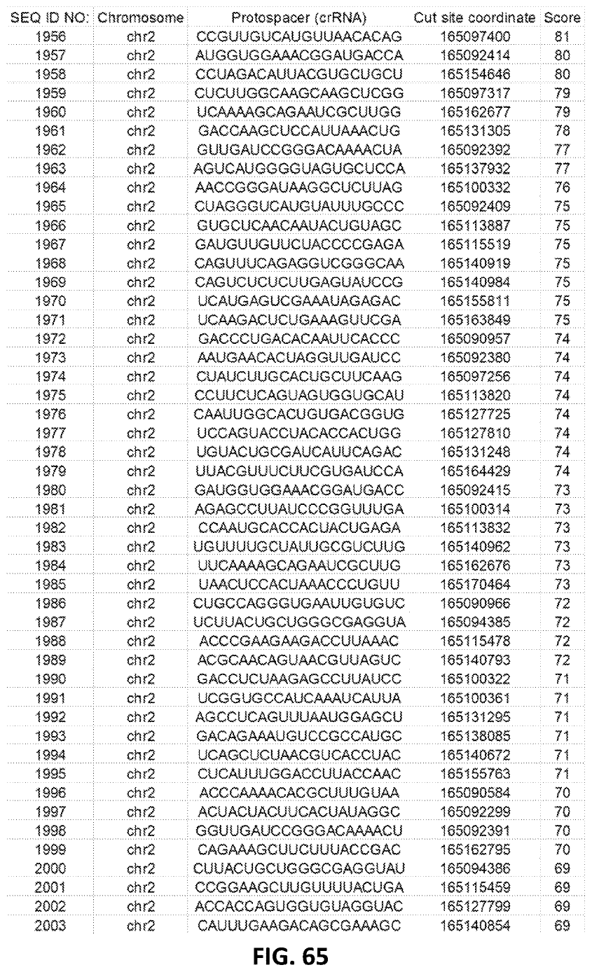

218. The pharmaceutical composition of claim 217, wherein the at least one guide RNA comprises a crRNA sequence selected from the group consisting of those sequences shown in FIG. 65 (SEQ ID NOS: 1956-2003).

219. The pharmaceutical composition of claim 217, wherein the at least one guide RNA comprises a crRNA sequence selected from the group consisting of SEQ ID NOS: 1956-1965.

220. The pharmaceutical composition of any one of claims 1-11, wherein the at least one guide RNA or a nucleic acid encoding at least one guide RNA targets a mammalian SCN4A gene.

221. The pharmaceutical composition of claim 220, wherein the mammalian SCN4A gene is a human SCN4A gene.

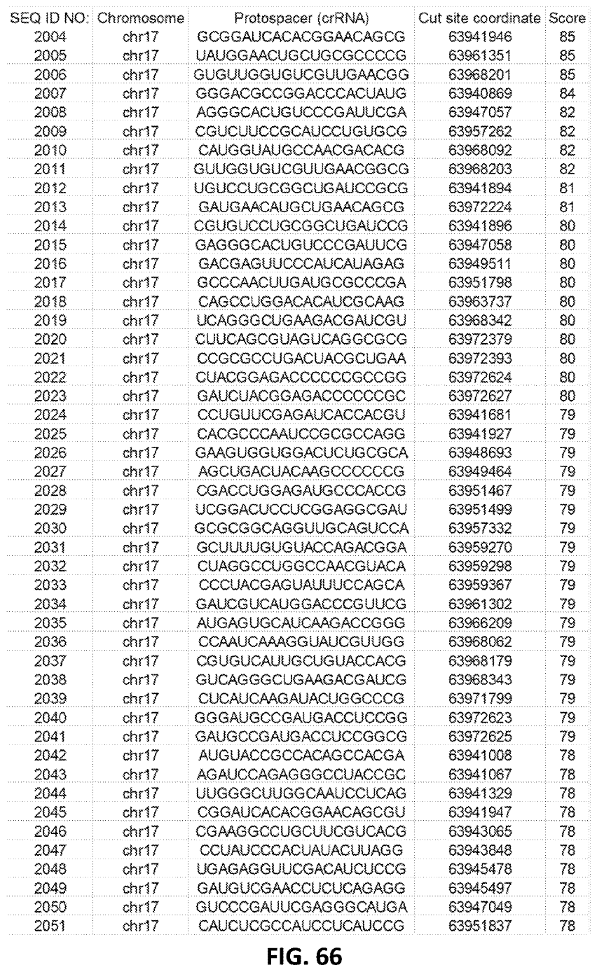

222. The pharmaceutical composition of claim 221, wherein the at least one guide RNA comprises a crRNA sequence selected from the group consisting of those sequences shown in FIG. 66 (SEQ ID NOS: 2004-2051).

223. The pharmaceutical composition of claim 221, wherein the at least one guide RNA comprises a crRNA sequence selected from the group consisting of SEQ ID NOS: 2004-2013.

224. The pharmaceutical composition of any one of claims 1-11, wherein the at least one guide RNA or a nucleic acid encoding at least one guide RNA targets a mammalian SCN5A gene.

225. The pharmaceutical composition of claim 224, wherein the mammalian SCN5A gene is a human SCN5A gene.

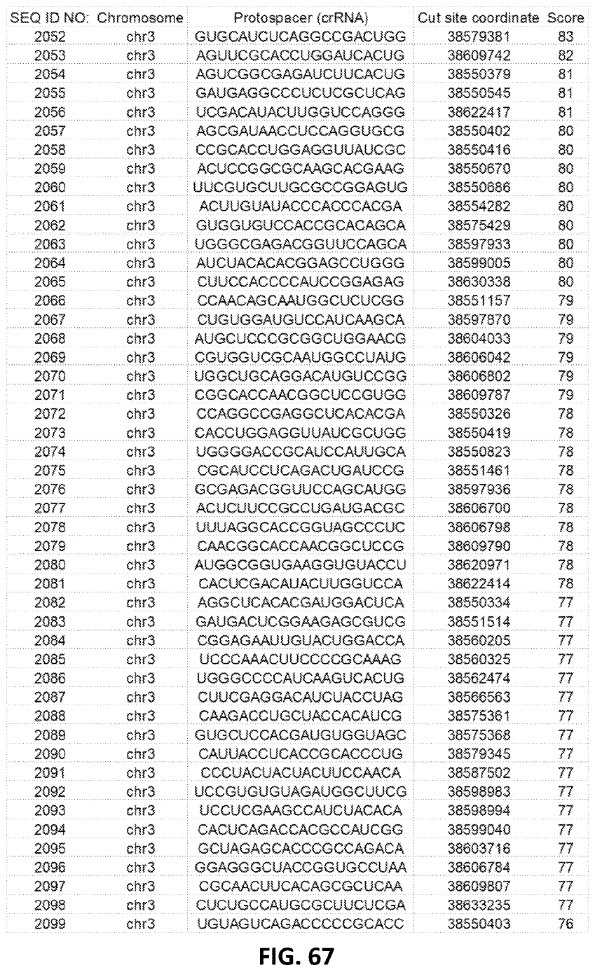

226. The pharmaceutical composition of claim 225, wherein the at least one guide RNA comprises a crRNA sequence selected from the group consisting of those sequences shown in FIG. 67 (SEQ ID NOS: 2052-2099).

227. The pharmaceutical composition of claim 225, wherein the at least one guide RNA comprises a crRNA sequence selected from the group consisting of SEQ ID NOS: 2052-2061.

228. The pharmaceutical composition of any one of claims 1-11, wherein the at least one guide RNA or a nucleic acid encoding at least one guide RNA targets a mammalian SCN8A gene.

229. The pharmaceutical composition of claim 228, wherein the mammalian SCN8A gene is a human SCN8A gene.

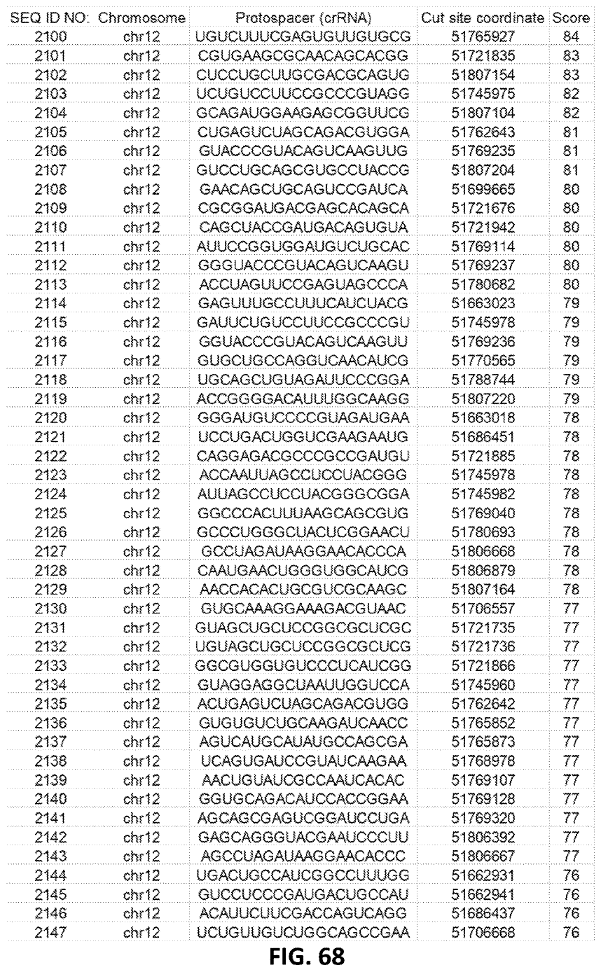

230. The pharmaceutical composition of claim 229, wherein the at least one guide RNA comprises a crRNA sequence selected from the group consisting of those sequences shown in FIG. 68 (SEQ ID NOS: 2100-2147).

231. The pharmaceutical composition of claim 229, wherein the at least one guide RNA comprises a crRNA sequence selected from the group consisting of SEQ ID NOS: 2100-2109.

232. The pharmaceutical composition of any one of claims 1-11, wherein the at least one guide RNA or a nucleic acid encoding at least one guide RNA targets a mammalian SCN9A gene.

233. The pharmaceutical composition of claim 232, wherein the mammalian SCN9A gene is a human SCN9A gene.

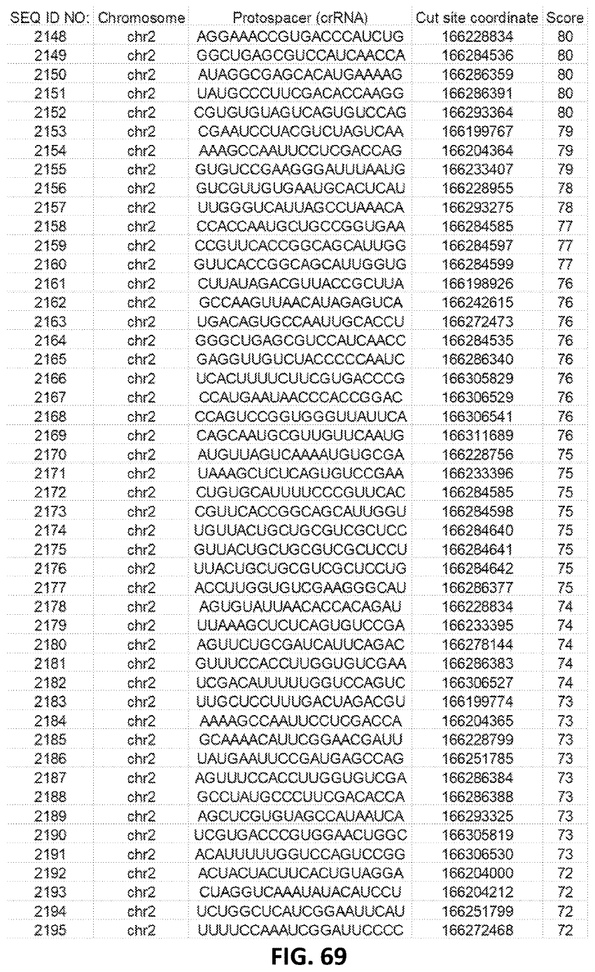

234. The pharmaceutical composition of claim 233, wherein the at least one guide RNA comprises a crRNA sequence selected from the group consisting of those sequences shown in FIG. 69 (SEQ ID NOS: 2148-2195).

235. The pharmaceutical composition of claim 233, wherein the at least one guide RNA comprises a crRNA sequence selected from the group consisting of SEQ ID NOS: 2148-2157.

236. The pharmaceutical composition of any one of claims 1-11, wherein the at least one guide RNA or a nucleic acid encoding at least one guide RNA targets a mammalian SCN10A gene.

237. The pharmaceutical composition of claim 236, wherein the mammalian SCN10A gene is a human SCN10A gene.

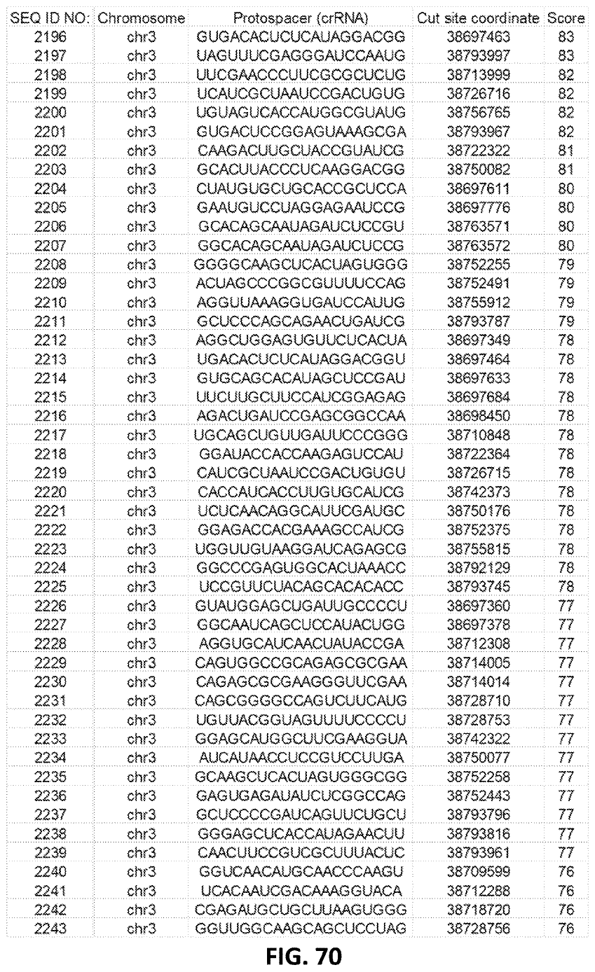

238. The pharmaceutical composition of claim 237, wherein the at least one guide RNA comprises a crRNA sequence selected from the group consisting of those sequences shown in FIG. 70 (SEQ ID NOS: 2196-2243).

239. The pharmaceutical composition of claim 237, wherein the at least one guide RNA comprises a crRNA sequence selected from the group consisting of SEQ ID NOS: 2196-2205.

240. The pharmaceutical composition of any one of claims 1-11, wherein the at least one guide RNA or a nucleic acid encoding at least one guide RNA targets a mammalian SCN11A gene.

241. The pharmaceutical composition of claim 240, wherein the mammalian SCN11A gene is a human SCN11A gene.

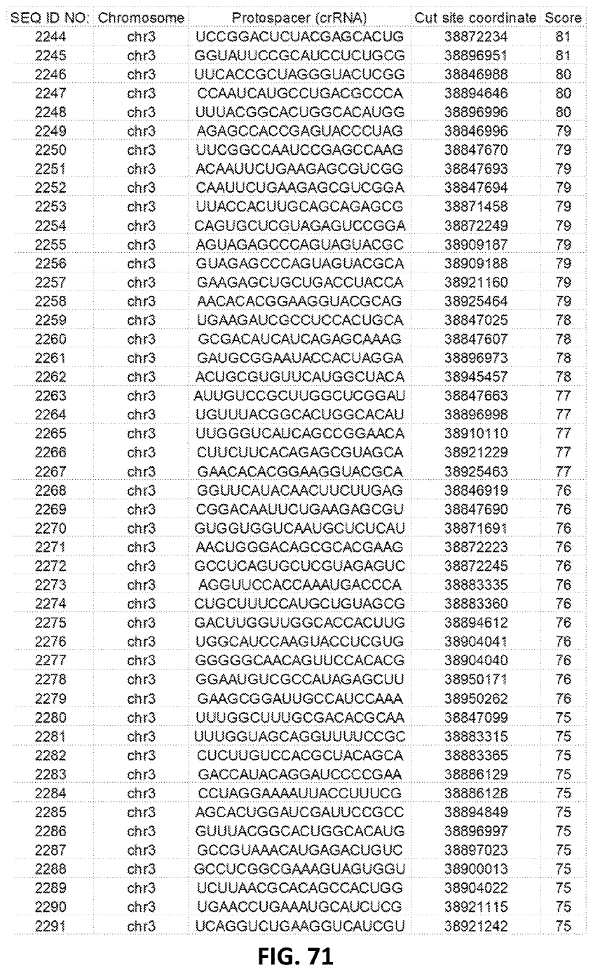

242. The pharmaceutical composition of claim 241, wherein the at least one guide RNA comprises a crRNA sequence selected from the group consisting of those sequences shown in FIG. 71 (SEQ ID NOS: 2244-2291).

243. The pharmaceutical composition of claim 241, wherein the at least one guide RNA comprises a crRNA sequence selected from the group consisting of SEQ ID NOS: 2244-2253.

244. The pharmaceutical composition of any one of claims 1-11, wherein the at least one guide RNA or a nucleic acid encoding at least one guide RNA targets a mammalian TAC1 gene.

245. The pharmaceutical composition of claim 244, wherein the mammalian TAC1 gene is a human TAC1 gene.

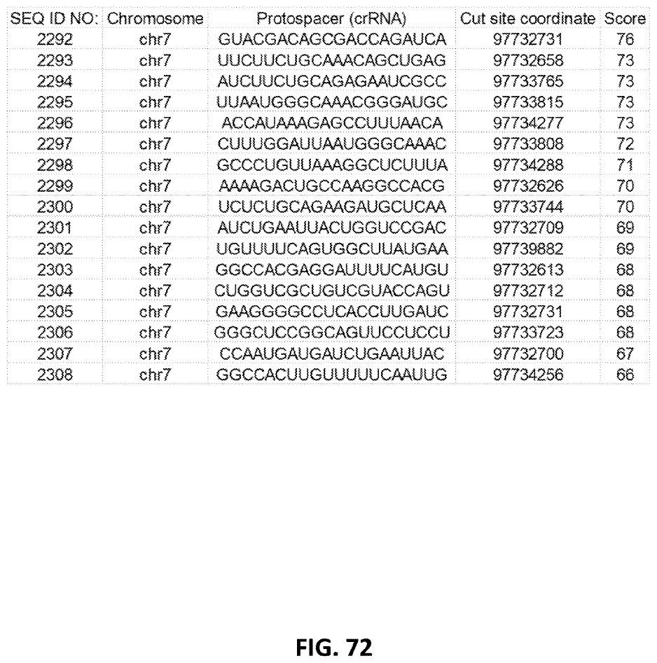

246. The pharmaceutical composition of claim 245, wherein the at least one guide RNA comprises a crRNA sequence selected from the group consisting of those sequences shown in FIG. 72 (SEQ ID NOS: 2292-2308).

247. The pharmaceutical composition of claim 245, wherein the at least one guide RNA comprises a crRNA sequence selected from the group consisting of SEQ ID NOS: 2292-2308.

248. The pharmaceutical composition of any one of claims 1-11, wherein the at least one guide RNA or a nucleic acid encoding at least one guide RNA targets a mammalian TACR1 gene.

249. The pharmaceutical composition of claim 248, wherein the mammalian TACR1 gene is a human TACR1 gene.

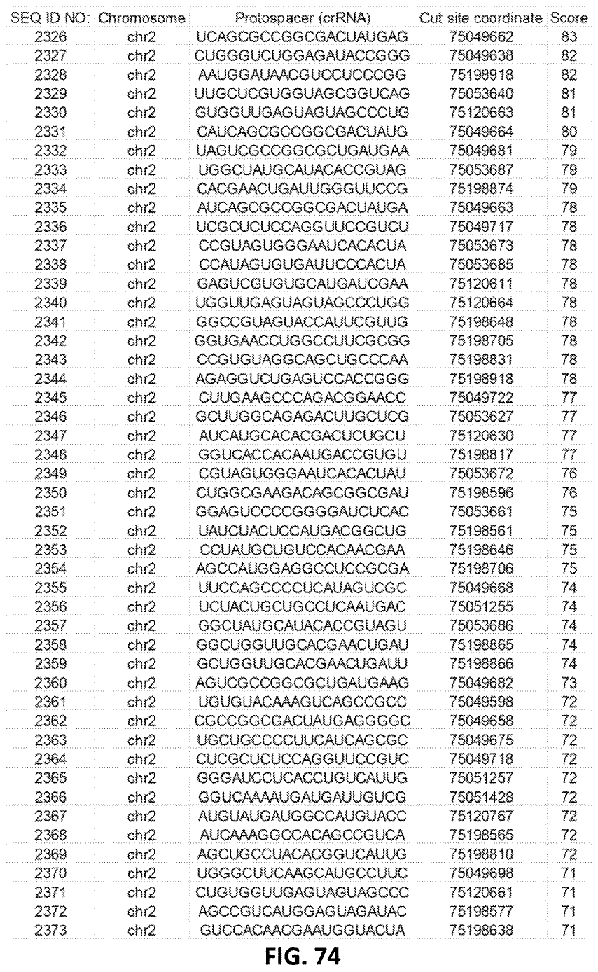

250. The pharmaceutical composition of claim 249, wherein the at least one guide RNA comprises a crRNA sequence selected from the group consisting of those sequences shown in FIG. 74 (SEQ ID NOS: 2326-2373).

251. The pharmaceutical composition of claim 249, wherein the at least one guide RNA comprises a crRNA sequence selected from the group consisting of SEQ ID NOS: 2326-2335.

252. The pharmaceutical composition of any one of claims 1-11, wherein the at least one guide RNA or a nucleic acid encoding at least one guide RNA targets a mammalian MRGPRX gene.

253. The pharmaceutical composition of claim 252, wherein the mammalian MRGPRX gene is a human MRGPRX gene.

254. The pharmaceutical composition of claim 253, wherein the at least one guide RNA comprises a crRNA sequence selected from the group consisting of those sequences shown in FIGS. $61a (SEQ ID NOS:$61b).

255. The pharmaceutical composition of claim 253, wherein the at least one guide RNA comprises a crRNA sequence selected from the group consisting of SEQ ID NOS:$61c.

256. The pharmaceutical composition of any one of claims 1-11, wherein the at least one guide RNA or a nucleic acid encoding at least one guide RNA targets a mammalian ATP1A1 gene.

257. The pharmaceutical composition of claim 256, wherein the mammalian ATP1A1 gene is a human ATP1A1 gene.

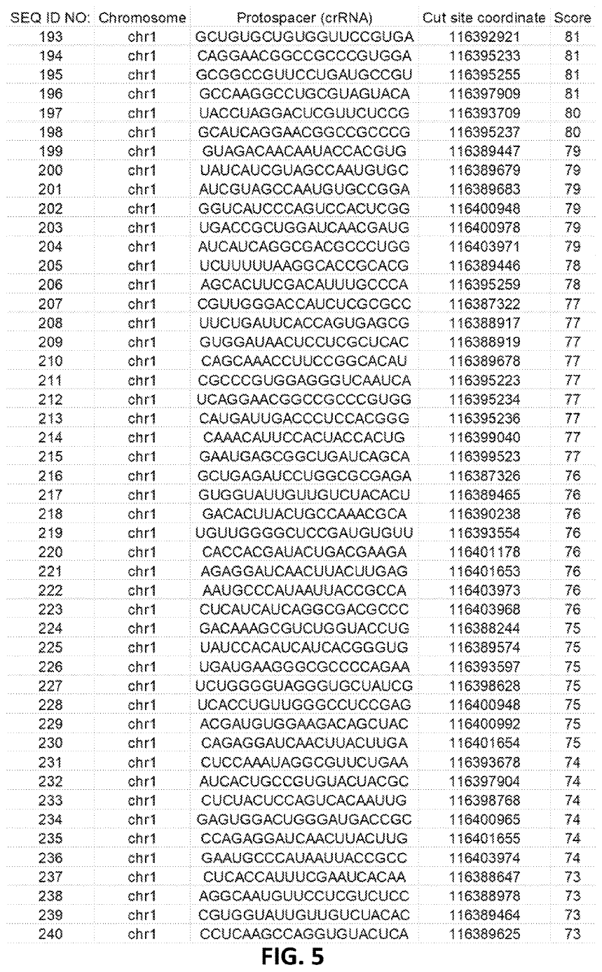

258. The pharmaceutical composition of claim 257, wherein the at least one guide RNA comprises a crRNA sequence selected from the group consisting of those sequences shown in FIG. 5 (SEQ ID NOS:193-240).

259. The pharmaceutical composition of claim 257, wherein the at least one guide RNA comprises a crRNA sequence selected from the group consisting of SEQ ID NOS:193-202.

260. The pharmaceutical composition of any one of claims 1-11, wherein the at least one guide RNA or a nucleic acid encoding at least one guide RNA targets a mammalian TACR2 gene.

261. The pharmaceutical composition of claim 260, wherein the mammalian TACR2 gene is a human TACR2 gene.

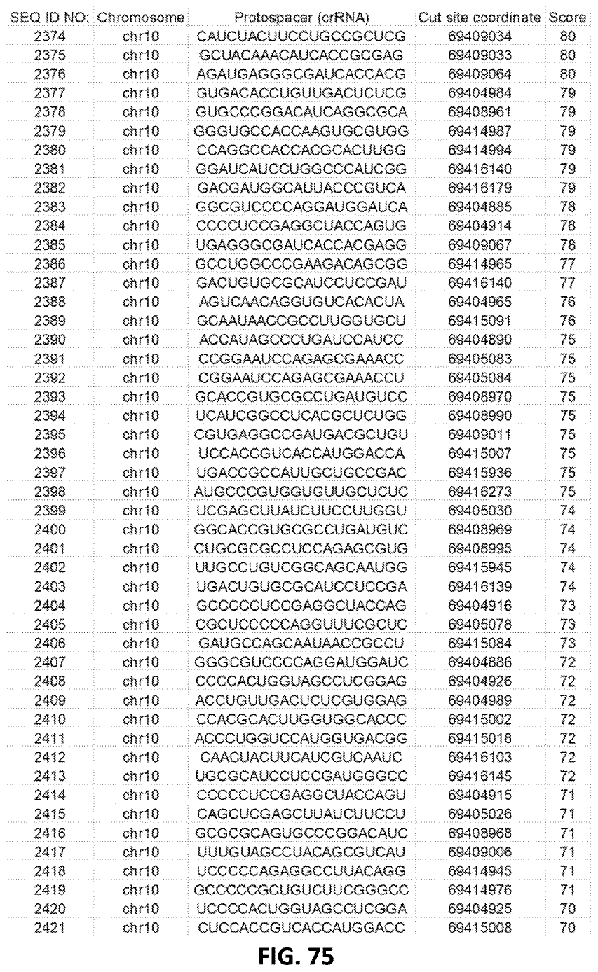

262. The pharmaceutical composition of claim 261, wherein the at least one guide RNA comprises a crRNA sequence selected from the group consisting of those sequences shown in FIG. 75 (SEQ ID NOS: 2374-2421).

263. The pharmaceutical composition of claim 261, wherein the at least one guide RNA comprises a crRNA sequence selected from the group consisting of SEQ ID NOS: 2374-2383.

264. The pharmaceutical composition of any one of claims 1-11, wherein the at least one guide RNA or a nucleic acid encoding at least one guide RNA targets a mammalian TAC3 gene.

265. The pharmaceutical composition of claim 264, wherein the mammalian TAC3 gene is a human TAC3 gene.

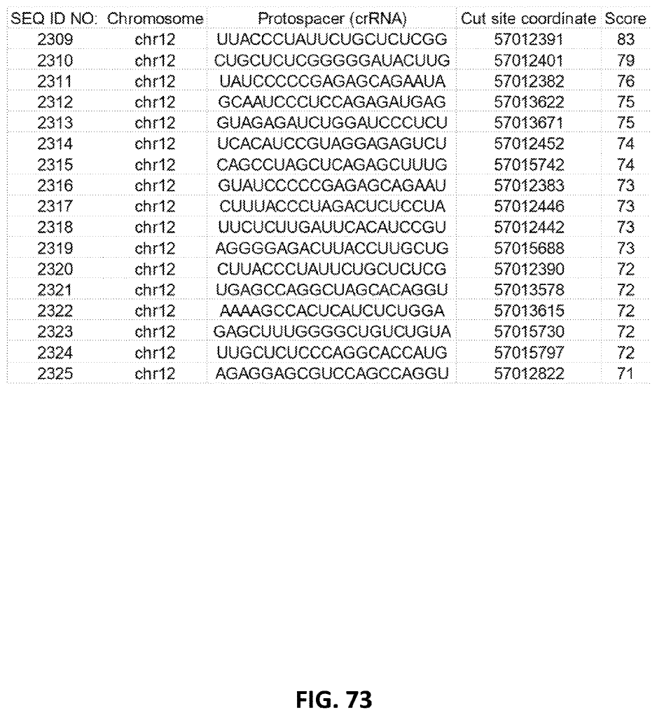

266. The pharmaceutical composition of claim 265, wherein the at least one guide RNA comprises a crRNA sequence selected from the group consisting of those sequences shown in FIG. 73 (SEQ ID NOS: 2309-2325).

267. The pharmaceutical composition of claim 265, wherein the at least one guide RNA comprises a crRNA sequence selected from the group consisting of SEQ ID NOS: 2309-2318.

268. The pharmaceutical composition of any one of claims 1-11, wherein the at least one guide RNA or a nucleic acid encoding at least one guide RNA targets a mammalian TACR3 gene.

269. The pharmaceutical composition of claim 268, wherein the mammalian TACR3 gene is a human TACR3 gene.

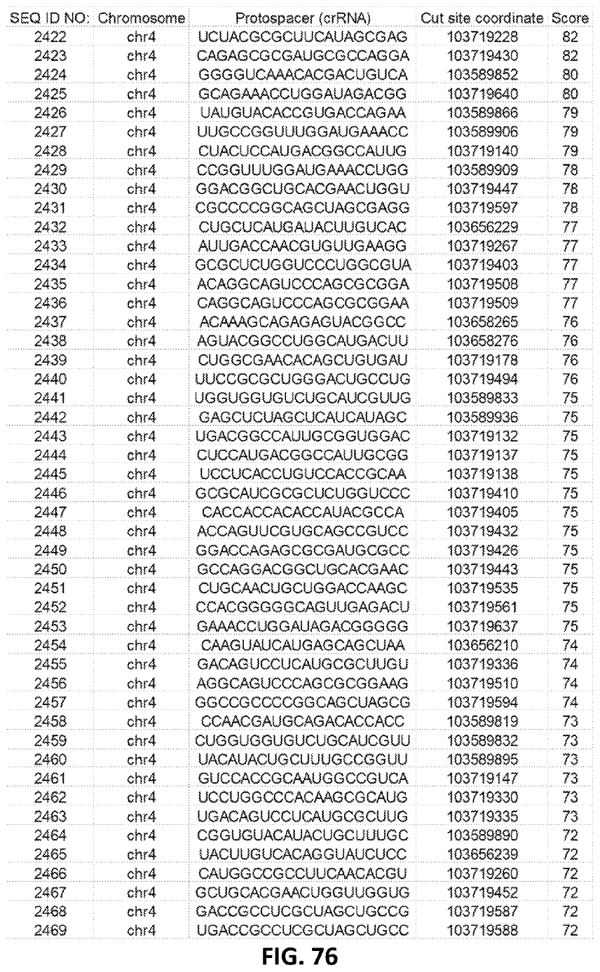

270. The pharmaceutical composition of claim 269, wherein the at least one guide RNA comprises a crRNA sequence selected from the group consisting of those sequences shown in FIG. 76 (SEQ ID NOS: 2422-2469).

271. The pharmaceutical composition of claim 269, wherein the at least one guide RNA comprises a crRNA sequence selected from the group consisting of SEQ ID NOS: 2422-2431.

272. The pharmaceutical composition of any one of claims 1-11, wherein the at least one guide RNA or a nucleic acid encoding at least one guide RNA targets a mammalian CALCA gene.

273. The pharmaceutical composition of claim 272, wherein the mammalian CALCA gene is a human CALCA gene.

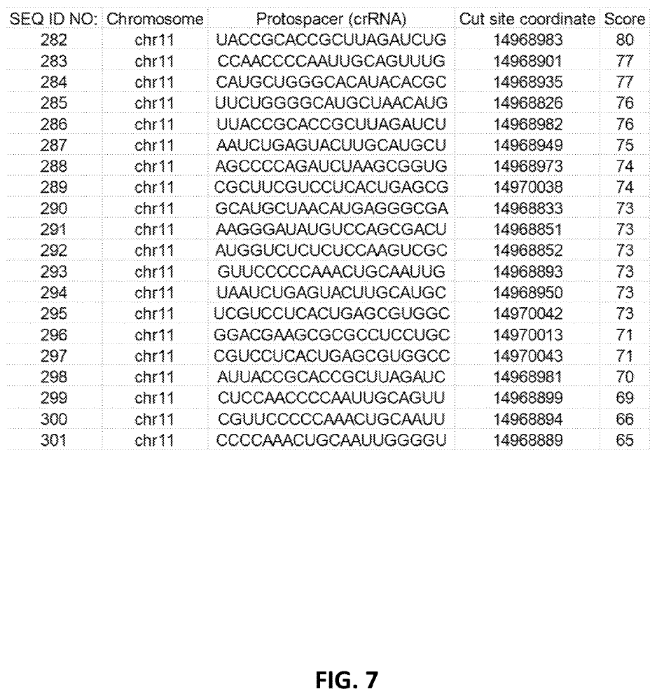

274. The pharmaceutical composition of claim 273, wherein the at least one guide RNA comprises a crRNA sequence selected from the group consisting of those sequences shown in FIG. 7 (SEQ ID NOS:282-301).

275. The pharmaceutical composition of claim 273, wherein the at least one guide RNA comprises a crRNA sequence selected from the group consisting of SEQ ID NOS:282-291.

276. The pharmaceutical composition of any one of claims 1-11, wherein the at least one guide RNA or a nucleic acid encoding at least one guide RNA targets a mammalian CALCB gene.

277. The pharmaceutical composition of claim 276, wherein the mammalian CALCB gene is a human CALCB gene.

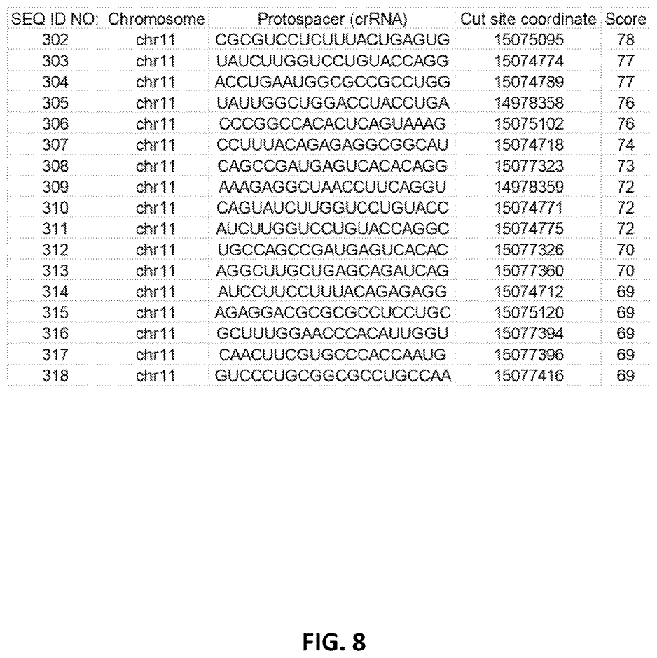

278. The pharmaceutical composition of claim 277, wherein the at least one guide RNA comprises a crRNA sequence selected from the group consisting of those sequences shown in FIG. 8 (SEQ ID NOS:302-318).

279. The pharmaceutical composition of claim 277, wherein the at least one guide RNA comprises a crRNA sequence selected from the group consisting of SEQ ID NOS:302-312.

280. The pharmaceutical composition of any one of claims 1-11, wherein the at least one guide RNA or a nucleic acid encoding at least one guide RNA targets a mammalian RAMP1 gene.

281. The pharmaceutical composition of claim 280, wherein the mammalian RAMP1 gene is a human RAMP1 gene.

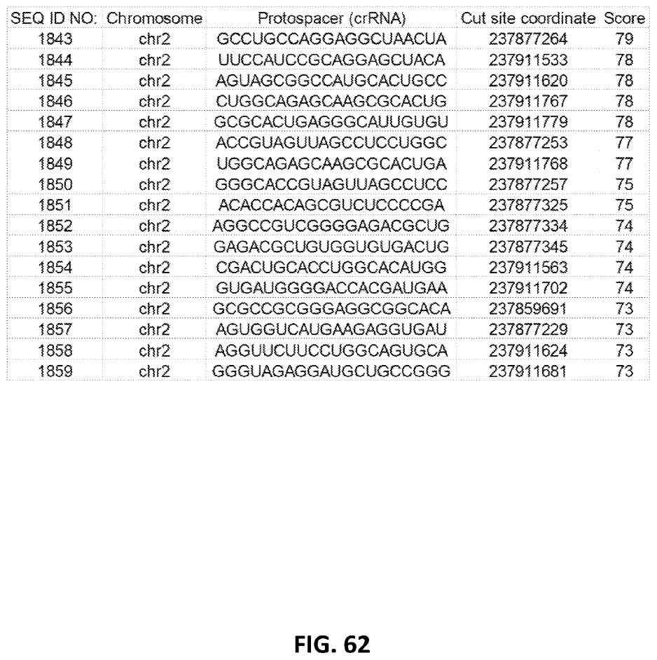

282. The pharmaceutical composition of claim 281, wherein the at least one guide RNA comprises a crRNA sequence selected from the group consisting of those sequences shown in FIG. 62 (SEQ ID NOS: 1843-1859).

283. The pharmaceutical composition of claim 281, wherein the at least one guide RNA comprises a crRNA sequence selected from the group consisting of SEQ ID NOS: 1843-1852.

284. The pharmaceutical composition of any one of claims 1-11, wherein the at least one guide RNA or a nucleic acid encoding at least one guide RNA targets a mammalian CALCRL gene.

285. The pharmaceutical composition of claim 284, wherein the mammalian CALCRL gene is a human CALCRL gene.

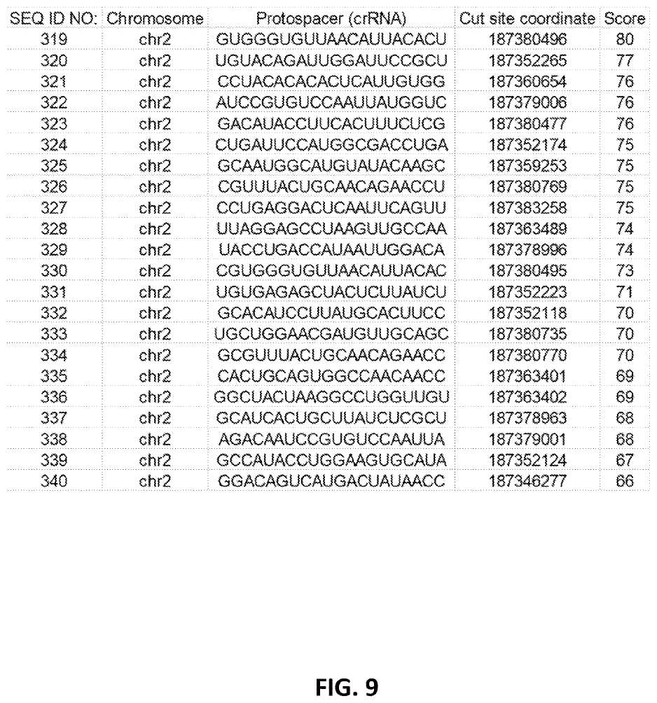

286. The pharmaceutical composition of claim 285, wherein the at least one guide RNA comprises a crRNA sequence selected from the group consisting of those sequences shown in FIG. 9 (SEQ ID NOS:319-340).

287. The pharmaceutical composition of claim 285, wherein the at least one guide RNA comprises a crRNA sequence selected from the group consisting of SEQ ID NOS:319-328.

288. The pharmaceutical composition of any one of claims 1-11, wherein the at least one guide RNA or a nucleic acid encoding at least one guide RNA targets a mammalian ADM gene.

289. The pharmaceutical composition of claim 284, wherein the mammalian ADM gene is a human ADM gene.

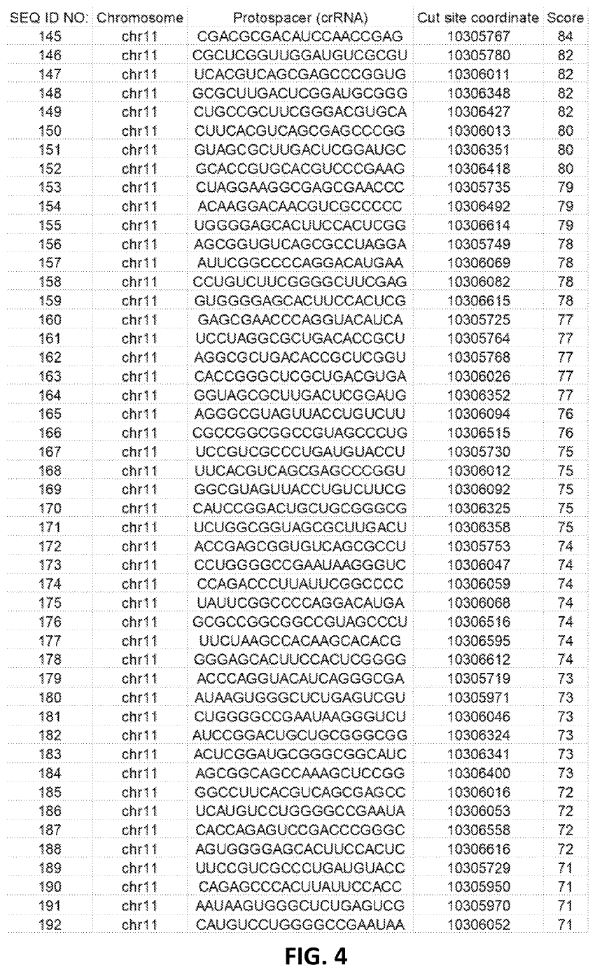

290. The pharmaceutical composition of claim 285, wherein the at least one guide RNA comprises a crRNA sequence selected from the group consisting of those sequences shown in FIG. 4 (SEQ ID NOS:145-192).

291. The pharmaceutical composition of claim 285, wherein the at least one guide RNA comprises a crRNA sequence selected from the group consisting of SEQ ID NOS: 145-1542.

292. The pharmaceutical composition of any one of claims 1-11, wherein the at least one guide RNA or a nucleic acid encoding at least one guide RNA targets a mammalian CRCP gene.

293. The pharmaceutical composition of claim 292, wherein the mammalian CRCP gene is a human CRCP gene.

294. The pharmaceutical composition of claim 293, wherein the at least one guide RNA comprises a crRNA sequence selected from the group consisting of those sequences shown in FIG. 17 (SEQ ID NOS: 518-534).

295. The pharmaceutical composition of claim 293, wherein the at least one guide RNA comprises a crRNA sequence selected from the group consisting of SEQ ID NOS: 518-527.

296. The pharmaceutical composition of any one of claims 1-11, wherein the at least one guide RNA or a nucleic acid encoding at least one guide RNA targets a mammalian NGF gene.

297. The pharmaceutical composition of claim 296, wherein the mammalian NGF gene is a human NGF gene.

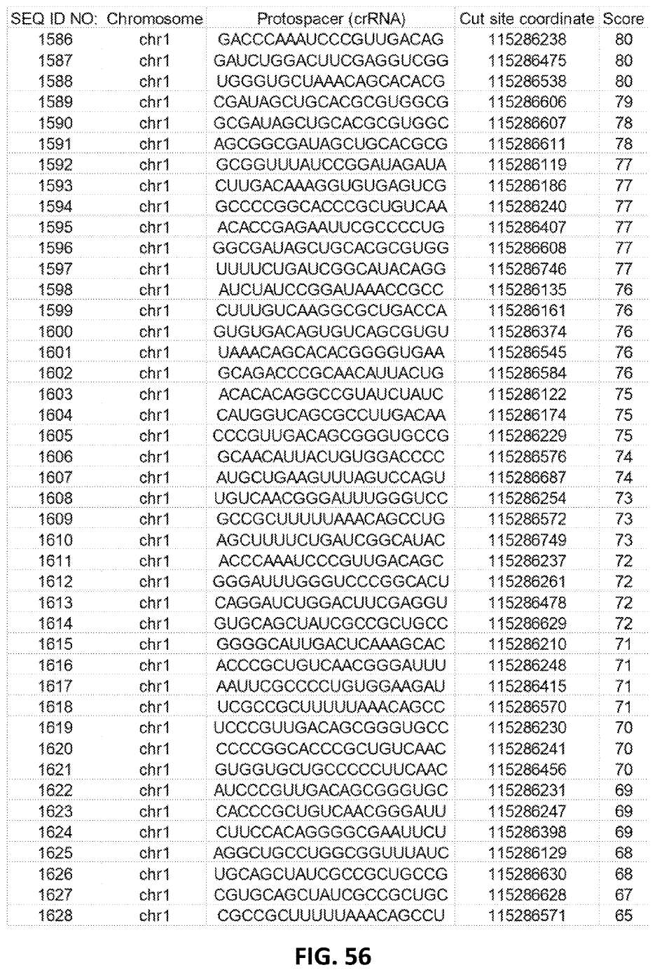

298. The pharmaceutical composition of claim 297, wherein the at least one guide RNA comprises a crRNA sequence selected from the group consisting of those sequences shown in FIG. 56 (SEQ ID NOS: 1586-1628).

299. The pharmaceutical composition of claim 297, wherein the at least one guide RNA comprises a crRNA sequence selected from the group consisting of SEQ ID NOS: 1586-1595.

300. The pharmaceutical composition of any one of claims 1-11, wherein the at least one guide RNA or a nucleic acid encoding at least one guide RNA targets a mammalian NGFR gene.

301. The pharmaceutical composition of claim 300, wherein the mammalian NGFR gene is a human NGFR gene.

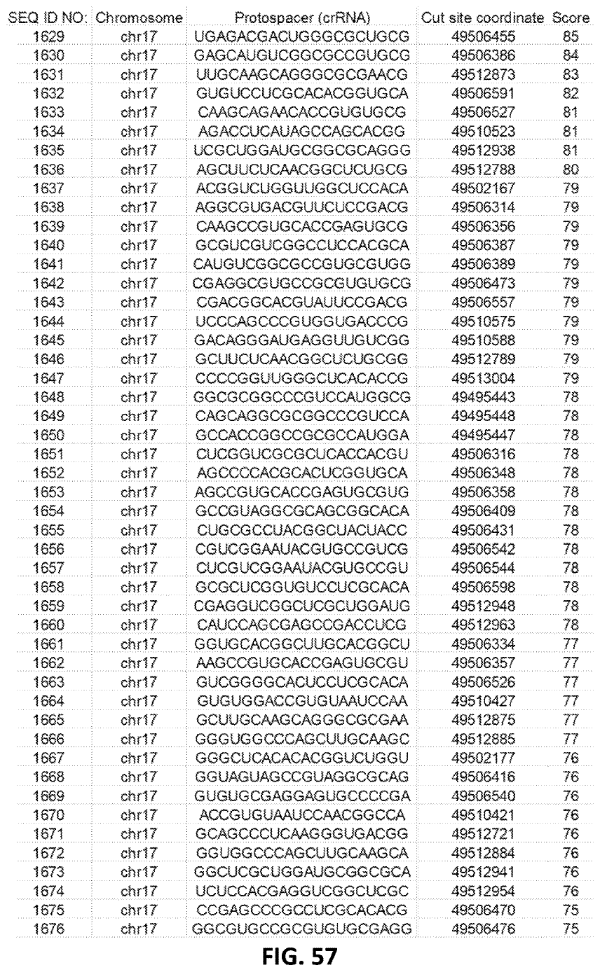

302. The pharmaceutical composition of claim 301, wherein the at least one guide RNA comprises a crRNA sequence selected from the group consisting of those sequences shown in FIG. 57 (SEQ ID NOS: 1629-1676).

303. The pharmaceutical composition of claim 301, wherein the at least one guide RNA comprises a crRNA sequence selected from the group consisting of SEQ ID NOS: 1629-1638.

304. The pharmaceutical composition of any one of claims 1-11, wherein the at least one guide RNA or a nucleic acid encoding at least one guide RNA targets a mammalian NTRK1 gene.

305. The pharmaceutical composition of claim 304, wherein the mammalian NTRK11 gene is a human NTRK1 gene.

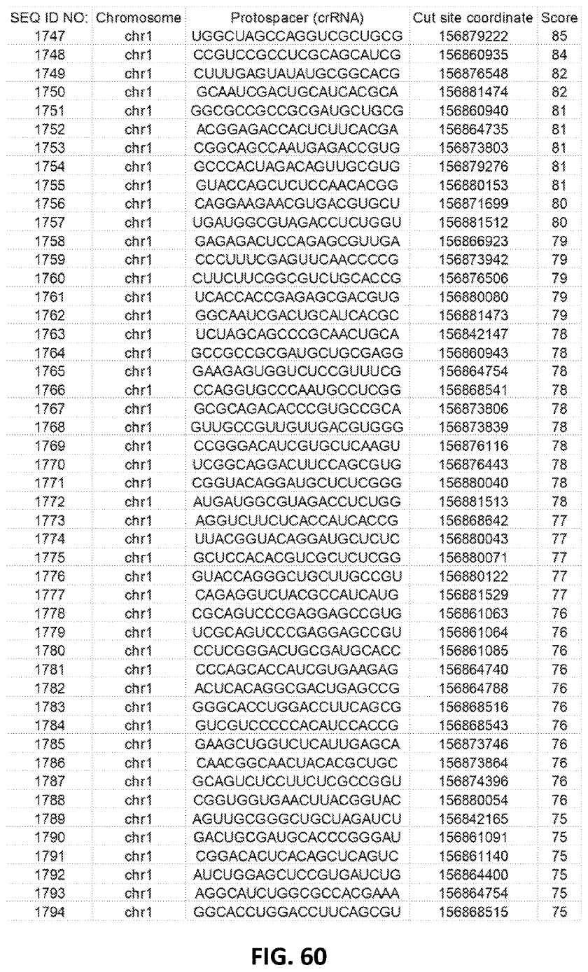

306. The pharmaceutical composition of claim 305, wherein the at least one guide RNA comprises a crRNA sequence selected from the group consisting of those sequences shown in FIG. 60 (SEQ ID NOS: 1747-1794).

307. The pharmaceutical composition of claim 305, wherein the at least one guide RNA comprises a crRNA sequence selected from the group consisting of SEQ ID NOS: 1747-1756.

308. The pharmaceutical composition of any one of claims 1-11, wherein the at least one guide RNA or a nucleic acid encoding at least one guide RNA targets a mammalian NTF3 gene.

309. The pharmaceutical composition of claim 308, wherein the mammalian NTF3 gene is a human NTF3 gene.

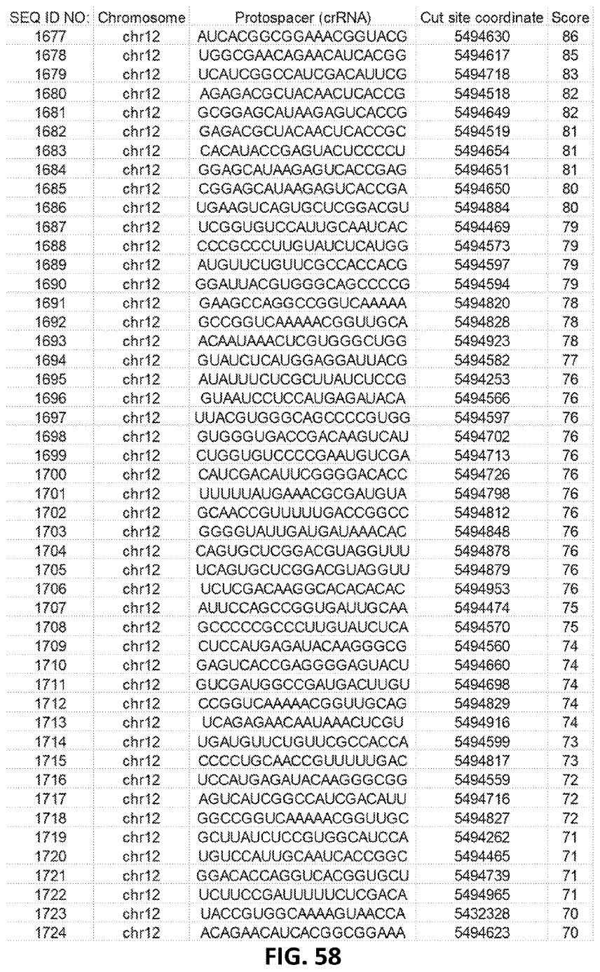

310. The pharmaceutical composition of claim 309, wherein the at least one guide RNA comprises a crRNA sequence selected from the group consisting of those sequences shown in FIG. 58 (SEQ ID NOS: 1677-1724).

311. The pharmaceutical composition of claim 309, wherein the at least one guide RNA comprises a crRNA sequence selected from the group consisting of SEQ ID NOS: 1677-1686.

312. The pharmaceutical composition of any one of claims 1-11, wherein the at least one guide RNA or a nucleic acid encoding at least one guide RNA targets a mammalian NTF4 gene.

313. The pharmaceutical composition of claim 312, wherein the mammalian NTF4 gene is a human NTF4 gene.

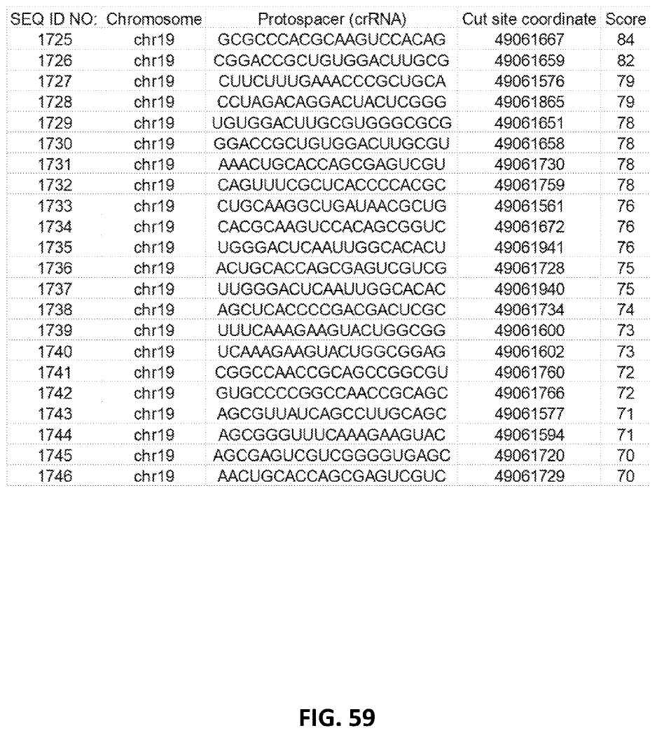

314. The pharmaceutical composition of claim 313, wherein the at least one guide RNA comprises a crRNA sequence selected from the group consisting of those sequences shown in FIG. 59 (SEQ ID NOS: 1725-1746).

315. The pharmaceutical composition of claim 313, wherein the at least one guide RNA comprises a crRNA sequence selected from the group consisting of SEQ ID NOS: 1725-1734.

316. The pharmaceutical composition of any one of claims 1-11, wherein the at least one guide RNA or a nucleic acid encoding at least one guide RNA targets a mammalian NTRK2 gene.

317. The pharmaceutical composition of claim 316, wherein the mammalian NTRK2 gene is a human NTRK2 gene.

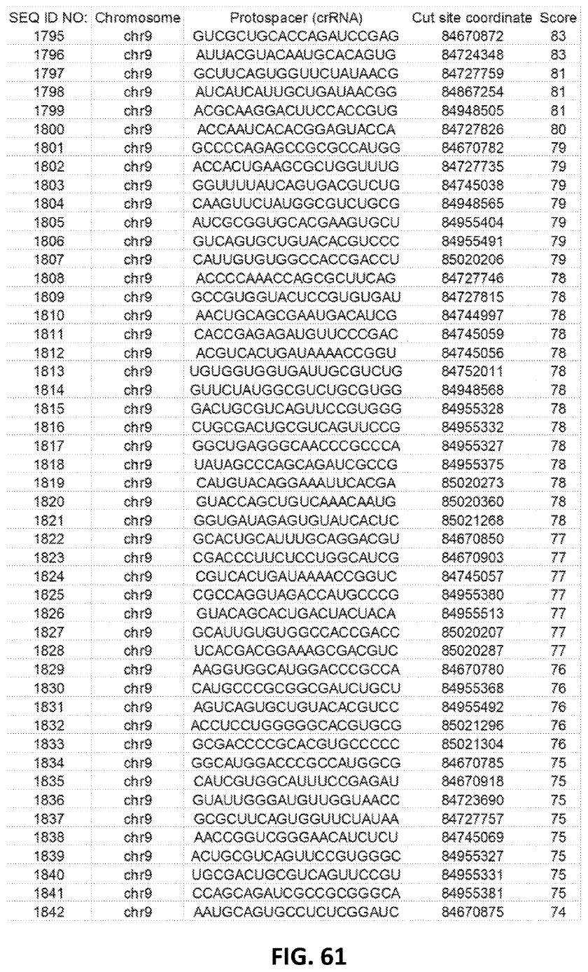

318. The pharmaceutical composition of claim 317, wherein the at least one guide RNA comprises a crRNA sequence selected from the group consisting of those sequences shown in FIG. 61 (SEQ ID NOS: 1795-1842).

319. The pharmaceutical composition of claim 317, wherein the at least one guide RNA comprises a crRNA sequence selected from the group consisting of SEQ ID NOS: 1795-1804.

320. The pharmaceutical composition of any one of claims 1-11, wherein the at least one guide RNA or a nucleic acid encoding at least one guide RNA targets a mammalian IL1RAP gene.

321. The pharmaceutical composition of claim 320, wherein the mammalian IL1RAP gene is a human IL1RAP gene.

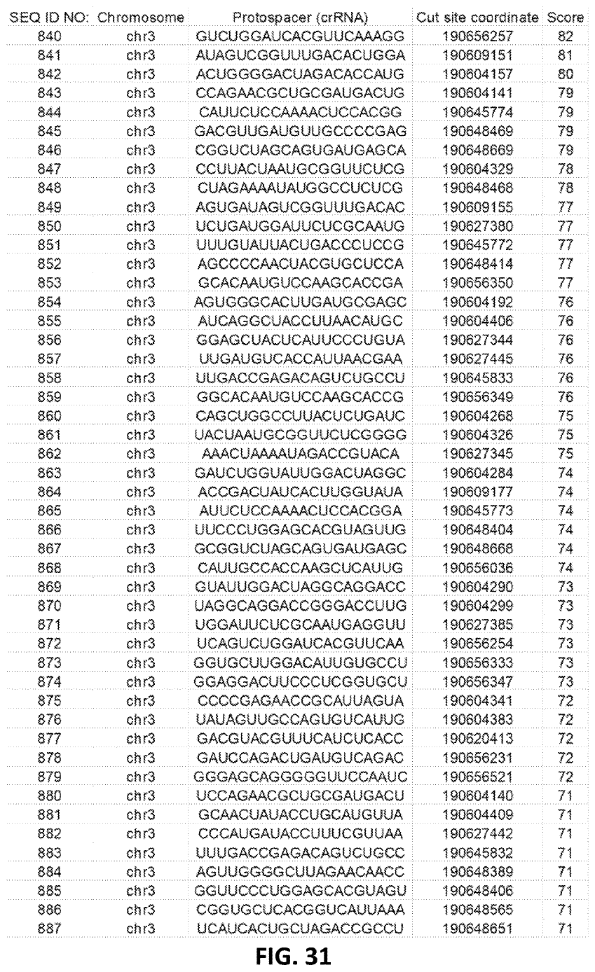

322. The pharmaceutical composition of claim 321, wherein the at least one guide RNA comprises a crRNA sequence selected from the group consisting of those sequences shown in FIG. 31 (SEQ ID NOS: 840-887).

323. The pharmaceutical composition of claim 321, wherein the at least one guide RNA comprises a crRNA sequence selected from the group consisting of SEQ ID NOS: 840-849.

324. The pharmaceutical composition of any one of claims 1-11, wherein the at least one guide RNA or a nucleic acid encoding at least one guide RNA targets a mammalian IL1α gene.

325. The pharmaceutical composition of claim 324, wherein the mammalian IL1α gene is a human IL1α gene.

326. The pharmaceutical composition of claim 325, wherein the at least one guide RNA comprises a crRNA sequence selected from the group consisting of those sequences shown in FIG. $80a (SEQ ID NOS:$80b).

327. The pharmaceutical composition of claim 325, wherein the at least one guide RNA comprises a crRNA sequence selected from the group consisting of SEQ ID NOS:$80c.

328. The pharmaceutical composition of any one of claims 1-11, wherein the at least one guide RNA or a nucleic acid encoding at least one guide RNA targets a mammalian IL1(gene.

329. The pharmaceutical composition of claim 328, wherein the mammalian IL1P gene is a human IL1I3 gene.

330. The pharmaceutical composition of claim 329, wherein the at least one guide RNA comprises a crRNA sequence selected from the group consisting of those sequences shown in FIG. $81a (SEQ ID NOS:$81b).

331. The pharmaceutical composition of claim 329, wherein the at least one guide RNA comprises a crRNA sequence selected from the group consisting of SEQ ID NOS:$81c.

332. The pharmaceutical composition of any one of claims 1-11, wherein the at least one guide RNA or a nucleic acid encoding at least one guide RNA targets a mammalian BDNF gene.

333. The pharmaceutical composition of claim 332, wherein the mammalian BDNF gene is a human BDNF gene.

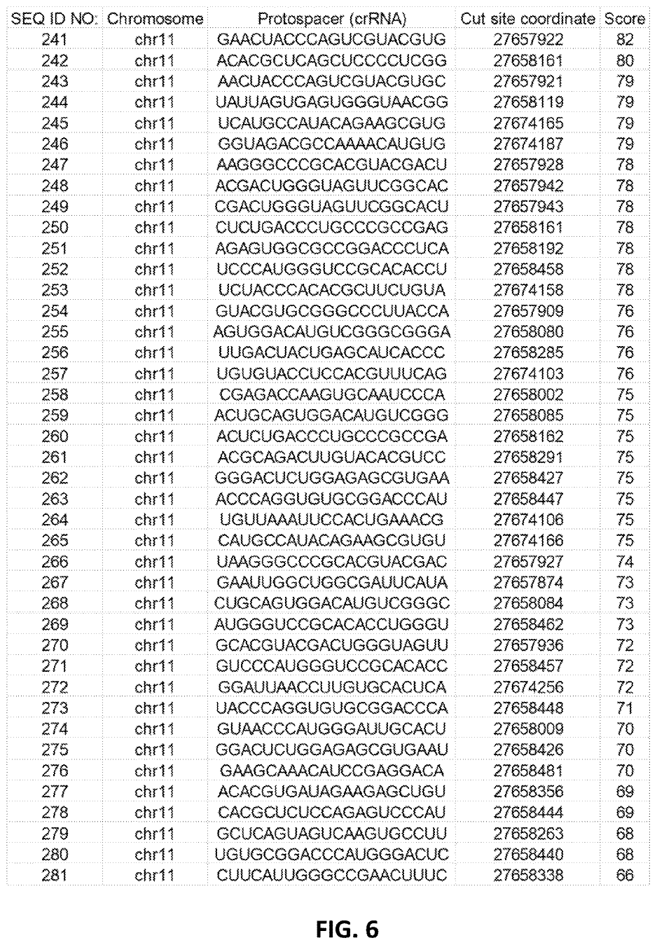

334. The pharmaceutical composition of claim 333, wherein the at least one guide RNA comprises a crRNA sequence selected from the group consisting of those sequences shown in FIG. 6 (SEQ ID NOS:241-281).

335. The pharmaceutical composition of claim 333, wherein the at least one guide RNA comprises a crRNA sequence selected from the group consisting of SEQ ID NOS:241-250.

336. [IL6ST] The pharmaceutical composition of any one of claims 1-11, wherein the at least one guide RNA or a nucleic acid encoding at least one guide RNA targets a mammalian IL6ST gene.

337. The pharmaceutical composition of claim 336, wherein the mammalian BDNF gene is a human IL6ST gene.

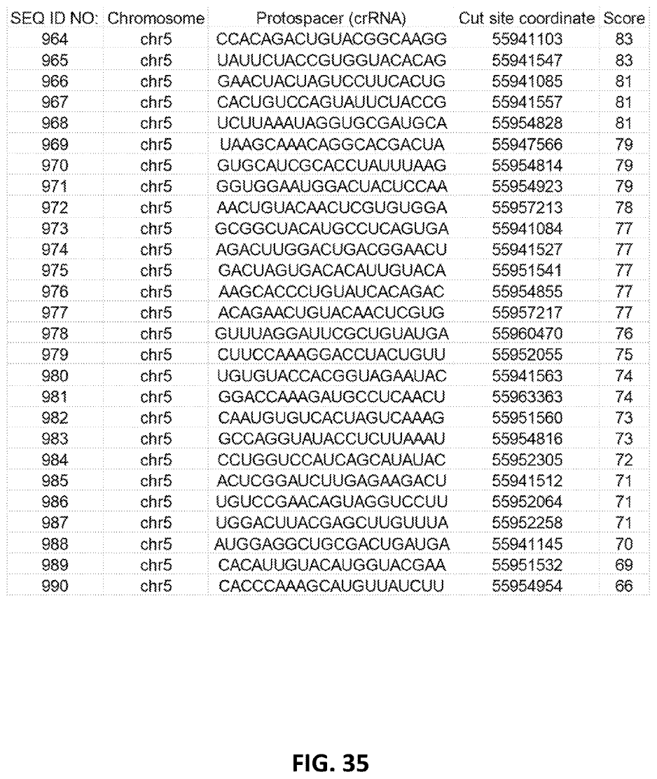

338. The pharmaceutical composition of claim 336, wherein the at least one guide RNA comprises a crRNA sequence selected from the group consisting of those sequences shown in FIG. 35 (SEQ ID NOS: 964-990).

339. The pharmaceutical composition of claim 336, wherein the at least one guide RNA comprises a crRNA sequence selected from the group consisting of SEQ ID NOS: 964-973.

340. The pharmaceutical composition of any one of claims 16-19, 116-123, 136-143, 148-151, 156-163, 168-175, 180-187, 192-195, 200-207, 248-255, 260-263, 268-271, 280-287, 300-307, and 316-323, wherein the targeted gene is a transmembrane receptor and the at least one guide RNA targets a portion of the targeted gene encoding a transmembrane spanning portion of the transmembrane receptor.

341. The pharmaceutical composition of any one of claims 16-19, 116-123, 136-143, 148-151, 156-163, 168-175, 180-187, 192-195, 200-207, 248-255, 260-263, 268-271, 280-287, 300-307, and 316-323, wherein the targeted gene is a transmembrane receptor and the at least one guide RNA targets a portion of the targeted gene encoding a cytoplasmic portion of the transmembrane receptor.

342. The pharmaceutical composition of any one of claims 1-341, wherein the RNA-guided nuclease or a nucleic acid encoding an RNA-guided nuclease is the RNA-guided nuclease.

343. The pharmaceutical composition of any one of claims 1-341, wherein the RNA-guided nuclease or a nucleic acid encoding an RNA-guided nuclease is DNA encoding the RNA-guided nuclease.

344. The pharmaceutical composition of any one of claims 1-341, wherein the RNA-guided nuclease or a nucleic acid encoding an RNA-guided nuclease is mRNA encoding the RNA-guided nuclease.

345. The pharmaceutical composition of any one of claims 1-344, wherein the RNA-guided nuclease is a Cas protein.

346. The pharmaceutical composition of claim 345, wherein the Cas protein is a Cas9 protein.

347. The pharmaceutical composition of claim 345, wherein the Cas9 protein is an S. pyogenes Cas9 polypeptide.

348. The pharmaceutical composition of claim 347, wherein the S. pyogenes Cas9 polypeptide comprises an R691A amino acid substitution.

349. The pharmaceutical composition of claim 347, wherein the S. pyogenes Cas9 polypeptide comprises K848A, K1003A, and R1060A amino acid substitutions.

350. The pharmaceutical composition of claim 347, wherein the S. pyogenes Cas9 polypeptide comprises a set of one or more amino acid substitutions selected from the group consisting of D10A, H840A, D1135E, D1135E, K810A/K1003A/R1060A, K848A/K1003A/R1060A, N497A/R661A/Q695A/Q926A, N692NA/M694A/Q695A/H698A, R691A, E108G/S217A/A262T, S409I/E480K/E543D/M694I/E1219V, F539S/M763I/K890N, and M495V/Y515N/K526E/R661Q.

351. The pharmaceutical composition of any one of claims 1-350, wherein the at least one guide RNA or a nucleic acid encoding at least one guide RNA is the at least one guide RNA.

352. The pharmaceutical composition of any one of claims 1-350, wherein the at least one guide RNA or a nucleic acid encoding at least one guide RNA is DNA encoding the at least one guide RNA.

353. The pharmaceutical composition of any one of claims 1-350, comprising a nucleic acid encoding both the RNA-guided nuclease and the at least one guide RNA.

354. The pharmaceutical composition of any one of claims 1-350, wherein the at least one guide RNA is a single guide RNA (sgRNA).

355. The pharmaceutical composition of any one of claims 1-354, wherein the composition comprises one or more viral vectors collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

356. The pharmaceutical composition of claim 355, wherein the one of more viral vectors comprise a recombinant virus selected from a retrovirus, an adenovirus, an adeno-associated virus, a lentivirus, and a herpes simplex virus-1.

357. The pharmaceutical composition of claim 355, wherein the one of more viral vectors comprise a recombinant adeno-associated virus (AAV).

358. The pharmaceutical composition of claim 357, wherein the recombinant AAV is of serotype 5 (AAV5).

359. The pharmaceutical composition of claim 357, wherein the recombinant AAV is of serotype 6 (AAV6).

360. The pharmaceutical composition of any one of claims 1-354, wherein the composition comprises one or more lipid nanoparticles (LNP) collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

361. The pharmaceutical composition of claim 360, wherein the one or more LNP comprises:
a first plurality of LNP encapsulating the RNA-guided nuclease or a nucleic acid encoding an RNA-guided nuclease; and
a second plurality of LNP encapsulating the at least one guide RNA or a nucleic acid encoding at least one guide RNA.

362. The pharmaceutical composition of claim 360, wherein the one or more LNP comprises a plurality of LNP encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

363. The pharmaceutical composition of any one of claims 360-362, wherein the one or more LNP comprises a component selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)--octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and a mixture thereof.

364. The pharmaceutical composition of any one of claims 360-363, wherein the LNP comprises a component selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and a mixture thereof.

365. The pharmaceutical composition of any one of claims 360-364, wherein the LNP comprises a component selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA, a PEG-DSPE lipid, and a mixture thereof.

366. The pharmaceutical composition of any one of claims 360-365, wherein the LNP comprises a component selected from the group consisting of a cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9,11-dehydroergosterol, tomatidine, tomatine, α-tocopherol, and a mixture thereof.

367. The pharmaceutical composition of any one of claims 1-354, wherein the composition comprises one or more liposomes collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

368. The pharmaceutical composition of claim 367, wherein the one or more liposomes comprises:
a first plurality of liposomes encapsulating the RNA-guided nuclease or a nucleic acid encoding an RNA-guided nuclease; and
a second plurality of liposomes encapsulating the at least one guide RNA or a nucleic acid encoding at least one guide RNA.

369. The pharmaceutical composition of claim 367, wherein the one or more liposomes comprises a plurality of liposomes encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

370. The pharmaceutical composition of any one of claims 1-354, wherein the composition comprises one or more virus-like particles collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

371. The pharmaceutical composition of claim 370, wherein the one or more virus-like particles comprises:

372. a first plurality of virus-like particles encapsulating the RNA-guided nuclease or a nucleic acid encoding an RNA-guided nuclease; and

373. a second plurality of virus-like particles encapsulating the at least one guide RNA or a nucleic acid encoding at least one guide RNA.

374. The pharmaceutical composition of claim 370, wherein the one or more virus-like particles comprises a plurality of virus-like particles encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

375. The pharmaceutical composition of any one of claims 1-374, wherein the composition is formulated for parenteral administration.

376. The pharmaceutical composition of any one of claims 1-374, wherein the composition is formulated for intradiscal injection.

377. A method for treating a spinal disorder in a mammalian subject in need thereof, comprising administering a therapeutically effective amount of a pharmaceutical composition according to any one of claims 1-376 to the subject.

378. The method of claim 377, wherein the spinal disorder is intervertebral disc degeneration.

379. The method of claim 377, wherein the spinal disorder is disc herniation.

380. The method of claim 377, wherein the spinal disorder is spinal stenosis.

381. The method of claim 377, wherein the spinal disorder is spondylosis.

382. The method of claim 377, wherein the spinal disorder is spondylolisthesis.

383. The method of claim 377, wherein the spinal disorder is a spinal infection.

384. The method of claim 383, wherein the spinal infection is discospondylitis.

385. The method of claim 377, wherein the spinal disorder is a spinal neuropathy.

386. The method of claim 385, wherein the spinal neuropathy is discogenic pain, radiculopathy, sciatica, or post-herpetic neuralgia.

387. The method of any one of claims 377-386, wherein the method is for treating low back pain or neck pain associated with the spinal disorder.

388. The method of any one of claims 377-387, wherein the administering comprises parenteral administration.

389. The method of any one of claims 377-387, wherein the administering comprises intradiscal injection.

390. A pharmaceutical composition according to any one of claims 1-376 for use in a method for the treatment of a spinal disorder.

391. Use of a pharmaceutical composition according to any one of claims 1-376 for the manufacture of a medicament for treatment of a spinal disorder.
